(19) United States
(12) Patent Application Publication
(10) Pub. No.: US 2011/0052674 A1
(43) Pub. Date: Mar. 3, 2011

(54) ANGIOTENSIN II RECEPTOR BLOCKER DERIVATIVES

(75) Inventors: Nicoletta Almirante, Milano (IT); Alessia Nicotra, Grandate (Como) (IT); Valentino Mandelli, Monza (Milano) (IT); Stefano Biondi, Pero (Milano) (IT); Silvia Stefanini, San Donato Milanese (Milano) (IT); Iyassu K. Sebhat, Jersey City, NJ (US); Michael Man-Chu Lo, Edison, NJ (US)

(73) Assignees: MERCK SHARP & DOHME CORP., Rahway, NJ (US); NICOX S.A., Sophia Antipolis - Valbonne (FR)

(21) Appl. No.: 12/866,688

(22) PCT Filed: Feb. 19, 2009
(86) PCT No.: PCT/EP2009/051943

§ 371 Date: Aug. 6, 2010

Related U.S. Application Data
(60) Provisional application No. 61/064,276, filed on Feb. 26, 2008.
(60) Provisional application No. 61/193,354, filed on Nov. 20, 2008.

(51) Int. Cl. A61K 9/127; C07D 403/10; A61K 31/41; C07D 403/14; C07D 471/04; A61K 31/519; C07D 257/04; A61K 31/4184; C07D 409/06; A61K 31/4178; A61K 9/12; A61K 9/28; A61K 9/48; A61P 9/00; A61P 13/12; A61P 27/06; A61P 25/00

(52) U.S. Cl. 424/450; 548/253; 514/381; 544/279; 514/264.1; 548/305.4; 514/394; 548/315.1; 514/397; 424/45; 424/474; 424/463

(57) ABSTRACT

New angiotensin II receptor blocker nitroderivatives of general formula (I) and pharmaceutically acceptable salts or stereoisomers thereof: and their use for treating cardiovascular, renal and chronic liver diseases, inflammatory processes and metabolic syndromes.

The present invention relates to new Angiotensin II Receptor Blocker (ARB) derivatives. More particularly, the present invention relates to new ARB nitroderivatives, pharmaceutical compositions containing them and their use for the treatment of cardiovascular, renal and chronic liver diseases, inflammatory processes and metabolic syndromes.

With the angiotensin II receptor blockers a class of compounds is intended, comprising as main components Losartan, EXP3174, Candesartan, Telmisartan, Valsartan, Eprosartan, Irbesartan and Olmesartan.

ARBs are approved for the treatment of hypertension, post-myocardial infarction and heart failure, the antihypertensive activity is due mainly to selective blockade of AT₁ receptors and the consequent reduced pressor effect of angiotensin II. Angiotensin II stimulates the synthesis and secretion of aldosterone and raises blood pressure via a potent direct vasoconstrictor effect.

Now, it has been reported that angiotensin II receptor blockers have side-effects such as for example hypotension, hyperkalaemia, myalgia, respiratory-tract disorders, renal disorders, back pain, gastrointestinal disturbances, fatigue, and neutropenia (Martindale, Thirty-third edition, p. 921).

WO 2005/011646 describes angiotensin II receptor blocker nitroderivatives, pharmaceutical compositions containing them and their use for the treatment of cardiovascular, renal and chronic liver diseases, inflammatory processes and metabolic syndromes. The publication describes a variety of angiotensin II receptor blocker compounds each of which are covalently linked to a bivalent radical capable to release nitric oxide. Specific examples include angiotensin II receptor blockers with one or two nitric oxide-releasing moieties directly linked to the angiotensin II receptor blocker compound through esters or carbonates.

WO 2005/023182 describes nitrosated and nitrosylated cardiovascular compounds, and compositions comprising at least one nitrosated and nitrosylated cardiovascular compound and optionally at least one nitric oxide donor. The cardiovascular compound which is nitrosated or nitrosylated may be an aldosterone antagonist, an angiotensin II receptor antagonist, a calcium channel blocker, an endothelin antagonist, a hydralazine compound, a neutral endopeptidase inhibitor or a renin inhibitor. The nitric oxide donor may be selected from S-nitrosothiols, nitrites, nitrates, N-oxo-N-nitrosamines, furoxans, and sydnonimines.

WO 2006/093864 discloses novel cardiovascular compounds comprising at least one nitric oxide enhancing group, and pharmaceutically acceptable salts thereof. The cardiovascular compounds can be, for example, aldosterone antagonists, angiotensin II antagonists, endothelin antagonists, hydralazine compounds, neutral endopeptidase inhibitors and renin inhibitors. The nitric oxide enhancing groups are nitroxides and/or heterocyclic nitric oxide donor groups such as furoxans, sydnonimines, oxatriazole-5-ones and/or oxatriazole-5-imines.

WO 2007/019448 describes novel nitric oxide enhancing angiotensin II antagonist compounds comprising at least one nitric oxide enhancing group directly or indirectly linked to the angiotensin II antagonist compound through one or more sites such as carbon, oxygen and/or nitrogen via a bond or moiety that cannot be hydrolyzed.

It was now object of the present invention to provide new derivatives of ARBs containing at least a nitric oxide-releasing moiety linked to the angiotensin II receptor blocker through an amino acid bridge.

The Applicant has surprisingly and unexpectedly found a specific class of nitric oxide-releasing ARBs with good pharmacological profile and oral bioavailability, associated with prolonged duration of action.

In particular, it has been recognized that the angiotensin II receptor blocker nitroderivatives of the present invention exhibit a strong anti-inflammatory, antithrombotic and antiplatelet activity and can be furthermore employed for treating or preventing hypertension, congestive heart failure, pulmonary hypertension, renal insufficiency, renal ischemia, renal failure, renal fibrosis, liver fibrosis, portal hypertension, cardiac insufficiency, cardiac hypertrophy, cardiac fibrosis, myocardial ischemia, cardiomyopathy, glomerulonephritis, complications resulting from diabetes such as nephropathy, vasculopathy and neuropathy, glaucoma, elevated intra-ocular pressure, atherosclerosis, restenosis post-angioplasty, complications following vascular or cardiac surgery, erectile dysfunction, hyperaldosteronism, lung fibrosis, scleroderma, anxiety, cognitive disorders, complications of treatments with immunosuppressive agents, metabolic syndromes and other diseases known to be related to the renin-angiotensin system.

Object of the present invention are, therefore, new angiotensin II receptor blocker nitroderivatives of general formula (I) and pharmaceutically acceptable salts or stereoisomers thereof:

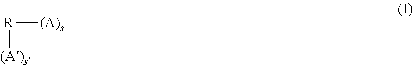

wherein:
A and A′ are independently selected from the group consisting of —(Y—ONO2), —(Y′—ONO2) or (1a)

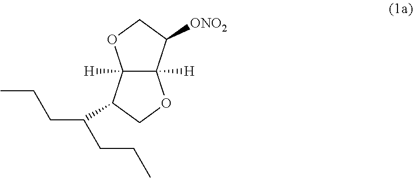

s is 1 or 2;
s′ is 0, 1 or 2;
R is selected from the following residues of formula (II) or (III):

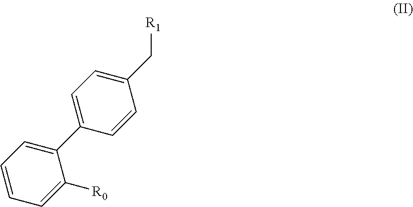

wherein:
R₀ is the group

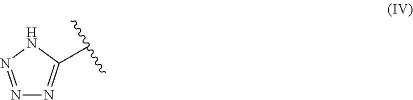

or N₀ which is a moiety capable to bind the groups A and A′ as defined hereinafter;
R₁ is selected from the groups (Va-Ve):

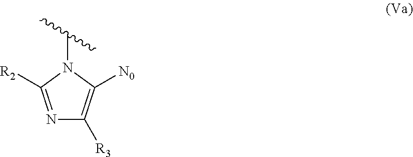

wherein R₂ is C₁-C₅ linear or branched alkyl, preferably n-propyl or n-butyl;
R₃ is an halogen atom such as Cl, Br, I, or a perfluorurated C₁-C₄ alkyl chain, preferably C₂F₅, or the group —C(CH₃)₂OH;

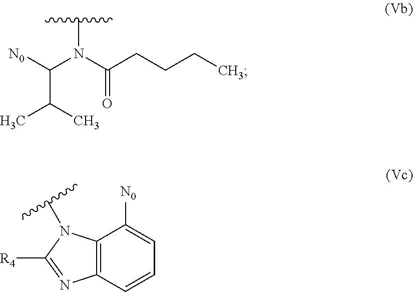

wherein R₄ is n-Bu or —OEt;

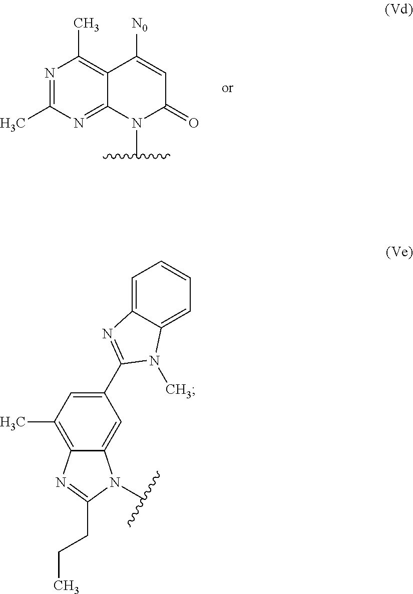

or R is the residue of formula (III):

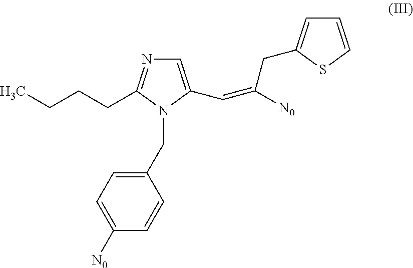

wherein N₀ is a moiety capable to bind the groups A and A′, having one of the following meanings:
1)

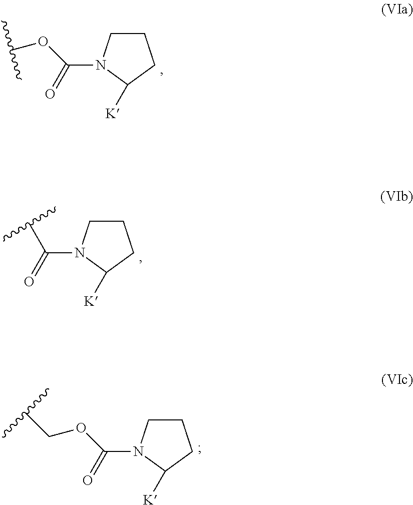

wherein K′ is equal to —COO—, —CONH—, —CH₂—O—CO—, —CH₂—O—COO— or —CH₂—O—CONH— and K′ is bound to the group A wherein A is —(Y—ONO₂) or (1a), with the proviso that when A is (1a), then K′ is —COO— or —CH₂—OCOO—;
2) —OCO—NH-J-K′, —CO—NH-J-K′ or —CH₂—O—CO—NH-J-K′ wherein J is selected among (VIIa-VIIk):

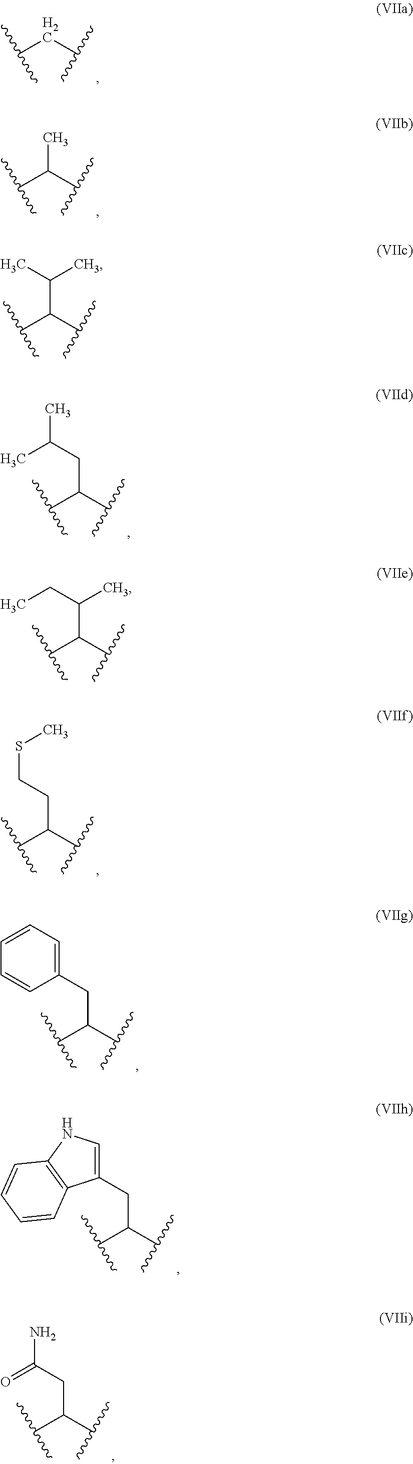

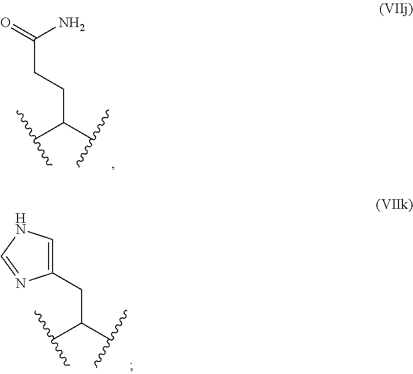

wherein K′ is equal to —COO—, —CONH—, —CH₂—O—CO—, —CH₂—O—COO— or —CH₂—O—CONH— and K′ is bound to the group A wherein A is —(Y—ONO₂) or (1a), with the proviso that when A is (1a), then K′ is —COO— or —CH₂—OCOO—;
3) —O—CO—NH—K—K*, —CH₂—O—CO—NH—K—K* or —CO—NH—K—K* wherein K is selected among K₁, K₂ or K₃ wherein:
K₁ is selected among (VIIIa-VIIId):

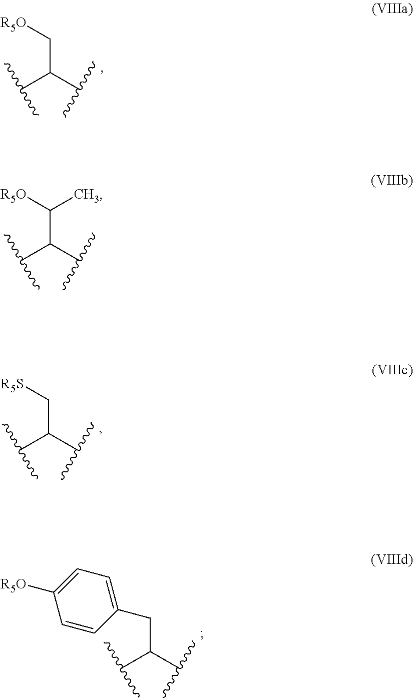

wherein R₅ is H or a group selected from —CO—, —COO— or —CONH— capable to bind a group A′ wherein A′ is —(Y′—ONO₂);
K₂ is selected among (VIIIe-VIIIf):

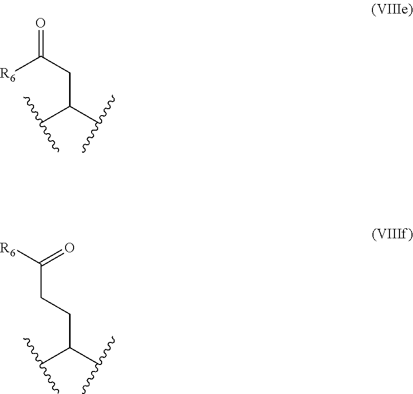

wherein R₆ is —OH or a group selected from —O— or —NH capable to bind a group A′, with the proviso that when A′ is (1a), then R₆ is —O—;
K₃ is selected among (VIIIg-VIIIh):

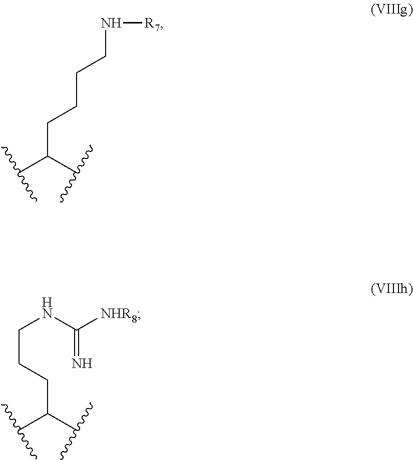

wherein R₇ and R₈ are H or a group selected from —CO— or —COO— capable to bind a group A′ wherein A′ is —(Y′—ONO₂);
K* is equal to K′ as above defined or —COOH and when K* is equal to K′ is bound to the group A, with the proviso that when A is (1a), then K′ is —COO— or —CH₂—OCOO—;
4)

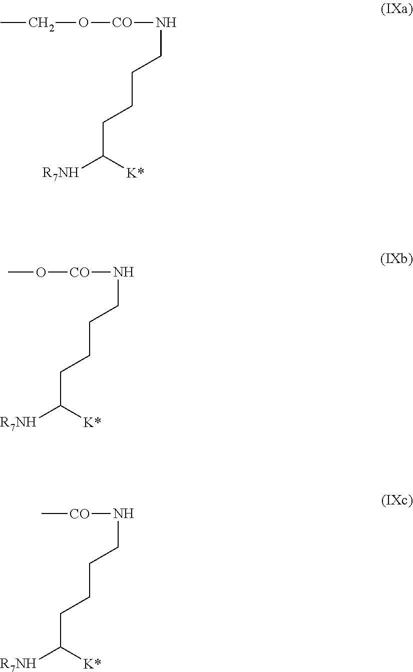

wherein R₇ and K* are as above defined;
with the proviso that:
i) when R₁ is the group (Va), then N₀ is selected from the group consisting of (VIb), (VIc) —CO—NH-J-K′, —CH₂—O—CO—NH-J-K′, —CO—NH—K—K*, —CH₂—O—CO—NH—K—K*, (IXc) and (IXa);
ii) when R₁ is selected from the groups (Vb), (Vc) or (Ve), then N₀ is selected from the group consisting of (VIb), —CO—NH-J-K′, —CO—NH—K—K* and (IXc);
iii) when R₁ is the group (Vd), then N₀ is selected from the group consisting of (VIa), —OCO—NH-J-K′, —O—CO—NH—K—K* and (IXb);
iv) when R is selected from the residue (III), then N₀ is selected from the group consisting of (VIb), —CO—NH-J-K′, —CO—NH—K—K* and (IXc);
v) when R is selected from the residue (II) and R₀ is N₀, then R₁ is the group (Vₑ)
vi) when R is selected from the residue (II), then s is 1 and s′ is 0 or 1;
vii) when R is selected from the residue (III), then s is 2 and s′ is 0 or 2.
   Y and Y′ independently are bivalent radicals having the following meaning:
   a)

straight or branched C₁-C₂₀ alkylene, preferably C₁-C₁₀, being optionally substituted with one or more of the substituents selected from the group consisting of: halogen atoms, hydroxy, —ONO₂ or R¹, wherein R¹ is —OC(O) (C₁-C₁₀ alkyl)-ONO₂ or —O(C₁-C₁₀ alkyl)-ONO₂;

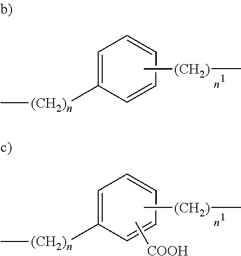

wherein n is an integer from 0 to 20, and n¹ is an integer from 1 to 20;

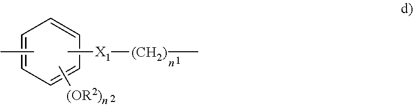

wherein:
n¹ is as defined above and n² is an integer from 0 to 2;

### X₁=—OCO— or —COO— and R² is H or CH₃;

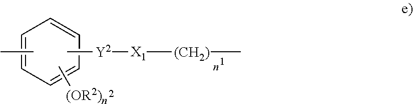

wherein:
n¹, n², R² and X₁ are as defined above;
Y² is —CH₂—CH₂— or —CH═CH—(CH₂)ₙ²—;
with the proviso that when Y or Y′ is selected from the bivalent radicals mentioned under b)-e), the —ONO₂ group is linked to a —(CH₂)ₙ¹ group;

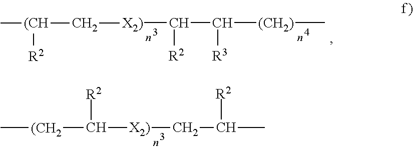

wherein X₂ is —O— or —S—, n³ is an integer from 1 to 6, preferably from 1 to 4, R² is as defined above,
R³ is H or —ONO₂ and n⁴ is 0 or 1.

The term “C₁-C₂₀ alkylene” as used herein refers to branched or straight chain C₁-C₂₀ hydrocarbon, preferably having from 1 to 10 carbon atoms such as methylene, ethylene, propylene, isopropylene, n-butylene, pentylene, n-hexylene and the like.

The term “C₁-C₁₀ alkyl” as used herein refers to branched or straight chain alkyl groups comprising one to ten carbon atoms, including methyl, ethyl, n-propyl, isopropyl, n-butyl, isobutyl, t-butyl, pentyl, hexyl, octyl and the like.

Another aspect of the present invention provides the use of the compounds of formula (I) in combination with at least a compound used to treat cardiovascular disease selected from the group consisting of: aldosterone antagonists, renin inhibitors, ACE inhibitors, HMGCoA reductase inhibitors, beta-adrenergic blockers, alpha-adrenergic antagonists, sympatholytics, calcium channel blockers, endothelin antagonists, neutral endopeptidase inhibitors, potassium activators, diuretics, vasodilators, antithrombotics such as aspirin. Also it is contemplated the combination with nitrosated compounds of the above reported compounds.

Suitable aldosterone antagonists, renin inhibitors, ACE inhibitors, HMGCoA reductase inhibitors, beta-adrenergic blockers, alpha-adrenergic antagonists, sympatholytics, calcium channel blockers, endothelin antagonists, neutral endopeptidase inhibitors, potassium activators, diuretics, vasodilators and antithrombotics are described in the literature such as The Merck Index (13^{th} edition).

Suitable nitrosated compounds are disclosed in WO 98/21193, WO 97/16405, WO 98/09948, WO 2004/105754, WO 2004/106300, WO 2004/110432, WO 2005/011646, WO 2005/053685, WO 2005/054218, WO 2007/045551.

The administration of the compounds above reported can be carried out simultaneously or successively.

The present invention also provides pharmaceutical kits comprising one or more containers filled with one or more of the compounds and/or compositions of the present invention and one or more of the compounds used to treat cardiovascular diseases reported above.

As stated above, the invention includes also the pharmaceutically acceptable salts of the compounds of formula (I) and stereoisomers thereof.

Examples of pharmaceutically acceptable salts are either those with inorganic bases, such as sodium, potassium, calcium and aluminium hydroxides, or with organic bases, such as lysine, arginine, triethylamine, dibenzylamine, piperidine and other acceptable organic amines.

The compounds according to the present invention, when they contain in the molecule one salifiable nitrogen atom, can be transformed into the corresponding salts by reaction in an organic solvent such as acetonitrile, tetrahydrofuran with the corresponding organic or inorganic acids.

Examples of organic acids are: oxalic, tartaric, maleic, succinic, citric acids. Examples of inorganic acids are: nitric, hydrochloric, sulphuric, phosphoric acids. Salts with nitric acid are preferred.

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the object of the invention are also all the possible isomers, stereoisomers and their mixtures of the compounds of formula (I).

In one embodiment, R is the residue of formula (II) wherein R₀ is the group of formula (IV), R₁ is the group of formula (Va), R₂ is n-butyl, R₃ is Cl and all other variables are as above defined.

In another embodiment, R is the residue of formula (II) wherein R₀ is the group of formula (IV), R₁ is the group of formula (Va), R₂ is n-propyl, R₃ is the group —C(CH₃)₂OH and all other variables are as above defined.

In another embodiment, R is the residue of formula (II) wherein R₀ is the group of formula (IV) as defined above, R₁ is the group of formula (Vc) as defined above, R₄ is —OEt, and all other variables are as above defined.

In another embodiment s₁ is 0 and A is the group (VIₐ) or (VI_{b}) or (VI_{c}) as defined above wherein K′ is —COO—, and all other variables are as above defined.

In another embodiment s₁ is 0 and A is —CO—NH-J-K′ or —CH₂—O—CO—NH-J-K′ wherein J is the group (VIIa) or (VIIb) as defined above, wherein K′ is —COO—, and all other variables are as above defined.

In another embodiment s₁ is 0 and A is —CH₂—O—CO—NH—K—K* or —CO—NH—K—K* wherein K is K₃ which is the group (VIIIg) or (VIIIₕ) as defined above, and all other variables are as above defined.

In another embodiment of the invention Y and Y′ independently are bivalent radicals having the following meaning:

a)

straight or branched C₁-C₁₀ alkylene, being optionally substituted with one or more —ONO₂;

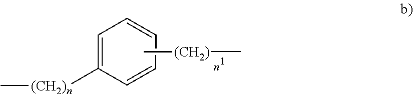

wherein n is an integer from 0 to 5, and n¹ is an integer from 1 to 5;
with the proviso that when Y or Y′ is selected from the bivalent radical b), the —ONO₂ group is linked to a —(CH₂)ₙ¹ group;

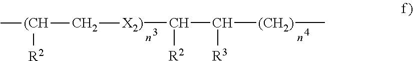

wherein X₂ is —O— or —S—, n³ is 1, R² is H, R³ is H or —ONO₂ and n⁴ is 0 or 1.

The following are preferred compounds according to the present invention:

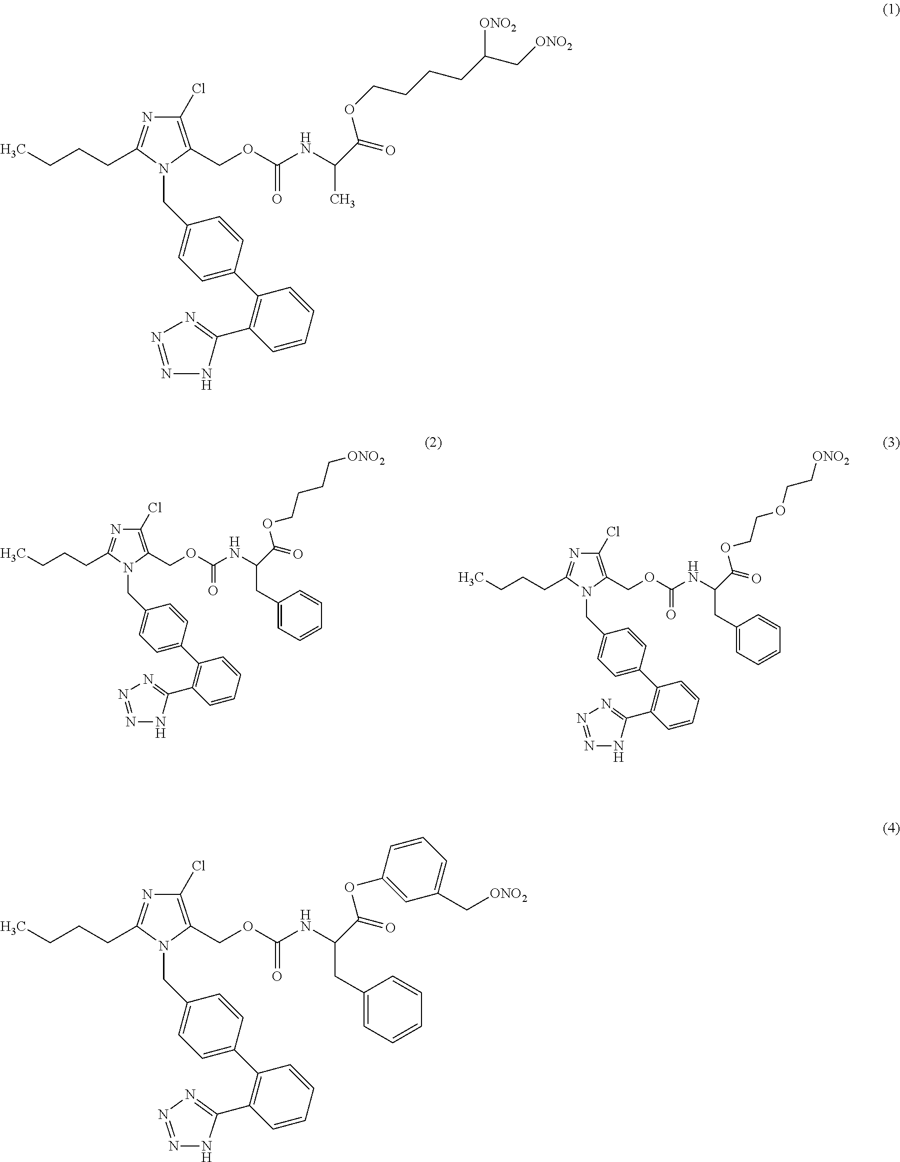

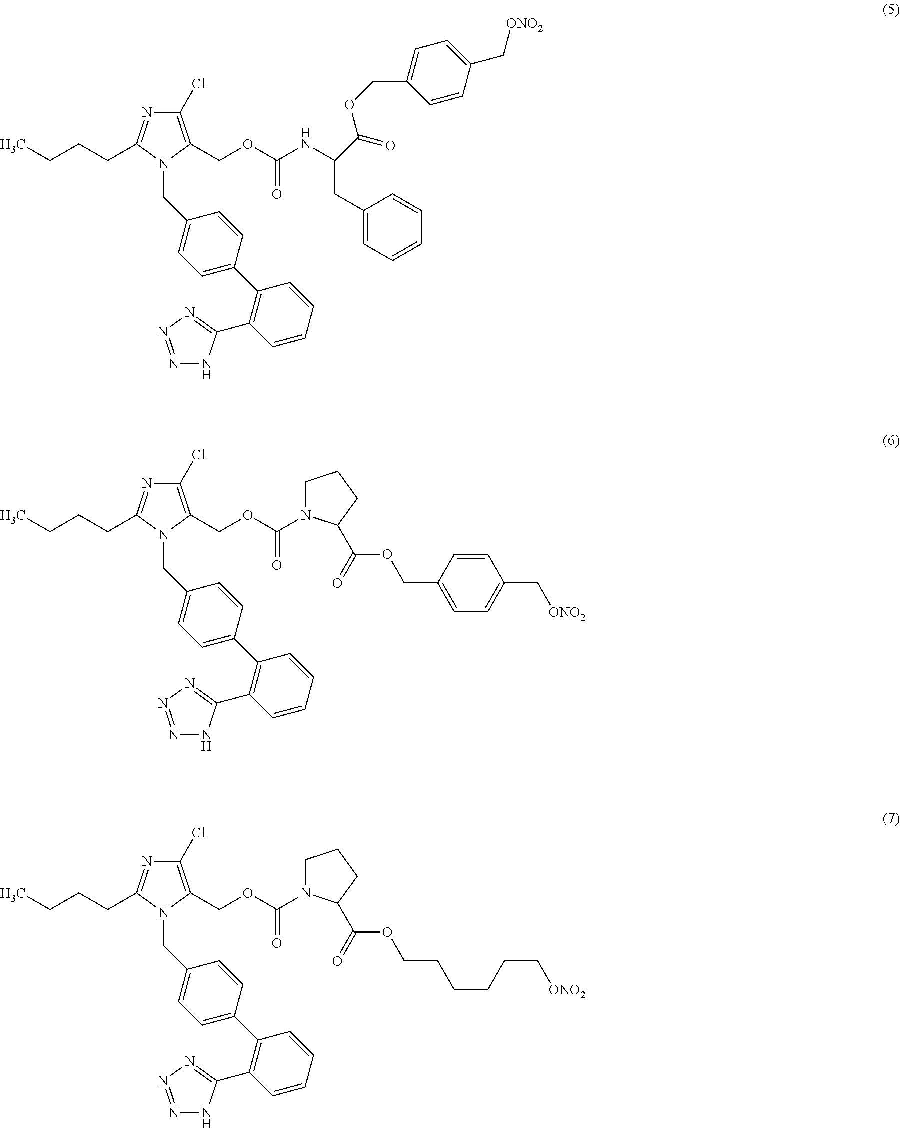

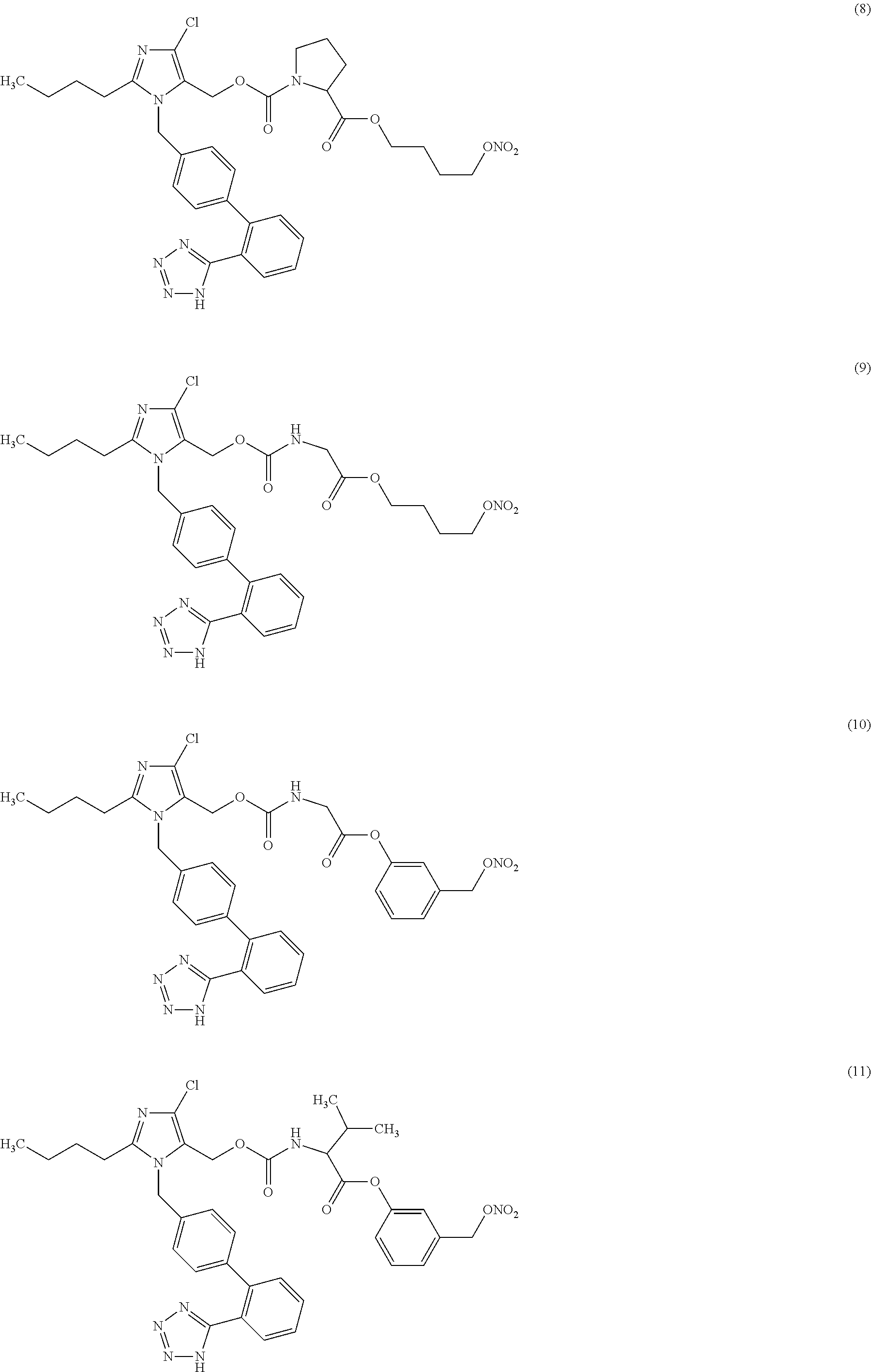

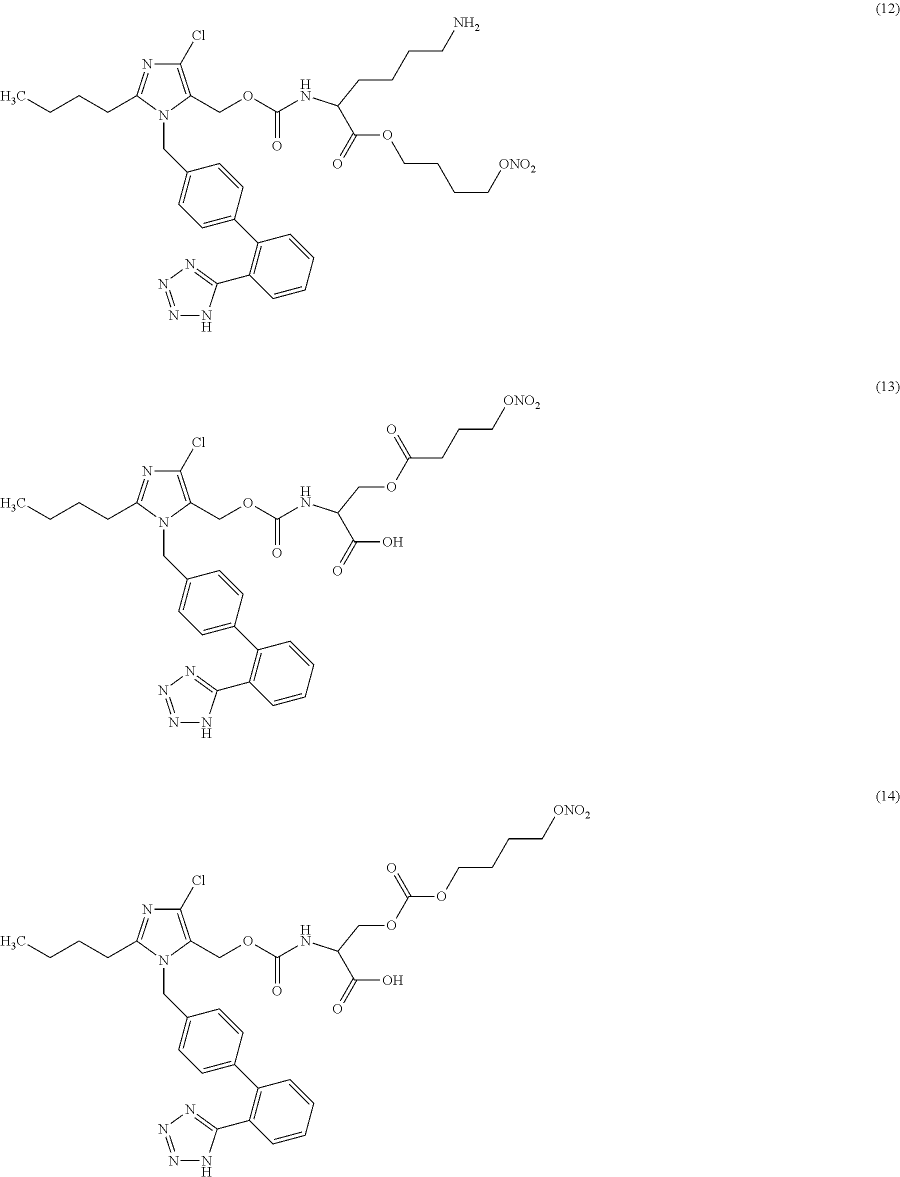

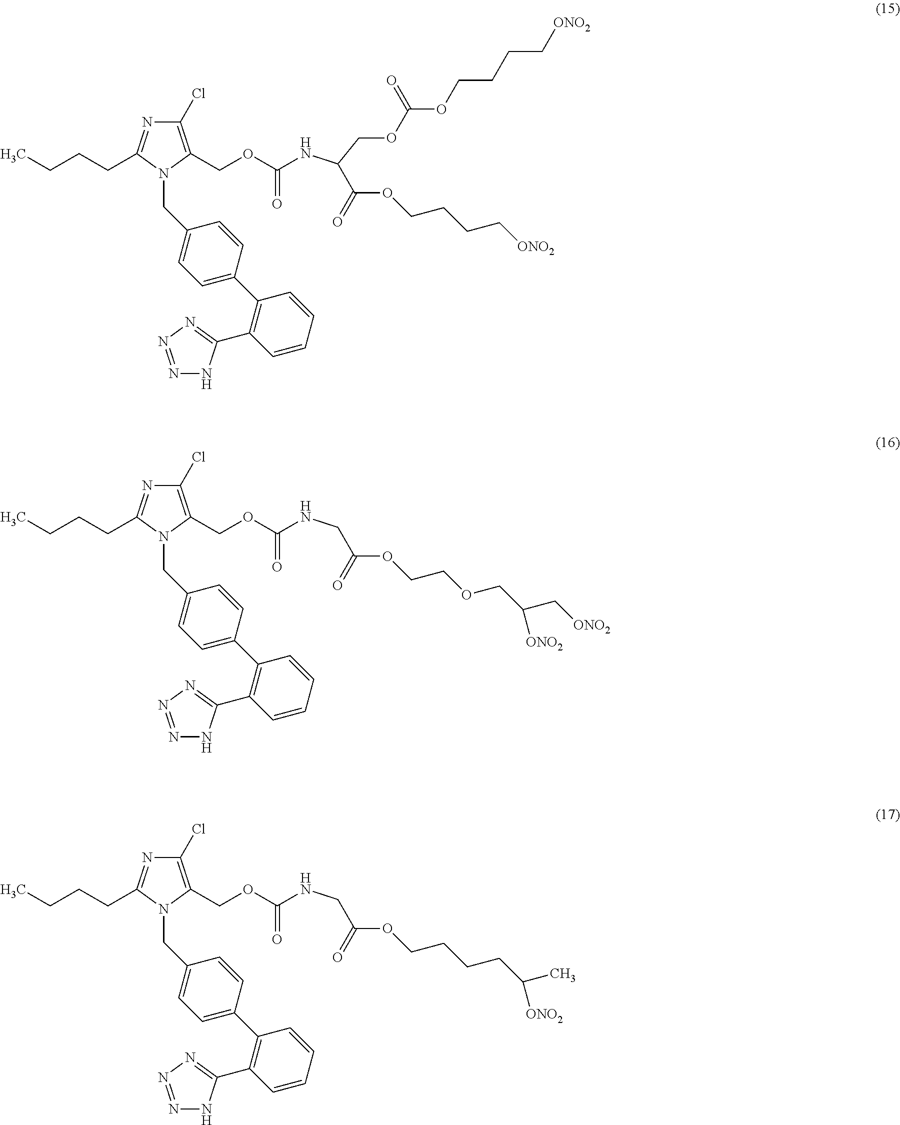

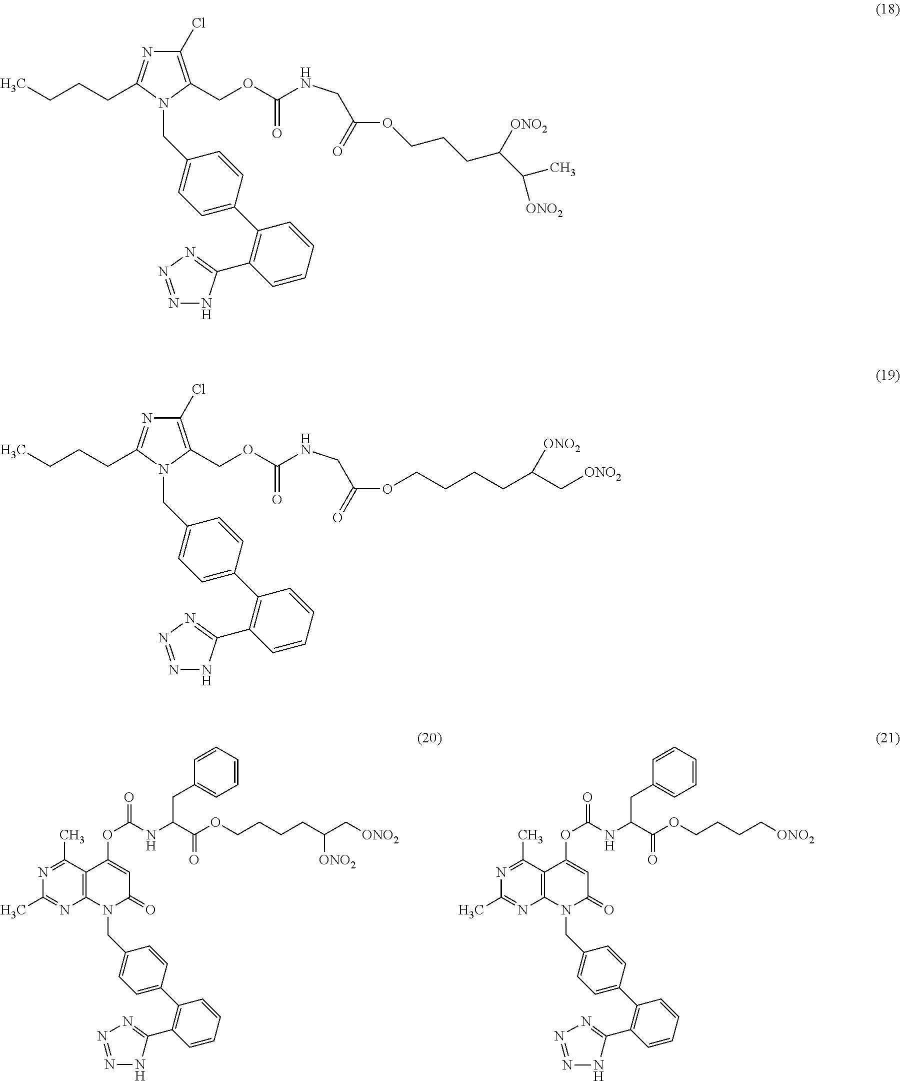

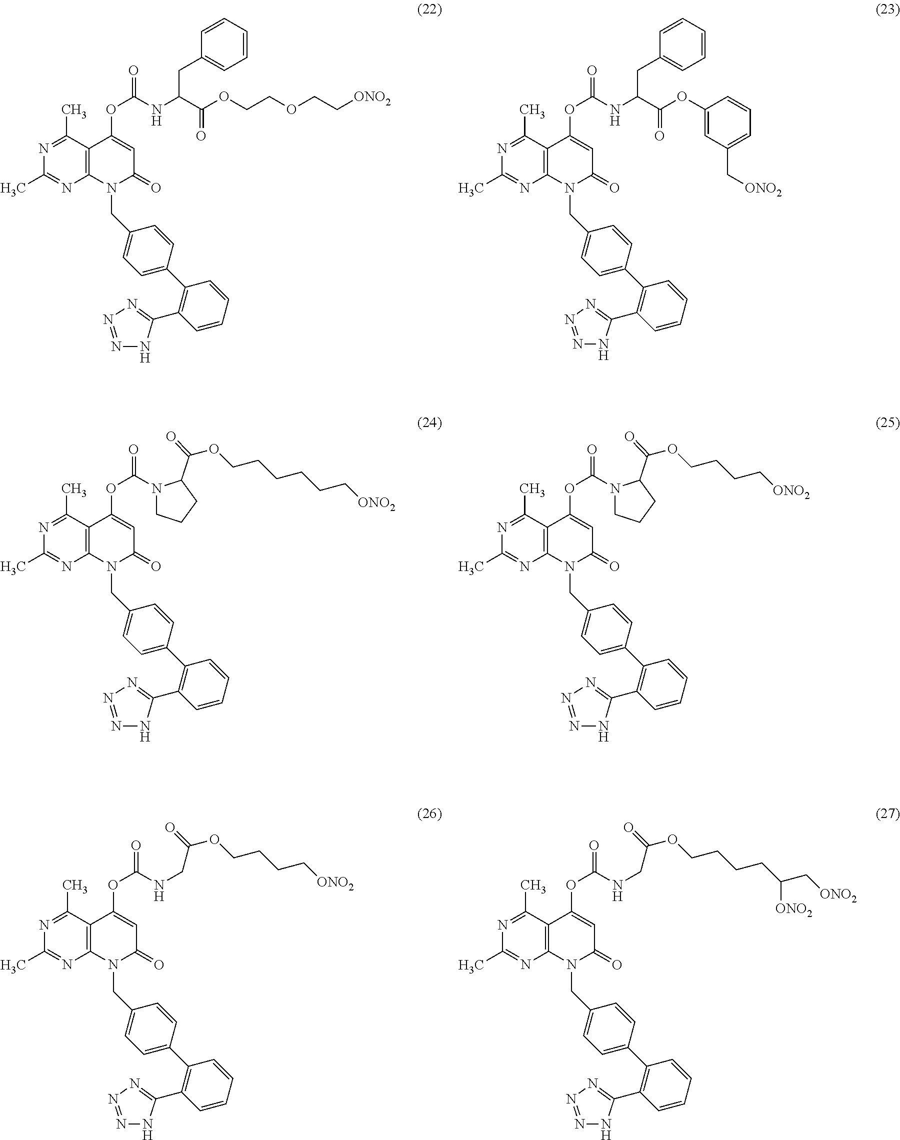

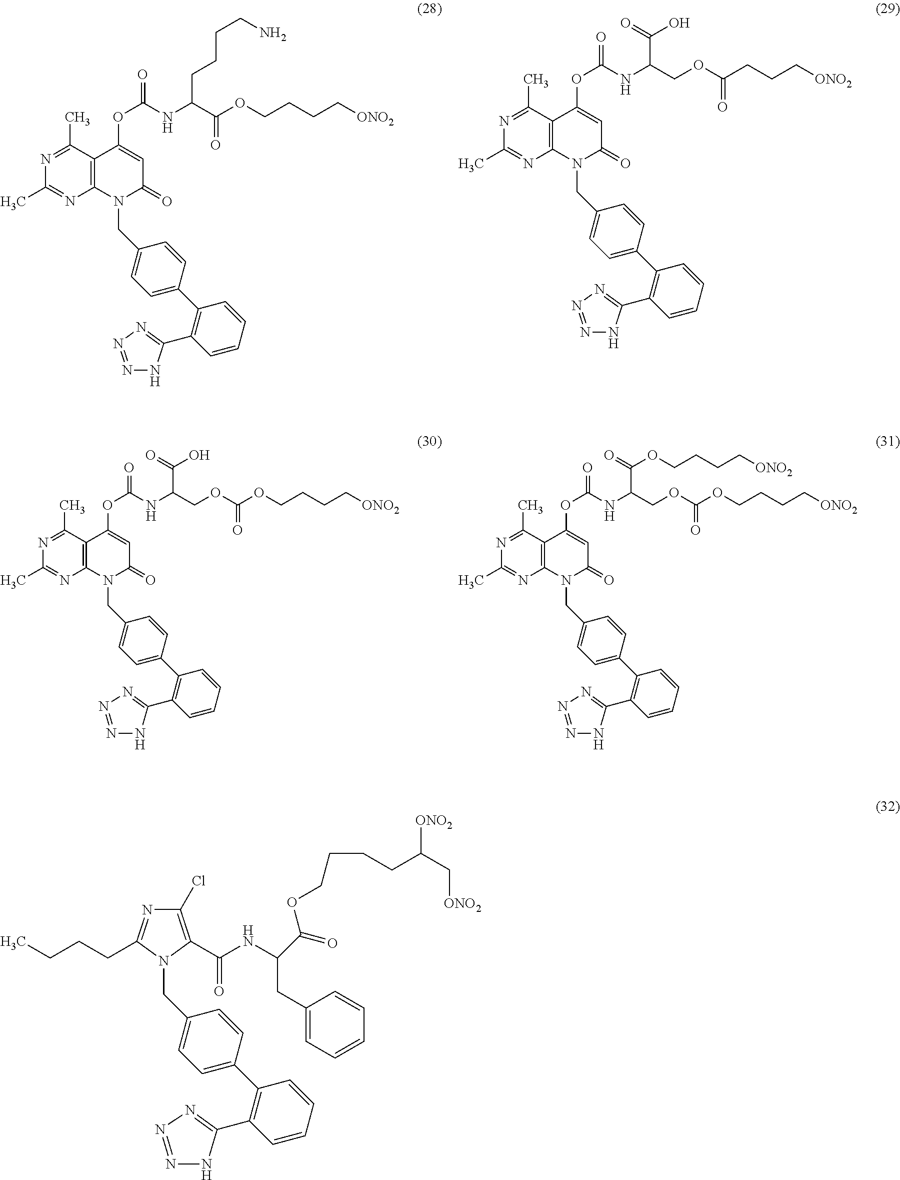

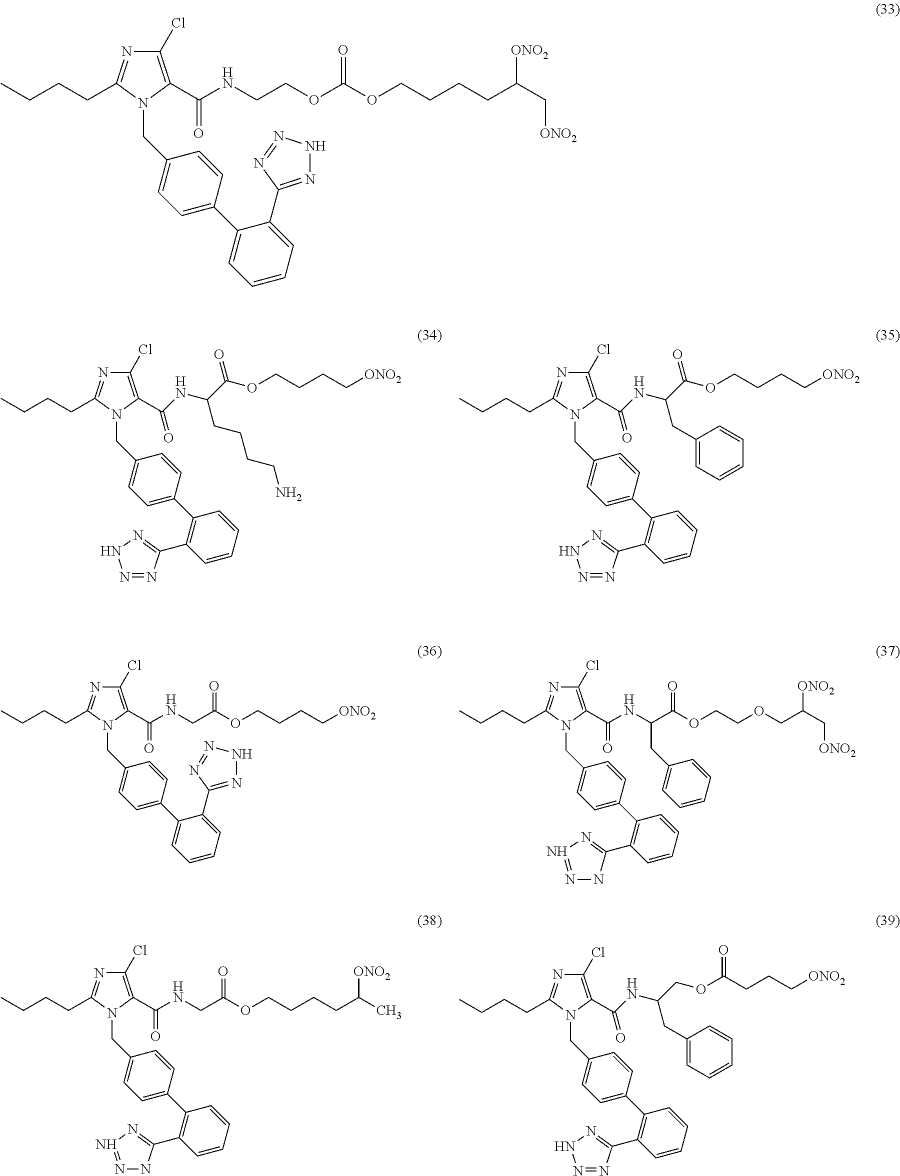

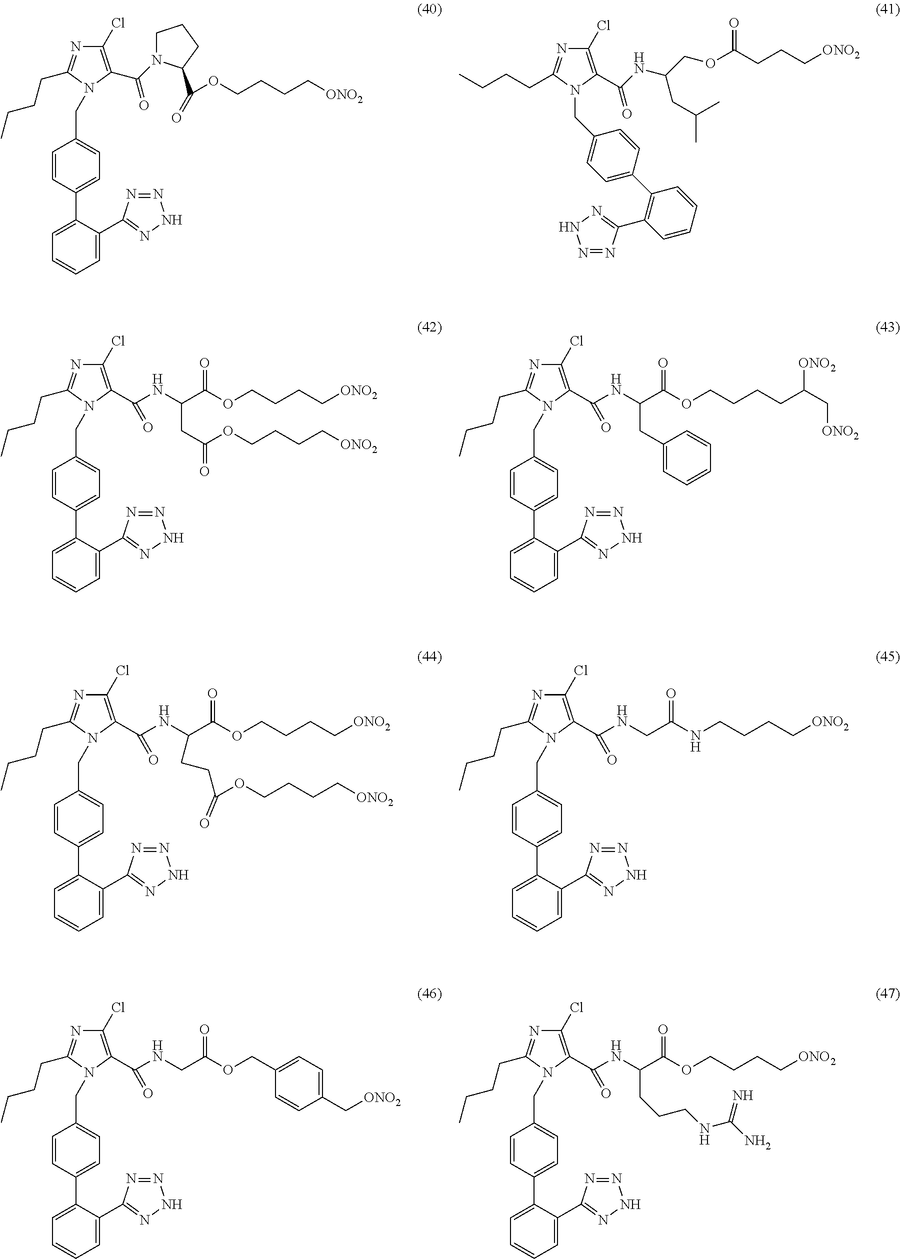

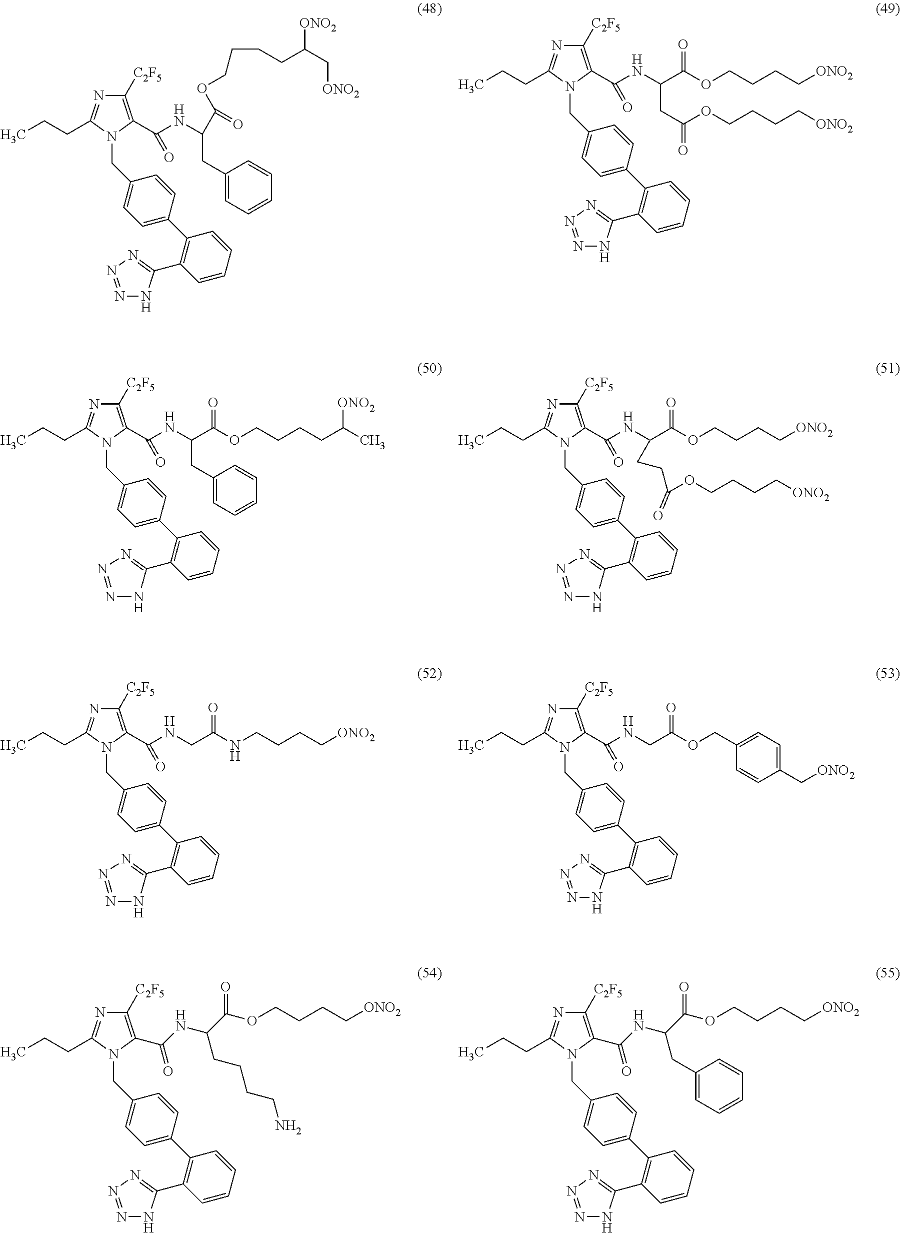

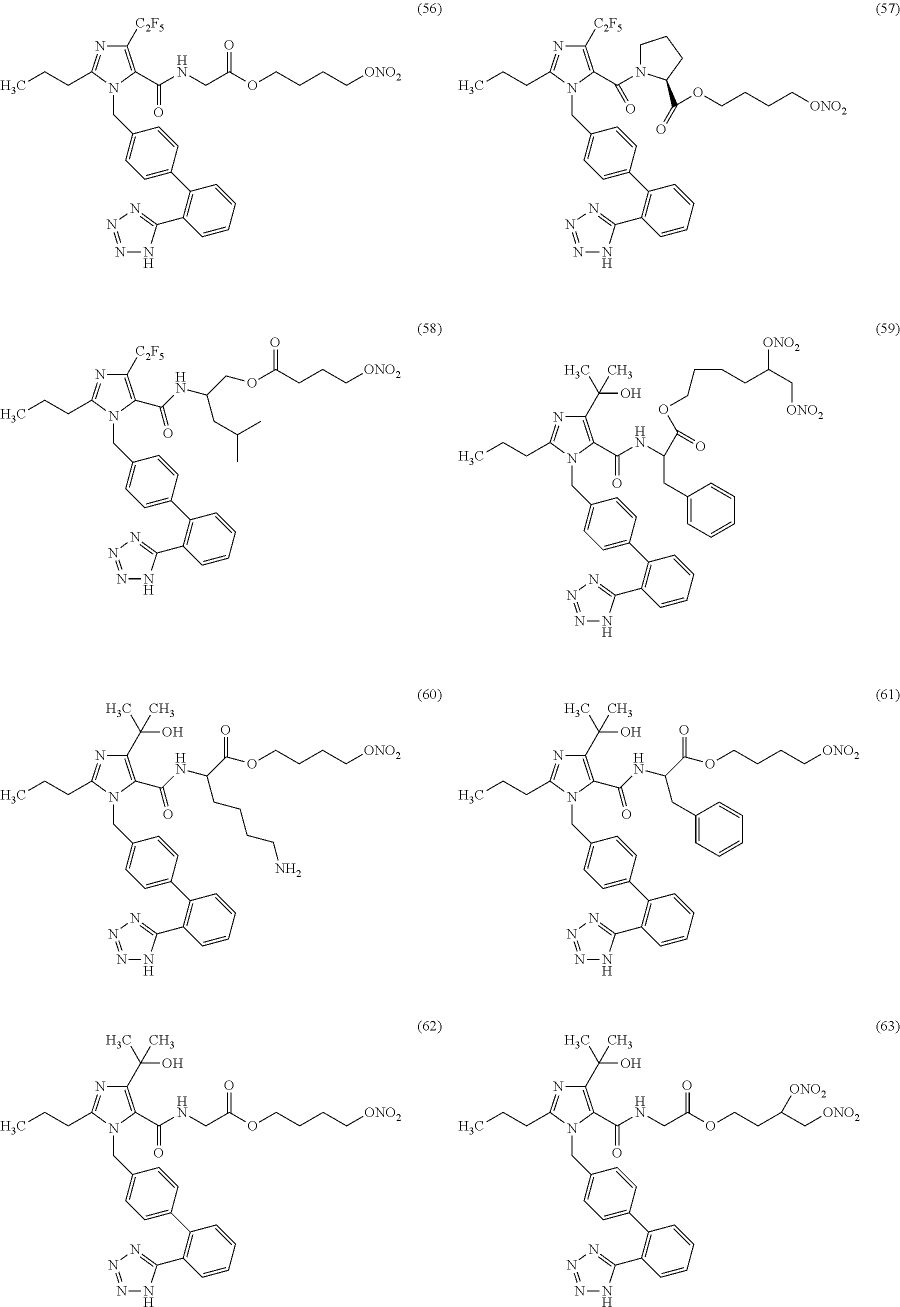

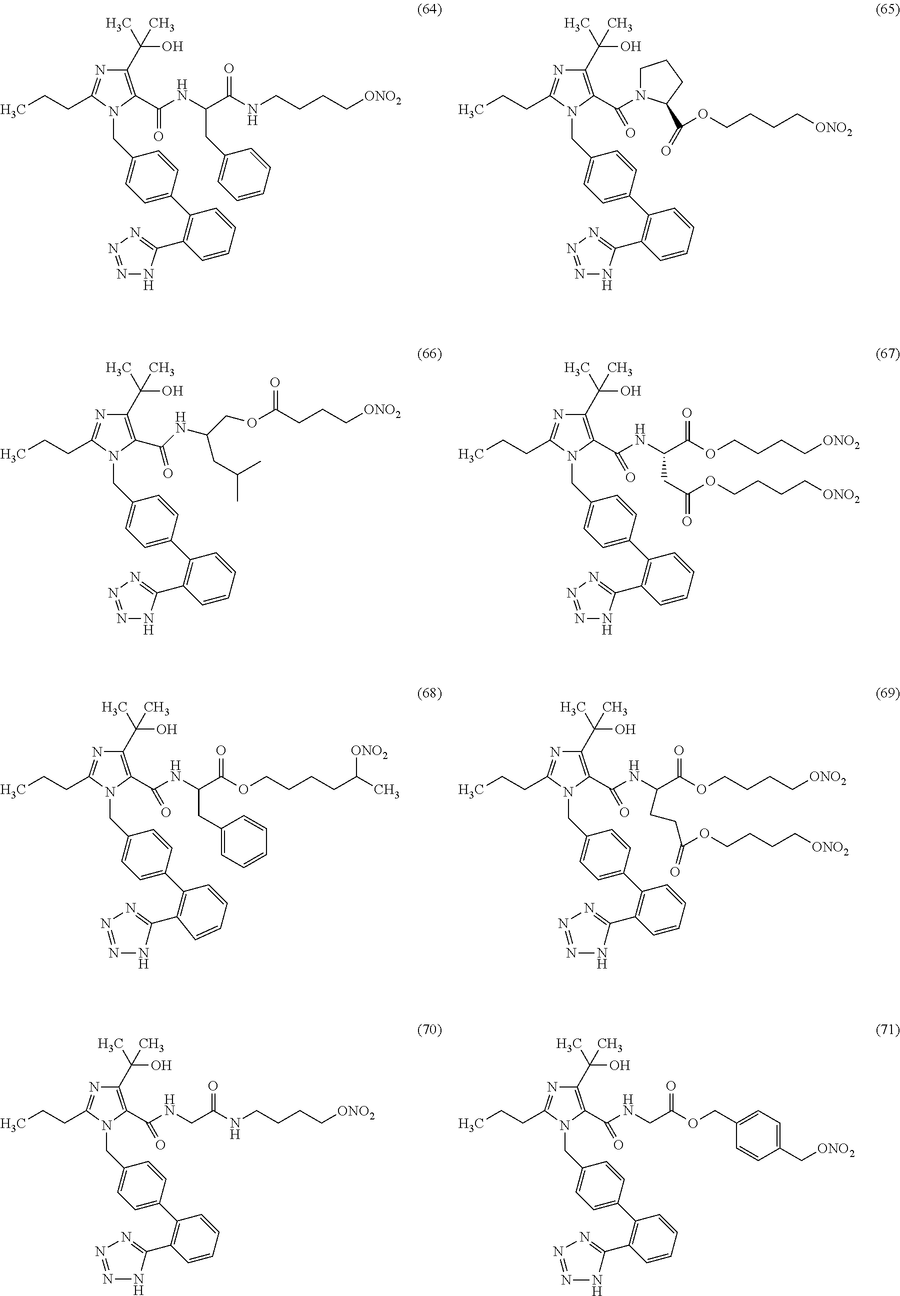

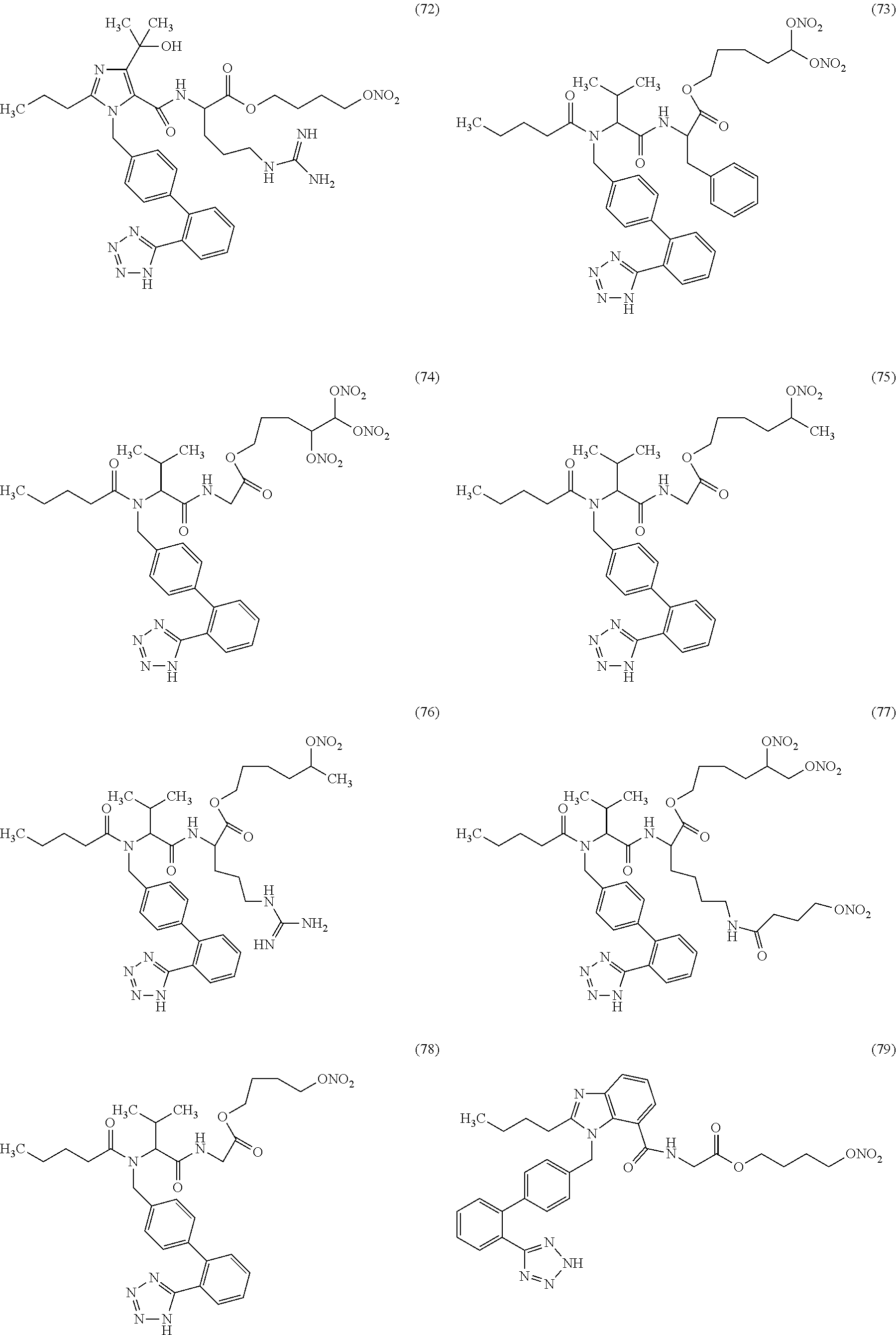

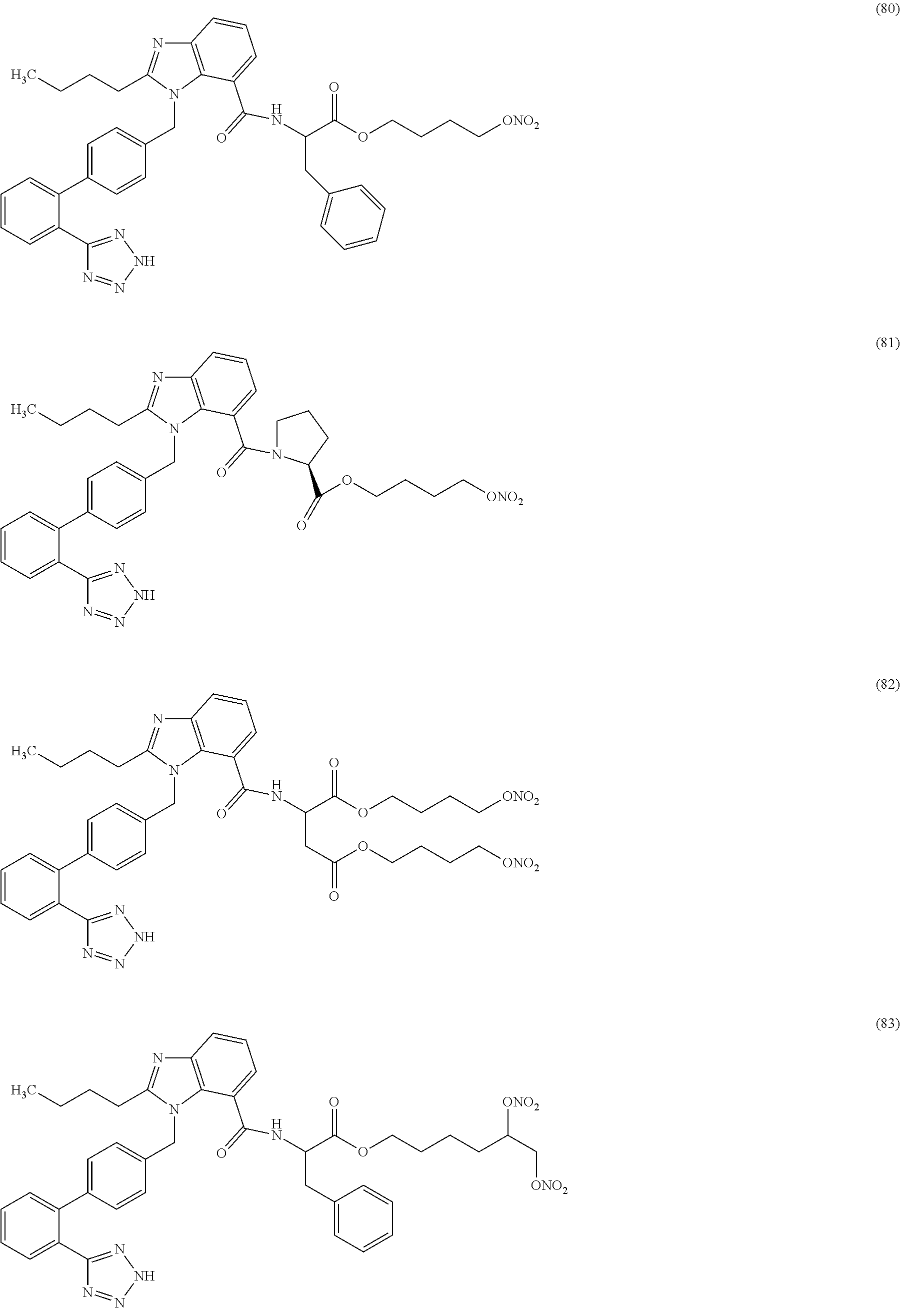

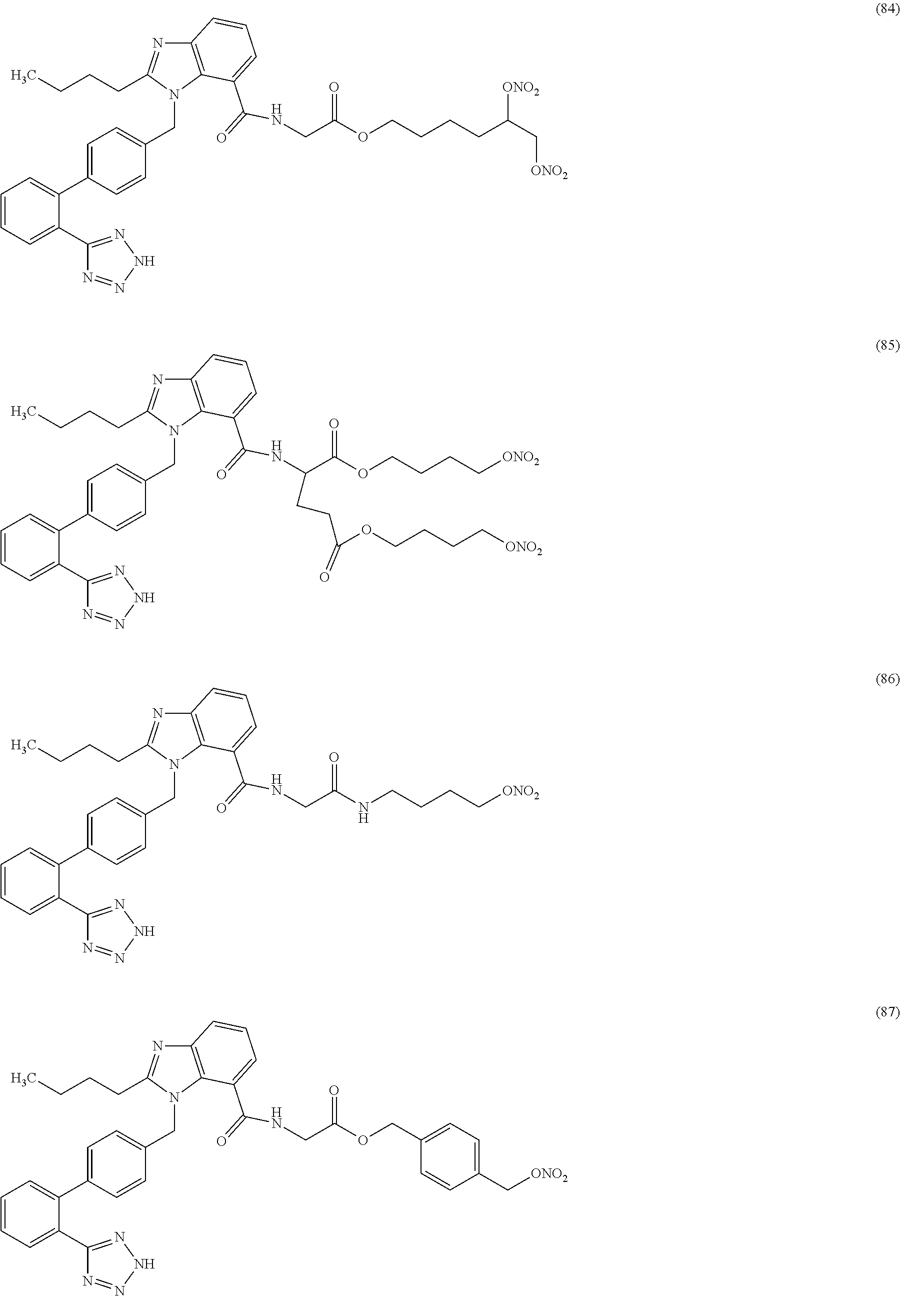

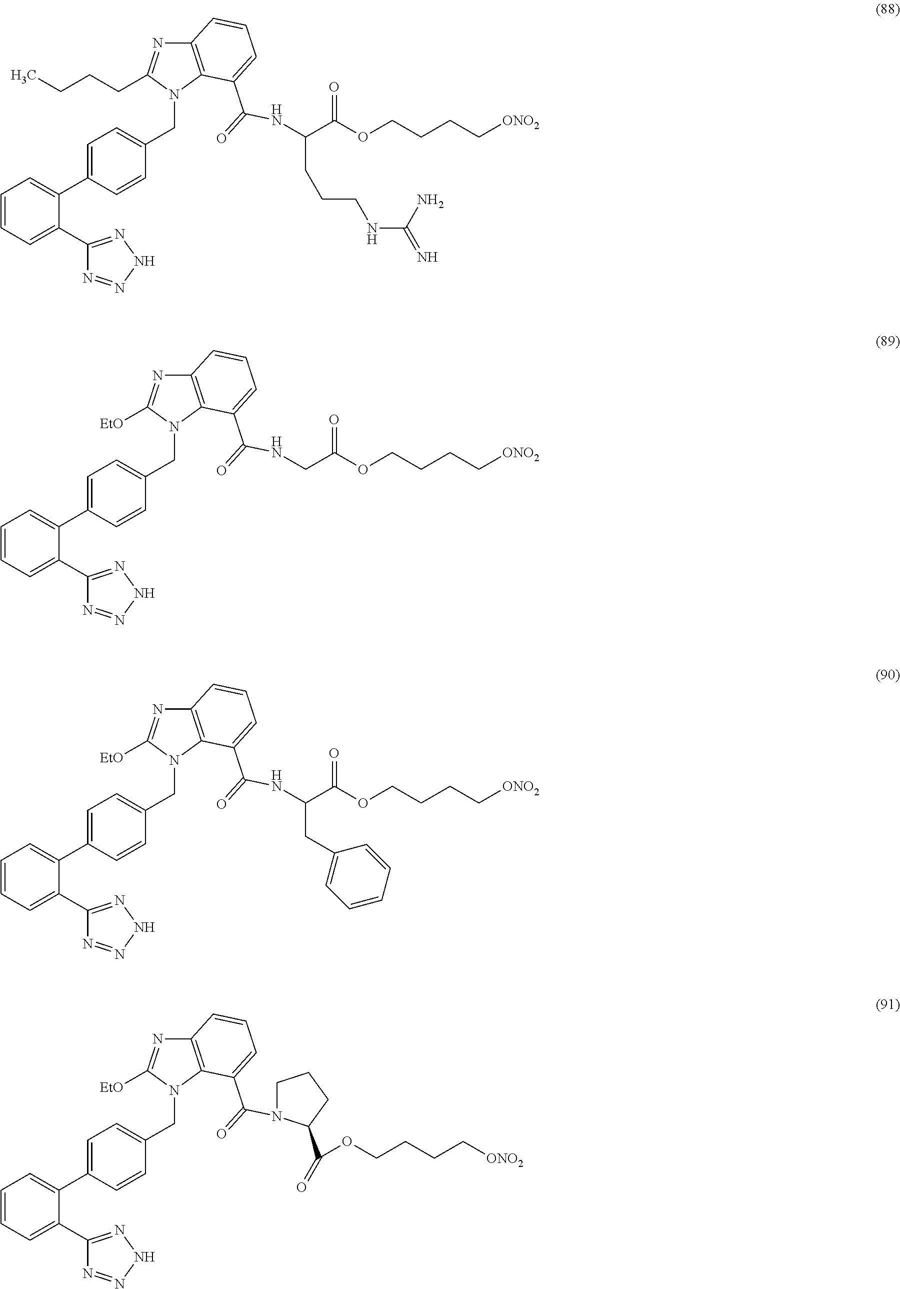

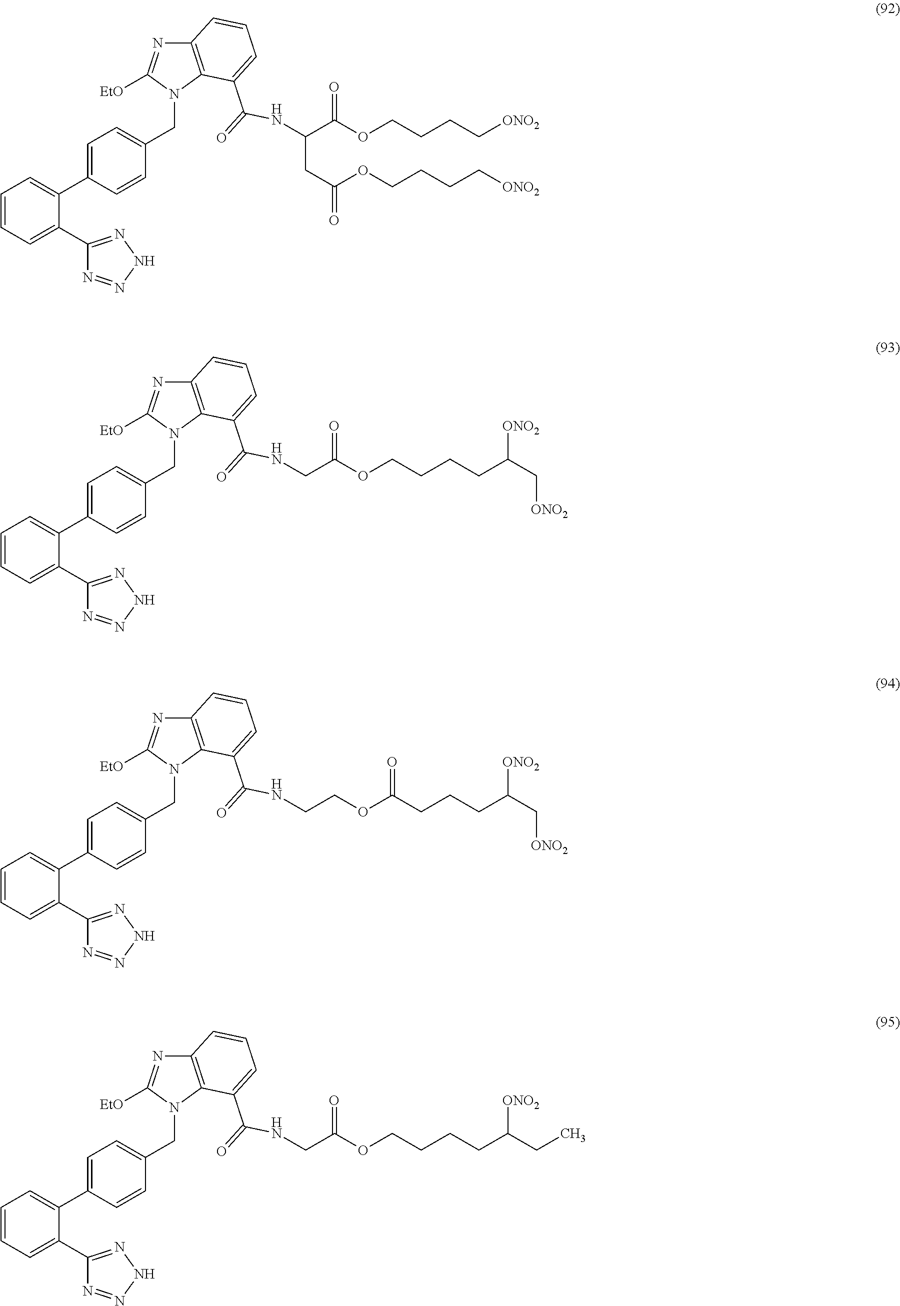

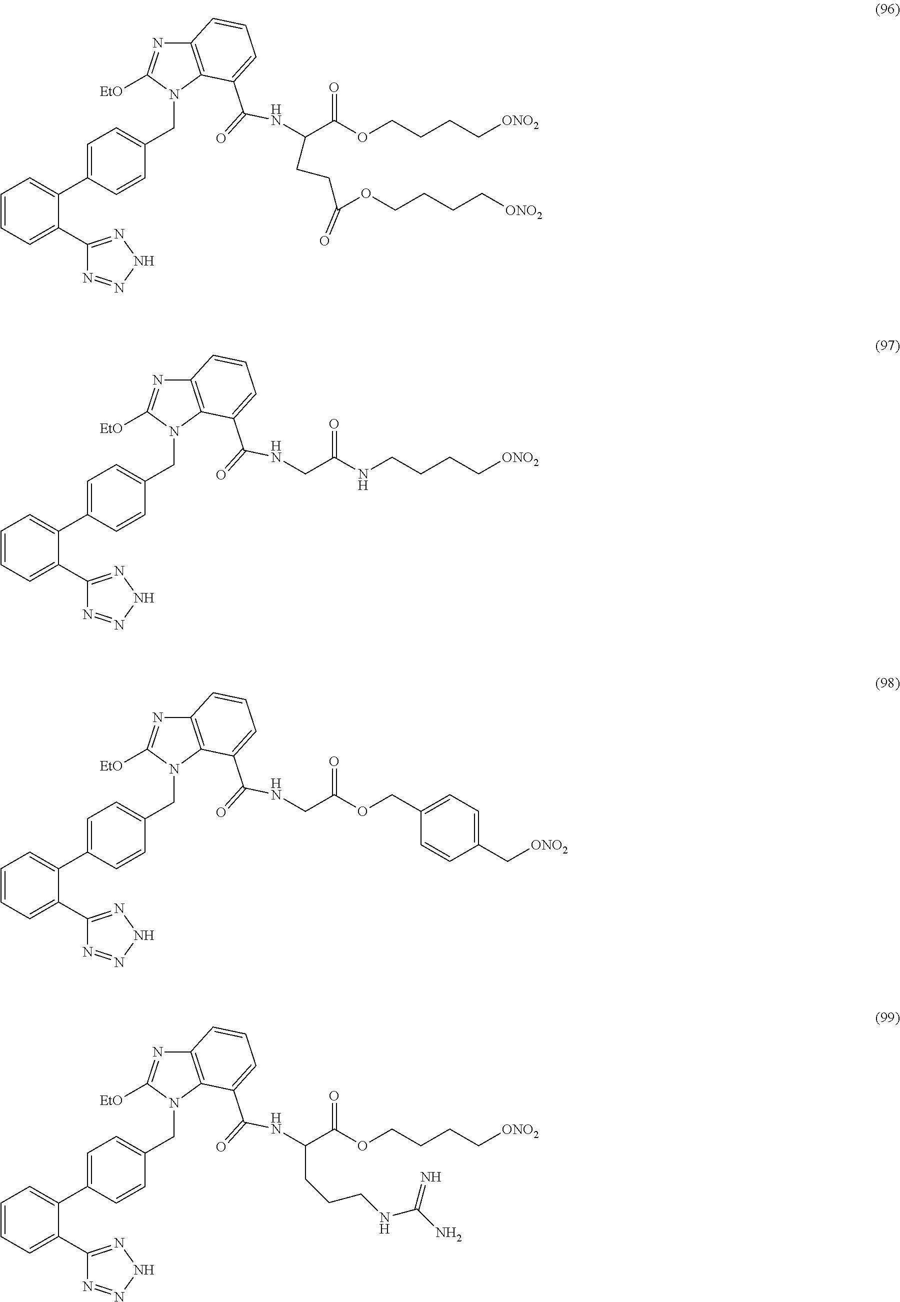

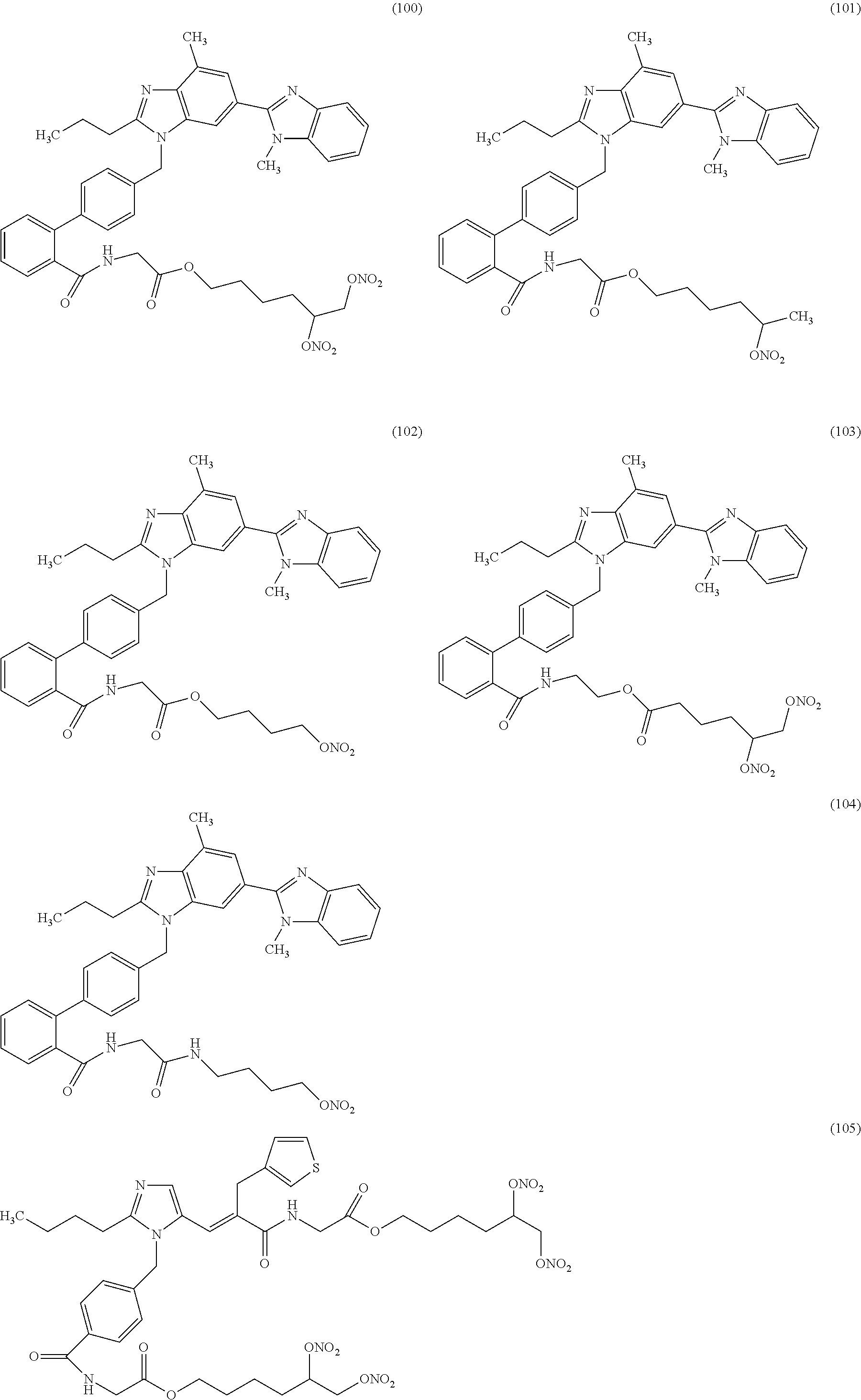

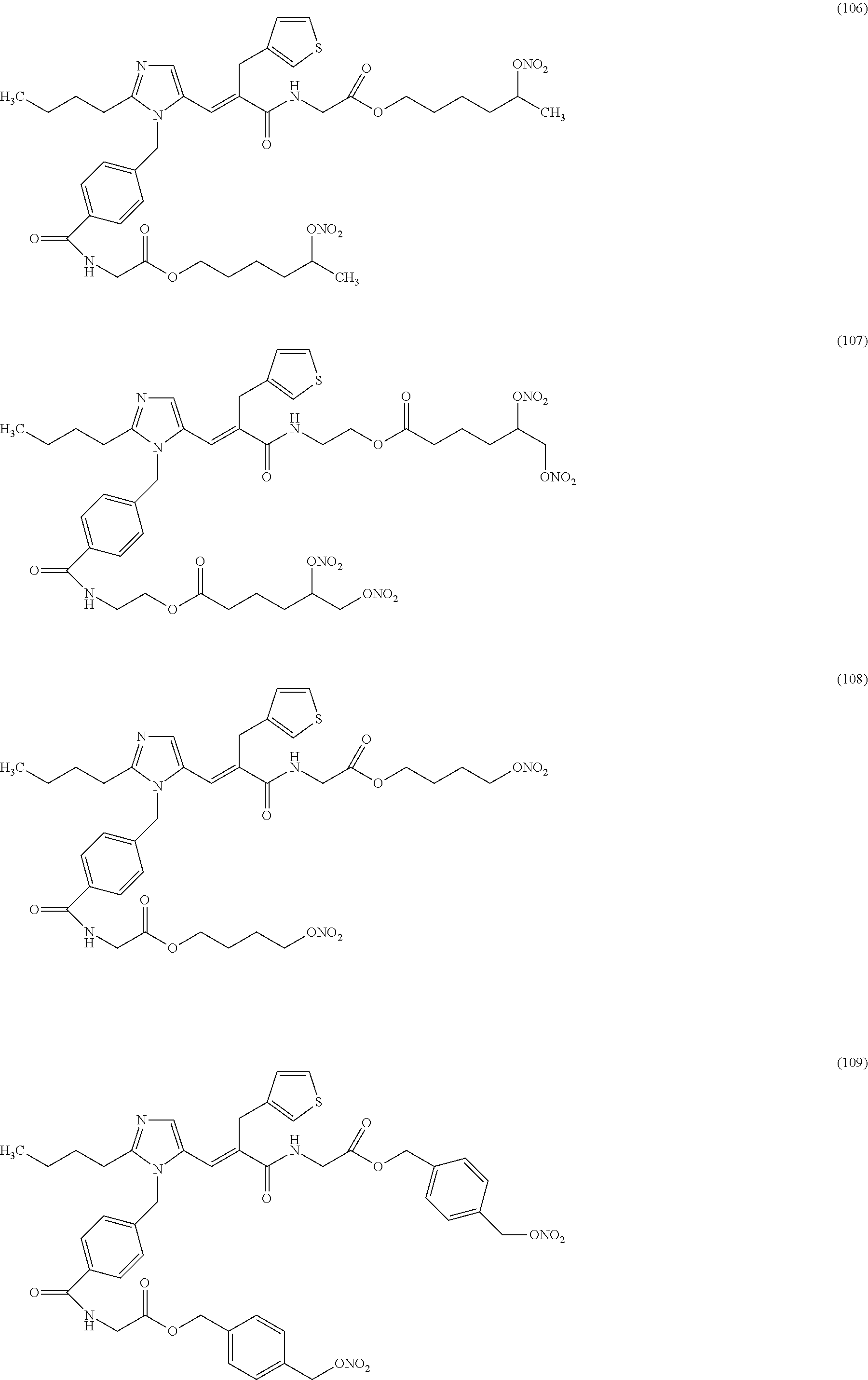

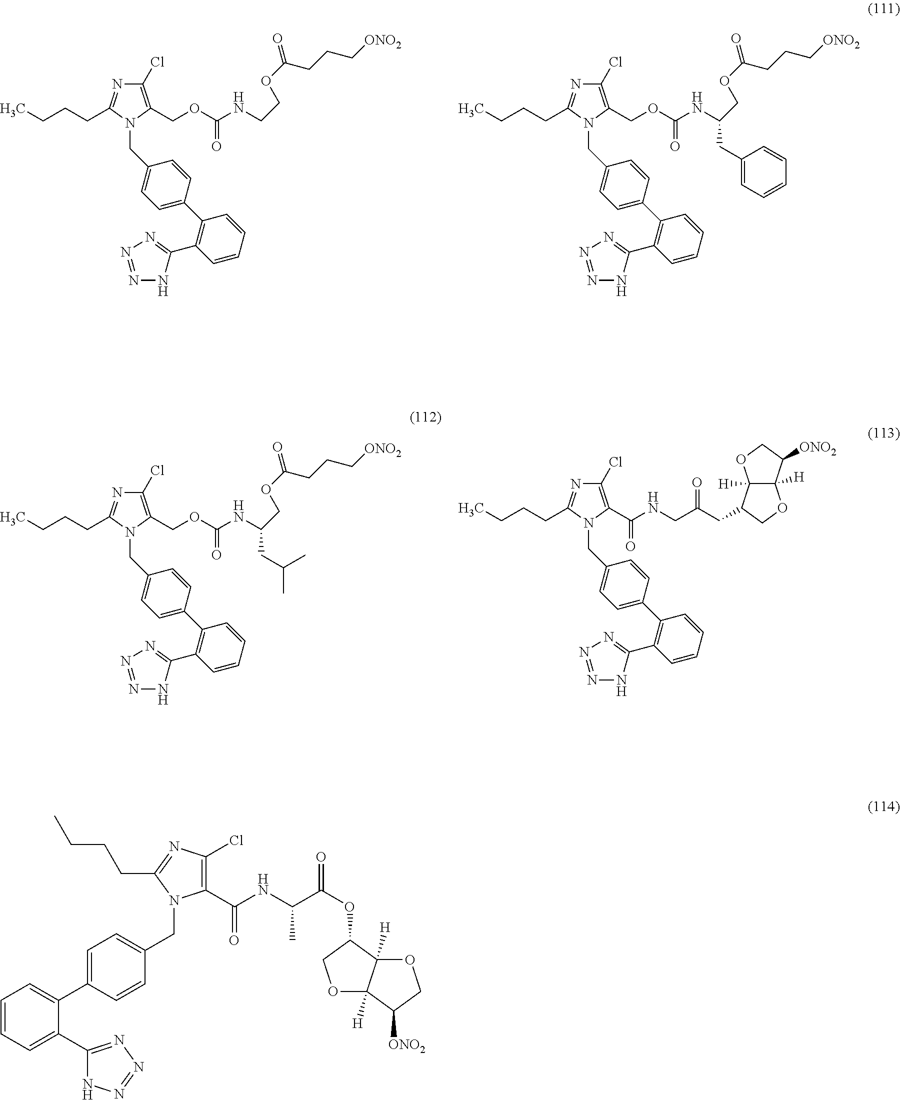

As mentioned above, object of the present invention are also pharmaceutical compositions containing at least a compound of the present invention of formula (I) together with non toxic adiuvants and/or carriers usually employed in the pharmaceutical field.

The daily dose of active ingredient that should be administered can be a single dose or it can be an effective amount divided into several smaller doses that are to be administered throughout the day. Usually, total daily dose may be in amounts preferably from 50 to 500 mg. The dosage regimen and administration frequency for treating the mentioned diseases with the compound of the invention and/or with the pharmaceutical compositions of the present invention will be selected in accordance with a variety of factors, including for example age, body weight, sex and medical condition of the patient as well as severity of the disease, route of administration, pharmacological considerations and eventual concomitant therapy with other drugs. In some instances, dosage levels below or above the aforesaid range and/or more frequent may be adequate, and this logically will be within the judgment of the physician and will depend on the disease state.

The compounds of the invention may be administered orally, parenterally, rectally or topically, by inhalation or aerosol, in formulations eventually containing conventional non-toxic pharmaceutically acceptable carriers, adjuvants and vehicles as desired. Topical administration may also involve the use of transdermal administration such as transdermal patches or iontophoresis devices. The term “parenteral” as used herein, includes subcutaneous injections, intravenous, intramuscular, intrasternal injection or infusion techniques.

Injectable preparations, for example sterile injectable aqueous or oleaginous suspensions may be formulated according to known art using suitable dispersing or wetting agents and suspending agents. The sterile injectable preparation may also be a sterile injectable solution or suspension in a non-toxic parenterally acceptable diluent or solvent. Among the acceptable vehicles and solvents are water, Ringer's solution and isotonic sodium chloride. In addition, sterile, fixed oils are conventionally employed as a solvent or suspending medium. For this purpose any bland fixed oil may be employed including synthetic mono or diglycerides, in addition fatty acids such as oleic acid find use in the preparation of injectables.

Suppositories for rectal administration of the drug can be prepared by mixing the active ingredient with a suitable non-irritating excipient, such as cocoa butter and polyethylene glycols.

Solid dosage forms for oral administration may include capsules, tablets, pills, powders, granules and gels. In such solid dosage forms, the active compound may be admixed with at least one inert diluent such as sucrose, lactose or starch. Such dosage forms may also comprise, as in normal practice, additional substances other than inert diluents, e.g. lubricating agents such as magnesium stearate. In the case of capsules, tablets and pills, the dosage forms may also comprise buffering agents. Tablets and pills can additionally be prepared with enteric coatings.

Liquid dosage forms for oral administration may include pharmaceutically acceptable emulsions, solutions, suspensions, syrups and elixirs containing inert diluents commonly used in the art, such as water. Such compositions may also comprise adjuvants, such as wetting agents, emulsifying and suspending agents, and sweetening, flavouring and the like.

The compounds of the present invention can be synthesized as follows.

### Synthesis Procedure

1. The compounds of general formula (I)

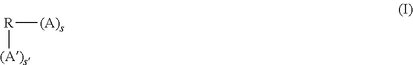

wherein:
s is equal to 1;
s′ is equal to 0
A is —(Y—ONO₂) wherein Y is as above defined;
R is selected from the residue of formula (II) wherein R₀ is (IV) and R₁ is selected from the group (Va) wherein:
R₂ is n-butyl and R₃ is Cl, and N₀ is selected from:
1) (VIc), wherein K′ is equal to —COO—, —CONH—, —CH₂—O—CO—, —CH₂—O—COO— or —CH₂—O—CONH— and K′ is bound to the group —(Y—ONO₂);
2) —CH₂—O—CO—NH-J-K′ wherein J is selected among (VIIa-VIIk) and K′ is as above defined;
can be prepared as follows:
1a) by reacting a compound of formula (Ia)

Rₐ—[Y—ONO₂]ₛ (Ia)

wherein s and Y are as above defined in 1., Rₐ is selected from the residue of formula (II) wherein R₁, R₂, R₃ and N₀ are as above defined in 1.; R₀ is equal to (IVa) and is equal to:

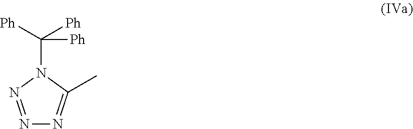

with anhydrous or aqueous organic or inorganic acid to hydrolyze the trityl protective group following procedure well known in the literature; alternatively the trityl protective group can be removed treating with an alcoholic solvent such is metanol or ethanol at temperature from 20-100° C. for 1-48 hrs;
1b) by reacting a compound of formula R_{IIa} or R_{IIa′}

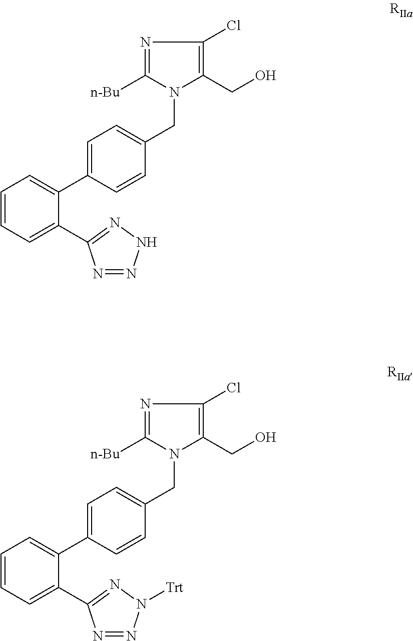

with:
1b.1) a compound of formula (Xaₐ)-(Xaₑ) depending on the meaning of K′

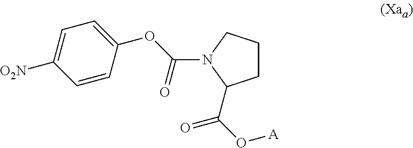

wherein A is —(Y—ONO₂)

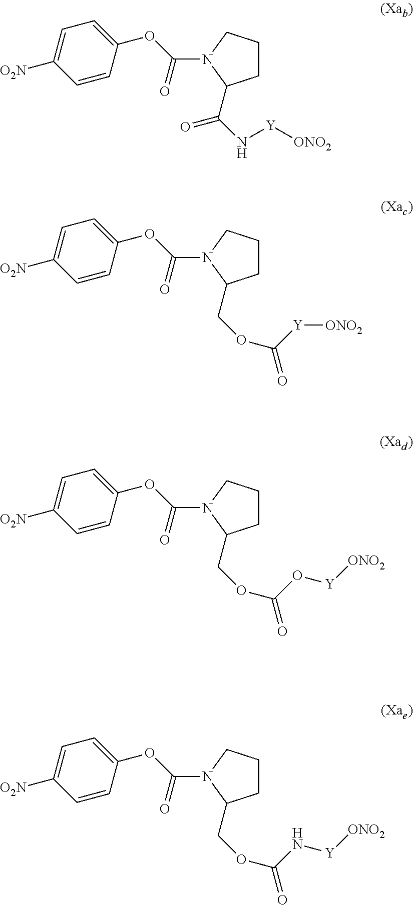

wherein Y is as above defined;
9
or
1b.2) compounds of formula (Xbₐ)-(Xbₑ) depending on the meaning of K′:

p-NO₂—C₆H₄—O—CO—NH-J-COO-A (Xbₐ)

p-NO₂—C₆H₄—O—CO—NH-J-CONH—Y—ONO₂ (Xb_{b})

p-NO₂—C₆H₄—O—CO—NH-J-CH₂O—CO—Y—ONO₂ (Xb_{c})

p-NO₂—C₆H₄—O—CO—NH-J-CH₂O—COO—Y—ONO₂ (Xb_{d})

p-NO₂—C₆H₄—O—CO—NH-J-CH₂O—CONH—Y—ONO₂ (Xbₑ)

wherein A is —(Y—ONO₂), Y and J are as above defined;
in the presence of a inorganic or organic base in an aprotic polar/non-polar solvent such as DMF, THF or CH₂Cl₂ at temperatures range between 0°-100° C. for time range of 1-60 hrs, or under microwave irradiation in the presence of DMAP and a Lewis acid such as Sc(OTf)₃ or Bi(OTf)₃ in solvents such as DMF, CH₂Cl₂ at temperatures range between 60°-120° C. for time range of 1-120 min;
Compound R_{IIa} is Losartan and compound R_{IIa′} is trityl losartan: both compounds are known and commercially available. When R_{IIa} is used the deprotection step described in 1a) is required.

For the preparation of compounds of formula (Xaₐ)-(Xaₑ) and (Xbₐ)-(Xbₑ) see Appendix 1: preparations A1-A6.

2. The compounds of general formula (I)

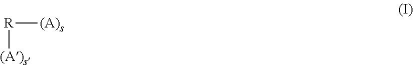

wherein:
s is equal to 1;
s′ is equal to 0
A is —(Y—ONO₂) wherein Y is as above defined;
R is selected from the residue of formula (II) wherein R₀ is (IV) and R₁ is selected from the group (Vd) and N₀ is selected from:
1) (VIa), wherein K′ is equal to —COO—, —CONH—, —CH₂—O—CO—, —CH₂—O—COO— or —CH₂—O—CONH— and K′ is bound to the group —(Y—ONO₂);
2) —O—CO—NH-J-K′ wherein J is selected among (VIIa-VIIk) and K′ is as above defined;
can be prepared as follows
2a) by reacting a compound of formula (Ib)

R_{b}—[Y—ONO₂]ₛ (Ib)

wherein s and Y are as above defined in 1., R_{b} is selected from the residue of formula (II) wherein R₁ and N₀ are as above defined in 2.; R₀ is equal to (IVb) and is equal to:

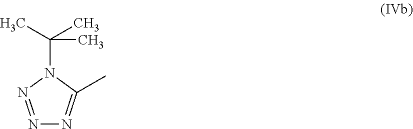

with anhydrous or aqueous organic or inorganic acid to hydrolyze the t-butyl protective group following the procedure described in 1a);
2b) by reacting a compound of formula R_{IIb}

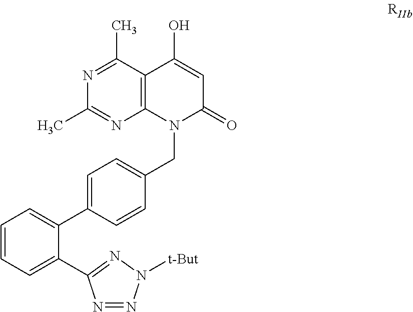

with:
2b.1) a compound of formula (Xaₐ)-(Xaₑ), described in 1b), depending on the meaning of K′; or
2b.2) a compound of formula (Xbₐ)-(Xbₑ) above defined 1b), depending on the meaning of K′;
using the procedure already described in 1b). Compound R_{IIb} is the t-Butyl protected Enoltasosartan (the active metabolite of tasosartan) and can be synthesized as described by John W. Ellingboe et al. in J. Med. Chem. 1998, 41, 4251-4260.

3. The compounds of general formula (I)

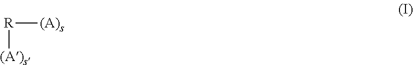

wherein:
s is equal to 1;
s′ is equal to 0
A is —(Y—ONO₂) wherein Y is as above defined,
R is selected from the residue of formula (II) wherein R₀ is (IV), R₁ is selected from the group:
i) (Va) wherein R₂ is n-butyl and R₃ is Cl;
ii) (Va) wherein R₂ is n-propyl and R₃ is C₂F₅;
iii) (Va) wherein R₂ is n-propyl and R₃ is the group C(CH₃)₂OH;
iv) (Vb);
v) (Vc) wherein R₄ is n-butyl;
vi) (Vc) wherein R₄ is —OEt;
   wherein N₀ is selected from:
1) (VIb), wherein K′ is equal to —COO—, —CONH—, —CH₂—O—CO—, —CH₂—O—COO— or —CH₂—O—CONH— and K′ is bound to the group —(Y—ONO₂);
2) —CO—NH-J-K′ wherein J is selected among (VIIa-VIIk) and K′ is as above defined;
   and
   can be prepared as follows
   3a) by reacting a compound of formula (Ic)

R_{c}—[Y—ONO₂]ₛ (Ic)

wherein s and Y are as above defined in 1., R_{c} is selected from the residue of formula (II) wherein N₀ is as above defined in 3. and R₁ is as above defined in the points i)-vi); R₀ is equal to (IVa) and is as previously defined, following the same procedure described in 1a);
3b) by reacting:
i) a compound of formula R_{IIc}

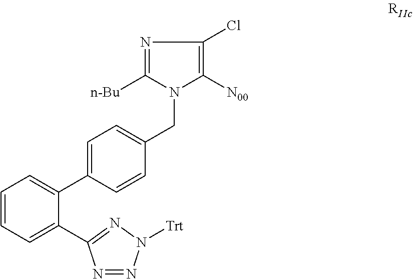

Wherein N₀₀ is —COOH, —COHal or —COOAct wherein Hal is an halogen atom such as Cl, Br, F; Act is a carboxylic acid activating group used in peptide chemistry such as:

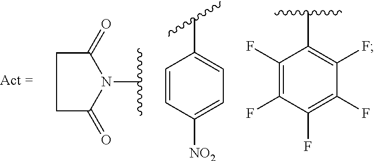

or
ii) a compound of formula R_{IId}

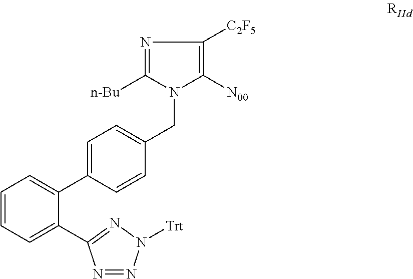

wherein N₀₀ is as previously defined; or
iii) a compound of formula R_{IIe}

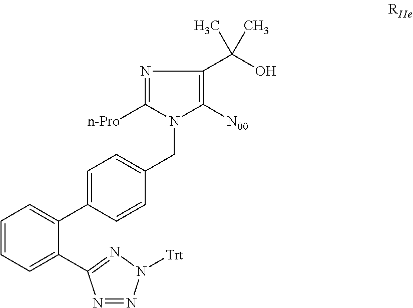

wherein N₀₁ is as previously defined; or
iv) a compound of formula R_{IIf}

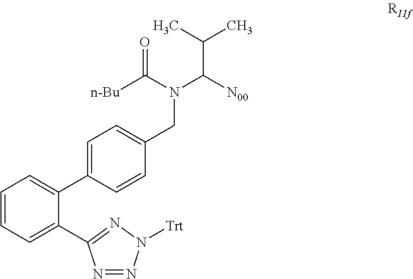

wherein N₀₀ is as previously defined; or
v) a compound of formula R_{IIg}

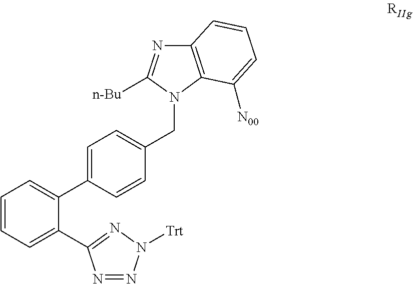

wherein N₀₀ is as previously defined; or
vi) a compound of formula R_{IIh}

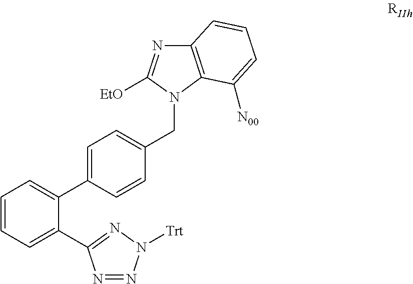

wherein N₀₀ is as previously defined;
with
3b.1) a compound of formula (Xcₐ)-(Xcₑ) depending on the meaning of K′

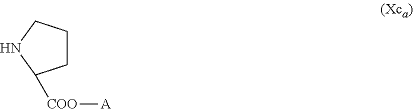

wherein A is —(Y—ONO₂)

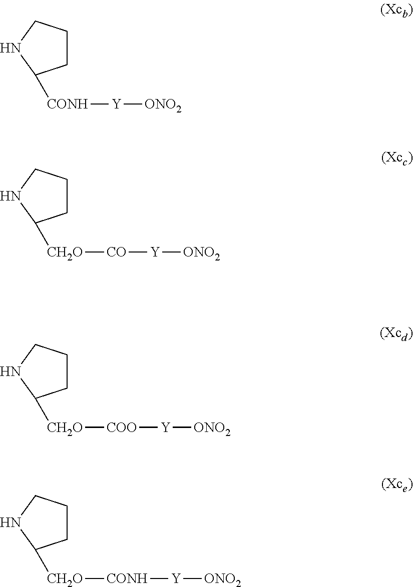

wherein Y is as above defined;
or
3b.2) a compound of formula (Xdₐ)-(Xdₑ), depending on the meaning of K′

NH₂-J-COO-A (Xdₐ)

wherein A is —(Y—ONO₂)

NH₂-J-CONH—Y—ONO₂ (Xd_{b})

NH₂-J-CH₂O—Y—ONO₂ (Xd_{c})

NH₂-J-CH₂O—CO—O—Y—ONO₂ (Xd_{d})

NH₂-J-CH₂O—CO—NH—Y—ONO₂ (Xdₑ)

wherein Y and J are as above defined;
by reaction with:

### 1) if N₀₀=—COOH:

a condensing agent such as dicyclohexylcarbodiimide (DCC) or N,N′-carbonyldiimidazol (CDI) or other known condensing reagents such as O-(7-azabenzotriazol-1-yl)-N,N,N′,N′-tetramethyluronium hexafluorophosphate (HATU), in the presence or not of 1-Hydroxybenzotriazole (HOBT) in solvent such as DMF, THF, chloroform at a temperature in the range from −5° C. to 80° C. in the presence or not of a base as for example DMAP.

### 2) if N₀₀=—COHal or —COOAct:

the reaction is generally carried out in presence of a inorganic or organic base in an aprotic polar/non-polar solvent such as DMF, THF or CH₂Cl₂ at temperatures range between 0°-80° C. or in a double phase system H₂O/Et₂O at temperatures range between 20°-40° C.; or in the presence of DMAP and a Lewis acid such as Sc(OTf)₃ or Bi(OTf)₃ in solvents such as DMF, CH₂Cl₂.

For the preparation of compounds of formula (Xcₐ)-(Xcₑ) and (Xdₐ)-(Xdₑ) see Appendix 1, preparations A1-A6.

Compounds R_{IIc}-R_{IIh} wherein —N₀₀ is —COHal or —COOAct were obtained transforming a compound R_{IIc}-R_{IIh} wherein —N₀₀ is —COOH by known procedures.

Alternatively compounds R_{IIc} wherein —N₀₀ is —COOAct and preferably —N₀₀ is the N-hydroxysuccinimido ester:

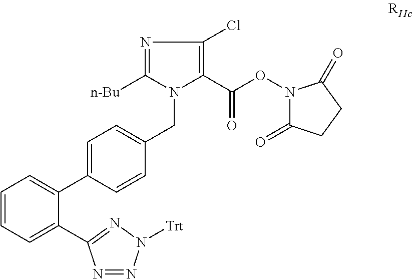

can be prepared more efficiently by reacting compound A with the commercially available compound B:

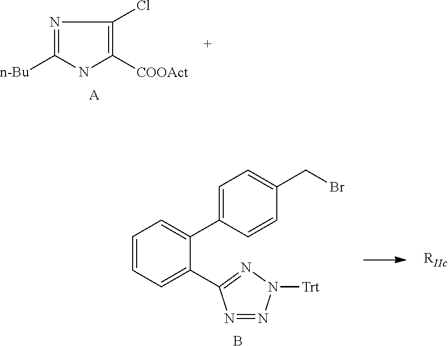

The reaction is generally carried out in presence of a base in an aprotic polar/non-polar solvent such as DMF, THF or CH₂Cl₂ at temperatures range between −15°-+80° C. or in a double phase system H₂O/Et₂O at temperatures range between 20°-40° C.

Compounds of formula A can be obtained by reacting compounds of formula C with compound Act-OH, and preferably with N-hydroxysuccinimide following known procedures:

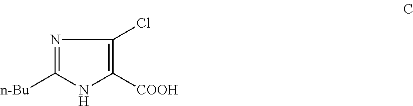

Compound C can be prepared by KMnO₄ oxidation as described in WO 2005/011646 of the corresponding aldehyde D, commercially available:

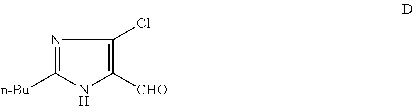

R_{IIc} wherein —N₀₀ is —COOH can be prepared from compound R_{IIcc} known as EXP 3174:

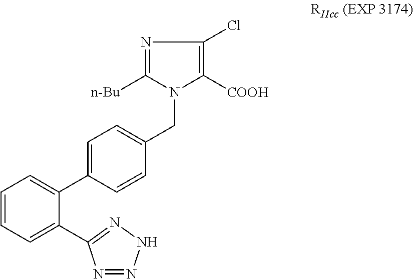

by reacting with trityl chloride and TEA in CH₂Cl₂ following known procedures;
R_{IId} wherein —N₀₀ is —COOH is known as trityl DuP 532 and can be prepared as described by Michael E. Pierce in J. Org. Chem. 1993, 58, 4642-4645;
R_{IIe} wherein —N₀₀ is —COOH is known as trityl olmesartan and is commercially available;
R_{IIf} wherein —N₀₀ is —COOH is known as trityl valsartan and is commercially available
R_{IIg} wherein —N₀₀ is —COOH can be prepared from compound R_{IIgg} known as CV 11194 by reacting with trityl chloride as already described for R_{IIcc};

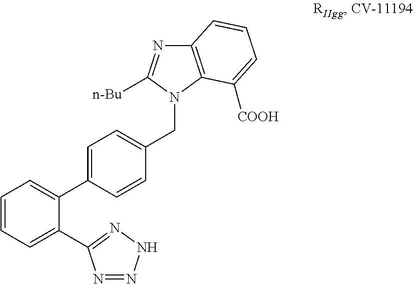

R_{IIgg} can be prepared as described by Kubo, K. et al, in J. Med. Chem. 1993, 36, 1772-1784.
R_{IIh} wherein —N₀₀ is —COOH is known as trityl candesartan and is commercially available.

4. The compounds of general formula (I)

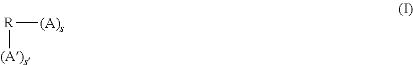

wherein:
s is equal to 1 or 2;
s′ is equal to 0,
A is —(Y—ONO₂) wherein Y is as above defined,
R is selected from:
i) s=1: the residue of formula (II) wherein R₁ is selected from (Ve) and R₀ is N₀ and is selected from:
1) (VIb), wherein K′ is equal to —COO—, —CONH—, —CH₂—O—CO—, —CH₂—O—COO— or —CH₂—O—CONH— and K′ is bound to the group —(Y—ONO₂);
2) —CO—NH-J-K′ wherein J is selected among (VIIa-VIIk) and K′ is as above defined;
   ii) s=2: the residue of formula (III) wherein N₀ is selected from:
1) (VIb), wherein K′ is equal to —COO—, —CONH—, —CH₂—O—CO—, —CH₂—O—COO— or —CH₂—O—CONH— and K′ is bound to the group —(Y—ONO₂);
2) —CO—NH-J-K′ wherein J is selected among (VIIa-VIIk) and K′ is as above defined;
   can be prepared as follows:
   4a) by reacting a compound of formula R_{IIi} or a compound of formula R_{IIIa} with compounds (Xcₐ)-(Xcₑ) or (Xdₐ)-(Xdₑ) already described, following the same procedure described in 3b) using a ratio of (Xcₐ)-(Xcₑ) or (Xdₐ)-(Xdₑ) 1:1 or 2:1 if more than one group —COOH is present.

Compound R_{IIi} is known as telmisartan and is commercially available:

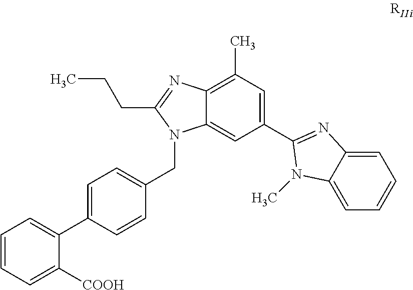

Compound R_{IIIa} is known as eprosartan and is commercially available:

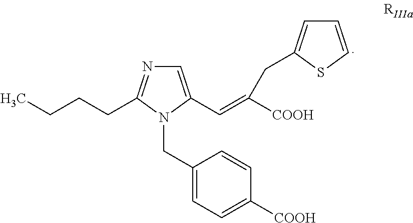

5. The compounds of general formula (I)

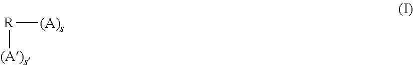

wherein:
s is equal to 1;
s′ is equal to 0;
A is —(Y—ONO₂) wherein Y is as above defined,
R is selected from the residue of formula (II) wherein R₀ is (IV) and R₁ is selected from the group (Va) wherein:
R₂ is n-butyl, R₃ is Cl, and N₀ is selected from:
1) —CH₂—O—CO—NH—K—K* wherein K* is equal to K′ and is equal to —COO—, —CONH—, —CH₂—O—CO—, —CH₂—O—COO— or —CH₂—O—CONH— and K* is bound to the group —(Y—ONO₂); K is selected from K₁, K₂ or K₃ and is selected from (VIIIa)-(VIIIh) wherein R₅, R₇ and R₈ are —H, R₆ is —OH
2) (IXa) wherein K* is equal to K′ and is as above defined and R₇ is —H;
can be prepared as follows:
5a) by reacting a compound of formula (Id)

R_{d}—[Y—ONO₂]ₛ (Id)

wherein s and Y are as above defined in 5., R_{d} is selected from the residue of formula (II) wherein R₁, R₂ and R₃ are as above defined in 5.; R₀ is equal to (IVa) and is as defined in 1a); N₀ is equal to N₀ₐ and is equal to:
5a.1) —CH₂—O—CO—NH—Kₐ—K* wherein K* is as above defined in 5. and K* is bound to the group —(—Y—ONO₂); Kₐ is selected from (VIIIaa)-(VIIIha) (see Appendix 1, preparation A7);
or
5a.2) N₀ₐ is the group IXaₐ

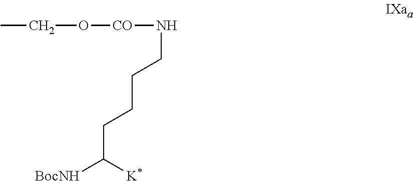

wherein K* and Boc are as previously defined;
with anhydrous or aqueous organic or inorganic acid to hydrolyze the trityl and the other protective groups following procedure well known in the literature;
5b) by reacting a compound of formula R_{IIa′} above described:

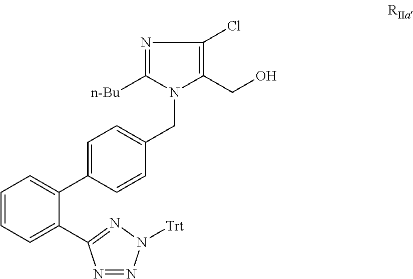

with:
5b.1) a compound of formula (Xeₐ)-(Xeₑ) depending on the meaning of K*:

p-NO₂—C₆H₄—O—CO—NH—Kₐ—COO-A (Xeₐ)

p-NO₂—C₆H₄—O—CO—NH—Kₐ—CONH—Y—ONO₂ (Xe_{b})

p-NO₂—C₆H₄—O—CO—NH—Kₐ—CH₂O—CO—Y—ONO₂ (Xe_{c})

p-NO₂—C₆H₄—O—CO—NH—Kₐ—CH₂O—CO—O—Y—ONO₂ (Xe_{d})

p-NO₂—C₆H₄—O—CO—NH—Kₐ—CH₂O—CO—NH—Y—ONO₂ (Xeₑ)

wherein A is —(Y—ONO₂) and Y and Kₐ are as above defined;
or
5b.2) a compound of formula (Xfₐ)-(Xfₑ):

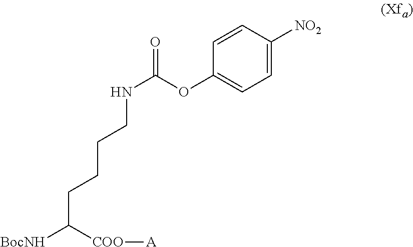

wherein A is —(Y—ONO₂)

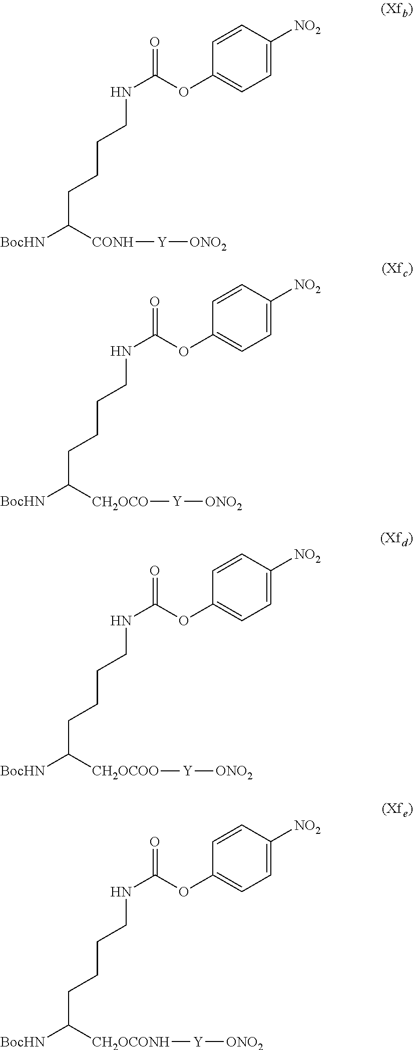

wherein Y is as above defined; following the procedure described in 1b).

For the preparation of compounds of formula (Xeₐ)-(Xeₑ) and (Xfₐ)-(Xfₑ) see Appendix 1, preparations A7 and A8.

6. The compounds of general formula (I)

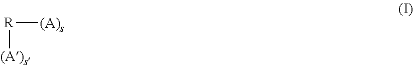

wherein:
s is equal to 1;
s′ is equal to 0
A is —(Y—ONO₂) wherein Y is as above defined,
R is selected from the residue of formula (II) wherein R₀ is (IV) and R₁ is selected from the group (Vd) and N₀ is selected from:
1) —O—CO—NH—K—K* wherein K* is equal to K′ and is equal to —COO—, —CONH—, —CH₂—O—CO—, —CH₂—O—COO— or —CH₂—O—CONH— and K* is bound to the group —(Y—ONO₂); K is selected from (VIIIa)-(VIIIh) wherein R₅, R₇ and R₈ are —H, R₆ is —OH
2) (IXb) wherein K* is as above defined and R₇ is —H;
can be prepared as follows
6a) by reacting a compound of formula (Ie)

Rₑ—[Y—ONO₂]ₛ (Ie)

wherein s and Y are as above defined in 6., Rₑ is selected from the residue of formula (II) wherein R₁ and N₀ are as above defined in 6.; R₀ is equal to (IVb) and is the t-butyl protecting group as defined in 2a); N₀ is equal to N_{0b} and is equal to:
6a.1) —O—CO—NH—Kₐ—K* wherein K* is as above defined in 6. and K* is bound to the group —(Y—ONO₂); Kₐ is selected from (VIIIaa)-(VIIIha) as already described in 5a); or
6a.2) N_{0b} is the group IXbₐ:

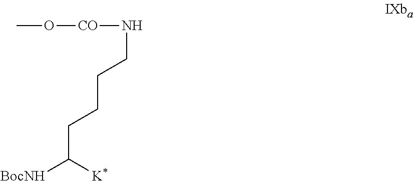

wherein K* and Boc are as previously defined;
with anhydrous or aqueous organic or inorganic acid to hydrolyze the t-butyl and the other protective groups following procedure well known in the literature;
6b) by reacting a compound of formula R_{IIb}:

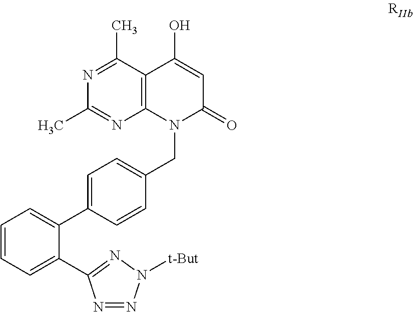

with:
6b.1) compounds of formula (Xeₐ)-(Xeₑ) above described, depending on the meaning of K*; or
6b.2) compounds of formula (Xfₐ)-(Xfₑ) above described, depending on the meaning of K*;
following the procedure described in 1b).

7. The compounds of general formula (I)

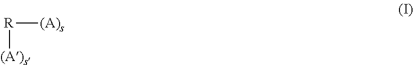

wherein:
s is equal to 1;
s′ is equal to 0;
A is —(Y—ONO₂) wherein Y is as above defined,
R is selected from the residue of formula (II) wherein R₀ is (IV), R₁ is selected from the group:
i) (Va) wherein R₂ is n-butyl and R₃ is Cl;
ii) (Va) wherein R₂ is n-propyl and R₃ is C₂F₅,
iii) (Va) wherein R₂ is n-propyl and R₃ is the group —C(CH₃)₂OH;
iv) (Vb);
v) (Vc) wherein R₄ is n-butyl;
vi) (Vc) wherein R₄ is —OEt;
   wherein N₀ is selected from:
   1) —CO—NH—K—K* wherein K* is equal to K′ and is equal to —COO—, —CONH—, —CH₂—O—CO—, —CH₂—O—COO— or —CH₂—O—CONH— and K* is bound to the group —(Y—ONO₂); K is selected from (VIIIa)-(VIIIh) wherein R₅, R₇ and R₈ are —H, R₆ is —OH;
   2) (IXc) wherein K* is as above defined and R₇ is —H;
   can be prepared as follows:
   7a) by reacting a compound of formula (If)

R_{f}—[Y—ONO₂]ₛ (If)

wherein s and Y are as above defined in 7., R_{f} is selected from the residue of formula (II) wherein R₁ is as above defined in 7. in the points i)-vi); R₀ is equal to (IVa) and is as previously defined, N₀ is equal to N_{0c} and is equal to:
7a.1) —CO—NH—Kₐ—K* wherein K* is as above defined in 7. and K* is bound to the group —(Y—ONO₂); Kₐ is as defined in 5a); or
7a.2) N_{0c} is the group IXcₐ:

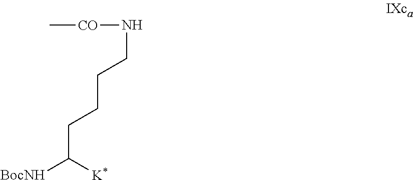

wherein K* and Boc are as previously defined;
with anhydrous or aqueous organic or inorganic acid to hydrolyze the trityl and the other protective groups following procedure well known in the literature;
7b) by reacting compounds of formula R_{IIc}-R_{IIh} already defined in 3b) with:
7b.1) a compound of formula (Xgₐ)-(Xgₑ), depending on the meaning of K*:

NH₂—Kₐ—COO-A (Xgₐ)

NH₂—Kₐ—CONH—Y—ONO₂ (Xg_{b})

NH₂—Kₐ—CH₂O—CO—Y—ONO₂ (Xg_{c})

NH₂—Kₐ—CH₂O—CO—O—Y—ONO₂ (Xg_{d})

NH₂—Kₐ—CH₂O—CO—NH—Y—ONO₂ (Xgₑ)

wherein A is —(Y—ONO₂), Y and Kₐ are as above defined; or
7b.2) a compound of formula (Xhₐ)-(Xhₑ), depending on the meaning of K*:

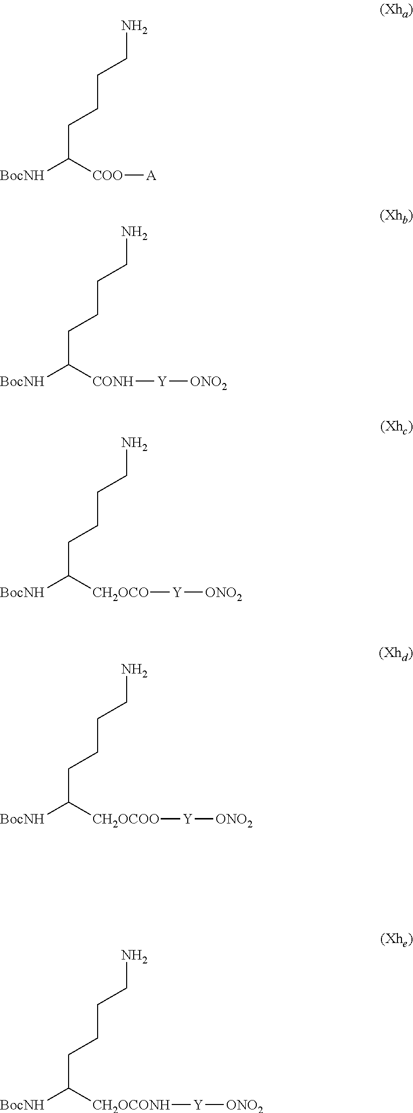

wherein A is —(Y—ONO₂) and Y is as above defined;
following the procedures reported in 3c) for:

### 1) N₀₀=—COOH:

### 2) N₀₀=—COHal or —COOAct:

For the preparation of compounds of formula (Xgₐ)-(Xgₑ) and (Xhₐ)-(Xhₑ) see Appendix 1, preparations A7 and A8.

8. The compounds of general formula (I)

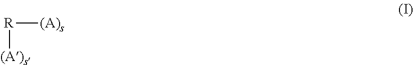

wherein:
s is equal to 1 or 2;
s′ is equal to 0;
A is —(Y—ONO₂) wherein Y is as above defined,
R is selected from:
i) s=1: the residue of formula (II) wherein R₁ is selected from (Ve) and R₀ is N₀ and is selected from:
1) —CO—NH—K—K* wherein K* is equal to K′ and is equal to —COO—, —CONH—, —CH₂—O—CO—, —CH₂—O—COO— or —CH₂—O—CONH— and K* is bound to the group —(Y—ONO₂); K is selected from (VIIIa)-(VIIIh) wherein R₅, R₇ and R₈ are —H, R₆ is —OH;
2) (IXc) wherein K* is as above defined and R₇ is —H;
ii) s=2: the residue of formula (III) wherein N₀ is selected from:
1) —CO—NH—K—K* wherein K* is as above defined and is equal to —COO—, —CONH—, —CH₂—O—CO—, —CH₂—O—COO— or —CH₂—O—CONH— and K* is bound to the group —(Y—ONO₂); K is selected from (VIIIa)-(VIIIh) wherein R₅, R₇ and R₈ are —H, R₆ is —OH;
2) (IXc) wherein K* is as above defined and R₇ is —H;
can be prepared as follows:
8a) by reacting the compounds of formula R_{IIi} or R_{IIIa} described in 4a) with compounds (Xgₐ)-(Xgₑ) and (Xhₐ)-(Xhₑ) depending on the meaning of K* and described in 7b) following the same procedure described in 7b) using a ratio of (Xgₐ)-(Xgₑ) and (Xhₐ)-(Xhₑ)) 1:1 or 2:1 if more than one group —COOH is present.

9. The compounds of general formula (I)

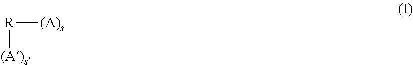

wherein:
s and s′ are equal to 1;
A and A′ are —(Y—ONO₂) or —(Y′—ONO₂) wherein Y and Y′ are equal or different and are as above defined,
R is selected from the residue of formula (II) wherein R₀ is (IV) and R₁ is selected from the group (Va) wherein:
R₂ is n-butyl, R₃ is Cl, and N₀ is selected from:
1) —CH₂—O—CO—NH—K—K* wherein K* is equal to K′ is equal to —COO—, —CONH—, —CH₂—O—CO—, —CH₂—O—COO— or —CH₂—O—CONH— and K* is bound to the group —(Y—ONO₂); K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
2) (IXa) wherein K* is as above defined and R₇ is selected from —CO— or —COO— and R₇ is bound to the group —(Y′—ONO₂);
can be prepared as follows:
9a) by reacting a compound of formula (Ig)

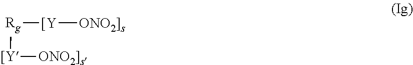

wherein s, s′, Y and Y′ are as above defined in 9., R_{g} is selected from the residue of formula (II) wherein R₁, R₂ and R₃ are as above defined in 9.; R₀ is equal to (IVa) and is as defined in 1a); N₀ is equal to:
9a.1) —CH₂—O—CO—NH—K—K* wherein K and K* are as above defined in 9.;
9a.2) (IXa) wherein K* and R₇ are as above defined in 9.; with anhydrous or aqueous organic or inorganic acid to hydrolyze the trityl protective groups following procedure well known in the literature;
9b) by reacting a compound of formula R_{IIa′}:

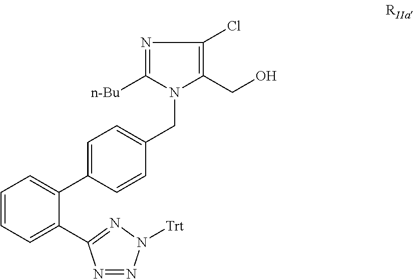

with:
9b.1) compound of general formula (Xiₐ)-(Xiₒ):

p-NO₂—C₆H₄—O—CO—NH—K—K* (Xiₐ)-(Xiₒ)

wherein K* and K are as above defined in 9.
For a complete description and for preparations of (Xiₐ)-(Xiₒ) see Appendix 1, preparations A9-A23; or
9b.2) a compound of general formula (Xjₐ)-(Xjₑ)

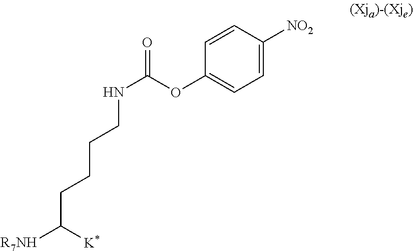

wherein K* is equal to —COO—, —CONH—, —CH₂—O—CO—, —CH₂—O—COO— or —CH₂—O—CONH— and K* is bound to the group —(Y—ONO₂); R₇ is selected from —CO— or —COO— and R₇ is bound to the group —(Y′—ONO₂).
following the procedure described in 1b);

For the preparation of compounds of formula (Xjₐ)-(Xjₑ) see Appendix 1, preparation A24.

10. The compounds of general formula (I)

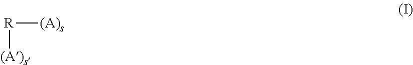

wherein:
s and s′ are equal to 1;
A and A′ are —(Y—ONO₂) or —(Y′—ONO₂) wherein Y and Y′ are equal or different and are as above defined,
R is selected from the residue of formula (II) wherein R₀ is (IV) and R₁ is selected from the group (Vd) and N₀ is selected from:
1) —O—CO—NH—K—K* wherein K* is equal to K′ and is equal to —COO—, —CONH—, —CH₂—O—CO—, —CH₂—O—COO— or —CH₂—O—CONH— and K* is bound to the group —(Y—ONO₂); K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
2) (IXb) wherein K* is as above defined and R₇ is selected from —CO— or —COO— and R₇ is bound to the group —(Y′—ONO₂); can be prepared as follows:
10a) by reacting a compound of formula (Ih)

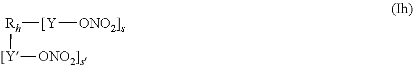

wherein s, s′ Y and Y are as above defined in 10., Rₕ is selected from the residue of formula (II) wherein R₁ and N₀ are as above defined in 10.; R₀ is equal to (IVb) and is the t-butyl protecting group as defined in 2a); N₀ is equal to:
10a.1) —O—CO—NH—K—K* wherein K is selected from K₁, K₂ or K₃ and K and K* are as above defined in 10.; K₁, K₂, K₃ and K* are respectively bound to the group —(Y′—ONO₂) and —(Y—ONO₂);
10a.2) (IXb) wherein K* and R₇ are as above defined in 10.; R₇ is bound to the group —(Y′—ONO₂);
with anhydrous or aqueous organic or inorganic acid to hydrolyze the t-butyl group following procedure well known in the literature;
10b) by reacting a compound of formula R_{IIb}:

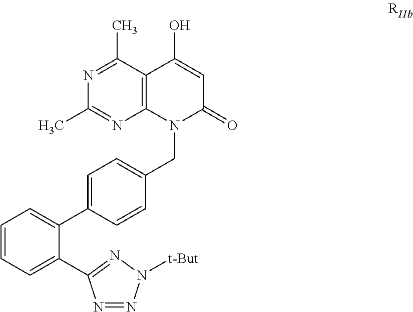

with:
10b.1) a compound of general formula (Xiₐ)-(Xiₒ) as above defined

p-NO₂—C₆H₄—O—CO—NH—K—K* (Xiₐ)-(Xiₒ)

following the procedure described in 1b); or
10b.2) a compound of general formula (Xjₐ)-(Xjₑ) defined in 9b), following the procedure described in 1b).

11. The compounds of general formula (I)

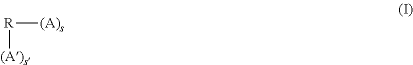

wherein:
s is equal to 1;
s′ is equal to 1;
A and A′ are —(Y—ONO₂) or —(Y′—ONO₂) wherein Y and Y′ are as above defined,
R is selected from the residue of formula (II) wherein R₀ is (IV), R₁ is selected from the group:
i) (Va) wherein R₂ is n-butyl and R₃ is Cl;
ii) (Va) wherein R₂ is n-propyl and R₃ is C₂F₅,
iii) (Va) wherein R₂ is n-propyl and R₃ is the group —C(CH₃)₂OH;
iv) (Vb);
v) (Vc) wherein R₄ is n-butyl;
vi) (Vc) wherein R₄ is —OEt;
   wherein N₀ is selected from:
   1) —CO—NH—K—K* wherein K* is equal to K′ and is equal to —COO—, —CONH—, —CH₂—O—CO—, —CH₂—O—COO— or —CH₂—O—CONH— and K* is bound to the group —(Y—ONO₂); K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
   2) (IXc) wherein K* is as above defined and R₇ is selected from —CO— or —COO— and R₇ is bound to the group-(Y′—ONO₂);
   can be prepared as follows:
   11a) by reacting a compound of formula (Ii)

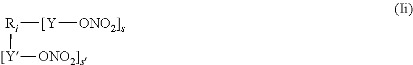

wherein s, s′, Y and Y′ are as above defined in 11., Rᵢ is selected from the residue of formula (II) wherein N₀ is as above defined, R₁ is as above defined in 11. in the points i)-vi); R₀ is equal to (IVa) and is as previously defined, with anhydrous or aqueous organic or inorganic acid to hydrolyze the trityl and the other protective groups following procedure well known in the literature;
11b) by reacting compounds of formula R_{IIc}-R_{IIh} already defined in 3b) with
11b.1) compounds of general formula (Xkₐ)-(Xkₒ)

NH₂—K—K* (Xkₐ)-(Xkₒ)

wherein K* and K are as defined in 11.;
or
11b.2) compound of formula (XLₐ)-(XLₑ), depending on the meaning of K*:

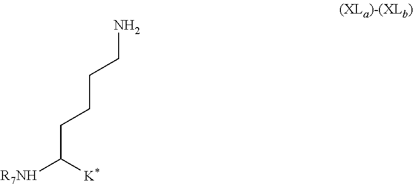

Wherein R₇ is selected from —CO— or —COO— and R₇ is bound to the group —(Y′—ONO₂);
following the procedures reported in 3c) for:

### 1) N₀₀=—COOH:

### 2) N₀₀=—COHal or —COOAct:

For the preparation of compounds of formula (Xkₐ)-(Xkₒ) and (XLₐ)-(XLₑ) see Appendix 1, preparations A9-A24.

12. The compounds of general formula (I)

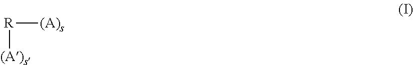

wherein:
s is equal to 1 or 2;
s′ is equal to 1 or 2;
A and A′ are —(Y—ONO₂) or —(Y′—ONO₂) wherein Y and Y′ are as above defined,
R is selected from:
i) s=1, s′=1: the residue of formula (II) wherein R₁ is selected from (Ve) and R₀ is N₀ and is selected from:
1) —CO—NH—K—K* wherein K* is equal to K′ and is equal to —COO—, —CONH—, —CH₂—O—CO—, —CH₂—O—COO— or —CH₂—O—CONH— and K* is bound to the group —(Y—ONO₂); K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group-(Y′—ONO₂).
2) (IXc) wherein K* is as above defined and R₇ is selected from —CO— or —COO— and R₇ is bound to the group —(Y′—ONO₂);
ii) s=2, s′=2: the residue of formula (III) wherein N₀ is selected from:
1) —CO—NH—K—K* wherein K* is equal to K′ and is equal to —COO—, —CONH—, —CH₂—O—CO—, —CH₂—O—COO— or —CH₂—O—CONH— and K* is bound to the groups —(Y—ONO₂); K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the groups —(Y′—ONO₂).
2) (IXc) wherein K* is as above defined and R₇ is selected from —CO— or —COO— and R₇ is bound to the group —(Y′—ONO₂);
can be prepared as follows:
12a) by reacting the compounds of formula R_{IIi} or R_{IIIa} described in 4a) with:
12a.1) compounds of general formula (Xkₐ)-(Xkₒ)

NH₂—K—K* (Xkₐ)-(Xkₒ)

defined in 11b); or
12a.2) compound of formula (XLₐ)-(XLₑ), defined in 11b); following the same procedure described in 7b) using a ratio of (Xkₐ)-(Xkₒ) and (XLₐ)-(XLₑ) 1:1 or 2:1 if more than one group —COOH is present.

13. The compounds of general formula (I)

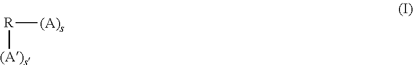

wherein:
s is equal to 0;
s′ is equal to 1;
A′ is —(Y′—ONO₂) wherein Y′ is as above defined;
R is selected from the residue of formula (II) wherein R₀ is (IV) and R₁ is selected from the group (Va) wherein:
R₂ is n-butyl, R₃ is Cl, and N₀ is selected from:
1) —CH₂—O—CO—NH—K—K* wherein K* is equal to —COOH; K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
2) (IXa) wherein K* is equal to —COOH and R₇ is —CO— or —COO— and R₇ is bound to the group —(Y′—ONO₂);
can be prepared as follows:
13a) by reacting a compound of formula (Ij)

Rⱼ—[Y′—ONO₂]_{s′} (Ij)

wherein s′ and Y′ are as above defined in 13., Rⱼ is selected from the residue of formula (II) wherein R₁, R₂ and R₃ are as above defined in 13.; R₀ is equal to (IVa) and is as defined in 1a); N₀ is equal to N₀ₐ and is equal to:
13a.1) —CH₂—O—CO—NH—K—Kₓ* wherein Kₓ* is equal to —COOt-But; K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
or
13a.2) N₀ₐ is the group IXa_{b}

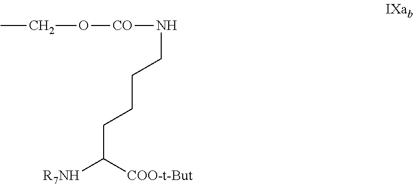

wherein t-But is the tert-butyl protecting group and R₇ is selected from —CO— or —COO— and binds the group —(Y′—ONO₂); with anhydrous or aqueous organic or inorganic acid to hydrolyze the trityl and the other protective groups following procedure well known in the literature;
13b) by reacting a compound of formula R_{IIa′} above described:

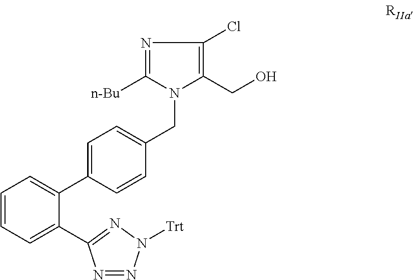

depending on the meaning of K, with:
13b.1) compound of formula (Xiₐ₄)-(Xiₐ₆)

p-NO₂—C₆H₄—O—CO—NH—K₁—COOtBut (Xiₐ₄)-(Xiₐ₆)

wherein K₁ is selected from (VIIIa)-(VIIId) wherein R₅ is selected from respectively:

### i) —CO— (Xiₐ₄);

### ii) —COO— (Xiₐ₅); or

iii) —CONH— (Xiₐ₆);
and K₁ is bound to the group —(Y′—ONO₂) (see Appendix 1, preparation A9/2);
13b.2) compound of formula (Xi_{f3}), (Xi_{f4}):

p-NO₂—C₆H₄—O—CO—NH—K₂—COOtBut (Xi_{f3}), (Xi_{f4})

wherein K₂ is selected from (VIIIe), (VIIIf) wherein R₆ is selected from respectively:
i) —O— (Xi_{f3}) or
ii) —NH— (Xi_{f4})
   and K₂ is bound to the group —(Y′—ONO₂) (see Appendix 1, preparation A14/2);
   13b.3) compound of formula (Xiₖ₃), (Xiₖ₄):

p-NO₂—C₆H₄—O—CO—NH—K₃—COOtBut (Xiₖ₃), (Xiₖ₄)

wherein K₃ is selected from (VIIIh), (VIIIg) wherein R₇ and R₈ are selected from respectively:
i) —CO— (Xiₖ₃) or
ii) —COO— (Xiₖ₄)
   and K₃ is bound to the group —(Y′—ONO₂) (see Appendix 1, preparation A19/2);
   13b.4) compounds of formula (Xjₐ₃)-(Xjₐ₄:

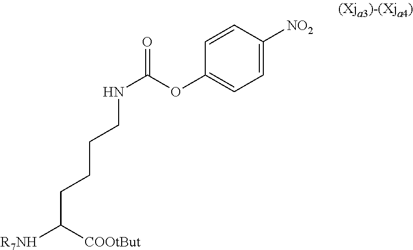

wherein R₇ is selected from respectively:
i) —CO— (Xjₐ₃),
ii) —COO— (Xjₐ₄) and R₇ is bound to the group —(Y′—ONO₂) (see Appendix 1, preparation A24/2);
   following the procedure described in 1b).

14. The compounds of general formula (I)

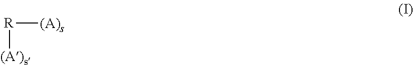

wherein:
s is equal to 0;
s′ is equal to 1
A′ is —(Y′—ONO₂) wherein Y′ is as above defined;
R is selected from the residue of formula (II) wherein R₀ is (IV) and R₁ is selected from the group (Vd) and N₀ is selected from:
1) —O—CO—NH—K—K* wherein K* is equal to —COOH; K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
2) (IXb) wherein K* is equal to —COOH and R₇ is —CO— or —COO— and R₇ is bound to the group —(Y′—ONO₂);
can be prepared as follows:
14a) by reacting a compound of formula (Ik)

Rₖ—[Y′—ONO₂]_{s′} (Ik)

wherein s′ and Y′ are as above defined in 14., Rₖ is selected from the residue of formula (II) wherein R₁ is as above defined in 14.; R₀ is equal to (IVb) and is as defined in 2a); N₀ is equal to N_{0b} and is equal to:
14a.1) —O—CO—NH—K—Kₓ* wherein Kₓ* is equal to —COOt-But; K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
or
14a.2) N_{0b} is the group IXb_{b}

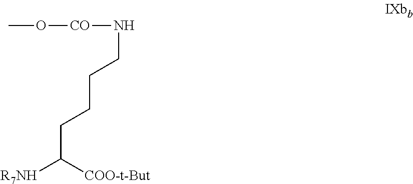

wherein t-But is the tert-butyl protecting group and R₇ is selected from —CO— or —COO— and binds the group —(Y′—ONO₂); with anhydrous or aqueous organic or inorganic acid to hydrolyze the trityl and the other protective groups following procedure well known in the literature;
14b) by reacting a compound of formula R_{IIb}:

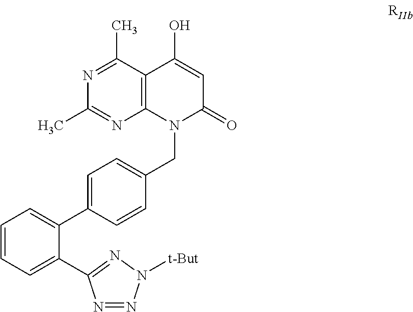

depending on the meaning of K, with:
14b.1) compound of formula (Xiₐ₄)-(Xiₐ₆)

p-NO₂—C₆H₄—O—CO—NH—K₁—COOtBut (Xiₐ₄)-(Xiₐ₆)

described in 13b.1)
14b.2) compound of formula (Xi_{f3}), (Xi_{f4}):

p-NO₂—C₆H₄—O—CO—NH—K₂—COOtBut (Xi_{f3}), (Xi_{f4})

described in 14b.2)
14b.3) compound of formula (Xiₖ₃), (Xiₖ₄):

p-NO₂—C₆H₄—O—CO—NH—K₃—COOtBut (Xiₖ₃), (Xiₖ₄)

described in 13b.3)
14b.4) compounds of formula (Xjₐ₃)-(Xjₐ₄:

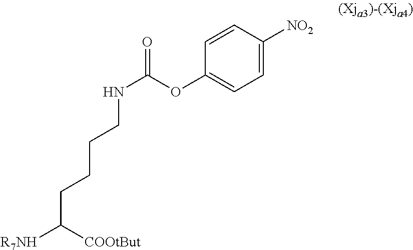

described in 13b.4);
following the procedure described in 1b).

15. The compounds of general formula (I)

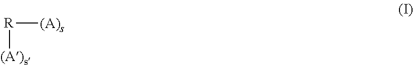

wherein:
s is equal to 0;
s′ is equal to 1;
A′ is —(Y′—ONO₂) wherein Y′ is as above defined;
R is selected from the residue of formula (II) wherein R₀ is (IV), R₁ is selected from the group:
i) (Va) wherein R₂ is n-butyl and R₃ is Cl;
ii) (Va) wherein R₂ is n-propyl and R₃ is C₂F₅,
iii) (Va) wherein R₂ is n-propyl and R₃ is the group —C(CH₃)₂OH;
iv) (Vb);
v) (Vc) wherein R₄ is n-butyl;
vi) (Vc) wherein R₄ is —OEt;
   wherein N₀ is selected from:
   1) —CO—NH—K—K* wherein K* is equal to —COOH; K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
   2) (IXc) wherein K* is equal to —COOH; and R₇ is selected from —CO— or —COO— and R₇ is bound to the group —(Y′—ONO₂);
   can be prepared as follows:
   15a) by reacting a compound of formula (IL)

R_{L}—[Y′—ONO₂]_{s′} (IL)

wherein s′ and Y′ are as above defined in 15., R_{L} is selected from the residue of formula (II) wherein R₁ is as above defined in 15. in the points i)-vi); R₀ is equal to (IVa) and is as previously defined, N₀ is equal to N_{0c} and is equal to:
7a.1) —CO—NH—K—Kₓ* wherein Kₓ* is equal to —COOt-But; K is as defined in 15.; or
7a.2) N_{0c} is the group IXc_{b}:

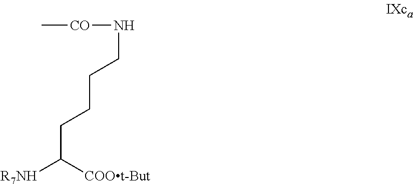

wherein R₇ and t-But are as previously defined;
with anhydrous or aqueous organic or inorganic acid to hydrolyze the trityl and the other protective groups following procedure well known in the literature;
15b) by reacting compounds of formula R_{IIc}-R_{IIh} already defined in 3b), depending on the meaning of K with:
15b.1) compounds of formula (Xkₐ₄)-(Xkₐ₆):

NH₂—K₁—COOtBut (Xkₐ₄)-(Xkₐ₆)

wherein t-But is as above defined and K₁ is selected from (VIIIa)-(VIIId) wherein R₅ is selected from respectively:
i) —CO— (Xkₐ₄);
ii) —COO— (Xkₐ₅); or
iii) —CONH— (Xkₐ₆);
   and K₁ is bound to the group —(Y′—ONO₂) (see Appendix 1, preparation A9/2); or
   15b.2) compounds of formula (Xk_{f3}), (Xk_{f4}):

NH₂—K₂—COOtBut (Xk_{f3})-(Xk_{f4})

wherein K₂ is selected from:
i) —O— (Xk_{f3}) or
ii) —NH— (Xk_{f4})
   and K₂ is bound to the group —(Y′—ONO₂) (see Appendix 1, preparation A14/2); or
   15b.3) compounds of formula (Xkₖ₃), (Xkₖ₄):

NH₂—K₃—COOtBut (Xkₖ₃)-(Xkₖ₄)

wherein K₃ is selected from (VIIIh), (VIIIg) wherein R₇ and R₈ are selected from respectively:
i) —CO— (Xkₖ₃) or
ii) —COO— (Xkₖ₄)
   and K₃ is bound to the group —(Y′—ONO₂) (see Appendix 1, preparation A19/2); or
   15b.4) compounds of formula (XLₐ₃), (XLₐ₄):

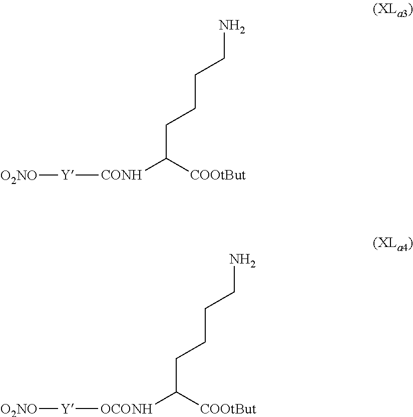

wherein Y′ and t-But are as previously defined; (see Appendix 1, preparation A24/2);
following the procedures reported in 3c) for:

### 1) N₀₀=—COOH:

### 2) N₀₀=—COHal or —COOAct.

16. The compounds of general formula (I)

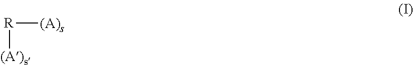

wherein:
s is equal to 0;
s′ is equal to 1 or 2;
A′ is —(Y′—ONO₂) wherein Y′ is as above defined;
R is selected from:
i) s=1: the residue of formula (II) wherein R₁ is selected from (Ve) and R₀ is N₀ and is selected from:
1) —CO—NH—K—K* wherein K* is equal to —COOH; K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
2) (IXc) wherein K* is equal to —COOH; and R₇ is selected from —CO— or —COO— and R₇ is bound to the group —(Y′—ONO₂);
ii) s=2: the residue of formula (III) wherein N₀ is selected from:
1) —CO—NH—K—K* wherein K* is equal to —COOH; K is selected from K₁, K₂ or K₃ and is selected from (VIIIa)-(VIIIh) wherein R₅ is selected from —CO—, —COO— or —CONH—; R₆ is selected from —O— or —NH; R₇ and R₈ are selected from —CO— or —COO— and K is bound to the group —(Y′—ONO₂);
2) (IXc) wherein K* is equal to —COOH; and R₇ is selected from —CO— or —COO— and R₇ is bound to the group —(Y′—ONO₂); can be prepared as follows:
16a) by reacting the compounds of formula R_{IIi} or R_{IIIa} described in 4a) with compounds (Xkₐ₄)-(Xkₐ₆), or (Xk_{f3}), (Xk_{f4}), or (Xkₖ₃)-(Xkₖ₄)
depending on the meaning of K and described in 15b.1-15b.3 or compounds of formula (XLₐ₃), (XLₐ₄) described in 15b.4; following the same procedure described in 3c) using a ratio of (Xkₐ₄)-(Xkₐ₆), or (Xk_{f3}), (Xk_{f4}), or (Xkₖ₃)-(Xkₖ₄), or (XLₐ₃), (XLₐ₄) 1:1 or 2:1 if more than one group —COOH is present.

17. The compounds of general formula (I)

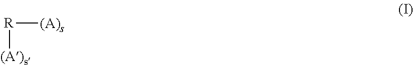

wherein:
s is equal to 1;
s′ is equal to 0
A is equal to (1a) and is the group

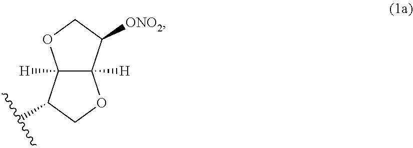

R is selected from the residue of formula (II) wherein R₀ is (IV), R₁ is selected from the group:
i) (Va) wherein R₂ is n-butyl and R₃ is Cl;
iii) (Va) wherein R₂ is n-propyl and R₃ is the group C(CH₃)₂OH;
iv) (Vb);
vi) (Vc) wherein R₄ is —OEt;
   wherein N₀ is selected from:
1) (VIb), wherein K′ is equal to —COO—, and K′ is bound to the group (1a)
2) —CO—NH-J-K′ wherein J is selected among (VIIa-VIIk) and K′ is as above defined;
   can be prepared as follows
   17a) by reacting a compound of formula (Ic′)

R_{c}-[A]ₛ (Ic′)

wherein A and s are as defined in 17. and R_{c} is selected from the residue of formula (II) wherein N₀ is as above defined in 17. and R₁ is as above defined in the points i), iii),iv) and vi); R₀ is equal to (IVa) and is as previously defined, following the same procedure described in 1a);
17b) by reacting:
i) a compound of formula R_{IIc}

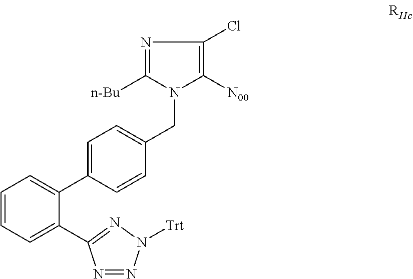

Wherein N₀₀ is —COOH, —COHal or —COOAct wherein Hal and Act are as previously defined in 3.; or
iii) a compound of formula R_{IIe}

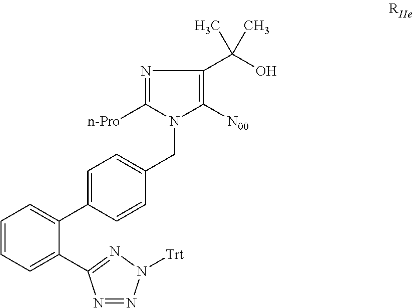

wherein N₀₀ is as previously defined; or
iv) a compound of formula R_{IIf}

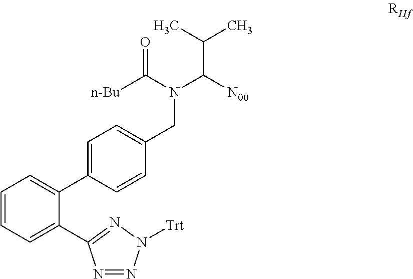

wherein N₀₀ is as previously defined; or
vi) a compound of formula R_{IIh}

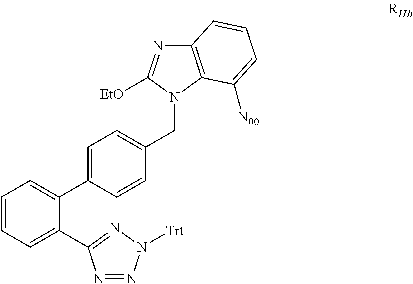

wherein N₀₀ is as previously defined;
with
3b.1) a compound of formula (Xcₐ)

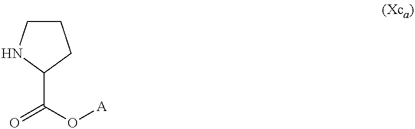

wherein A is the group (1a),

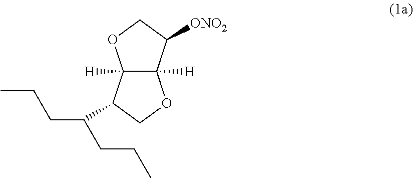

or
3b.2) a compound of formula (Xdₐ)

NH₂-J-COO-A (Xdₐ)

wherein A is (1a) and J is as above defined;
following the procedure described in 3.
17b′) alternatively by reacting:
i) a compound of formula R_{IIca} or R_{IIcb}

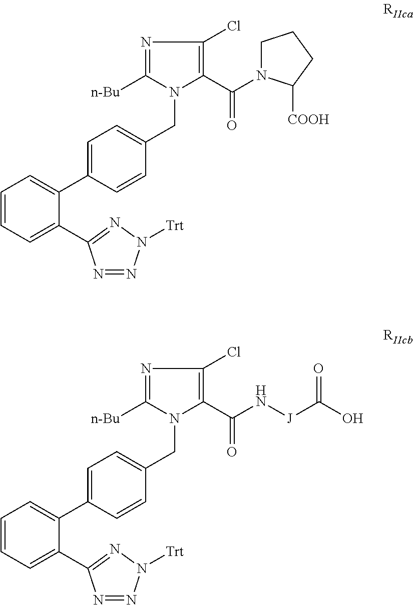

Wherein J is as above defined, with compound (XIa) (see Appendix 1, preparation A1.

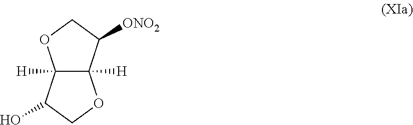

and a condensing agent such as dicyclohexylcarbodiimide (DCC) or N,N′-carbonyldiimidazol (CDI) or 1-ethyl-3-(3′-dimethylaminopropyl)carbodiimide hydrochloride (EDC) or other known condensing reagents such as O-(7-azabenzotriazol-1-yl)-N,N,N′,N′-tetramethyluronium hexafluorophosphate (HATU), in the presence or not of 1-Hydroxybenzotriazole (HOBT) in solvent such as CH₂Cl₂, DMF, THF, chloroform at a temperature in the range from −5° C. to 80° C. in the presence or not of a base as for example DMAP.
17c) by reacting compound R_{IIc} defined in 3b)

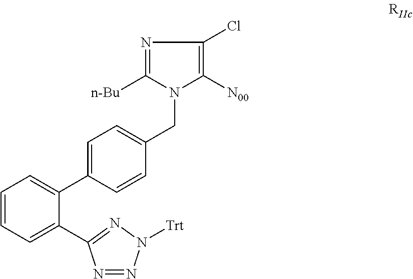

wherein N₀₀ is as previously defined, with compounds (1b) or (1c)

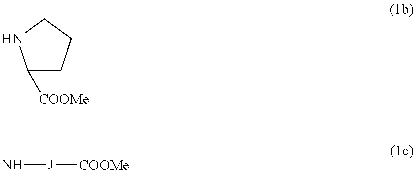

wherein J is as previously defined using a condensing agent as 1-ethyl-3-(3′-dimethylaminopropyl)carbodiimide hydrochloride (EDC) and 1-hydroxybenzotriazole as known in the literature and hydrolysing the final methyl ester with NaOH in solvent such as MeOH as well known in the literature.

### APPENDIX 1

### A1. Synthesis of Compounds (Xaₐ) and (Xcₐ)

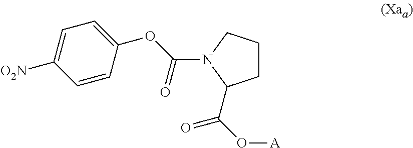

wherein A is —(Y—ONO₂), and Y is as above defined, or the group (1a)

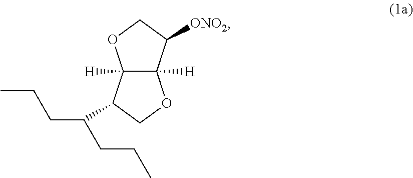

can be obtained by reacting compound (Xcₐ) wherein A is as above defined with commercially available p-nitrophenylchlorocarbonate in the presence of a base following procedures well known in the literature.

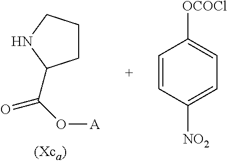

Compounds (Xcₐ) can be obtained from commercially available Boc-proline by the procedures depicted in the scheme 1 following procedures well known in the literature:

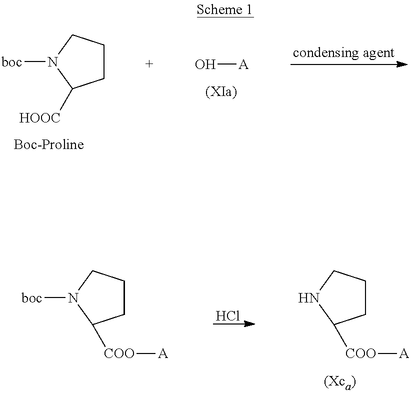

Compounds of formula (XIa) wherein A is the group (1a) are commercially available.

Compounds of formula (XIa) wherein A is the group —(Y—ONO₂) are obtained by reacting compounds of formula HO—Y-Hal (XIIa) wherein Y is as above defined and Hal is an halogen atom such as Cl, Br, I or compounds of formula HO—Y-OTs (XIIIa) wherein Ts is the tosyl group, with AgNO₂ or MetalNO₃ wherein Metal is K⁺, Na⁺ Li⁺ in a suitable organic solvent such as acetonitrile or tetrahydrofuran (THF) under nitrogen in the dark at temperatures range between 20°-80° C. using a nitrate ammonium salt as catalyst; alternatively the reaction with AgNO₂ or MetalNO₃ can be performed under microwave irradiation in solvents such acetonitrile or THF at temperatures in the range between about 100-180° C. for time range about 1-120 min. The compounds of formula (XIIa) and (XIIIa) are commercially available or can be obtained from commercially available compounds HO—Y—OH (XIIIb) with methods well known in the literature.

### A2. Synthesis of Compounds (Xa_{b}) and (Xc_{b})

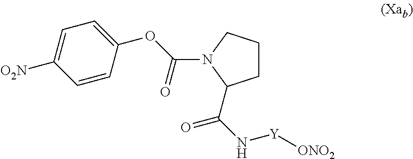

wherein Y is as above defined, can be obtained by reacting compound (Xc_{b}) with commercially available p-nitrophenylchlorocarbonate in the presence of a base following procedures well known in the literature.

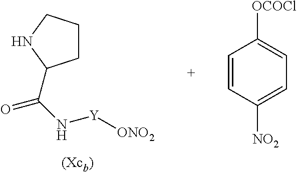

Compounds (Xc_{b}) can be obtained from commercially available Boc-proline by the procedures depicted in the Scheme 2 following procedures well known in the literature:

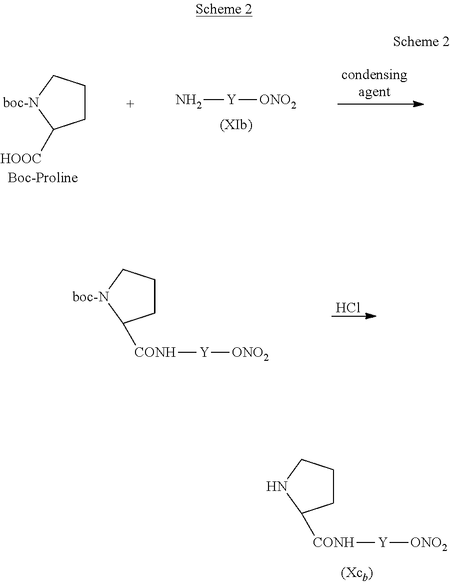

The compounds of formula (XIb) are obtained by reacting compounds of formula BocNH—Y-Hal (XIIb) wherein Y and Hal are as above defined or compounds of formula BocNH—Y—OTs (XIVa) wherein Ts is the tosyl group, with AgNO₃ or MetalNO₃ as already described in A1, eventually acid hydrolysing the Boc protective group. The compounds of formula (XIIb) and (XIVa) are commercially available or can be obtained from commercially available compounds BocNH—Y—OH (XIVb) with methods well known in the literature.

### A3. Synthesis of Compounds (Xaₐ) and (Xc_{c})

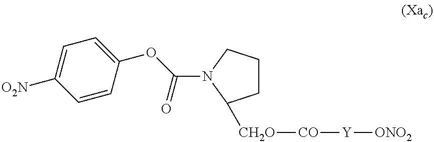

wherein Y is as above defined, can be obtained by reacting compound (Xc_{c}) with commercially available p-nitrophenylchlorocarbonate as already described:

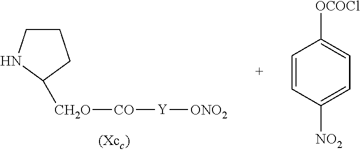

Compounds (Xc_{c}) can be obtained from commercially available Boc-prolinol by the procedures depicted in the Scheme 3 following methods analogues to those described in WO 2006/008196.

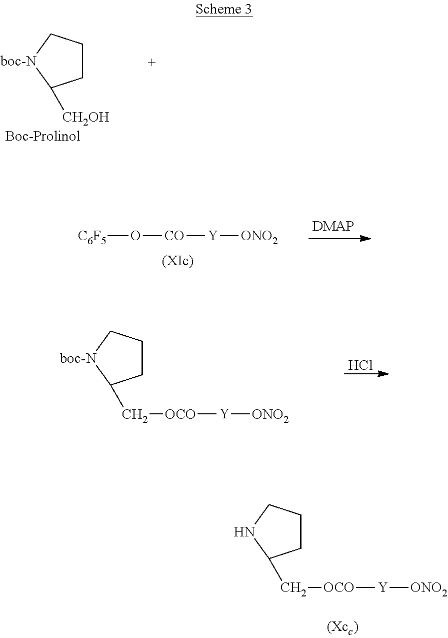

The compounds of formula (XIc) are obtained as described in WO 2006/008196.

### A4. Synthesis of Compounds (Xa_{d}) and (Xc_{d})

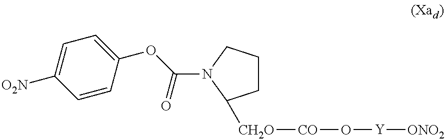

wherein Y is as above defined, can be obtained by reacting compound (Xc_{d}) with commercially available p-nitrophenylchlorocarbonate as already described:

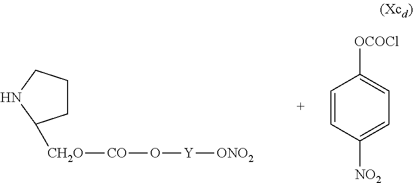

Compounds (Xc_{d}) can be obtained from commercially available Boc-prolinol by the procedures depicted in the Scheme 4 following methods analogues to those described in WO 2006/008196.

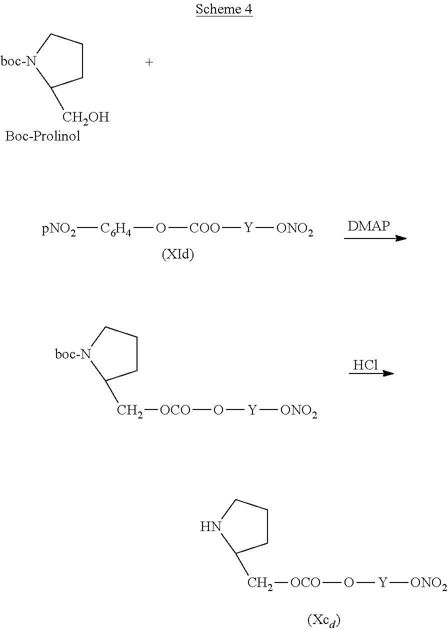

The compounds of formula (XId) are obtained as described in WO 2006/008196.

### A5. Synthesis of Compounds (Xaₑ) and (Xcₑ)

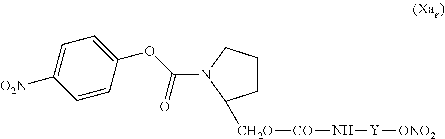

wherein Y is as above defined, can be obtained by reacting compound (Xcₑ) with commercially available p-nitrophenylchlorocarbonate as already described:

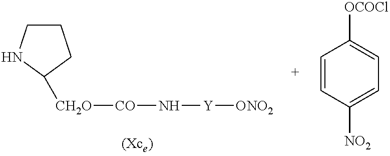

Compounds (Xcₑ) can be obtained from commercially available Boc-prolinol by the procedures depicted in the Scheme 5 following methods well known in the literature:

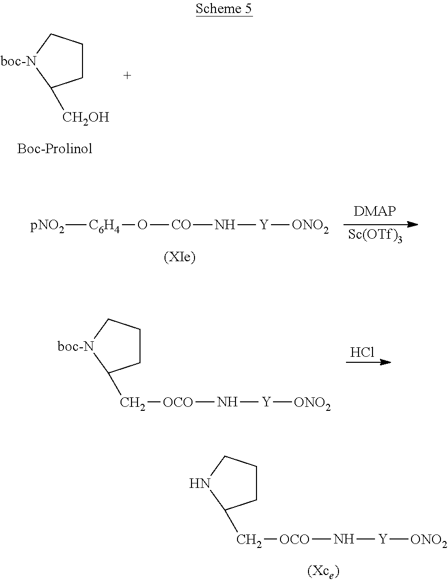

The compounds of formula (XIe) are new compounds and are obtained from (XIb) described in A2 following procedure described for analogues compounds:

NH₂—Y—ONO₂+pNO₂—C₆H₄—OCOCl (XIb)

### A6. Synthesis of Compounds (Xbₐ)-(Xbₑ) and (Xdₐ)-(Xdₑ)

The compounds of formula (Xbₐ)-(Xbₑ) wherein J is as above defined depending on the meaning of K′:

pNO₂—C₆H₄—OCONH-J-COO-A (Xbₐ)

wherein J is as previously defined and A is —(Y—ONO₂) or the group (1a)

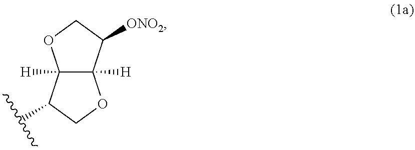

pNO₂—C₆H₄—OCONH-J-CONH—Y—ONO₂ (Xb_{b})

pNO₂—C₆H₄—OCONH-J-CH₂O—CO—Y—ONO₂ (Xb_{c})

pNO₂—C₆H₄—OCONH-J-CH₂O—CO—O—Y—ONO₂ (Xb_{d})

pNO₂—C₆H₄—OCONH-J-CH₂O—CO—NH—Y—ONO₂ (Xbₑ)

Wherein J and Y are as previously defined, are obtained by reaction with pNO₂—C₆H₄—OCOCl of the corresponding compounds (Xdₐ)-(Xdₑ):

NH₂-J-COO-A+pNO₂—C₆H₄—OCOCl (Xdₐ)

Wherein A is as previously defined;

NH₂-J-CONH—Y—ONO₂+pNO₂—C₆H₄—OCOCl (Xd_{b})

NH₂-J-CH₂OCO—Y—ONO₂+pNO₂—C₆H₄—OCOCl (Xd_{c})

NH₂-J-CH₂OCO—O—Y—ONO₂+pNO₂—C₆H₄—OCOCl (Xd_{d})

NH₂-J-CH₂OCO—NH—Y—ONO₂+pNO₂—C₆H₄—OCOCl (Xdₑ)

Wherein J and y are as previously defined.

Compounds of formula (Xdₐ)-(Xdₑ) depending on the meaning of J can be obtained starting from the corresponding Boc protected amino acid or Boc protected amino alcohol following the procedure described in A1-A5 depending on the meaning of K′.

### A7. Synthesis of Compounds (Xeₐ)-(Xeₑ) and (Xgₐ)-(Xgₑ)

The compounds of formula (Xeₐ)-(Xeₑ)

p-NO₂—C₆H₄—O—CO—NH—Kₐ—COO-A (Xeₐ)

wherein A is —(Y—ONO₂) or the group (1a)

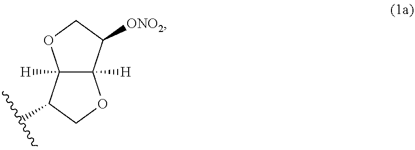

p-NO₂—C₆H₄—O—CO—NH—Kₐ—CONH—Y—ONO₂ (Xe_{b})

p-NO₂—C₆H₄—O—CO—NH—Kₐ—CH₂O—CO—Y—ONO₂ (Xe_{c})

p-NO₂—C₆H₄—O—CO—NH—Kₐ—CH₂O—CO—O—Y—ONO₂ (Xe_{d})

p-NO₂—C₆H₄—O—CO—NH—Kₐ—CH₂O—CO—NH—Y—ONO₂ (Xeₑ)

wherein:
Y is as previously defined, and Kₐ is selected from (VIIIaa)-(VIIIha):

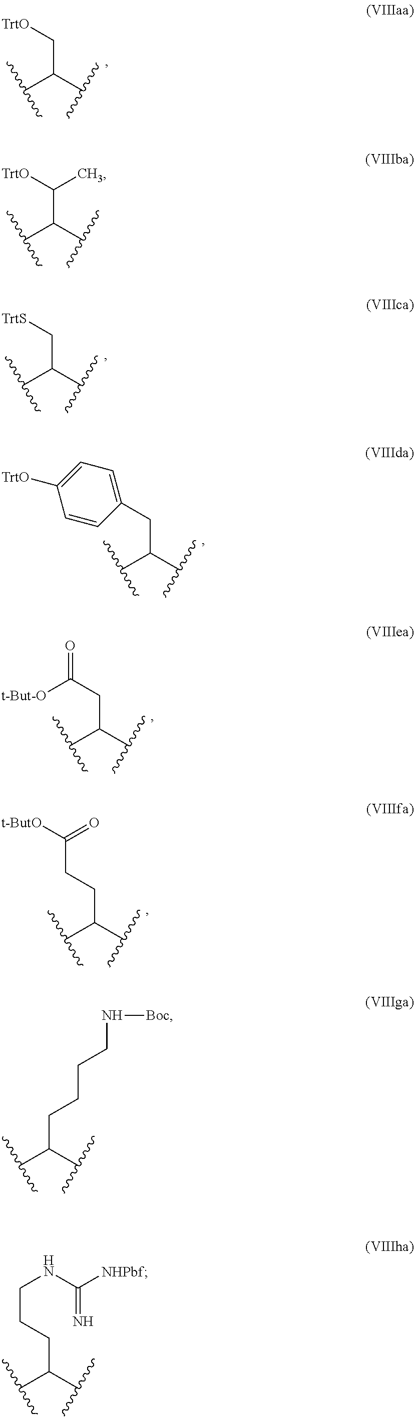

wherein Trt is the trityl protecting group; t-But is the t-Butyl protecting group; Boc is the Boc protecting group; Pfb is the (2,2,4,6,7-pentamethyl-dihydrobenzofuran-5-sulfonyl)—protecting group;
are obtained by reaction with pNO₂—C₆H₄—OCOCl of the corresponding compounds (Xgₐ)-(Xgₑ):

NH₂—Kₐ—COO-A+pNO₂—C₆H₄—OCOCl (Xgₐ)

NH₂—Kₐ—CONH—Y—ONO₂+pNO₂—C₆H₄—OCOCl (Xg_{b})

NH₂—Kₐ—CH₂O—CO—Y—ONO₂+pNO₂—C₆H₄—OCOCl (Xg_{c})

NH₂—Kₐ—CH₂O—CO—O—Y—ONO₂+pNO₂—C₆H₄—OCOCl (Xg_{d})

NH₂—Kₐ—CH₂O—CO—NH—Y—ONO₂+pNO₂—C₆H₄—OCOCl (Xgₑ)

wherein A, Y and Kₐ are as previously defined.

Compounds of formula (Xgₐ)-(Xgₑ) depending on the meaning of Kₐ can be obtained starting from the corresponding N-Fmoc protected amino acid (VIIIab)-(VIIIhb):

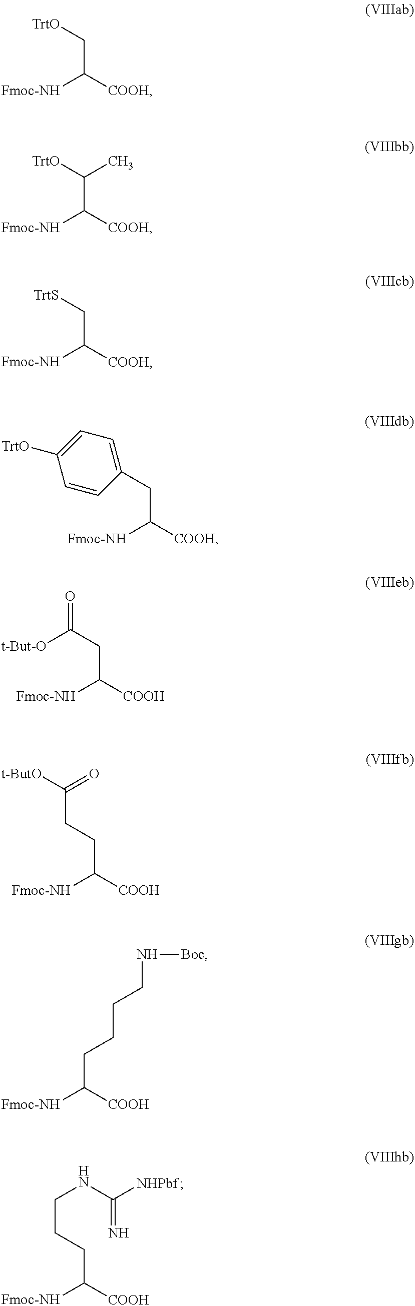

or corresponding N-Fmoc protected amino alcohol (VIIIac)-(VIIIhc):

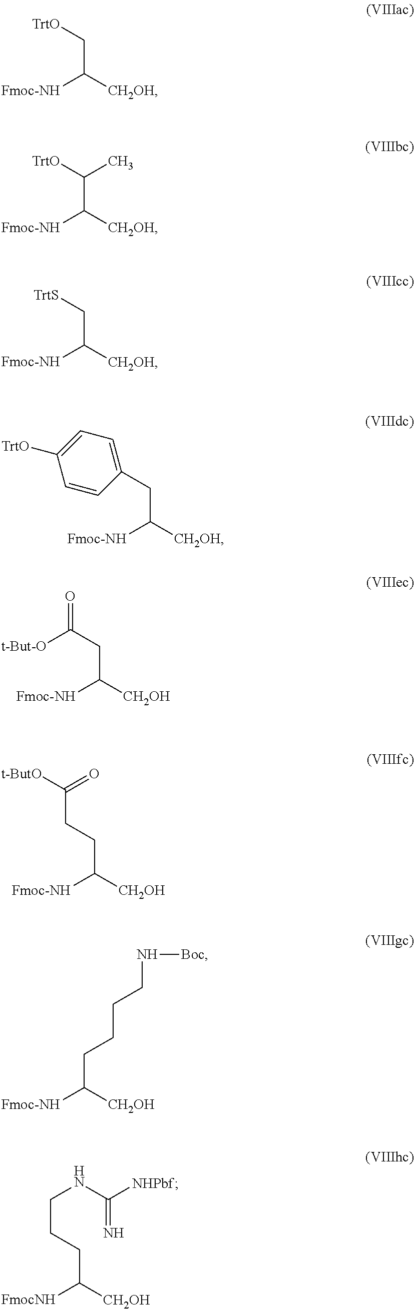

following the procedure described in A1-A5 depending on the meaning of K* that is equal to K′, eventually hydrolyzing the Fmoc protected group following methods well known in the literature.

### A8. Synthesis of Compounds (Xfₐ)-(Xfₑ) and (Xhₐ)-(Xhₑ)

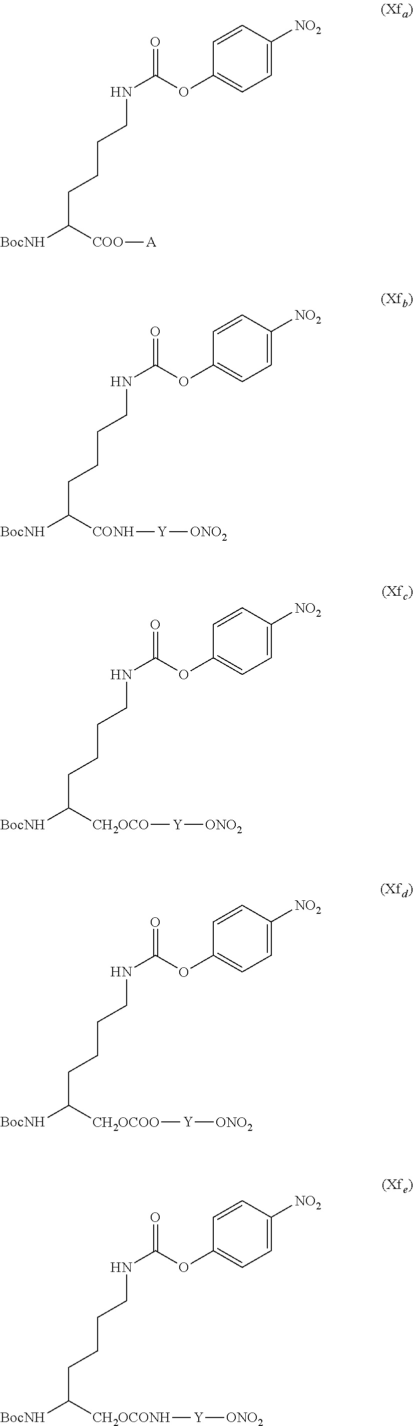

Wherein A, Y and are as previously defined in A1.

The compounds of formula (Xfₐ)-(Xfₑ) wherein A and Y are as previously defined in A1., are obtained by reaction with pNO₂—C₆H₄—OCOCl of the corresponding compounds (Xhₐ)-(Xhₑ):

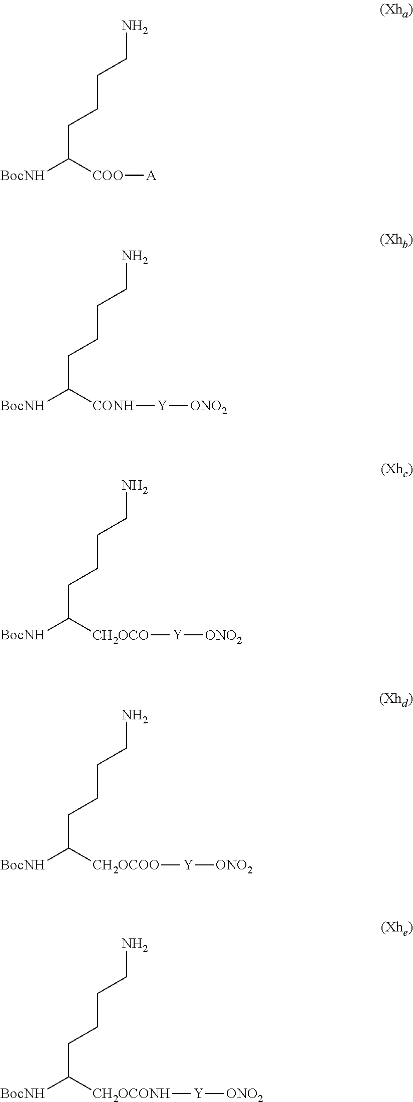

wherein A, Y and Boc are as previously defined.

Compounds of formula (Xhₐ)-(Xhₑ) depending on the meaning of K* that is equal to K′ can be obtained starting from the corresponding N-Fmoc protected derivatives (IXaa) or (IXab):

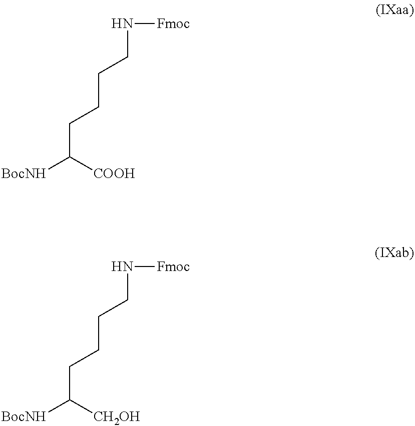

following the procedure described in A1-A5 depending on the meaning of K*, eventually hydrolyzing the Fmoc protected group following methods well known in the literature.

### A9/1. Synthesis of Compounds (Xiₐ₁), (Xiₐ₂), (Xiₐ₃) and (Xkₐ₁), (Xkₐ₂) and (Xkₐ₃)

Compounds of formula

p-NO₂—C₆H₄—O—CO—NH—K₁—COO—Y—ONO₂ (Xiₐ₁)-(Xiₐ₃)

wherein Y is as above described and K₁ is selected from (VIIIa)-(VIIId) wherein R₅ is selected from respectively:
i) —CO— (Xiₐ₁);
ii) —COO— (Xiₐ₂); or
iii) —CONH— (Xiₐ₃);
   and K₁ is bound to the group —(Y′—ONO₂) are obtained by reaction with pNO₂—C₆H₄—OCOCl of the corresponding compounds (Xkₐ₁)-(Xkₐ₃):

NH₂—K₁—COO—Y—ONO₂+pNO₂—C₆H₄—OCOCl (Xkₐ₁)-(Xkₐ₃)

wherein Y is as previously defined and K₁ is selected from (VIIIa)-(VIIId) wherein R₅ is selected from respectively:
i) —CO— (Xkₐ₁);
ii) —COO— (Xkₐ₂); or
iii) —CONH— (Xkₐ₃);

Compounds of formula (Xkₐ₁)-(Xkₐ₃) can be obtained as follows:

1) (Xkₐ₁): wherein K₁ is selected from (VIIIa)-(VIIId) wherein R₅ is —CO:
1a) by reacting Fmoc protected compounds of formula (XVa₁)-(XVd₁) (Scheme 6a) with compounds (XIa) applying the same procedure described in A1, eventually hydrolyzing the Fmoc protective groups of the resulting (XVIa₁)-(XVId₁) following methods known in the literature.

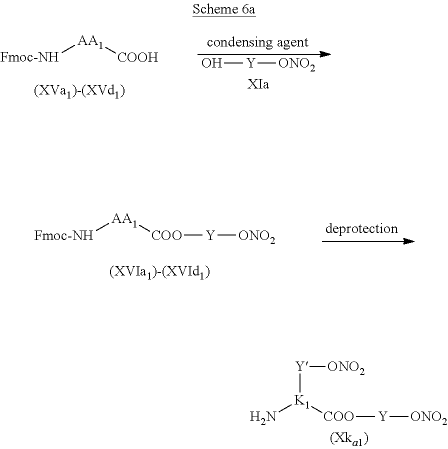

wherein Y is as above described; AA₁ are respectively the residue of formula (XVaa)-(XVad):

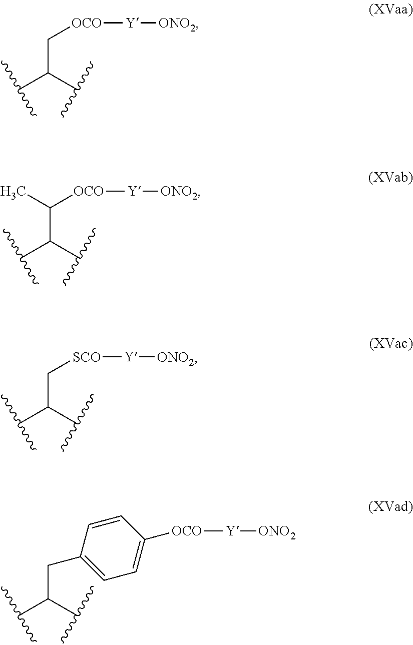

wherein Y′ as above defined; following the procedure already described in A1;
1b) Compounds of formula (XVa₁)-(XVd₁) are obtained by reacting respectively the corresponding commercially available Fmoc-serine, Fmoc-treonine, Fmoc-cysteine, or Fmoc-tyrosine with compounds (XIc):

C₆F₅O—CO—Y′—ONO₂ (XIc)

following procedure described in WO 2006/008196. Compounds (XIc) have been already described in A3.
2) (Xkₐ₂): wherein K₁ is selected from (VIIIa)-(VIIId) wherein R₅ is —COO:
2a) by reacting Fmoc protected compounds of formula (XVa₂)-(XVd₂) with compounds (XIa) applying the procedure described in Scheme 6b:

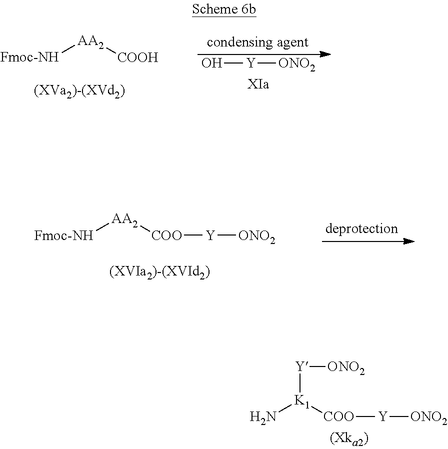

wherein Y is as above described; AA₂ are respectively the residue of formula (XVba)-(XVbd):

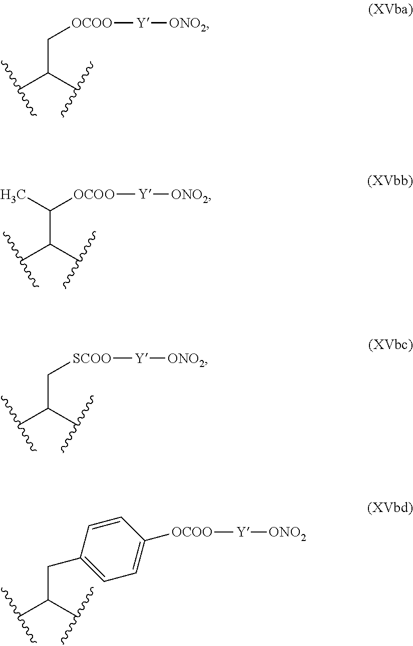

wherein Y′ is as above defined;
2b) Compounds of formula (XVa₂)-(XVd₂) are obtained by reacting respectively the corresponding commercially available Fmoc-serine, Fmoc-treonine, Fmoc-cysteine, or Fmoc-tyrosine with compounds (XId):

p-NO₂—C₆H₄O—COO—Y′—ONO₂ (XId)

following procedure described in WO 2006/008196. Compounds (XId) have been already described in A4.
3) (Xkₐ₃): wherein K₁ is selected from (VIIIa)-(VIIId) wherein R₅ is —CONH—:
3a) by reacting Fmoc protected compounds of formula (XVa₃)-(XVd₃) with compounds (XIa) applying the procedure described in Scheme 6c:

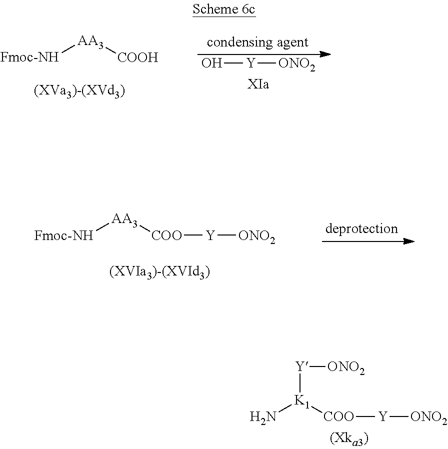

wherein Y is as above described; AA₃ are respectively the residue of formula (XVca)-(XVcd):

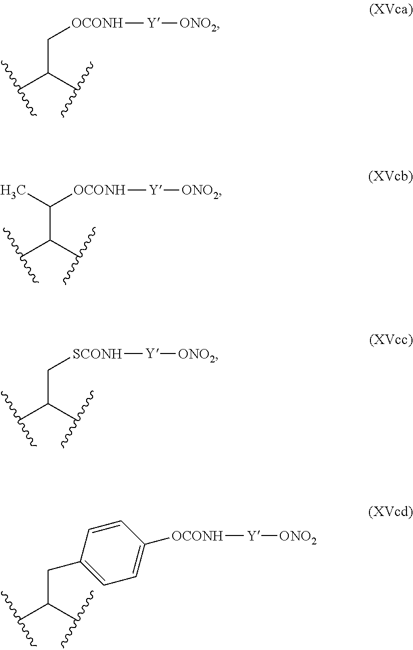

wherein Y′ is as above defined;
3b) Compounds of formula (XVa₃)-(XVd₃) as above defined are obtained by reacting respectively the corresponding commercially available Fmoc-serine, Fmoc-treonine, Fmoc-cysteine, or Fmoc-tyrosine with compounds (XIe):

p-NO₂—C₆H₄O—CONH—Y′—ONO₂ (XIe)

following procedure described in A5.

### A9/2. Synthesis of Compounds (Xiₐ₄), (Xiₐ₅), (Xiₐ₆) and (Xkₐ₄), (Xkₐ₅) and (Ckₐ₆)

Compounds of formula

p-NO₂—C₆H₄—O—CO—NH—K₁—COOtBut (Xiₐ₄)-(Xiₐ₆)

wherein K₁ is selected from (VIIIa)-(VIIId) wherein R₅ is selected from respectively:
i) —CO— (Xiₐ₄);
ii) —COO— (Xiₐ₅); or
iii) —CONH— (Xiₐ₆);
   and K₁ is bound to the group —(Y′—ONO₂) are obtained by reaction with pNO₂—C₆H₄—OCOCl of the corresponding compounds (Xkₐ₄)-(Xkₐ₆):

NH₂—K₁—COOtBut+pNO₂—C₆H₄—OCOCl (Xkₐ₄)-(Xkₐ₆)

wherein K₁ is selected from (VIIIa)-(VIIId) wherein R₅ is selected from respectively:
i) —CO— (Xkₐ₄);
ii) —COO— (Xkₐ₅); or
iii) —CONH— (Xkₐ₆);

Compounds of formula (Xkₐ₄)-(Xkₐ₆) can be obtained

1) (Xkₐ₄): wherein K₁ is selected from (VIIIa)-(VIIId) wherein R₅ is —CO are obtained:
by reacting respectively the corresponding commercially available compound (VIIIad), (VIIIbd), (VIIIcd) and (VIIIdd) with compounds (XIc) (Scheme 6d):

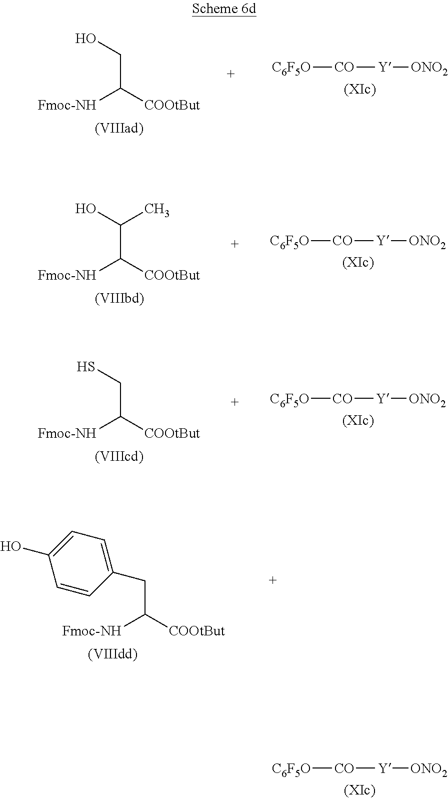

wherein Y′ is as above described, following procedure described A3 and eventually deprotecting the Fmoc group by known methods.
2) (Xkₐ₅): wherein K₁ is selected from (VIIIa)-(VIIId) wherein R₅ is —COO:
are obtained by reacting respectively the corresponding commercially available compound (VIIIad), (VIIIbd), (VIIIcd) and (VIIIdd) with compounds (XId) (Scheme 6e):

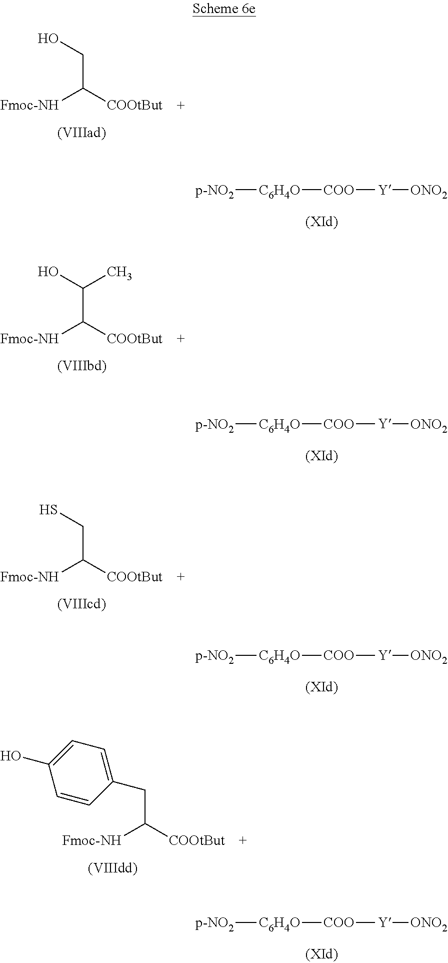

wherein Y′ is as above described, following procedure described A4 and eventually deprotecting the Fmoc group by known methods.
3) (Xkₐ₆): wherein K₁ is selected from (VIIIa)-(VIIId) wherein R₅ is —CONH—:
are obtained by reacting respectively the corresponding commercially available compound (VIIIad), (VIIIbd), (VIIIcd) and (VIIIdd) with compounds (XIe) (Scheme 6f):

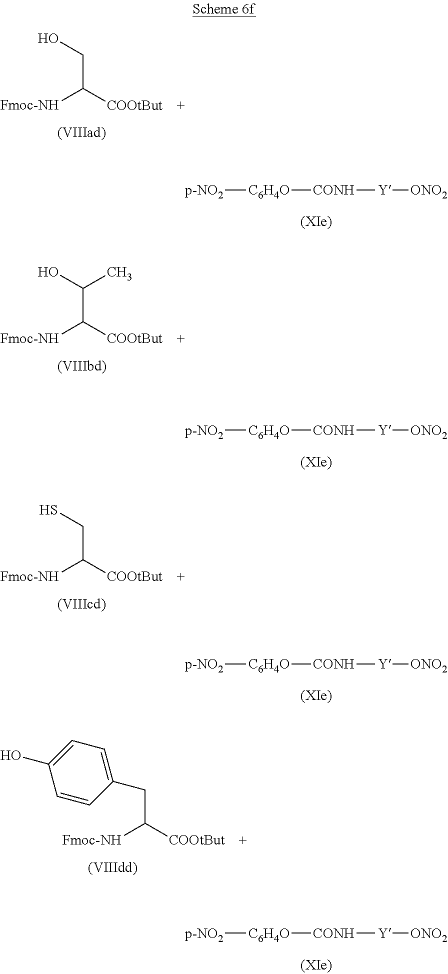

wherein Y′ is as above described, following procedure described A5 and eventually deprotecting the Fmoc group by known methods.

### A10. Synthesis of Compounds (Xi_{b1}), (Xi_{b2}), (Xi_{b3}) and (Xk_{b1}), (Xk_{b2}) and (Xk_{b3})

Compounds of formula

p-NO₂—C₆H₄—O—CO—NH—K₁—CONH—Y—ONO₂ (Xi_{b1})-(Xi_{b3})

wherein Y is as above described and K₁ is selected from (VIIIa)-(VIIId) wherein R₅ is selected from respectively:
i) —CO— (Xi_{b1});
ii) —COO— (Xi_{b2}); or
iii) —CONH— (Xi_{b3});
   and K₁ is bound to the group —(Y′—ONO₂) are obtained by reaction with pNO₂—C₆H₄—OCOCl of the corresponding compounds (Xk_{b1})-(Xk_{b3})

NH₂—K₁—CONH—Y—ONO₂+pNO₂—C₆H₄—OCOCl (Xk_{b1})-(Xk_{b3})

wherein Y and K₁ are as above defined;

Compounds of formula (Xk_{b1})-(Xk_{b3}) can be obtained as follow:

1) (Xk_{b1}): wherein K₁ is selected from (VIIIa)-(VIIId) wherein R₅ is —CO:
1a) by reacting compounds of formula (XVa₁)-(XVd₁) obtained as described in A9 with compounds (XIb) applying the same procedure described in A2, eventually hydrolyzing the Fmoc protective groups of the resulting (XVIa₄)-(XVId₄) following methods known in the literature (Scheme 7a):

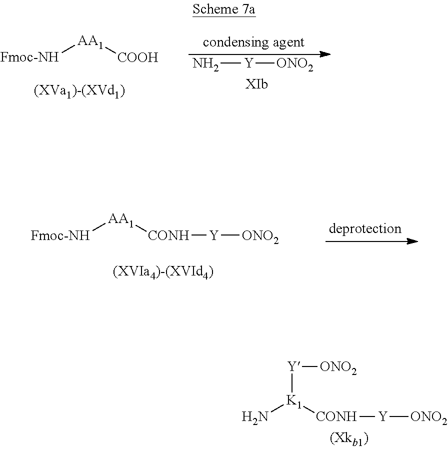

wherein Y and AA₁ are as previously defined.
2) (Xk_{b2}): wherein K₁ is selected from (VIIIa)-(VIIId) wherein R₅ is —COO:
2a) by reacting Fmoc protected compounds of formula (XVa₂)-(XVd₂) obtained as described in A9 with compounds (XIb) then deprotecting the obtained compounds (XVIa₅)-(XVId₅) applying procedures already described (Scheme 7b):

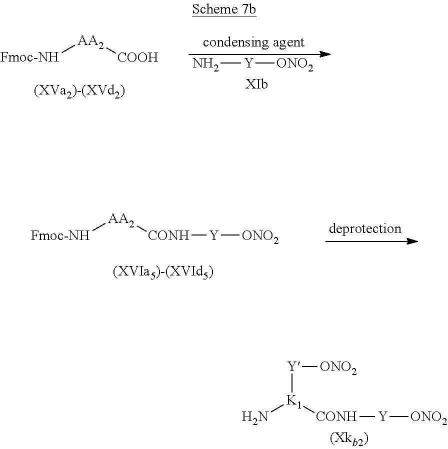

wherein Y and AA₂ are as already described;
3) (Xk_{b3}): wherein K₁ is selected from (VIIIa)-(VIIId) wherein R₅ is —CONH—:
3a) by reacting Fmoc protected compounds of formula (XVa₃)-(XVd₃) with compounds (XIb) applying the same procedure described above (Scheme 7c):

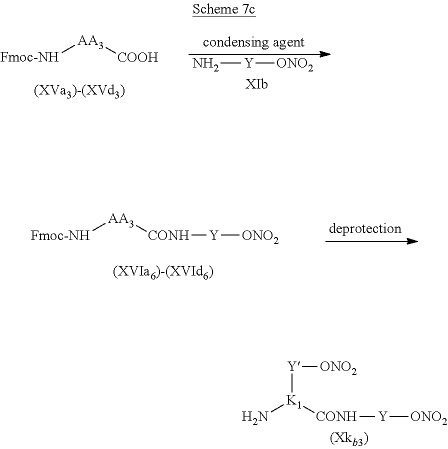

wherein Y and AA₃ are as previously defined;

### A11. Synthesis of Compounds (Xi_{c1}), (Xi_{c2}), (Xi_{c3}) and (Xk_{c1}), (Xk_{c2}) and (Xk_{c3})

Compounds of formula

p-NO₂—C₆H₄—O—CO—NH—K₁—CH₂O—CO—Y—ONO₂ (Xi_{c1})-(Xi_{c3})

wherein Y is as above described and K₁ is selected from (VIIIa)-(VIIId) wherein R₅ is selected from respectively:
i) —CO— (Xi_{c1});
ii) —COO— (Xi_{c2}); or
iii) —CONH— (Xi_{c3});
   and K₁ is bound to the group —(Y′—ONO₂) are obtained by reaction with pNO₂—C₆H₄—OCOCl of the corresponding compounds (Xk_{c1})-(Xk_{c3}):

NH₂—K₁—CH₂O—CO—Y—ONO₂+pNO₂—C₆H₄—OCOCl (Xk_{c1})-(Xk_{c3})

wherein Y and K₁ are as above defined;

Compounds of formula (Xk_{c1})-(Xk_{c3}) can be obtained as follow:

1) (Xk_{c1}): wherein K₁ is selected from (VIIIa)-(VIIId) wherein R₅ is —CO:
1a) by reacting Fmoc protected compounds of formula (XVa₄)-(XVd₄) with compounds (XIc) applying the same procedure described in A3, eventually hydrolyzing the Fmoc protective groups of the resulting (XVIa₇)-(XVId₇) following methods known in the literature (Scheme 8a):

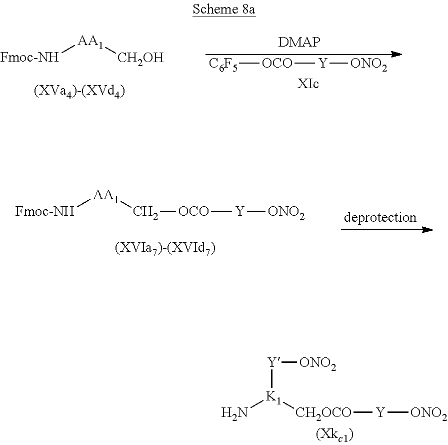

wherein Y is as above defined and AA₁ has been already described in A9;
2) (Xk_{c2}): wherein K₁ is selected from (VIIIa)-(VIIId) wherein R₅ is —COO:
2a) by reacting Fmoc protected compounds of formula (XVa₅)-(XVd₅) with compounds (XIc) applying the same procedure described above (Scheme 8b):

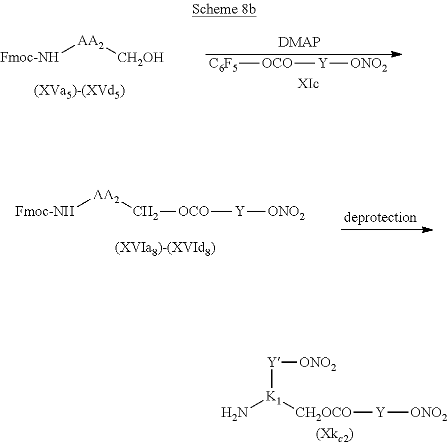

wherein Y and AA₂ hays been already defined;
3) (Xk_{c3}): wherein K₁ is selected from (VIIIa)-(VIIId) wherein R₅ is —CONH—:
3a) by reacting Fmoc protected compounds of formula (XVa₆)-(XVd₆) with compounds (XIb) applying the same procedure described above (Scheme 8c):

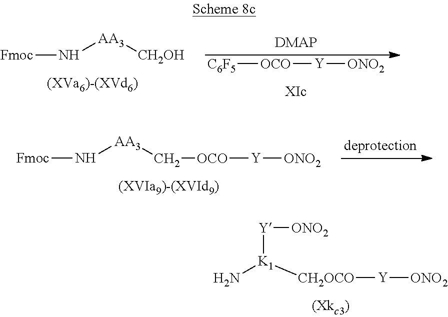

wherein Y and AA₃ have been already described.

### A12. Synthesis of Compounds (Xi_{d1}), (Xi_{d2}), (Xi_{d3}) and (Xk_{d1}), (Xk_{d2}) and (Xk_{d3})

Compounds of formula:

p-NO₂—C₆H₄—O—CO—NH—K₁—CH₂O—COO—Y—ONO₂ (Xi_{d1})-(Xi_{d3})

wherein Y is as above described and K₁ is selected from (VIIIa)-(VIIId) wherein R₅ is selected from respectively:
i) —CO— (Xi_{d1});
ii) —COO— (Xi_{d2}); or
iii) —CONH— (Xi_{d3});
   and K₁ is bound to the group —(Y′—ONO₂) are obtained by reaction with pNO₂—C₆H₄—OCOCl of the corresponding compounds (Xk_{d1})-(Xk_{d3}):

NH₂—K₁—CH₂O—COO—Y—ONO₂+pNO₂—C₆H₄—OCOCl (Xk_{d1})-(Xk_{d3})

wherein Y and K₁ are as above defined;

Compounds of formula (Xk_{d1})-(Xk_{d3}) can be obtained as follow:

1) (Xk_{d1}): wherein K₁ is selected from (VIIIa)-(VIIId) wherein R₅ is —CO:
1a) by reacting Fmoc protected compounds of formula (XVa₄)-(XVd₄) with compounds (XId) applying the same procedure described in A4, eventually hydrolyzing the Fmoc protective groups of the resulting (XVIa₁₀)-(XVId₁₀) following methods known in the literature (Scheme 9a):

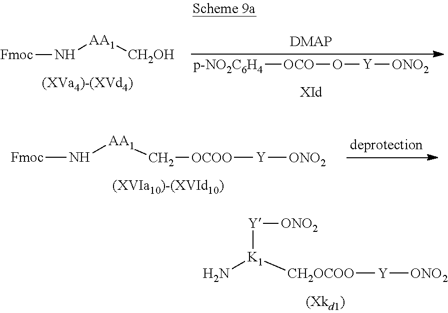

wherein Y and AA₁ have been already described in A9;
2) (Xk_{d2}): wherein K₁ is selected from (VIIIa)-(VIIId) wherein R₅ is —COO:
2a) by reacting Fmoc protected compounds of formula (XVa₅)-(XVd₅) with compounds (XId) applying the same procedure described above. (Scheme 9b):

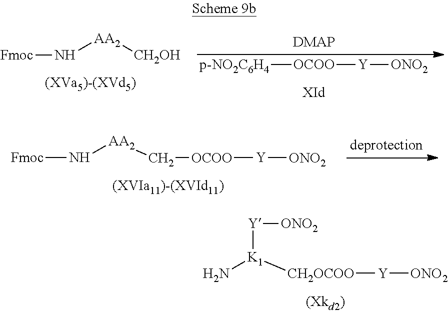

wherein Y and AA₂ have been already defined;
3) (Xkₐ₃): wherein K₁ is selected from (VIIIa)-(VIIId) wherein R₅ is —CONH—:
3a) by reacting Fmoc protected compounds of formula (XVa₆)-(XVd₆) with compounds (XId) applying the same procedure described above (Scheme 9c):

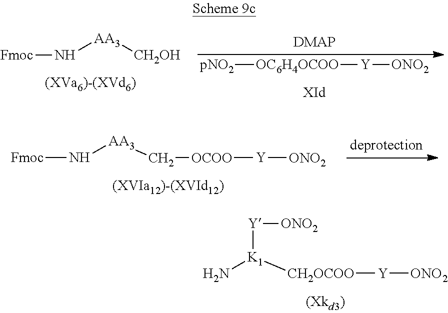

wherein Y and AA₃ have been already described.

### A13. Synthesis of Compounds (Xiₑ₁), (Xiₑ₂), (Xiₑ₃) and (Xkₑ₁), (Xkₑ₂) and (Xkₑ₃)

Compounds of formula

p-NO₂—C₆H₄—O—CO—NH—K₁—CH₂O—CONH—Y—ONO₂ (Xiₑ₁)-(Xiₑ₃)

wherein Y is as above described and K₁ is selected from (VIIIa)-(VIIId) wherein R₅ is selected from respectively:
i) —CO— (Xiₑ₁);
ii) —COO— (Xiₑ₂); or
iii) —CONH— (Xiₑ₃);
   and K₁ is bound to the group —(Y′—ONO₂) are obtained by reaction with pNO₂—C₆H₄—OCOCl of the corresponding compounds (Xkₑ₁)-(Xkₑ₃):

NH₂—K₁—CH₂O—CONH—Y—ONO₂+pNO₂—C₆H₄—OCOCl (Xkₑ₁)-(Xkₑ₃)

wherein Y and K₁ are as above defined;

Compounds of formula (Xkₑ₁)-(Xkₑ₃) can be obtained as follow:

1) (Xkₑ₁): wherein K₁ is selected from (VIIIa)-(VIIId) wherein R₅ is —CO:
1a) by reacting Fmoc protected compounds of formula (XVa₄)-(XVd₄) with compounds (XIe) applying the same procedure described in A5, eventually hydrolyzing the Fmoc protective groups of the resulting (XVIa₁₃)-(XVId₁₃) following methods known in the literature (Scheme 10a):

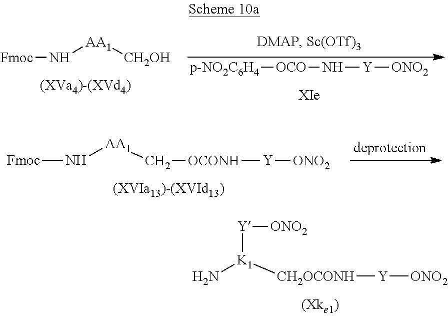

wherein Y and AA₁ have been already described;
2) (Xkₑ₂): wherein K₁ is selected from (VIIIa)-(VIIId) wherein R₅ is —COO:
2a) by reacting Fmoc protected compounds of formula (XVa₅)-(XVd₅) with compounds (XIe) applying the same procedure described above (Scheme 10b):

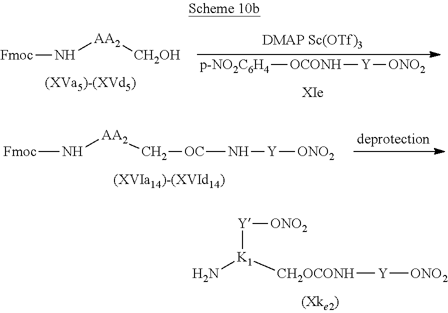

wherein Y and AA₂ have been already defined;
3) (Xkₑ₃): wherein K₁ is selected from (VIIIa)-(VIIId) wherein R₅ is —CONH—:
3a) by reacting Fmoc protected compounds of formula (XVa₆)-(XVd₆) with compounds (XIe) applying the same procedure described above (Scheme 10c):

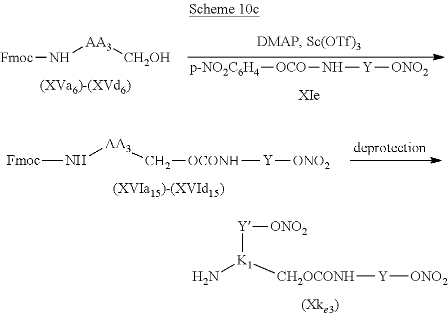

wherein Y and AA₃ have been already described.

### A14/1. Synthesis of Compounds (Xi_{f1}), (Xi_{f2}) and (Xk_{f1})-(Xk_{f2})

Compounds of formula

p-NO₂—C₆H₄—O—CO—NH—K₂—COO—Y—ONO₂ (Xi_{f1}), (Xi_{f2})

wherein Y is as above described and K₂ is selected from (VIIIe), (VIIIf) wherein R₆ is selected from respectively:
i) —O— (Xi_{f1}) or
ii) —NH— (Xi_{f2})
   and K₂ is bound to the group —(Y′—ONO₂) are obtained by reaction with pNO₂—C₆H₄—OCOCl of the corresponding compounds (Xk_{f1}), (Xk_{f2}):

NH₂—K₂—COO—Y—ONO₂+pNO₂—C₆H₄—OCOCl (Xk_{f1})-(Xk_{f2})

wherein Y is as previously defined and K₂ is selected from:
i) —O— (Xk_{f1}) or
ii) —NH— (Xk_{f2})

Compounds of formula (Xk_{f1}), (Xk_{f2}) can be obtained as follows:

1) (Xk_{f1}): wherein Y and Y′ are equal and K₂ is selected from (VIIIe), (VIIIf) wherein R₆ is —O—:
1a) by reacting the Fmoc-glutamic acid or Fmoc-glutaric acid with compounds (XIa) applying the same procedure described in A1 and using 2 equivalents of (XIa), eventually hydrolyzing the Fmoc protective groups following methods known in the literature.
1′) (Xk_{f1}): wherein Y and Y′ are different and K₂ is selected from (VIIIe), (VIIIf) wherein R₆ is —O—:
1a′) by reacting compounds of formula (XVIIa₁), (XVIIb₁) with compounds (XIa) following procedure already described in A1 (Scheme 11a):

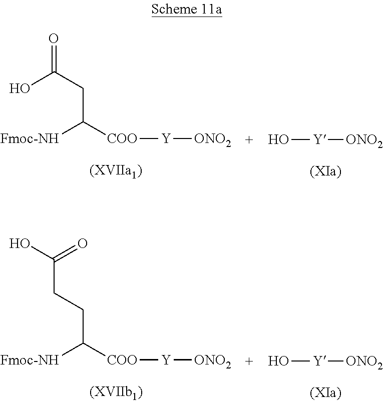

Eventually deprotecting the Fmoc group by known methods. Compounds (XVIIa₁) and (XVIIb₁) were obtained by reacting compounds (VIIIeb) and (VIIIfb) above described in A7 with compounds (XIa), eventually acid hydrolyzing the t-butyl ester group:

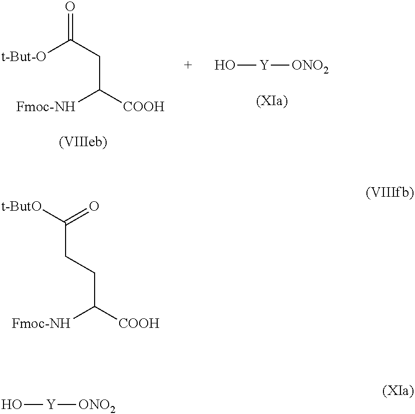

2) (Xk_{f2}): wherein Y is as above defined and K₂ is selected from (VIIIe), (VIIIf) wherein R₆ is —NH—:
2a) by reacting compounds of formula (XVIIa₁), (XVIIb₁) already defined with compounds (XIb) following procedure already described in A2 (Scheme 11b):

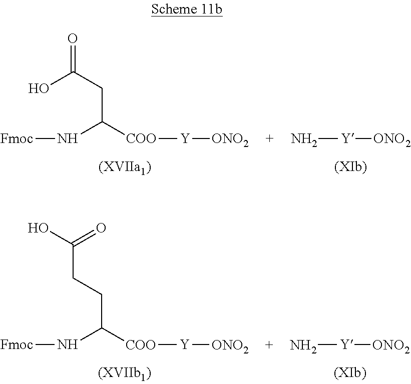

Eventually deprotecting the Fmoc group by known methods.

### A14/2. Synthesis of Compounds (Xi_{f3}), (Xi_{f4}) and (Xk_{f3})-(Xk_{f4})

Compounds of formula

p-NO₂—C₆H₄—O—CO—NH—K₂—COOtBut (Xi_{f3}), (Xi_{f4})

wherein K₂ is selected from (VIIIe), (VIIIf) wherein R₆ is selected from respectively:
i) —O— (Xi_{f3}) or
ii) —NH— (Xi_{f4})
   and K₂ is bound to the group —(Y′—ONO₂) are obtained by reaction with pNO₂—C₆H₄—OCOCl of the corresponding compounds (Xk_{f3}), (Xk_{f4}):

NH₂—K₂—COOtBut+pNO₂—C₆H₄—OCOCl (Xk_{f3})-(Xk_{f4})

wherein K₂ is selected from:
i) —O— (Xk_{f3}) or
ii) —NH— (Xk_{f4})

Compounds of formula (Xk_{f3}), (Xk_{f4}) can be obtained as follows:

1) (Xk_{f3}): wherein K₂ is selected from (VIIIe), (VIIIf) wherein R₆ is —O—:
1a) by reacting commercially available compounds of formula (VIIIde) and (VIIIdf) with compounds (XIa) applying the same procedure described in A1 (Scheme 11c) eventually hydrolyzing the Fmoc protective groups following methods known in the literature.

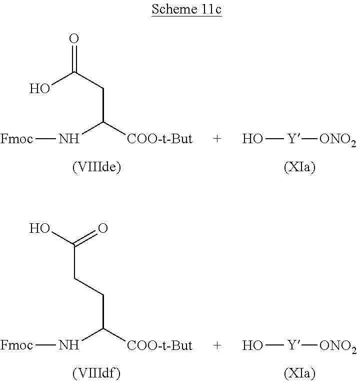

2) (Xk_{f4}): wherein K₂ is selected from (VIIIe), (VIIIf) wherein R₆ is —NH—:
2a) by reacting commercially available compounds of formula (VIIIde) and (VIIIdf) with compounds (XIb) applying the same procedure described in A2 (Scheme 11d) eventually hydrolyzing the Fmoc protective groups following methods known in the literature.

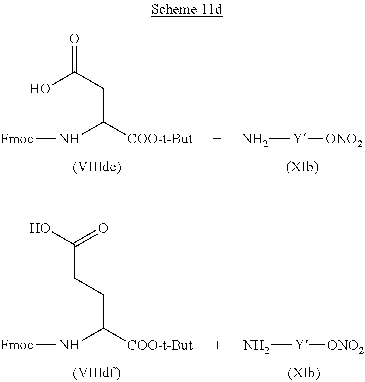

### A15. Synthesis of Compounds (Xi_{g1}), (Xi_{g2}) and (Xk_{g1})-(Xk_{g2})

Compounds of formula

p-NO₂—C₆H₄—O—CO—NH—K₂—CONH—Y—ONO₂ (Xi_{g1}), (Xi_{g2})

Wherein Y is as above described and K₂ is selected from (VIIIe), (VIIIf) wherein R₆ is selected from respectively:
i) —O— (Xi_{g1}) or
ii) —NH— (Xi_{g2})
   and K₂ is bound to the group —(Y′—ONO₂) are obtained by reaction with pNO₂—C₆H₄—OCOCl of the corresponding compounds (Xk_{g1}), (Xk_{g2})

NH₂—K₂—CONH—Y—ONO₂+pNO₂—C₆H₄—OCOCl (Xk_{g1})-(Xk_{g2})

Wherein Y is as previously defined and K₂ is selected from:
i) —O— (Xk_{g1}) or
ii) —NH— (Xk_{g2})

Compounds of formula (Xk_{g1}), (Xk_{g2}) can be obtained as follow:

1) (Xk_{g1}): wherein Y is as above defined and K₂ is selected from (VIIIe), (VIIIf) wherein R₆ is —O—:
1a) by reacting compounds of formula (XVIIa₂), (XVIIb₂) with compounds (XIa) following procedure already described in A1 (Scheme 12):

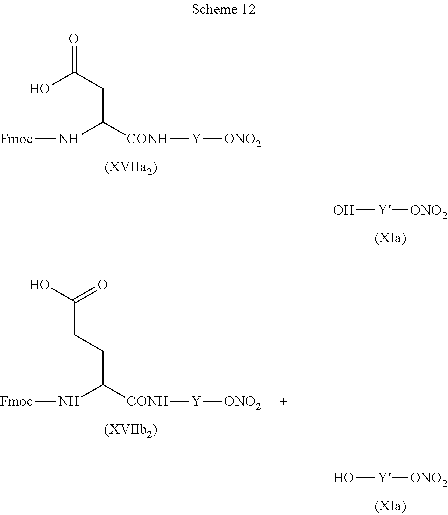

Eventually deprotecting the Fmoc group by known methods. Compounds (XVIIa₂) and (XVIIb₂) can be obtained by reacting compounds (VIIIeb) or (VIIIfb) with compounds (XIb) following procedure described in A2 (Scheme 15):

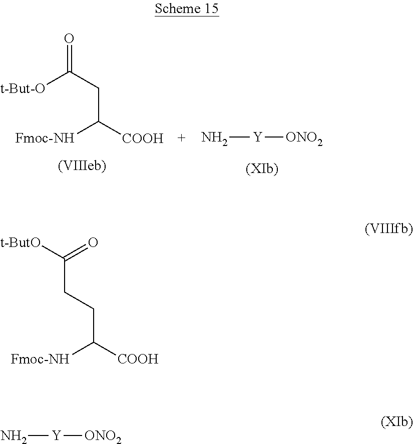

2) (Xk_{g2}): wherein Y and Y′ are equal or different and K₂ is selected from (VIIIe), (VIIIf) wherein R₆ is —NH—:
2a) wherein Y and Y′ are equal: by reacting the Fmoc-glutamic acid or Fmoc-glutaric acid with compounds (XIb) applying the same procedure described in A2 and using 2 equivalent of (XIb), eventually hydrolyzing the Fmoc protective groups following methods known in the literature.
2a′) wherein Y and Y′ are different: by reacting compounds (XVIIa₂) and (XVIIb₂) above described with compounds (XIb) as above described, (Scheme 16) eventually hydrolyzing the Fmoc protecting group:

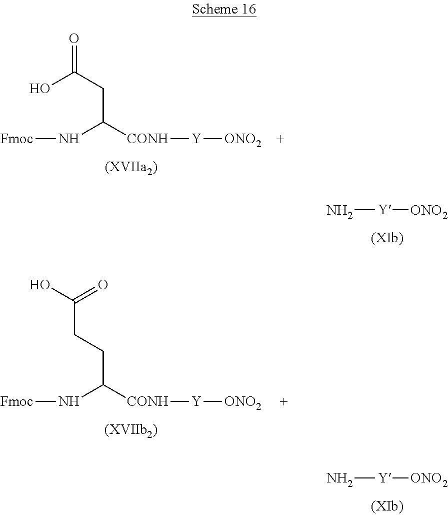

### A16. Synthesis of Compounds (Xiₕ₁), (Xiₕ₂) and (Xkₕ₁)-(Xkₕ₂)

Compounds of formula

p-NO₂—C₆H₄—O—CO—NH—K₂—CH₂OCO—Y—ONO₂ (Xiₕ₁), (Xiₕ₂)

Wherein Y is as above described and K₂ is selected from (VIIIe), (VIIIf) wherein R₆ is selected from respectively:
i) —O— (Xiₕ₁) or
ii) —NH— (Xiₕ₂)
   and K₂ is bound to the group —(Y′—ONO₂) are obtained by reaction with pNO₂—C₆H₄—OCOCl of the corresponding compounds (Xkₕ₁), (Xkₕ₂):

NH₂—K₂—CH₂OCO—Y—ONO₂+pNO₂—C₆H₄—OCOCl (Xkₕ₁)-(Xkₕ₂)

wherein Y is as previously defined and K₂ is selected from:
i) —O— (Xkₕ₁) or
ii) —NH— (Xkₕ₂)

Compounds of formula (Xkₕ₁), (Xkₕ₂) can be obtained as follow:

1) (Xkₕ₁): wherein Y is as above defined and K₂ is selected from (VIIIe), (VIIIf) wherein R₆ is —O— and K₂ binds a group —Y′—ONO₂:
1a) by reacting compounds of formula (XVIIa₃), (XVIIb₃) with compounds (XIa) following procedure already described in A3 (Scheme 15):

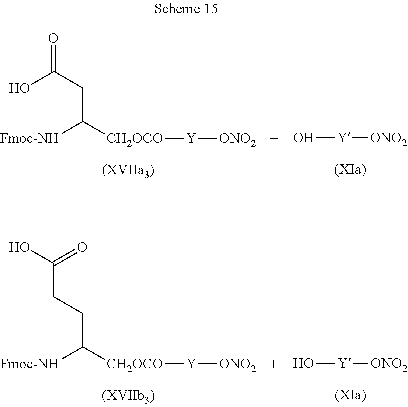

Eventually deprotecting the Fmoc group by known methods. Compounds (XVIIa₃) and (XVIIb₃) can be obtained by reacting compounds (VIIIec) or (VIIIfc) with compounds (XIc) following procedure described in A3 (Scheme 16)

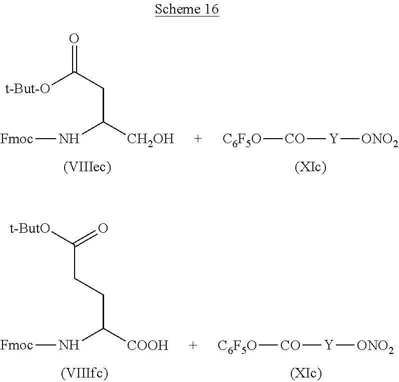

2) (Xkₕ₂): wherein Y is as above defined and K₂ is selected from (VIIIe), (VIIIf) wherein R₆ is —NH—:
2a) by reacting compounds (XVIIa₃) and (XVIIb₃) above described with compounds (XIb) as above described, (Scheme 17):

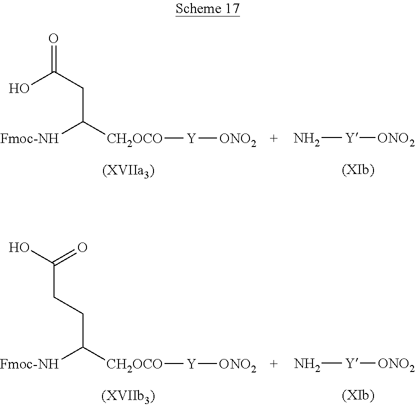

eventually hydrolyzing the Fmoc protecting group as already described.

### A17. Synthesis of Compounds (Xiᵢ₁), (Xiᵢ₂) and (Xkᵢ₁)-(Xkᵢ₂)

Compounds of formula

p-NO₂—C₆H₄—O—CO—NH—K₂—CH₂OCOO—Y—ONO₂ (Xiᵢ₁), (Xiᵢ₂)

wherein Y is as above described and K₂ is selected from (VIIIe), (VIIIf) wherein R₆ is selected from respectively:
i) —O— (Xiᵢ₁) or
ii) —-NH— (Xiᵢ₂)
   and K₂ is bound to the group —(Y′—ONO₂) are obtained by reaction with pNO₂—C₆H₄—OCOCl of the corresponding compounds (Xkᵢ₁), (Xkᵢ₂)

NH₂—K₂—CH₂OCOO—Y—ONO₂+pNO₂—C₆H₄—OCOCl (Xkᵢ₁)-(Xkᵢ₂)

Wherein Y is as previously defined and K₂ is selected from:
i) —O— (Xkᵢ₁) or
ii) —NH— (Xkᵢ₂)

Compounds of formula (Xkᵢ₁), (Xkᵢ₂) can be obtained as follow:

1) (Xkᵢ₁): wherein Y is as above defined and K₂ is selected from (VIIIe), (VIIIf) wherein R₆ is —O—:
1a) by reacting compounds of formula (XVIIa₄), (XVIIb₄) with compounds (XIa) following procedure already described in A3 (Scheme 18):

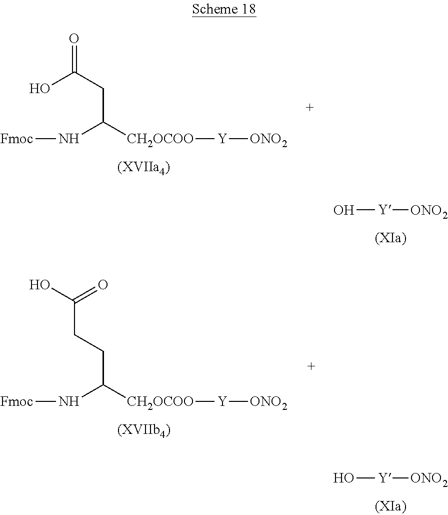

Eventually deprotecting the Fmoc group by known methods. Compounds (XVIIa₄) and (XVIIb₄) can be obtained by reacting compounds (VIIIec) or (VIIIfc) with compounds (XId) following procedure described in A4 (Scheme 19):

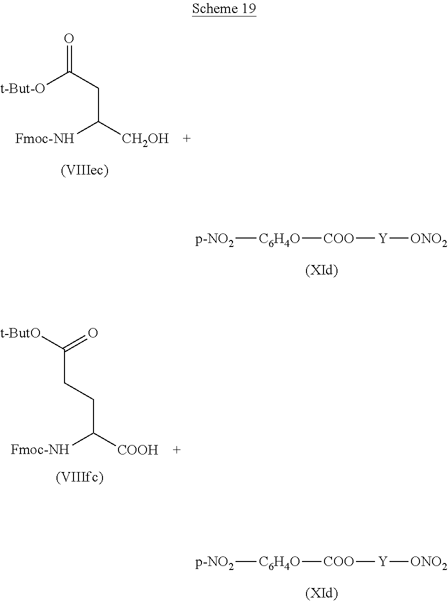

2) (Xkᵢ₂): wherein Y is as above defined and K₂ is selected from (VIIIe), (VIIIf) wherein R₆ is —NH—:
2a) by reacting compounds (XVIIa₄) and (XVIIb₄) above described with compounds (XIb) as above described, (Scheme 20):

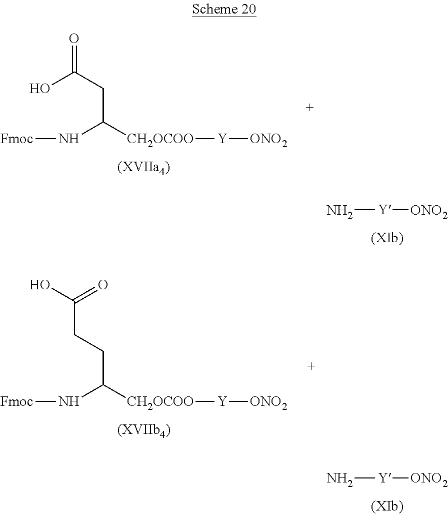

eventually hydrolyzing the Fmoc protecting group as already described.

### A18. Synthesis of Compounds (Xiⱼ₁), (Xiⱼ₂) and (Xkⱼ₁)-(Xkⱼ₂)

Compounds of formula

p-NO₂—C₆H₄—O—CO—NH—K₂—CH₂OCONH—Y—ONO₂ (Xiⱼ₁), (Xiⱼ₂)

wherein Y is as above described and K₂ is selected from (VIIIe), (VIIIf) wherein R₆ is selected from respectively:
i) —O— (Xiⱼ₁) or
ii) —NH— (Xiⱼ₂)
   and K₂ is bound to the group —(Y′—ONO₂) are obtained by reaction with pNO₂—C₆H₄—OCOCl of the corresponding compounds (Xkⱼ₁), (Xkⱼ₂):

NH₂—K₂—CH₂OCONH—Y—ONO₂+pNO₂—C₆H₄—OCOCl (Xkⱼ₁)-(Xkⱼ₂)

wherein Y is as previously defined and K₂ is selected from:
i) —O— (Xkⱼ₁) or
ii) —NH— (Xkⱼ₂)

Compounds of formula (Xkⱼ₁), (Xkⱼ₂) can be obtained as follow:

1) (Xkⱼ₁): wherein Y is as above defined and K₂ is selected from (VIIIe), (VIIIf) wherein R₆ is —O—:
1a) by reacting compounds of formula (XVIIa₅), (XVIIb₅) with compounds (XIa) following procedure already described in A3 (Scheme 21):

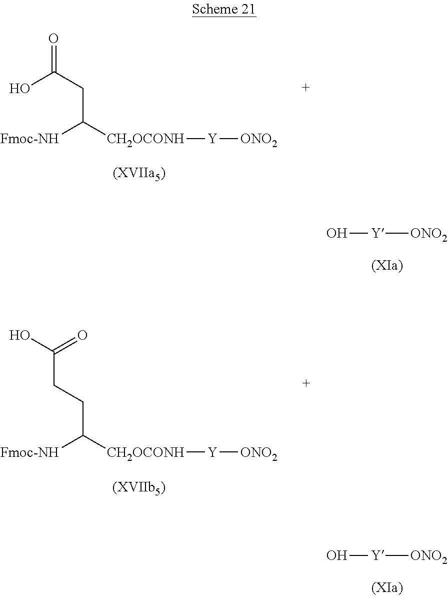

Eventually deprotecting the Fmoc group by known methods. Compounds (XVIIa₅) and (XVIIb₅) can be obtained by reacting compounds (VIIIec) or (VIIIfc) with compounds (XIe) following procedure described in A5 (Scheme 22):

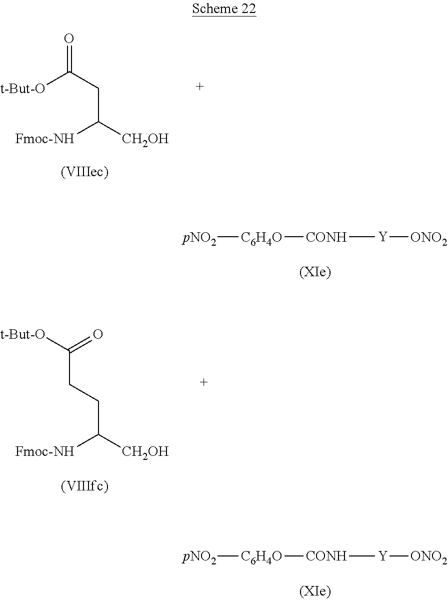

Eventually deprotecting the t-But ester by acid hydrolysis following known methods.
2) (Xkⱼ₂): wherein Y is as above defined and K₂ is selected from (VIIIe), (VIIIf) wherein R₆ is —NH—:
2a) by reacting compounds (XVIIa₅) and (XVIIb₅) above described with compounds (XIb) as above described, (Scheme 23):

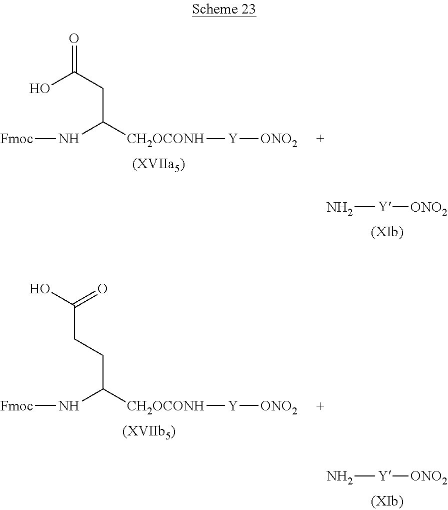

eventually hydrolyzing the Fmoc protecting group as already described.

### A19/1. Synthesis of Compounds (Xiₖᵢ₁), (Xiₖ₂) and (Xkₖ₁)-(Xkₖ₂)

Compounds of formula

p-NO₂—C₆H₄—O—CO—NH—K₃—COO—Y—ONO₂ (Xiₖ₁), (Xkₖ₂)

wherein Y is as above described and K₃ is selected from (VIIIh), (VIIIg) wherein R₇ and R₈ are selected from respectively:
i) —CO— (Xiₖ₁) or
ii) —COO— (Xiₖ₂)
   and K₃ is bound to the group —(Y′—ONO₂) are obtained by reaction with pNO₂—C₆H₄—OCOCl of the corresponding compounds (Xkₖ₁), (Xkₖ₂):

NH₂—K₃—COO—Y—ONO₂+pNO₂—C₆H₄—OCOCl (Xkₖ₁)-(Xkₖ₂)

Wherein Y is as previously defined and K₃ is selected from (VIIIh), (VIIIg) wherein R₇ and R₈ are selected from respectively:
i) —CO— (Xkₖ₁) or
ii) —COO— (Xkₖ₂)

Compounds of formula (Xkₖ₁), (Xkₖ₂) can be obtained as follow:

1) (Xkₖ₁): wherein Y is as above defined and K₃ is selected from (VIIIg), (VIIIh) wherein R₇ and R₈ are —CO—:
1a) by reacting compounds of formula (XVIIIa₁), (XVIIIb₁) with compounds (XIc) following procedure already described in A3 (Scheme 24a):

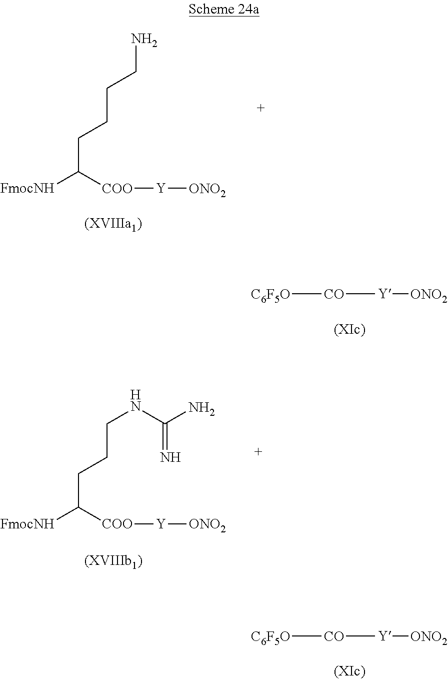

Eventually deprotecting the Fmoc group by known methods. Compounds (XVIIIa₁) and (XVIIIb₁) can be obtained by reacting compounds (VIIIgb) and (VIIIhb) already described in A7 with compounds (XIa) as already described, eventually acid hydrolyzing the Boc or Pfb protective groups (Scheme 25):

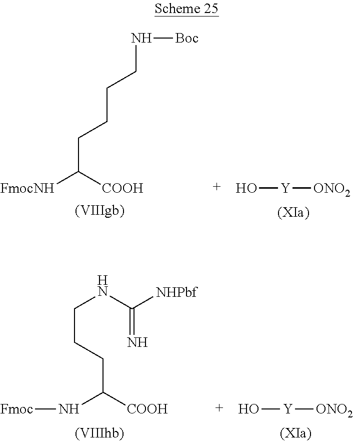

2) (Xkₖ₂): wherein Y is as above defined and K₃ is selected from (VIIIg), (VIIIh) wherein R₇ and R₈ are —COO—:
1a) by reacting compounds of formula (XVIIIa₁), (XVIIIb₁) already described above in A19 with compounds (XId) following procedure already described in A4 (Scheme 24b):

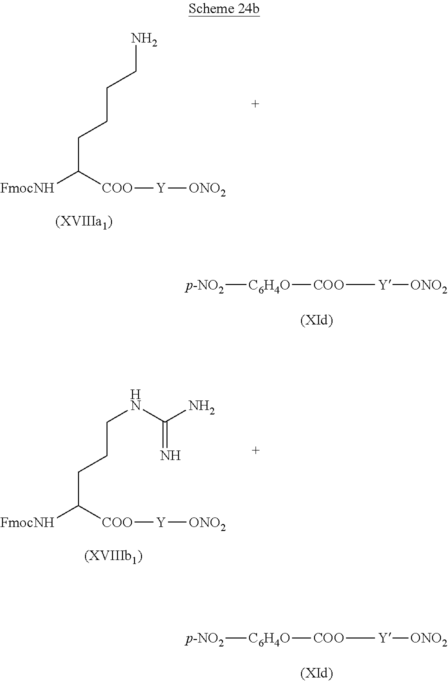

Eventually deprotecting the Fmoc group by known methods.

### A19/2. Synthesis of Compounds (Xiₖ₃), (Xiₖ₄) and (Xkₖ₃)-(Xkₖ₄)

Compounds of formula

p-NO₂—C₆H₄—O—CO—NH—K₃—COOtBut (Xiₖ₃), (Xiₖ₄)

wherein K₃ is selected from (VIIIh), (VIIIg) wherein R₇ and R₈ are selected from respectively:
i) —CO— (Xiₖ₃) or
ii) —COO— (Xiₖ₄)
   and K₃ is bound to the group —(Y′—ONO₂) are obtained by reaction with pNO₂—C₆H₄—OCOCl of the corresponding compounds (Xkₖ₃), (Xkₖ₄):

NH₂—K₃—COOtBut+pNO₂—C₆H₄—OCOCl (Xkₖ₃)-(Xkₖ₄)

Wherein K₃ is selected from (VIIIh), (VIIIg) wherein R₇ and R₈ are selected from respectively:
i) —CO— (Xkₖ₃) or
ii) —COO— (Xkₖ₄)
   and K₃ is bound to the group —(Y′—ONO₂)

Compounds of formula (Xkₖ₃), (Xkₖ₄) can be obtained as follow:

1) (Xkₖ₃): wherein K₃ is selected from (VIIIg), (VIIIh) wherein R₇ and R₈ are —CO— and K₃ is bound to the group —(Y′—ONO₂):
1a) by reacting commercially available compounds of formula (XVIIIc₁), (XVIIId₁) with compounds (XIc) following procedure already described in A3 (Scheme 24c):

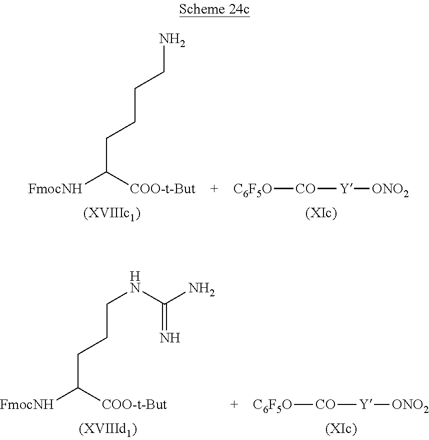

Eventually deprotecting the Fmoc group by known methods.
2) (Xkₖ₄): wherein K₃ is selected from (VIIIg), (VIIIh) wherein R₇ and R₈ are —COO— and K₃ is bound to the group —(Y′—ONO₂):
1a) by reacting compounds of formula (XVIIIc₁), (XVIIId₁) already described above with compounds (XId) following procedure already described in A4 (Scheme 24d):

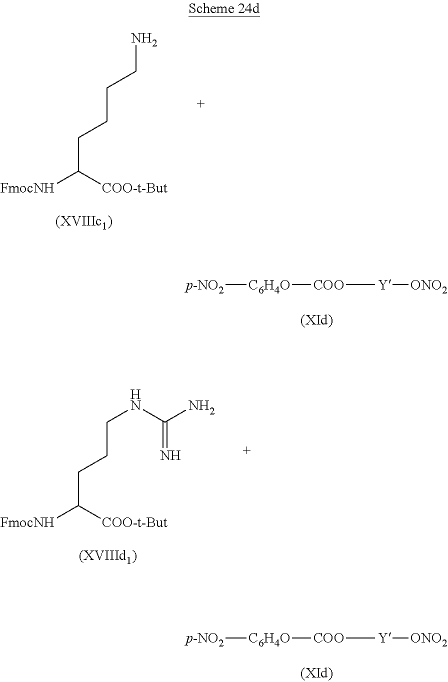

Eventually deprotecting the Fmoc group by known methods.

### A20. Synthesis of Compounds (Xi_{L1}), (Xi_{L2}) and (Xk_{L1})-(Xk_{L2})

Compounds of formula

p-NO₂—C₆H₄—O—CO—NH—K₃—CONH—Y—ONO₂ (Xi_{L1}), (Xi_{L2})

wherein Y is as above described and K₃ is selected from (VIIIh), (VIIIg) wherein R₇ and R₈ are selected from respectively:
i) —CO— (Xi_{L1}) or
ii) —COO— (Xi_{L2})
   and K₃ is bound to the group —(Y′—ONO₂) are obtained by reaction with pNO₂—C₆H₄—OCOCl of the corresponding compounds (Xk_{L1}), (Xk_{L2}):

NH₂—K₃—CONH—Y—ONO₂+pNO₂—C₆H₄—OCOCl (Xk_{L1})-(Xk_{L2})

Wherein Y is as previously defined and K₃ is selected from (VIIIh), (VIIIg) wherein R₇ and R₈ are selected from respectively:
i) —CO— (Xk_{L1}) or
ii) —COO— (Xk_{L2})

Compounds of formula (Xk_{L1}), (Xk_{L2}) can be obtained as follow:

1) (Xk_{L1}): wherein Y is as above defined and K₃ is selected from (VIIIg), (VIIIh) wherein R₇ and R₈ are —CO—:
1a) by reacting compounds of formula (XVIIIa₂), (XVIIIb₂) with compounds (XIc) following procedure already described in A3 (Scheme 26a):

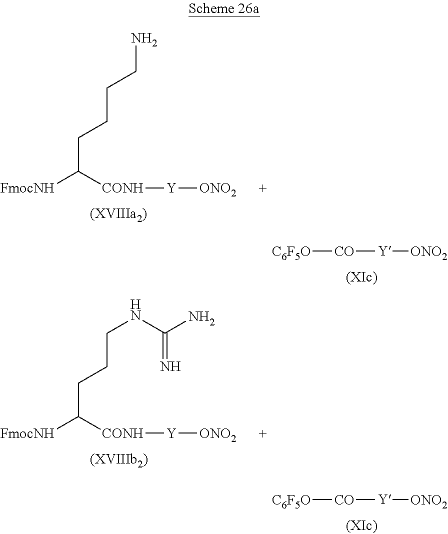

Eventually deprotecting the Fmoc group by known methods. Compounds (XVIIIa₂) and (XVIIIb₂) can be obtained by reacting compounds (VIIIgb) and (VIIIhb) already described in A7 with compounds (XIb) as already described, eventually acid hydrolyzing the Boc or Pfb protective groups (Scheme 27):

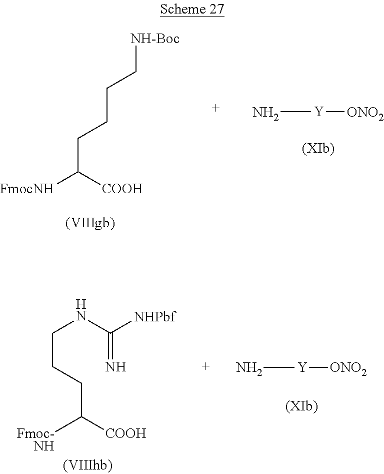

2) (Xk_{L2}): wherein Y is as above defined and K₃ is selected from (VIIIg), (VIIIh) wherein R₇ and R₈ are —COO—:
1a) by reacting compounds of formula (XVIIIa₂), (XVIIIb₂) already described above in A20 with compounds (XId) following procedure already described in A4 (Scheme 26b):

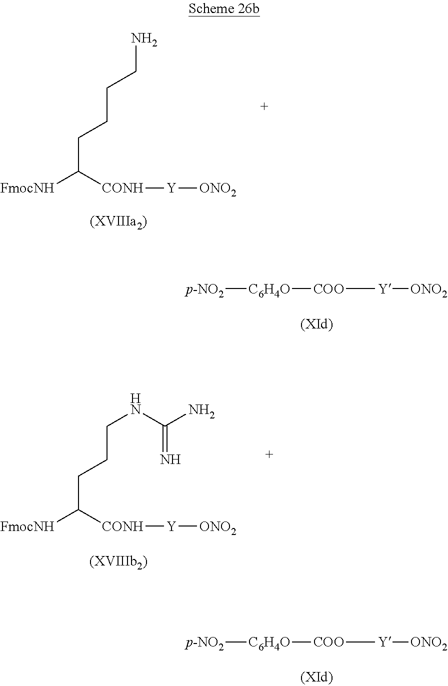

Eventually deprotecting the Fmoc group by known methods.

### A21. Synthesis of Compounds (Xiₘ₁), (Xiₘ₂) and (Xkₘ₁)-(Xkₘ₂)

Compounds of formula

p-NO₂—C₆H₄—O—CO—NH—K₃—CH₂O—CO—Y—ONO₂ (Xiₘ₁), (Xiₘ₂)

wherein Y is as above described and K₃ is selected from (VIIIh), (VIIIg) wherein R₇ and R₈ are selected from respectively:
i) —CO— (Xiₘ₁) or
ii) —COO— (Xiₘ₂)
   and K₃ is bound to the group —(Y′—ONO₂) are obtained by reaction with pNO₂—C₆H₄—OCOCl of the corresponding compounds (Xkₘ₁), (Xkₘ₂):

NH₂—K₃—CH₂O—CO—Y—ONO₂+pNO₂—C₆H₄—OCOCl (Xkₘ₁)-(Xkₘ₂)

wherein Y is as previously defined and K₃ is selected from (VIIIh), (VIIIg) wherein R₇ and R₈ are selected from respectively:
i) —CO— (Xkₘ₁) or
ii) —COO— (Xkₘ₂)

Compounds of formula (Xkₘ₁), (Xkₘ₂) can be obtained as follow:

1) (Xₘ₁): wherein Y is as above defined and K₃ is selected from (VIIIg), (VIIIh) wherein R₇ and R₈ are —CO—:
1a) by reacting compounds of formula (XVIIIa₃), (XVIIIb₃) with compounds (XIc) following procedure already described in A3 (Scheme 28a):

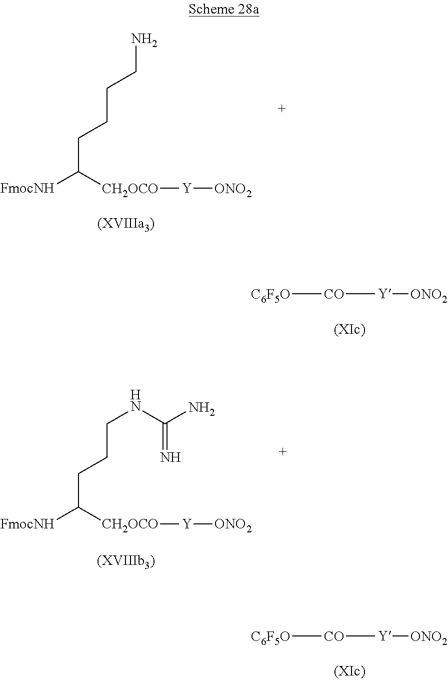

Eventually deprotecting the Fmoc group by known methods. Compounds (XVIIIa₃) and (XVIIIb₃) can be obtained by reacting compounds (VIIIgc) and (VIIIhc) already described in A7 with compounds (XIc) as already described, eventually acid hydrolyzing the Boc or Pfb protective groups (Scheme 29):

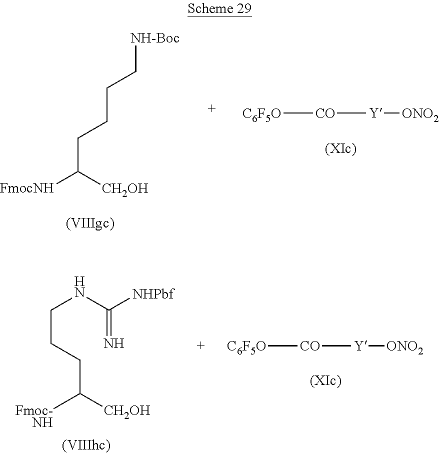

2) (Xkₘ₂): wherein Y is as above defined and K₃ is selected from (VIIIg), (VIIIh) wherein R₇ and R₈ are —COO—:
1a) by reacting compounds of formula (XVIIIa₃), (XVIIIb₃) already described above in A21 with compounds (XId) following procedure already described in A4 (Scheme 28b):

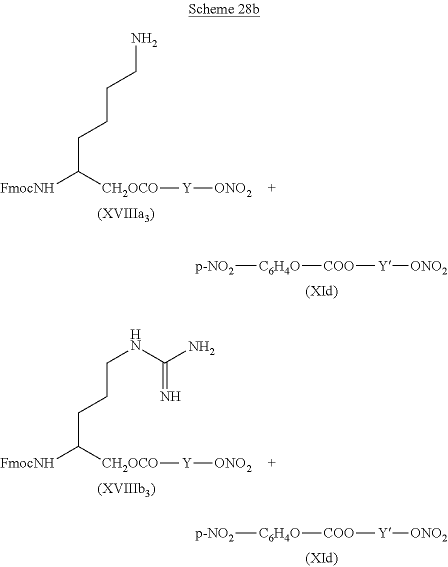

Eventually deprotecting the Fmoc group by known methods.

### A22. Synthesis of Compounds (Xiₙ₁), (Xiₙ₂) and (Xkₙ₁)-(Xkₙ₂)

Compounds of formula

p-NO₂—C₆H₄—O—CO—NH—K₃—CH₂O—COO—Y—ONO₂ (Xiₙ₁), (Xiₙ₂)

wherein Y is as above described and K₃ is selected from (VIIIh), (VIIIg) wherein R₇ and R₈ are selected from respectively:
i) —CO— (Xiₙ₁) or
ii) —COO— (Xiₙ₂)
   and K₃ is bound to the group —(Y′—ONO₂) are obtained by reaction with pNO₂—C₆H₄—OCOCl of the corresponding compounds (Xkₙ₁), (Xkₙ₂):

NH₂—K₃—CH₂O—COO—Y—ONO₂+pNO₂—C₆H₄—OCOCl (Xkₙ₁)-(Xkₙ₂)

Wherein Y is as previously defined and K₃ is selected from (VIIIh), (VIIIg) wherein R₇ and R₈ are selected from respectively:
i) —CO— (Xkₙ₁) or
ii) —COO— (Xkₙ₂)

Compounds of formula (Xkₙ₁), (Xkₙ₂) can be obtained as follow:

1) (Xkₙ₁): wherein Y is as above defined and K₃ is selected from (VIIIg), (VIIIh) wherein R₇ and R₈ are —CO—:
1a) by reacting compounds of formula (XVIIIa₄), (XVIIIb₄) with compounds (XIc) following procedure already described in A4 (Scheme 30a):

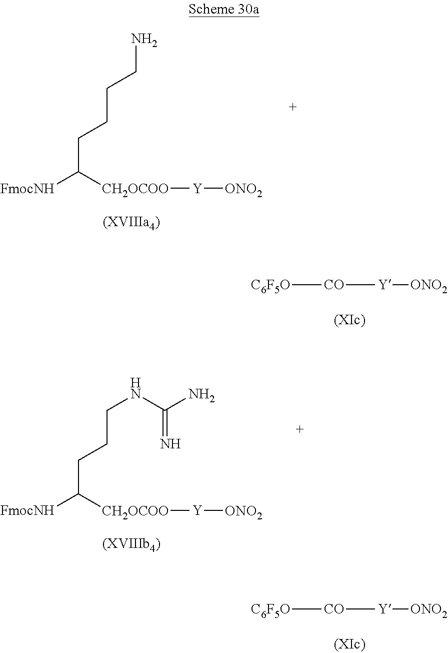

Eventually deprotecting the Fmoc group by known methods. Compounds (XVIIIa₄) and (XVIIIb₄) can be obtained by reacting compounds (VIIIgc) and (VIIIhc) already described in A7 with compounds (XId) as already described, eventually acid hydrolyzing the Boc or Pfb protective groups (Scheme 31):

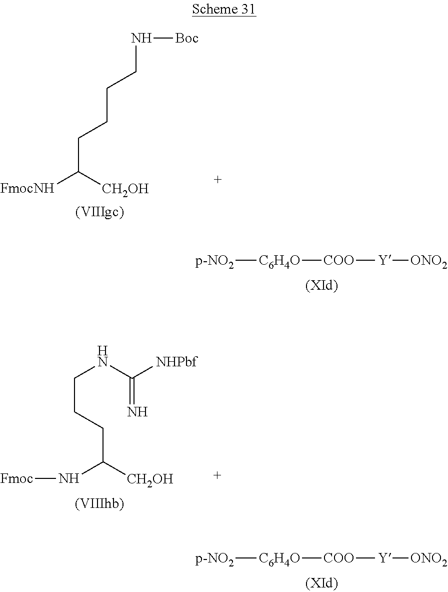

2) (Xkₙ₂): wherein Y is as above defined and K₃ is selected from (VIIIg), (VIIIh) wherein R₇ and R₈ are —COO—:
1a) by reacting compounds of formula (XVIIIa₄), (XVIIIb₄) already described above in A22 with compounds (XId) following procedure already described in A4 (Scheme 30b):

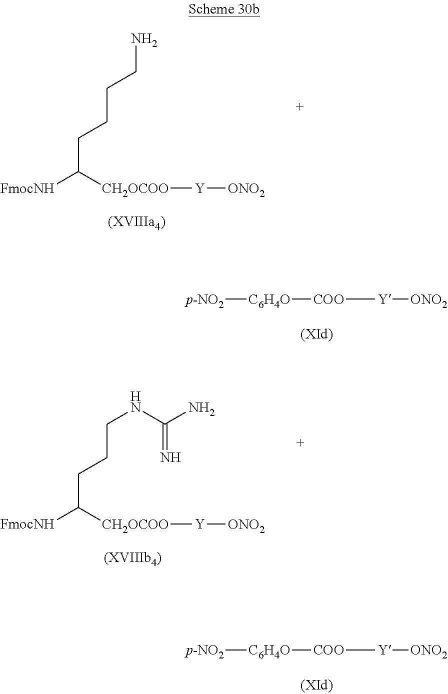

Eventually deprotecting the Fmoc group by known methods.

### A23. Synthesis of Compounds (Xiₒ₁), (Xiₒ₂) and (Xkₒ₁)-(Xkₒ₂)

Compounds of formula

p-NO₂—C₆H₄—O—CO—NH—K₃—CH₂O—CONH—Y—ONO₂ (Xiₒ₁), (Xiₒ₂)

wherein Y is as above described and K₃ is selected from (VIIIh), (VIIIg) wherein R₇ and R₈ are selected from respectively:
i) —CO— (Xiₒ₁) or
ii) —COO— (Xiₒ₂)
   and K₃ is bound to the group —(Y′—ONO₂) are obtained by reaction with pNO₂—C₆H₄—OCOCl of the corresponding compounds (Xkₒ₁), (Xkₒ₂):

NH₂—K₃—CH₂O—CONH—Y—ONO₂+pNO₂—C₆H₄—OCOCl (Xkₒ₁)-(Xkₒ₂)

wherein Y is as previously defined and K₃ is selected from (VIIIh), (VIIIg) wherein R₇ and R₈ are selected from respectively:
i) —CO— (Xkₒ₁) or
ii) —COO— (Xkₒ₂)

Compounds of formula (Xkₒ₁), (Xkₒ₂) can be obtained as follow:

1) (Xkₒ₁): wherein Y is as above defined and K₃ is selected from (VIIIg), (VIIIh) wherein R₇ and R₈ are —CO—:
1a) by reacting compounds of formula (XVIIIa₅), (XVIIIb₅) with compounds (XIc) following procedure already described in A4 (Scheme 32a):

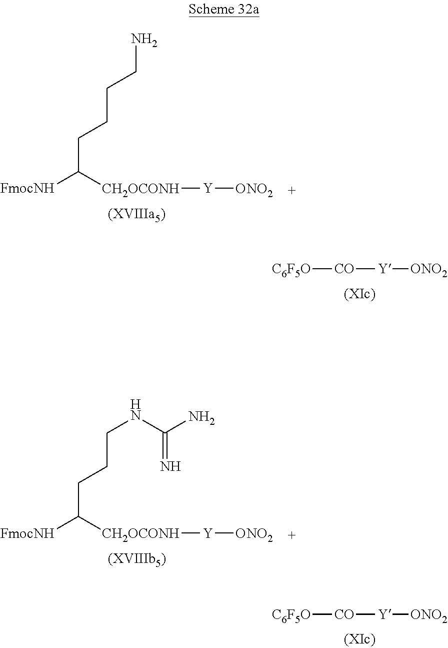

Eventually deprotecting the Fmoc group by known methods. Compounds (XVIIIa₅) and (XVIIIb₅) can be obtained by reacting compounds (VIIIgc) and (VIIIhc) already described in A7 with compounds (XIe) as already described, eventually acid hydrolyzing the Boc or Pfb protective groups (Scheme 29):

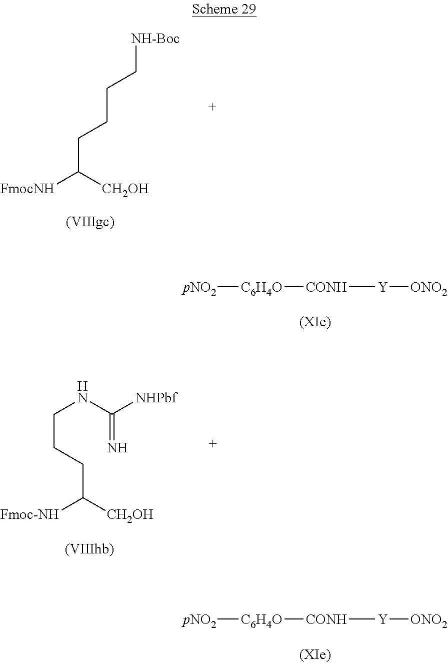

2) (Xkₒ₂): wherein Y is as above defined and K₃ is selected from (VIIIg), (VIIIh) wherein R₇ and R₈ are —COO—:
1a) by reacting compounds of formula (XVIIIa₅), (XVIIIb₅) already described above in A23 with compounds (XId) following procedure already described in A4 (Scheme 32b):

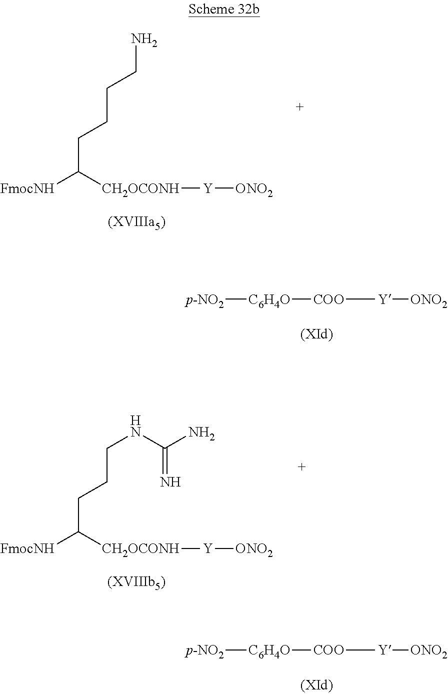

Eventually deprotecting the Fmoc group by known methods.

### A24/1. Synthesis of Compounds (Xjₐ₁), (Xjₐ₂) and (XLₐ₁)-(XLₐ₂); (Xj_{b1}), (Xj_{b2}) and (XL_{b1})-(XL_{b2}); (Xj_{c1}), (Xj_{c2}) and (XL_{c1})-(XL_{c2}); (Xj_{d1}), (Xj_{d2}) and (XL_{d1})-(XL_{d2}); (Xjₑ₁), (Xjₑ₂) and (XLₑ₁)-(XLₑ₂)

Compounds of formula

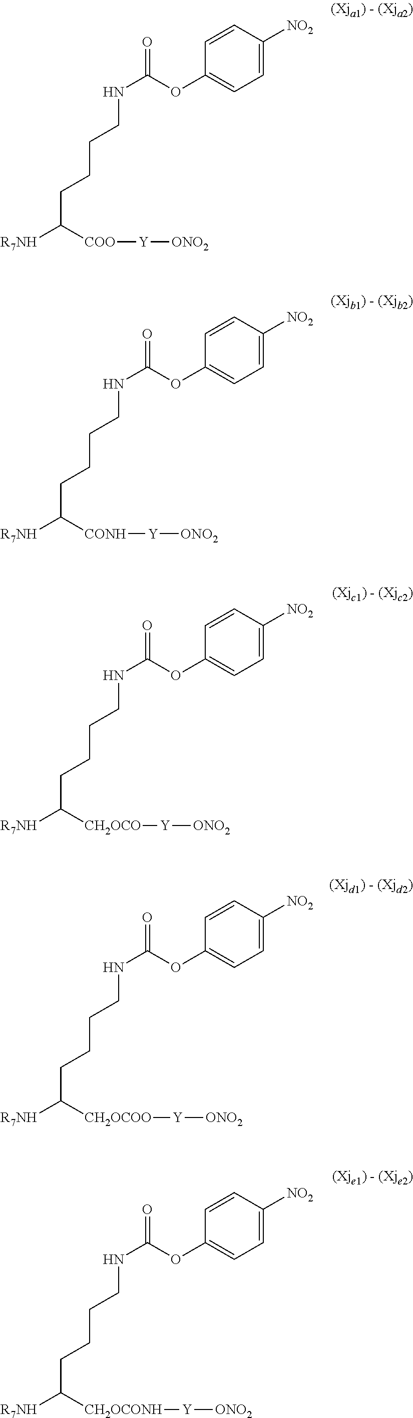

wherein Y is as above described and R₇ is selected from respectively:
i) —CO— (Xjₐ₁), (Xj_{b1}), (Xj_{c1}), (Xj_{d1}), (Xjₑ₁);
ii) —COO— (Xjₐ₂), (Xj_{b2}), (Xj_{c2}), (Xj_{d2}), (Xjₑ₂);
   and R₇ is bound to the group —(Y′—ONO₂) are obtained by reaction with pNO₂—C₆H₄—OCOCl of the corresponding compounds (XLₐ₁), (XLₐ₂); (XL_{b1})-(XL_{b2}); (XL_{c1})-(XL_{c2}); (XL_{d1})-(XL_{d2}); (XLₑ₁)-(XLₑ₂):

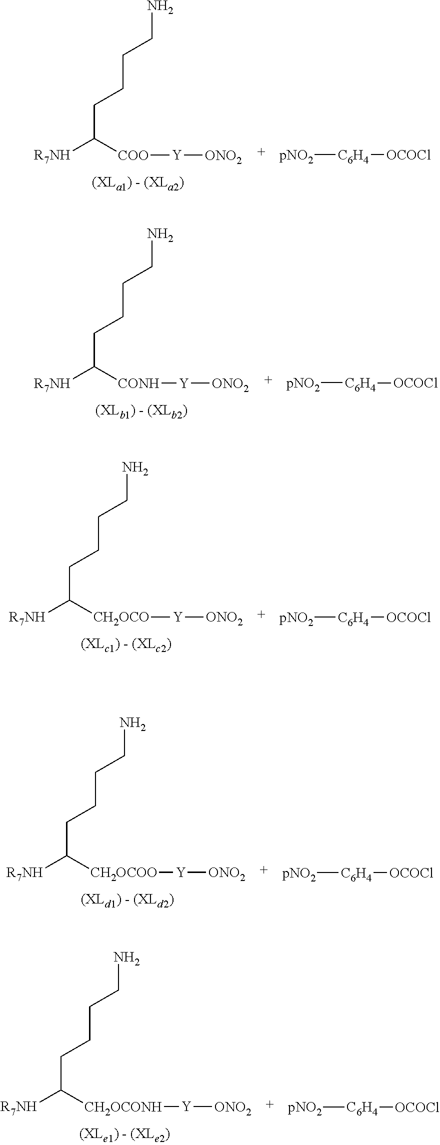

wherein Y is as previously defined and R₇ is selected from respectively:
i) —CO— (XLₐ₁), (XL_{b1}), (XL_{c1}), (XL_{d1}), (XLₑ₁);
ii) —COO— (XLₐ₂), (XL_{b2}), (XL_{c2}), (XL_{d2}), (XLₑ₂)

Compounds of formula (XLₐ₁), (XL_{b1}), (XL_{c1}), (XL_{d1}), (XLₑ₁), (XLₐ₂), (XL_{b2}), (XL_{c2}), (XL_{d2}) and (XLₑ₂)

can be obtained as follow:
1) (XLₐ₁), (XL_{b1}), (XL_{c1}), (XL_{d1}) and (XLₑ₁): wherein Y is as above defined and R₇ is —CO—:
1a) by reacting compounds of formula (XIXa₁)-(XIXa₅) with compounds (XIc) following procedure already described in A3 (Scheme 33):

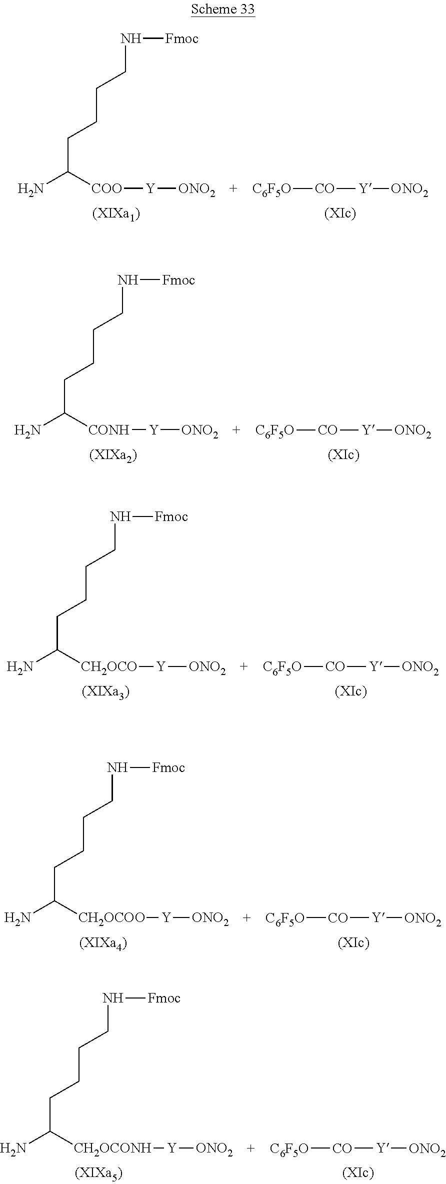

Eventually deprotecting the Fmoc group by known methods.

The compounds (XIXa₁)—(XIXa₅) can be obtained by acid hydrolyzing the Boc protective group of compounds (Xhₐ)-(Xhₑ) already prepared as described in A8;

2) (XLₐ₂), (XL_{b2}), (XL_{c2}), (XL_{d2}), (XLₑ₂): wherein Y is as above defined and R₇ is —COO—:
1a) by reacting compounds of formula (XIXa₁)—(XIXa₅) above described with compounds (XId):

p-NO₂—C₆H₄O—COO—Y′—ONO₂ (XId)

following procedure already described in A4 and eventually deprotecting the Fmoc group by known methods.

### A24/2. Synthesis of Compounds (Xjₐ₃), (Xjₐ₄) and (XLₐ₃)-(XLₐ₄)

Compounds of formula

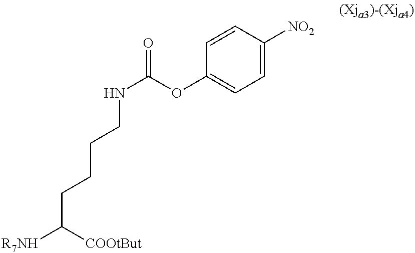

wherein R₇ is selected from respectively:
i) —CO— (Xjₐ₃)
ii) —COO— (Xjₐ₄) and R₇ is bound to the group —(Y′—ONO₂) are obtained by reaction with pNO₂—C₆H₄—OCOCl of the corresponding compounds (XLₐ₃), (XLₐ₄):

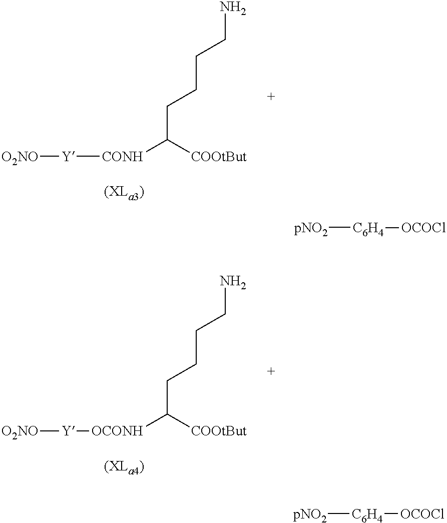

wherein Y′ is as previously defined;

Compounds of formula (XLₐ₃) and (XLₐ₄) can be obtained by reacting commercially available compound of formula (XIXa₆) with respectively compounds (XIc) and (XId) following procedure already described in A3 and A4 (Scheme 34):

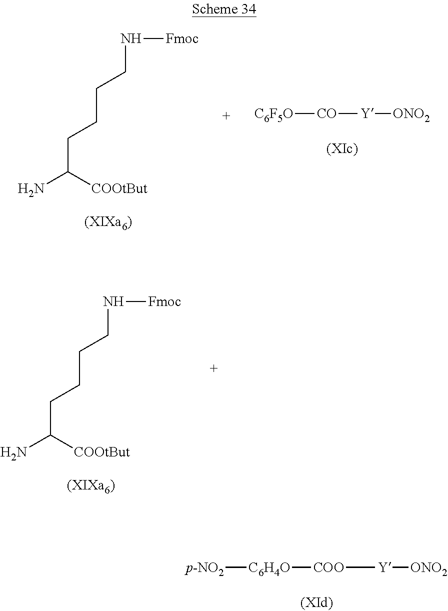

Eventually deprotecting the Fmoc group by known methods.

The following examples are to further illustrate the invention without limiting it.

### EXAMPLE 1

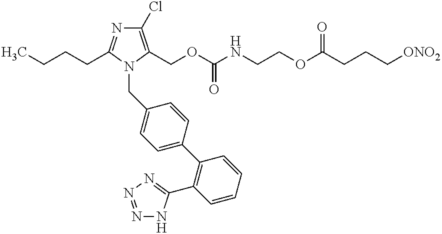

### 2-(((1-((2′-(1H-tetrazol-5-yl)biphenyl-4-yl)methyl)-2-butyl-4-chloro-1H-imidazol-5-yl)methoxy)carbonylamino)ethyl 4-(nitrooxy)butanoate (corresponding to compound (110)

A mixture of Losartan (2.20 g; 5.20 mmol), N,N-dimethylaminopyridine (DMAP) (1.16 g; 9.46 mmol) and scandium trifluoromethanesulfonate (0.466 g; 0.946 mmol) and 2-((4-nitrophenoxy)carbonylamino)ethyl 4-(nitrooxy)butanoate (Intermediate 2) (1.69 g; 4.73 mol) in CH₂Cl₂ (40 ml) was heated in a microwave apparatus (80° C., min). Then the mixture was diluted with CH₂Cl₂ and washed with NaH₂PO₄ (5%, 2×40 ml). The organic layer was dried over sodium sulfate and concentrated under reduced pressure.

The residue was purified by flash chromatography (CH₂Cl₂/MeOH: 98/2) yielding the title compound.

### EXAMPLE 2

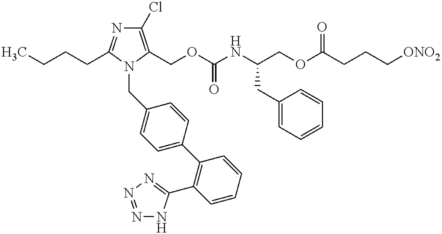

### (S)-2-(((1-((2′-(1H-tetrazol-5-yl)biphenyl-4-yl)methyl)-2-butyl-4-chloro-1H-imidazol-5-yl)methoxy)carbonylamino)-3-phenylpropyl 4-(nitrooxy)butanoate (corresponding to compound (III)

Starting from Losartan and (S)-2-((4-nitrophenoxy)carbonylamino)-3-phenylpropyl 4-(nitrooxy)butanoate (Intermediate 5) and following the same synthetic procedure described in Example 1 the title compound was obtained.

### EXAMPLE 3

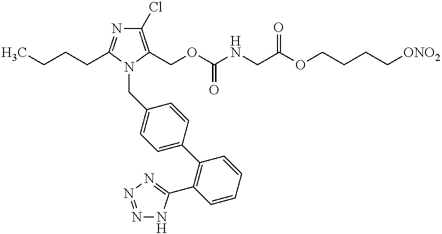

### 4-(nitrooxy)butyl 2-(((1-((2′-(1H-tetrazol-5-yl)biphenyl-4-yl)methyl)-2-butyl-4-chloro-1H-imidazol-5-yl)methoxy)carbonylamino)acetate (corresponding to compound (9)

Starting from Losartan and 4-(nitrooxy)butyl 2-((4-nitrophenoxy)carbonylamino)acetate (Intermediate 3) and following the same synthetic procedure described in Example 1 the title compound was obtained.

### EXAMPLE 4

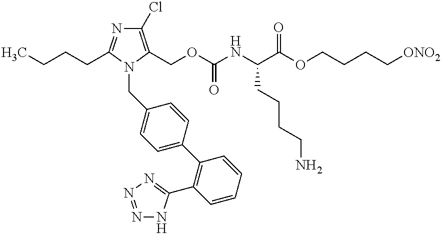

### 4-(nitrooxy)butyl 2-(((1-((2′-(1H-tetrazol-5-yl)biphenyl-4-yl)methyl)-2-butyl-4-chloro-1H-imidazol-5-yl)methoxy)carbonylamino)-6-aminohexanoate (Corresponding to compound (12)

### Step A: (2S)-4-(nitrooxy)butyl 6-(tert-butoxycarbonylamino)-2-(((2-butyl-4-chloro-1-((2′-(1-trityl-1H-tetrazol-5-yl)biphenyl-4-yl)methyl)-1H-imidazol-5-yl)methoxy)carbonylamino)hexanoate

A mixture of trityl Losartan (0.755 g; 1.14 mmol), N,N-dimethylaminopyridine (0.116 g; 0.946 mmol), scandium trifluoromethanesulfonate (0.093 g; 0.189 mmol) and 4-(nitrooxy)butyl 6-(tert-butoxycarbonylamino)-2-((4-nitrophenoxy)carbonylamino)hexanoate (Intermediate 4) (0.500 g; 0.946 mol) in CH₂Cl₂ (14 ml) was heated in a microwave apparatus (80° C., 40 min). Then the mixture was diluted with CH₂Cl₂ and washed with Na₂HPO₄ (5%, 2×40 ml). The organic layer was dried over sodium sulfate and concentrated under reduced pressure.

### Step B: (S)-4-(nitrooxy)butyl 2-(((1-((2′-(1H-tetrazol-5-yl)biphenyl-4-yl)methyl)-2-butyl-4-chloro-1H-imidazol-5-yl)methoxy)carbonylamino)-6-(tert-butoxycarbonylamino)hexanoate

The crude obtained in Step A was dissolved in MeOH (14 ml) and the reaction was heated in a microwave apparatus (90° C., min). Then the mixture was evaporated under reduced pressure and the residue was purified by flash chromatography (DCM/MeOH:98/2) yielding the title compound.

### Step C: 4-(nitrooxy)butyl 2-(((1-((2′-(1H-tetrazol-5-yl)biphenyl-4-yl)methyl)-2-butyl-4-chloro-1H-imidazol-5-yl)methoxy)carbonylamino)-6-aminohexanoate

Compound obtained in Step B was dissolved in CH₂Cl₂ (4 ml) and cooled to 0° C. Then HCl_{gas} was bubbled for 2 hours. At the end of the addition the solution was concentrated and the residue was treated with diethyl ether, affording the title compound.

### EXAMPLE 5

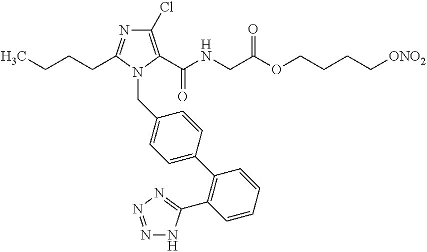

### 4-(Nitrooxy)butyl 2-(1-((2′-(1H-tetrazol-5-yl)biphenyl-4-yl)methyl)-2-butyl-4-chloro-1H-imidazole-5-carboxamido)acetate corresponding to compound (36)

### Step A: 4-(Nitrooxy)butyl 2-(2-butyl-4-chloro-1-((2′-(1-trityl-1H-tetrazol-5-yl)biphenyl-4-yl)methyl)-1H-imidazole-5-carboxamido)acetate

To a solution of 4-(nitrooxy)butyl 2-(tert-butoxycarbonylamino)acetate (Intermediate 6) (0.479 g, 2.07 mmol) and TEA (362 μl, 2.59 mmol) in CH₂Cl₂ (20 ml) a solution of 2,5-dioxopyrrolidin-1-yl 2-butyl-4-chloro-1-((2′-(2-trityl-2H-tetrazol-5-yl)biphenyl-4-yl)methyl)-1H-imidazole-5-carboxylate (1.20 g, 1.73 mmol) (Intermediate 1) in CH₂Cl₂ (10 ml) was added. The reaction was stirred at room temperature for 24 hours. Then the organic layer was washed with a solution of 5% NaH₂PO₄, brine and finally dried over sodium sulfate and concentrated under reduced pressure. The residue was purified by silica gel chromatography (n-hexane/EtOAc 8:2) affording the title compound.

### Step B: 4-(Nitrooxybutyl 2-(1-((2′-(2H-tetrazol-5-yl)biphenyl-4-yl)methyl)-2-butyl-4-chloro-1H-imidazole-5-carboxamido)acetate

A suspension of compound obtained in Step A (0.800 g, 0.94 mmol) in MeOH (18 ml) was heated in a microwave apparatus (90° C., 20 min). Then the mixture was concentrated under reduced pressure and the crude was purified by silica gel chromatography (CH₂Cl₂/MeOH 97:3) affording the title compound as a white solid.

¹H-NMR (DMSO-δ₆): 8.4 (1H, t); 7.71-7.49 (4H, m); 7.05 (4H, s); 5.47 (2H, s); 4.51 (2H, t); 4.09 (2H, t); 3.99 (2H, d); 2.55-2.51 (2H, m); 1.79-1.61 (4H, m); 1.54-1.40 (2H, m); 1.31-1.25 (2H, m); 0.82 (3H, t).

### EXAMPLE 6

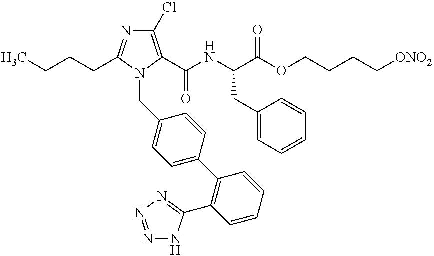

### S)-4-(nitrooxy)butyl 2-(1-((2′-(1H-tetrazol-5-yl)biphenyl-4-yl)methyl)-2-butyl-4-chloro-1H-imidazole-5-carboxamido)-3-phenylpropanoate (corresponding to compound (35))

Following the same synthetic procedure described in Example but starting from (S)-4-(nitrooxy)butyl 2-amino-3-phenylpropanoate (Intermediate 7) the title compound was obtained.

¹H-NMR (DMSO-δ₆): 8.40 (1H, d); 7.71-7.48 (4H, m); 7.31-7.13 (5H, m); 7.1-6.8 (4H, m); 5.34 (2H, s); 4.69-4.58 (1H, m); 4.47 (2H, t); 4.05 (2H, t); 3.21-3.0 (2H, m); 2.6-2.5 (2H, m); 1.71-1.52 (4H, m); 1.5-1.4 (2H, m); 1.35-1.12 (2H, m); 0.8 (3H, t).

### EXAMPLE 7

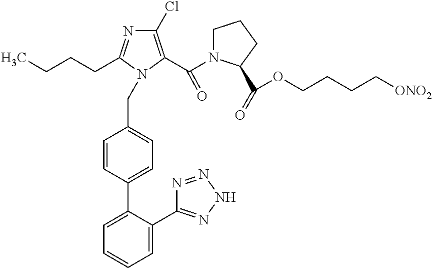

### (S)-4-(nitrooxy)butyl 1-(1-((2′-(2H-tetrazol-5-yl)biphenyl-4-yl)methyl)-2-butyl-4-chloro-1H-imidazole-5-carbonyl)pyrrolidine-2-carboxylate corresponding to compound (40)

Starting from (S)-4-(Nitrooxy)butyl pyrrolidine-2-carboxylate (Intermediate 8) and following the same synthetic procedure described in Example 5 the title compound was obtained.

¹H-NMR (DMSO-δ₆): 7.71-7.4 (3H, m): 7.4-7.3 (1H, m); 7.2-6.9 (4H, m); 5.18 (2H, dd); 4.5 (2H, m); 4.3 (1H, m); 4.07 (2H, M); 3.5-3.0 (2H, m); 2.67 (2H, m); 2.3-2.1 (1H, m); 1.9-1.4 (7H, m); 1. 1.2 (2H, m); 1.85 (3H, t).

### EXAMPLE 8

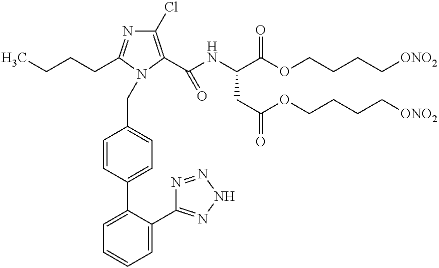

### (S)-bis(4-(nitrooxy)butyl) 2-(1-((2′-(2H-tetrazol-5-yl)biphenyl-4-yl)methyl)-2-butyl-4-chloro-1H-imidazole-5-carboxamido)succinate (corresponding to compound (42)

Starting from (S)-bis(4-(nitrooxy)butyl) 2-aminosuccinate (Intermediate 9) and following the same synthetic procedure described in Example 5 the title compound was obtained.

¹H-NMR (DMSO-δ₆): 8.49 (1H, d); 7.75-7.48 (4H, m); 7.05 (4H, s); 5.45 (2H, s); 4.80 (1H, q); 4.58-4.42 (4H, m); 4.15-3.9 (4H, m); 2.77 (2H, dq); 2.61-2.52 (2H, m); 1.79-1.55 (8H, m); 1.57-1.39 (2H, m); 1.31-1.28 (2H, m); 0.8 (3H, t).

### EXAMPLE 9

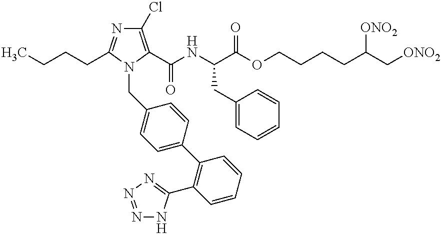

### (S)-(5,6-bis(nitrooxy)hexyl) 2-(1-((2′-(1H-tetrazol-5-yl)biphenyl-4-yl)methyl)-2-butyl-4-chloro-1H-imidazole-5-carboxamido)-3-phenylpropanoate (corresponding to compound (32))

Starting from (S)—((R)-5,6-bis(nitrooxy)hexyl) 2-amino-3-phenylpropanoate (Intermediate 10) and following the same synthetic procedure described in Example 5 the title compound was obtained.

¹H-NMR (DMSO-δ₆): 8.42 (1H, d); 7.71-7.48 (4H, m); 7.29-7.65 (5H, m); 7.11-6.9 (4H, m); 5.41-5.32 (3H, m); 4.95 (1H, m); 4.71-4.58 (2H, m); 4.03 (2H, t); 3.2-2.9 (2H, m); 2.55-2.47 (2H, m); 1.75-1.62 (2H, m); 1.6-1.3 (6H, m); 1.3-1.15 (2H, m); 0.8 (3H, t).

### EXAMPLE 9a

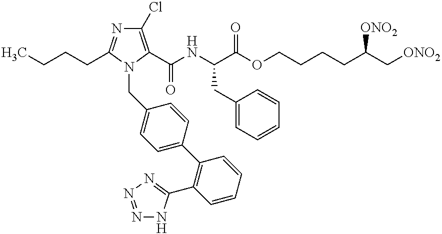

### (S)—((R)-5,6-bis(nitrooxy)hexyl) 2-(1-((2′-(1H-tetrazol-5-yl)biphenyl-4-yl)methyl)-2-butyl-4-chloro-1H-imidazole-5-carboxamido)-3-phenylpropanoate corresponding to compound (32), isomer 5R)

### Step A: methyl (2S)-2-{[(2-butyl-4-chloro-1-{[2′-(1H-tetrazol-5-yl)biphenyl-4-yl]methyl}-1H-imidazol-5-yl)carbonyl]amino}-3-phenylpropanoate

A mixture of 2-butyl-4-chloro-1-{[2′-(1H-tetrazol-5-yl)biphenyl-4-yl]methyl}-1H-imidazole-5-carboxylic acid (6.5 g, 14.9 mmol) (Intermediate 11) L-phenylalanine, methyl ester (4.16 g, 19.4 mmol), 1-ethyl-3-(3′-dimethylaminopropyl)carbodiimide hydrochloride (EDC) (4.28 g, 22.3 mmol), 1-hydroxybenzotriazole (3.02 g, 22.3 mmol) and triethylamine (7.5 g, 74.5 mmol) in N,N-dimethylformamide (80 mL) was stirred at room temperature overnight. The solvent was removed in vacuo, and the residue was partitioned between water (100 mL) and a mixture of chloroform and isopropanol (3:1) (40 mL). The aqueous layer was extracted with a mixture of chloroform and isopropanol (3:1) (40 mL×3). The organic layers were combined, dried (sodium sulfate), and concentrated in vacuo. The residue was purified by column chromatography to give the title compound, which was used in the next step without further purification. LC-MS: m/z 598 (M+H).

### Step B: (2S)-2-{[(2-butyl-4-chloro-1-{[2′-(1H-tetrazol-5-yl)biphenyl-4-yl]methyl}-1H-imidazol-5-yl)carbonyl]amino}-3-phenylpropanoic acid

To a methanol (100 mL) solution of methyl (2S)-2-{[(2-butyl-4-chloro-1-{[2′-(1H-tetrazol-5-yl)biphenyl-4-yl]methyl}-1H-imidazol-5-yl)carbonyl]amino}-3-phenylpropanoate (6.6 g, 11 mmol) was added 1N aqueous lithium hydroxide (33 mL), and the resulting mixture was stirred at room temperature for 2 hours. The reaction mixture was then concentrated in vacuo and added 100 mL of water. This mixture was washed with diethyl ether (50 mL×3), and the organic layers were discarded. The aqueous layer was acidified with diluted hydrochloric acid to pH 3, then extracted with a mixture of chloroform and isopropanol (3:1) (50 mL×3). The organic layers were combined, dried (sodium sulfate), and concentrated in vacuo. The residue was purified by column chromatography on silica gel to afford the title compound. ¹H NMR (400 MHz, CD₃OD): δ 7.67-7.50 (4H, m), 7.24-7.16 (5H, m), 7.10 (2H, d, J=8.4 Hz), 7.04 (2H, d, J=8.0 Hz), 5.49 (2H, s), 4.80 (1H, m), 3.29 (1H, m), 3.11 (1H, m), 2.62 (2H, t, J=8.0 Hz), 1.56 (2H, m), 1.34 (2H, m), 0.88 (3H, t, J=7.6 Hz). LC-MS: m/z 584 (M+H).

### Step C: (S)—((R)-5,6-bis(nitrooxy)hexyl) 2-(1-((2′-(1H-tetrazol-5-yl)biphenyl-4-yl)methyl)-2-butyl-4-chloro-1H-imidazole-5-carboxamido)-3-phenylpropanoate

(2S)-2-{[(2-Butyl-4-chloro-1-{[2′-(1H-tetrazol-5-yl)biphenyl-4-yl]methyl}-1H-imidazol-5-yl)carbonyl]amino}-3-phenylpropanoic acid (0.50 g, 0.86 mmol), (2R)-6-hydroxyhexane-1,2-diyl dinitrate (0.19 g, 0.86 mmol), 1-ethyl-3-(3′-dimethylaminopropyl)carbodiimide hydrochloride (EDC) (0.16 g, 0.86 mmol), and N,N-dimethylaminopyridine (0.11 g, 0.86 mmol) were mixed and stirred in dichloromethane (10 mL). After 3 days, the reaction mixture was concentrated in vacuo. Purification of the reaction mixture by reversed-phase mass-directed high-performance liquid chromatography afforded the title compound. ¹H-NMR (CDCl₃): 8.02 (1H, d, J=7.6 Hz); 7.63 (1H, t, J=7.5 Hz); 7.56 (1H, t, J=7.6 Hz); 7.47 (1H, d, J=7.6 Hz); 7.34-7.24 (3H, m); 7.22 (2H, d, J=7.3 Hz); 7.12 (2H, d, J=8.0 Hz); 6.91 (2H, d, J=7.8 Hz); 6.79 (1H, d, J=6.0 Hz); 5.56 (1H, d, J=15.8 Hz); 5.37 (1H, d, J=15.8 Hz); 5.33-5.24 (1H, m); 4.76 (1H, dd, J=2.6, 12.9 Hz); 4.67 (1H, q, J=6.1 Hz); 4.49 (1H, ddd, J=6.6, 8.0, 13.0 Hz); 4.18 (1H, td, J=5.9, 11.0 Hz); 4.02 (1H, td, J=5.7, 10.7 Hz); 3.19 (1H, dd, J=6.2, 13.7 Hz); 3.14 (1H, dd, J=8.0, 14.0 Hz); 2.70 (2H, t, J=7.6 Hz); 1.8-1.6 (m, 5H), 1.55-1.35 (m, 5H), 0.91 (3H, t, J=7.4 Hz). LC-MS: m/z 790 (M+H).

### EXAMPLE 10

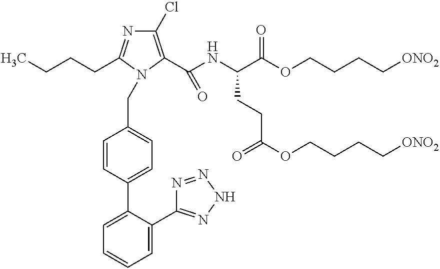

### ((S)-bis(4-(nitrooxy)butyl) 2-(1-((2′-(2H-tetrazol-5-yl)biphenyl-4-yl)methyl)-2-butyl-4-chloro-1H-imidazole-5-carboxamido)pentanedioate (corresponding to compound (44))

Starting from (S)-bis(4-(nitrooxy)butyl) 2-aminopentanedioate (Intermediate 12) and following the same synthetic procedure described in Example 5 the title compound was obtained

¹H-NMR (DMSO-δ₆): 8.6 (1H, d); 7.71-7.45 (4H, m); 7.11-7.05 (4H, m); 5.49-5.3 (2H, m); 4.52-4.38 (5H, m); 4.1-3.98 (4H, m); 2.6-2.5 (2H, m); 2.4 (2H, t); 2.15-1.89 (2H, dm); 1.71-1.52 (8H, m); 1.51-1.40 (2H, m); 1.3-1.19 (2H, m); 0.8 (3H, t).

### EXAMPLE 11

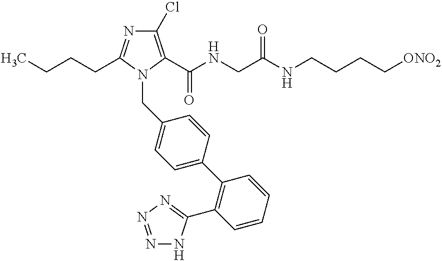

### 4-(2-(1-((2′-(1H-tetrazol-5-yl)biphenyl-4-yl)methyl)-2-butyl-4-chloro-1H-imidazole-5-carboxamido)acetamido)butyl nitrate (corresponding to compound (45))

Starting from 4-(2-aminoacetamido)butyl nitrate (Intermediate 13) and following the same synthetic procedure described in Example 5 the title compound was obtained.

¹H-NMR (DMSO-δ₆): 8.06 (1H, t); 7.92 (1H, t); 7.71-7.49 (4H, m); 7.05 (4H, s); 5.52 (2H, s); 4.49 (2H, t); 3.83 (2H, d); 3.15-3.075 (2H, m); 2.6-2.48 (2H, m); 1.7-1.59 (2H, m); 1.507-1.4 (4H, m); 1.31-1.18 (2H, m); 0.802 (3H, t).

### EXAMPLE 12

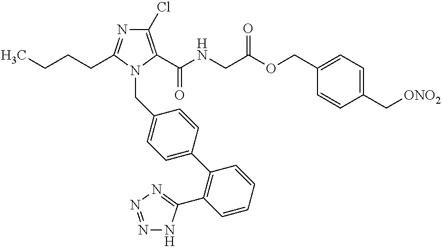

### 4-(nitrooxymethyl)benzyl 2-(1-((2′-(2H-tetrazol-5-yl)biphenyl-4-yl)methyl)-2-butyl-4-chloro-1H-imidazole-5-carboxamido)acetate (corresponding to compound (46))

Starting from 4-[(nitrooxy)methyl]benzyl 2-aminoacetate (Intermediate 14) and following the same synthetic procedure described in Example 5 the title compound was obtained.

¹H-NMR (DMSO-δ₆): 8.49 (1H, t); 7.71-7.41 (8H, m); 7.04 (4H, s); 5.56 (2H, s); 5.45 (2H, s); 5.16 (2H, s); 4.07 (2H, d); 2.59-2.49 (2H, m); 1.51-1.40 (2H, m); 1.32-1.19 (2H, M); 0.804 (3H, t).

### EXAMPLE 13

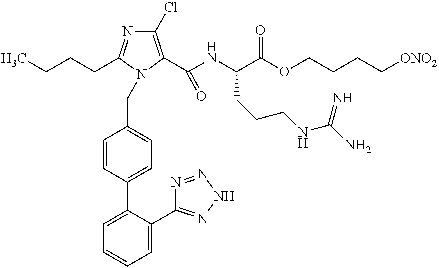

### (S)-4-(nitrooxy)butyl 2-(1-((2′-(2H-tetrazol-5-yl)biphenyl-4-yl)methyl)-2-butyl-4-chloro-1H-imidazole-5-carboxamido)-5-guanidinopentanoate (corresponding to compound (47))

Starting from (S)-4-(nitrooxy)butyl 2-amino-5-(3-(2,2,4,6,7-pentamethyl-2,3-dihydrobenzofuran-5-ylsulfonyl)guanidino)pentanoate (Intermediate 15) and following the same synthetic procedure described in Example 5 and then acid hydrolizing the Pfb protective group as described for analogue reaction in Example 4, the title compound was obtained.

### EXAMPLE 14

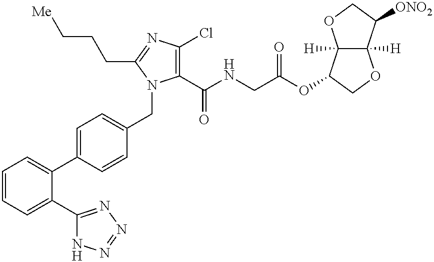

### (3S,3aR,6R,6aS)-6-(nitrooxy)hexahydrofuro[3,2-b]furan-3-yl {[(2-butyl-4-chloro-1-{[2′-(1H-tetrazol-5-yl)biphenyl-4-yl]methyl}-1H-imidazol-5-yl)carbonyl]amino}acetate (corresponding to compound (113))

### Step A: (3S,3aR,6R,6aS)-6-(nitrooxy)hexahydrofuro[3,2-b]furan-3-yl {[(9H-fluoren-9-ylmethoxy)carbonyl]amino}acetate

A mixture of Fmoc-glycine (10.0 g, 33.6 mmol), isosorbide-5-mononitrate (7.07 g, 37.0 mmol), 1-ethyl-3-(3′-dimethylaminopropyl)carbodiimide hydrochloride (7.74 g, 40.4 mmol), 1-hydroxybenzotriazole (6.18 g, 40.4 mmol), N-methylmorpholine (11.1 mL, 101 mmol) and 4-dimethylaminopyridine (0.411 g, 3.36 mmol) was stirred in dichloromethane (300 mL) at room temperature overnight. The solvent was removed in vacuo, and the residue was partitioned between water (300 mL) and ethyl acetate (300 mL). The aqueous layer was extracted with ethyl acetate (300 mL×3), and the combined organic layers were dried (sodium sulfate) and concentrated in vacuo. The residue was purified by column chromatography over silica gel to give the title compound. LC-MS: m/z 471 (M+H).

### Step B: (3S,3aR,6R,6aS)-6-(nitrooxy)hexahydrofuro[3,2-b]furan-3-yl {[(2-butyl-4-chloro-1-{[2′-(1H-tetrazol-5-yl)biphenyl-4-yl]methyl}-1H-imidazol-5-yl)carbonyl]amino}acetate

To a N,N-dimethylformamide solution (100 mL) of (3S,3aR,6R,6aS)-6-(nitrooxy)hexahydrofuro[3,2-b]furan-3-yl {[(9H-fluoren-9-ylmethoxy)carbonyl]amino}acetate (8.33 g, 17.7 mmol) was added piperidine (1.75 mL, 17.7 mmol). After 1 hour, 2-butyl-4-chloro-1-{[2′-(1H-tetrazol-5-yl)biphenyl-4-yl]methyl}-1H-imidazole-5-carboxylic acid (3.18 g, 7.27 mmol), (benzotriazol-1-yloxy)tripyrrolidinophosphonium hexafluorophosphate (4.06 g, 7.79 mmol) and triethylamine (4.0 mL, 28.7 mmol) were added, and the reaction mixture was stirred overnight. Purification of the reaction mixture by reversed-phase mass-directed high-performance liquid chromatography afforded the title compound.

¹H-NMR (CDCl₃): 7.77 (1H, d, J=7.6 Hz); 7.57 (1H, t, J=7.7 Hz); 7.44-7.52 (2H, m); 7.40 (1H, d, J=7.6 Hz); 7.01 (2H, d, J=8.0 Hz); 6.94 (2H, d, J=8.0 Hz); 5.50 (2H, s); 5.33 (1H, dt, J=2.6, 5.4 Hz); 5.20 (1H, d, J=2.8 Hz); 4.96 (1H, t, J=5.2 Hz); 4.48 (1H, d, J=4.8 Hz); 4.11 (2H, d, J=5.2 Hz); 3.98 (1H, t, J=10.9 Hz); 3.91-3.97 (2H, m); 3.84 (1H, dd, J=5.4, 11.3 Hz); 2.78 (2H, t, J=7.8 Hz); 1.61 (2H, quintet, J=7.7 Hz), 1.32 (2H, sextet, J=7.5 Hz), 0.84 (3H, t, J=7.4 Hz). LC-MS: m/z 667 (M+H).

### EXAMPLE 15

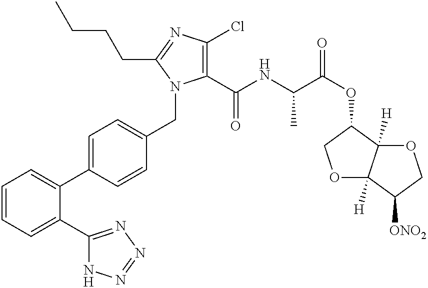

### (3S,3aR,6R,6aS)-6-(nitrooxy)hexahydrofuro[3,2-b]furan-3-yl (2S)-2-{[(2-butyl-4-chloro-1-{[2′-(1H-tetrazol-5-yl)biphenyl-4-yl]methyl}-1H-imidazol-5-yl)carbonyl]amino}propanoate (corresponding to compound (114)

The title compound was prepared by the procedure described for Example 14, except that in Step A the reagent Fmoc-glycine was replaced by Fmoc-L-alanine and in Step B the reagent (3S,3aR,6R,6aS)-6-(nitrooxy)hexahydrofuro[3,2-b]furan-3-yl {[(9H-fluoren-9-ylmethoxy)carbonyl]amino}acetate was replaced by(S)-((3S,3aR,6R,6aS)-6-(nitrooxy)hexahydrofuro[3,2-b]furan-3-yl) (2S)-2-(((9H-fluoren-9-yl)methoxy)carbonylamino)propanoate.

¹H NMR (500 MHz, CD₃CN). δ 7.81 (d, J=6.8 Hz, 1H), 7.72 (d, J=8.5 Hz, 1H), 7.65 (td, J=7.7, 1.2 Hz, 1H), 7.55 (t, J=7.1 Hz, 1H), 7.50 (d, J=7.8 Hz, 1H), 7.13 (d, J=8.5 Hz, 2H), 7.09 (d, J=8.2 Hz, 2H), 5.50 (s, 2H), 5.40 (td, J=5.4, 2.3 Hz, 1H), 5.14 (d, J=3.2 Hz, 1H), 4.91 (t, J=5.4 Hz, 1H), 4.45 (quintet, J=7.2 Hz, 1H), 4.40 (d, J=5.0 Hz, 1H), 3.98-3.92 (m, 2H), 3.89-3.83 (m, 2H), 2.87 (t, J=7.8 Hz, 2H), 1.59 (quintet, J=7.7 Hz, 2H), 1.35 (d, J=7.4 Hz, 3H), 1.32 (sextet, J=7.3 Hz, 2H), 0.85 (t, J=7.4 Hz, 3H); LC-MS: m/z 681.2 (M+H).

### INTERMEDIATE 1

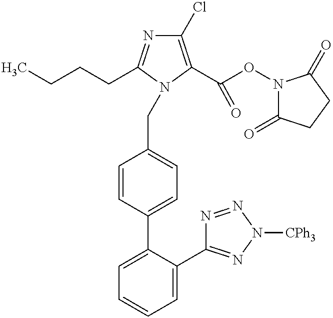

### 2,5-dioxopyrrolidin-1-yl 2-butyl-4-chloro-1-((2′-(2-trityl-2H-tetrazol-5-yl)biphenyl-4-yl)methyl)-1H-imidazole-5-carboxylate (Corresponding to compound of formula (R_{IIc}))

### Step A: 2,5-dioxopyrrolidin-1-yl 2-butyl-4-chloro-1H-imidazole-5-carboxylate

To a solution of 2-butyl-4-chloro-1H-imidazole-5-carboxylic acid (10.0 g, 49.4 mmol) in THF (100 mL), cooled to 0° C., 1,1-N,N-carbonyl diimidazole (12.0 g, 74.1 mmol) was added. The reaction was warmed to room temperature and stirred for 3 hours. Then N-hydroxysuccinimide (5.68 g, 49.4 mmol) and sodium ethylate (672 mg, 9.88 mmol) were added and the mixture was stirred overnight. The reaction mixture was partitioned between EtOAc (100 mL) and NaH₂PO₄ (5%, 100 mL). The organic phase was washed with NaH₂PO₄ (3×50 mL) and brine (3×50 mL), dried over Na₂SO₄ and concentrated. The crude was purified by flash chromatography (BIOTAGE equipment, column diameter 75+L, EtOAc/Hexane gradient —EtOAc 12%, 999 mL; EtOAc 12% to 100%, 9999 mL; EtOAc 100%, 1998 mL) affording the title compound as a white solid.

¹H-NMR (CDCl₃): 10.13 (1H, s); 2.91 (4H, s); 2.73 (2H, t); 1.73 (2H, m); 1.36 (2H, m); 0.93 (3H, t).

### Step B: 2,5-dioxopyrrolidin-1-yl 2-butyl-4-chloro-1-((2′-(2-trityl-2H-tetrazol-5-yl)biphenyl-4-yl)methyl)-1H-imidazole-5-carboxylate

2,5-dioxopyrrolidin-1-yl 2-butyl-4-chloro-1H-imidazole-5-carboxylate (2.13 g, 7.10 mmol), 5-(4′-(bromomethyl)biphenyl-2-yl)-1-trityl-1H-tetrazole (3.96 g, 7.10 mmol) and K₂CO₃ (1.18 g, 8.50 mmol) were dissolved in DMF (25 mL) and stirred overnight. The salts were filtered off and the mixture was partitioned between EtOAc (50 mL) and NaH₂PO₄ (5%, 50 mL). The organic phase was washed with NaH₂PO₄ (5%, 3×50 mL), and brine (3×50 mL), dried over Na₂SO₄ and concentrated. The crude was purified by flash chromatography (BIOTAGE equipment, column diameter 65i, EtOAc/Hexane gradient —EtOAc 12% 471 mL; EtOAc 12% to 80%, 4710 mL; EtOAc 80% 942 mL-) affording the title compound (R_{IIc}) as a white solid.

¹H-NMR (CDCl₃): 7.90 (1H, dd); 7.49 (2H, m); 7.46-7.22 (10H, m); 7.11 (2H, d); 6.92 (6H, m); 6.80 (2H, d); 5.34 (2H, s); 2.82 (4H, s); 2.52 (2H, t); 1.63 (2H, m); 1.26 (2H, m); 0.85 (3H, t)

### INTERMEDIATE 2

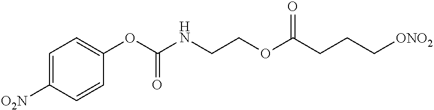

### 2-((4-nitrophenoxy)carbonylamino)ethyl 4-(nitrooxy)butanoate (following procedure described in Appendix 1, A3)

### Step A: 2-(tert-butoxycarbonylamino)ethyl 4-(nitrooxy)butanoate tert-butyl 2-hydroxyethylcarbamate

### Step B: 2-aminoethyl 4-(nitrooxy)butanoate hydrochloride

### Step C: 2-((4-nitrophenoxy)carbonylamino)ethyl 4-(nitrooxy)butanoate

To a solution of 2-aminoethyl 4-(nitrooxy)butanoate hydrochloride (1.16 g; 5.06 mmol) and NaHCO₃ (0.850 g; 10.1 mmol) in CH₃CN (32 ml) cooled to 0° C., was added dropwise a solution of 4-nitrophenyl chloroformate (1.02 g; 5.06 mmol) in CH₃CN (16 ml). The resulting mixture was stirred at room temperature for 18 hours. Then the mixture was diluted with EtOAc and washed with aqueous NaH₂PO₄ (5%, 2×50 ml) and brine (40 ml). The organic layer was dried over sodium sulfate and concentrated under reduced pressure.

The residue was purified by flash chromatography (hexane/EtOAc=6/4; R_{f}=0.21), yielding the title compound.

### INTERMEDIATE 3

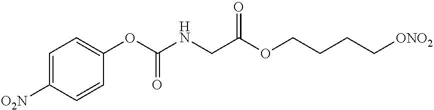

### 4-(nitrooxy)butyl 2-((4-nitrophenoxy)carbonylamino)acetate (following procedure described in Appendix 1, A1)

### Step A: 4-hydroxybutyl 2-(tert-butoxycarbonylamino)acetate

### Step B: 4-(nitrooxy)butyl 2-(tert-butoxycarbonylamino) acetate

### Step C: 4-(nitrooxy)butyl 2-aminoacetate

### Step D: 4-(nitrooxy)butyl 2-((4-nitrophenoxy)carbonylamino)acetate

The title compound was prepared from 4-(nitrooxy)butyl 2-aminoacetate and 4-nitrophenyl chloroformate as described in Intermediate 2 Step C.

### INTERMEDIATE 4

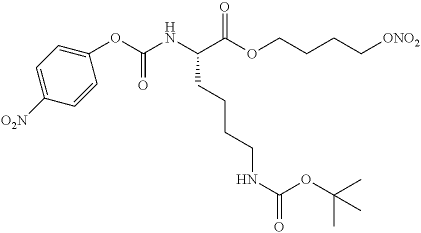

### (S)-4-(nitrooxy)butyl 6-(tert-butoxycarbonylamino)-2-((4-nitrophenoxy)carbonylamino)hexanoate (following procedure described in Appendix 1, A7)

### Step A: (S)-4-(nitrooxy)butyl 1-(9H-fluoren-9-yl)-13,13-dimethyl-3,11-dioxo-2,12-dioxa-4,10-diazatetradecane-5-carboxylate

Commercial N(α)-Fmoc-N(ε)-Boc-L-lysine pentafluorophenyl ester (6.51 mmol) and 4-(nitrooxy)-1-butanol (6.55 mmol) were dissolved in DMF (12 ml) and the mixture was cooled to 0° C. N,N-dimethylaminopyridine (DMAP) (6.55 mmol) were added and the reaction was slowly warmed to room temperature and stirred for 4 hours. Then the mixture was concentrate under reduced pressure and diluted with EtOAc, washed with 5% aqueous Na₂HPO₄ and brine. The organic layer was dried over sodium sulphate and concentrated under reduced pressure.

The residue was purified by flash chromatography (n-hexan/EtOAc 70:30 as eluent) yielding the title compound.

### Step B: (S)-4-(nitrooxy)butyl 2-amino-6-(tert-butoxycarbonylamino)hexanoate

To a solution of (S)-4-(nitrooxy)butyl 1-(9H-fluoren-9-yl)-13,13-dimethyl-3,11-dioxo-2,12-dioxa-4,10-diazatetradecane-5-carboxylate (2.52 g, 4.30 mmol) in CH₃CN (30 ml), piperidine (2.12 ml, 21.5 mmol) was added in the dark, the reaction was stirred at r.t. for 25 min. Then the mixture was concentrated to a small volume and diluted with EtOAc (150 ml) and washed with 5% aqueous NaHPO₄ (2×70 ml). The organic layer was dried over sodium sulphate and concentrated under reduced pressure.

The residue was purified by flash chromatography (CH₂Cl₂/MeOH 98:2 as eluent), yielding the title compound. ¹H-NMR (DMSO-d6): 6.78 (1H, t), 4.55 (2H, t), 4.07 (2H, m), 3.26 (2H, t), 2.87 (1H, m), 1.73-1.65 (4H, m), 1.64-1.40 (2H, m), 1.37 (9H, s), 1.36-1.22 (4H, s).

### Step C: (S)-4-(nitrooxy)butyl 6-(tert-butoxycarbonylamino)-2-((4-nitrophenoxy)carbonylamino)hexanoate

The title compound was prepared starting from (S)-4-(nitrooxy)butyl 2-amino-6-(tert-butoxycarbonylamino)hexanoate as described in Intermediate 2 Step C.

### INTERMEDIATE 5

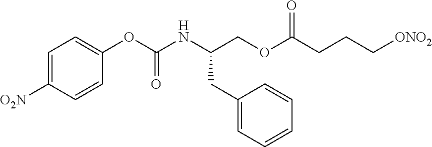

### (S)-2-((4-nitrophenoxy)carbonylamino)-3-phenylpropyl 4-(nitrooxy)butanoate

The title compound was obtained from (S)-tert-butyl 1-hydroxy-3-phenylpropan-2-ylcarbamate following the procedure described for Intermediate 2.

### INTERMEDIATE 6

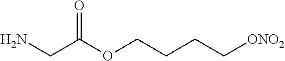

### 4-(nitrooxy)butyl 2-aminoacetate

### Step A 4-(nitrooxy)butyl 2-(tert-butoxycarbonylamino)acetate

The title compound was obtained from N-Boc-L-glycine, N-hydroxysuccinimido ester following procedures described in Intermediate 4, Step A.

### Step B: 4-(nitrooxy)butyl 2-aminoacetate

To a solution of 4-(nitrooxy)butyl 2-(tert-butoxycarbonylamino)acetate (3.00 g, 1.03 mmol) in CH₂Cl₂ (40 ml) cooled to 0° C., HCl_{gas} was bubbled for 2 hours. The solvent was concentrated and the residue was treated with diethyl ether, affording the title compound as a white solid.

### INTERMEDIATE 7

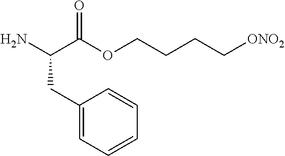

### (S)-4-(nitrooxy)butyl 2-amino-3-phenylpropanoate

The title compound was prepared from N-Boc-Phenylalanine following the procedure described in Intermediate 6.

### INTERMEDIATE 8

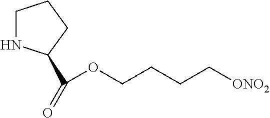

### (S)-4-(nitrooxy)butyl pyrrolidine-2-carboxylate

The title compound was prepared as an oil from N-Boc-L-Proline following the procedure described in Intermediate 6.

### INTERMEDIATE 9

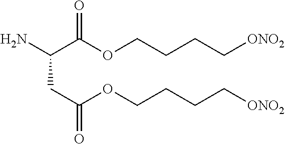

### (S)-bis(4-(nitrooxy)butyl) 2-aminosuccinate

The title compound was prepared from Boc-L-glutamic acid following the procedure described in Intermediate 6.

### INTERMEDIATE 10

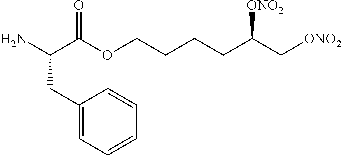

### (S)—((R)-5,6-bis(nitrooxy)hexyl) 2-amino-3-phenylpropanoate

The title compound was prepared from Boc-L-Phenylalanine and (2R)-6-hydroxyhexane-1,2-diyl dinitrate (prepared as described in WO2005070868(A1)) following the procedure described in Intermediate 6

### INTERMEDIATE 11

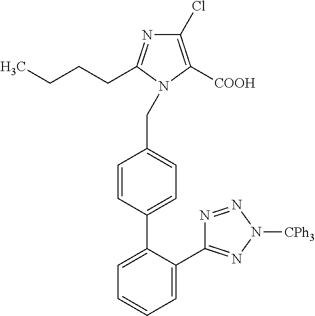

### 2-butyl-4-chloro-1-{[2′-(2-trityl-2H-tetrazol-5-yl)biphenyl-4-yl]methyl}-1H-imidazole-5-carboxylic acid

### Step A: 2-butyl-4-chloro-1-{[2′-(1H-tetrazol-5-yl)biphenyl-4-yl]methyl}-1H-imidazole-5-carboxylic acid

Water (10 L) was added to a 22 L 4-neck round bottom flask. The water was cooled to 0° C. At 0° C., potassium hydroxide (855 g, 15.24 mol) was added followed by losartan potassium (500 g, 1.09 mol)), sodium periodate (554 g, 2.59 mol) and ruthenium (III) chloride hydrate (12 g, 0.05 mol) and the reaction mixture was stirred at 0° C. overnight. The reaction mixture was filtered. IPA (90 mL) was added to the filtrate while stirring. The solution was warmed to 25° C. and stirred for 2.5 hrs. After 2.5 hrs., phosphoric acid (1200 mL) was added, maintaining the temperature below +30° C. The mixture was stirred for 30 min and the product was filtered, washing with water. The residue was dried in the vacuum oven at 55° C. overnight. The solid was dissolved in methanol (4 L) and isopropyl acetate (12 L), and charcoal (activated carbon) (100 g) was added. The mixture was stirred at rt for 3.5 hrs, filtered and concentrated. The product was redissolved in DCM/MeOH and precipitated with heptane to afford the title compound as a greenish/brown foam which was used in subsequent steps without further purification.

### Step B: 2-butyl-4-chloro-1-{[2′-(2-trityl-2H-tetrazol-5-yl)biphenyl-4-yl]methyl}-1H-imidazole-5-carboxylic acid

To a solution of E3174 (234.58 g, 0.54 mol) in DCM (4500 mL) was added triethylamine (85 mL, 0.59 mol) followed by a solution of trityl chloride (159 g, 0.56 mol) in DCM (800 mL) and the reaction mixture was stirred at rt overnight. The reaction mixture was washed with water, dried (MgSO4), filtered, and concentrated in vacuo. Chromatography over silica eluting with 20-80% acetone/heptane afforded the title compound as an orange solid.

### INTERMEDIATE 12

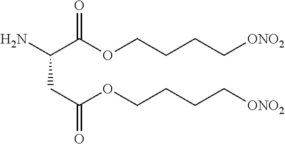

### (S)-bis(4-(nitrooxy)butyl) 2-aminosuccinate

The title compound was prepared from Boc-L-aspartic acid following the procedure described in Intermediate

### INTERMEDIATE 13

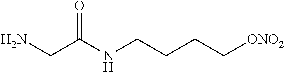

### 4-(2-aminoacetamido)butyl nitrate

### Step A: tert-butyl 2-(4-hydroxybutylamino)-2-oxoethylcarbamate

4-Amino-1-butanol (0.686 g, 7.70 mmol) and triethylamine (0.779 g, 7.70 mmol) were dissolved in CH₂Cl₂ (40 ml) and the mixture was cooled to 0° C. A suspension of commercial N-Boc-glycine N-hydroxysuccinimido estere (2.10 g; 7.70 mmol) in CH₂Cl₂ (40 ml) was added and the reaction was slowly warmed to room temperature and stirred for 24 hours. Then the mixture was diluted with CH₂Cl₂ (150 ml) and washed with 5% aqueous Na₂HPO₄ and brine. The aqueous layer was extracted twice with CH₂Cl₂ and twice with a mixture of EtOAc/MeOH 98:2. The organic layers were dried over sodium sulphate and concentrated under reduced pressure. The residue was purified by flash chromatography (n-hexane/i-prOH 80:20) affording the title compound.

### Step B: tert-butyl 2-(4-(nitrooxy)butylamino)-2-oxoethylcarbamate

To a solution of tert-butyl 2-(4-hydroxybutylamino)-2-oxoethylcarbamate (2.16 g, 8.77 mmol), tetraethylammonium nitrate (3.37 g, 17.54 mmol) and 2,6-di-tert-butyl-4-methylpyridine (2.71 g, 13.16 mmol) in CH₂Cl₂ (60 ml) cooled to −70° C. and under nitrogen, a solution of trifluoromethansulfonic anhydride (2.72 g, 9.65 mmol) in CH₂Cl₂ (40 ml) was added drop wise. The resulting mixture was stirred for 3 hours at −65° C. Then the mixture was slowly warmed to room temperature, diluted with CH₂Cl₂ and washed with 5% aqueous Na2HPO4. The organic layer was dried over sodium sulphate and concentrated under reduced pressure.

The residue was purified by flash chromatography CH₂Cl₂/CH3CN 70:30 as eluent) affording the title compound

### Step C: 4-(2-aminoacetamido)butyl nitrate

To a solution of tert-butyl 2-(4-(nitrooxy)butylamino)-2-oxoethylcarbamate (1.73 g 7.0 mmol) in CH₂Cl₂ (40 ml) cooled to 0° C., HCl_{gas} was bubbled for 2 hours. The solvent was concentrated and the residue was treated with diethyl ether, affording the title compound as a white solid.

### INTERMEDIATE 14

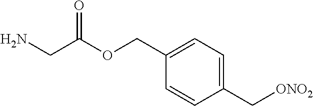

### 4-[(nitrooxy)methyl]benzyl 2-aminoacetate

### Step A 4-(chloromethyl)benzyl 2-(tert-butoxycarbonylamino)acetate

The title compound was prepared from N-Boc-Glycine N-hydroxysuccinimido ester and 4-(chloromethyl)benzyl alcohol following procedure reported in Intermediate 3 Step A.

### Step B: 4-[(nitrooxy)methyl]benzyl 2-(tert-butoxycarbonylamino)acetate

To a solution of 4-(chloromethyl)benzyl 2-(tert-butoxycarbonylamino)acetate ester (0.605 g, 1.78 mmol) in CH₃CN (13 ml), AgNO₃ (0.756 g, 4.45 mmol) was added and the reaction was heated in a microwave apparatus (150° C., 20 min). The formed salts were filtered off and the solvent was concentrated, then the residue was diluted with EtOAc (100 ml) and washed with brine. The organic layer was dried over sodium sulphate and concentrated under reduced pressure yielding the title compound.

### Step C: 4-[(nitrooxy)methyl]benzyl 2-aminoacetate

The title compound was obtained by acid hydrolysing 4-[(nitrooxy)methyl]benzyl 2-(tert-butoxycarbonylamino)acetate following procedure described in Intermediate 2 Step B.

### Studies on Vascular Tone

The ability of ARB nitroderivatives to induce vasorelaxation in comparison to native ARBs, was tested in vitro in isolated rabbit thoracic aorta preparations (Wanstall J. C. et al., Br. J. Pharmacol., 134:463-472, 2001). Male New Zealand rabbits were anaesthetized with thiopental-Na (50 mg/kg, iv), sacrificed by exsanguinations and then the thorax was opened and the aorta dissected. Aortic ring preparations (4 mm in length) were set up in physiological salt solution (PSS) at 37° C. in small organ chambers (5 ml). The composition of PSS was (mM): NaCl 130, NaHCO₃ 14.9, KH₂PO₄ 1.2, MgSO₄ 1.2, HEPES 10, CaCl₂, ascorbic acid 170 and glucose 1.1 (95% O₂/5% CO₂; pH 7.4). Each ring was mounted under 2 g passive tension. Isometric tension was recorded with a Grass transducer (Grass FT03) attached to a BIOPAC MP150 System. Preparations were allowed to equilibrate for 1 h, and then contracted submaximally with noradrenaline (NA, 1 μM) and, when the contraction was stable, acetylcholine (ACh, 10 μM) was added. A relaxant response to ACh indicated the presence of a functional endothelium. Vessels that were unable to contract NA or showed no relaxation to Ach were discarded. When a stable precontraction was reached, a cumulative concentration-response curve to either of the vasorelaxant agents was obtained in the presence of a functional endothelium. Each arterial ring was exposed to only one combination of inhibitor and vasorelaxant. Moreover, the effect of the soluble guanylyl cyclase inhibitor ODQ (1-H-(1,2,4)-oxadiazol(4,3-a)quinoxalin-1-one) on vasorelaxation elicited by the compounds was examined preincubating the aortic rings with ODQ (10 μM) for 20 min.

Responses to relaxing agents are expressed as a percentage of residual contraction and plotted against concentration of test compound. EC₅₀ values (where EC₅₀ is the concentration producing 50% of the maximum relaxation to the test compound) were interpolated from these plots. During the experimental period, the plateau obtained with NA was stable without significant spontaneous loss of contraction in the aortic rings. Under these experimental conditions, the parent compounds did not produce relaxation at any of the concentration tested, the curve being not different from that built up in the presence of vehicle alone.

As shown in Table 1, the compounds of the invention were able to induce relaxation in a concentration-dependent manner. Furthermore, in experiments performed in the presence of ODQ (10 μM), the vasorelaxant responses to tested compounds were inhibited.

TABLE 1

| Compound | EC₅₀ (μM) ± sem |
|---|---|
| Losartan | no effect |
| EXP 3174 | no effect |
| Candesartan | no effect |
| Compound of EX. 1 | 23.6 ± 3.9 |
| Compound of EX. 2 | 40.1 ± 2.7 |
| Compound of EX. 3 | 9.2 ± 4.1 |
| Compound of EX. 4 | 46 ± 7.2 |
| Compound of EX. 5 | 9.2 ± 2.5 |
| Compound of EX. 6 | 14.2 ± 8 |
| Compound of EX. 7 | 7.1 ± 1.9 |
| Compound of EX. 8 | 10.4 ± 2.5 |
| Compound of EX. 9 | 3.4 ± 0.8 |
| Compound of EX. 10 | 5.4 ± 1.8 |
| Compound of EX. 11 | 38.7 ± 5.3 |
| Compound of EX. 13 | 30.6 ± 4.7 |
| Compound (2) | 43.4 ± 11 |
| Compound (112) | 47.8 ± 16 |

### Study on Angiotensin Receptor Antagonism

### Tissue Preparation

Thoracic aorta was obtained from male rabbits. Each animal was sacrificed by exsanguinations under pentobarbital-Na anesthesia and the thoracic aorta was rapidly removed. After removing adhering fat and connecting tissues, the thoracic aorta was cut into 4-5 mm long rings. Each preparation was placed in a 5 ml organ bath containing physiological salt solution (PSS) at the following composition (mM): NaCl 130.0, KCl 3.7, NaHCO₃ 14.9, KH₂PO₄ 1.2, MgSO₄.7H₂O 1.2, Glucose 11.0, HEPES 10.0, CaCl₂.2H₂O 1.6. The solution in the bath was constantly aerated with 95% O₂ and 5% CO₂ and kept at 37° C. (pH 7.4). Contractile force was measured with isometric transducer (Grass FT03), connected to a BIOPAC MP150 System. After 1 h of equilibration with a appropriate resting tension (2 g), the rings were primed by exposure to 90 mM KCl (3 times) with intervening washings. Then each ring was contracted submaximally with methoxamine 3 μM and, when the contraction was stable, acetylcholine (ACh, 3 μM) was added. A relaxant response to ACh indicates the presence of a functional endothelium.

Interaction with Angiotensin II Receptors

The vascular preparations were exposed to increasing concentrations of Angiotensin II (AngII) until the maximal contractile effect was achieved. Then the vascular tissues were washed and recovered to baseline. After an incubation time of 30 minutes with antagonist at the selected concentration, a second concentration-response curve for agonist was constructed. In parallel control experiments, vehicle was given before the second agonist curve was constructed.

Responses are expressed as percentage maximal contractile effect achieved in the first cumulative curve.

Antagonist activity was assessed by the ability of compounds to produce rightward shifts in the dose response curve for angiotensin II-induced contraction of the isolated rabbit aorta.

All the compounds of the invention showed an angiotensin II receptor antagonism.

Study of Antihypertensive Activity of ARB Nitroderivatives In Vivo

The ability of the compounds of the invention to decrease blood pressure was evaluated in conscious spontaneously hypertensive rats (SHRs). SHRs (250-300 g) received a single oral dose of EXP 3174 or the corresponding nitroderivative. Systolic blood pressure (SBP) and heart rate were monitored by telemetry for 24 hours after dosing. SBP was evaluated before (baseline) and after (i.e. 6, 24 hours) treatment by oral administration of the compounds. The data were processed both as the absolute value or as a delta between the absolute value and its own baseline.

The Dataquest IV telemetry system (Data Sciences International) was used for measurement of systolic pressure, diastolic pressure, mean arterial pressure, heart rate, and motor activity. The monitoring system consists of a transmitter (radio frequency transducer model TA11PA), receiver panel, consolidation matrix, and personal computer with accompanying software. Before the device was implanted, calibrations were verified to be accurate within ±3 mmHg. Rats were anesthetized with ketamine/xylazine/acepromazine, and the flexible catheter of the transmitter was surgically secured in the abdominal aorta just below the renal arteries. The transmitter was sutured subcutaneously. Rats were housed in individual cages after the operation. Each cage was placed over the receiver panel that was connected to the personal computer for data acquisition. The rats were unrestrained and free to move within their cages. Hemodynamic data were sampled every 2 minutes for 10 seconds.

As shown in Table 2, the nitroderivatives of the invention were able to induce a reduction in blood pressure levels over the treatment period, associated with prolonged duration of action and resulted more effective than the parent compounds.

TABLE 2
Δ Systolic blood pressure (mmHg)

| Compound | 6 hrs | 24 hrs |
|---|---|---|
| EXP 3174 (10 mpk, po)* | −5 | 2 |
| Compound of EX. 5(10 mpk, po) | −7 | −11 |
| Compound of EX. 6(10 mpk, po) | −12 | −13 |
| Compound of EX. 10(10 mpk, po) | −6 | −11 |
| Compound of EX. 11(10 mpk, po) | −6 | −17 |

*Systolic blood pressure for EXP 3174 was evaluated with tail-cuff method (Whitesall S. E et Al.; Am. J. Physiol. Heart Circ. Physiol 286: H2408-H2415, 2004).

### Solubility Test

The solubility of the tested compounds in PEG 400/H₂O 7/3 was evaluated using a standard curve constructed of six calibration points by plotting the peak areas of the compounds of the invention versus the concentration.

Test solution: 5.0 mg of the nitroderivative were dissolved in 0.7 ml of PEG 400 in a glass volumetric flask and 0.3 ml of water were added. The solution was mixed for 1 hour and filtered through 0.45 μm Acrodisc filter.

As shown in Table 3, the nitroderivatives of the invention showed a very good solubility.

TABLE 3

| | Solubility |
|---|---|
| | PEG400/H₂O 70:30 (mg/ml) |
| Compound | target 5 mg/ml |
| (2) | >5 |
| Compound of EX. 3 | >5 |
| Compound of EX. 2 | >5 |
| (112) | >5 |
| Compound of EX. 1 | 4 |
| Compound of EX. 5 | 4.2 |
| Compound of EX. 6 | >5 |
| Compound of EX. 9 | 4.9 |
| Compound of EX. 7 | >5 |
| Compound of EX. 11 | 4.6 |
| (46) | 5.07 |

1. A compound of general formula (I) or a pharmaceutically acceptable salt or stereoisomer thereof: 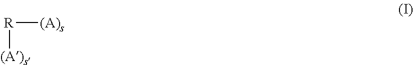
wherein:
A and A′ are independently selected from the group consisting of —(Y—ONO2), —(Y′—ONO2) or (1a) 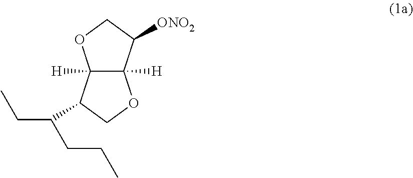
s is 1 or 2;
s′ is 0, 1 or 2;
R is selected from the following residues of formula (II) or (III): 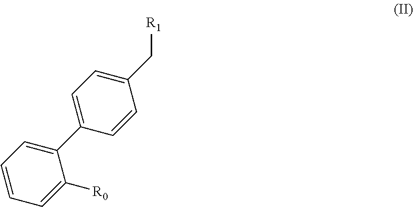
wherein: 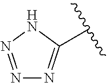
R₀ is the group (IV) or N₀ which is a moiety capable to bind the groups A and A′ as defined hereinafter;
R₁ is selected from the groups (Va-Ve): 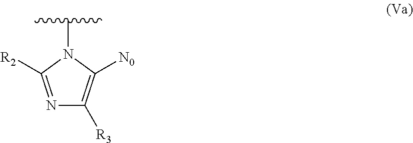
wherein R₂ is C₁-C₅ linear or branched alkyl, preferably n-propyl or n-butyl;
R₃ is an halogen atom such as C1, Br, I, or a perfluorurated C₁-C₄ alkyl chain, preferably C₂F₅, or the group —C(CH₃)₂OH; 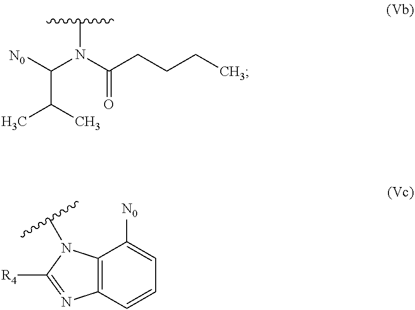
wherein R₄ is n-Bu or —OEt; 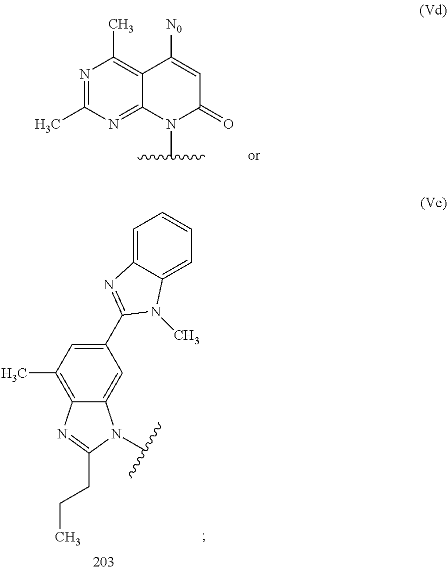
or R is the residue of formula (III): 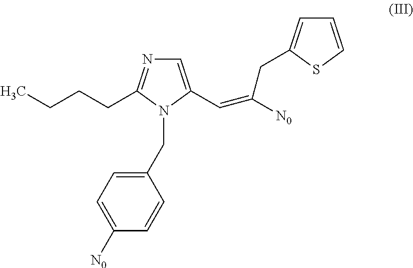
wherein N₀ is a moiety capable to bind the groups A and A′, having one of the following meanings:
1) 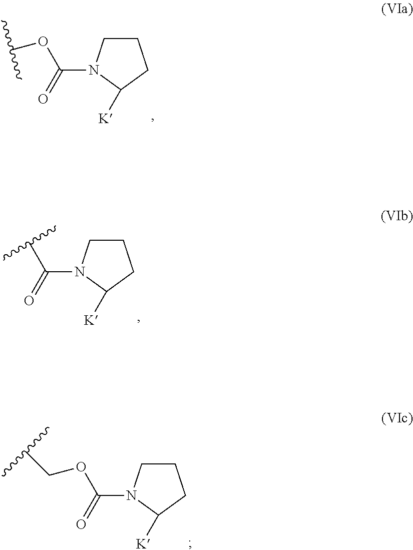
wherein K″ is equal to —COO—, —CONH—, —CH₂—O—CO—, —CH₂—O—COO— or —CH₂—O—CONH— and K″ is bound to the group A wherein A is —(Y—ONO₂) or (1a), with the proviso that when A is (1a), then K′ is —COO— or —CH₂—OCOO—;
2) —OCO—NH-J-K″, —CO—NH-J-K″ or —CH₂—O—CO—NH-J-K″ wherein J is selected among (VIIa-VIIk): 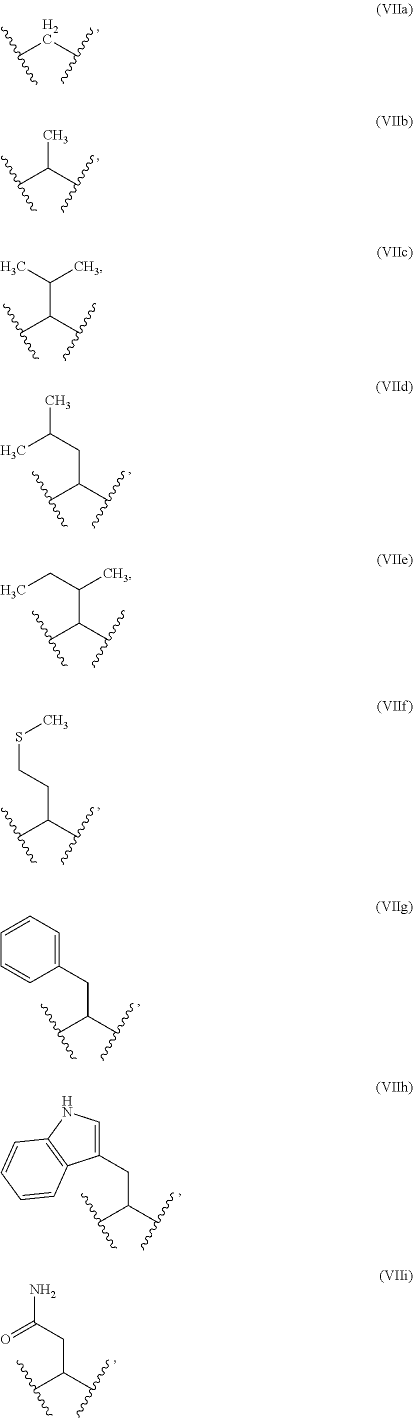 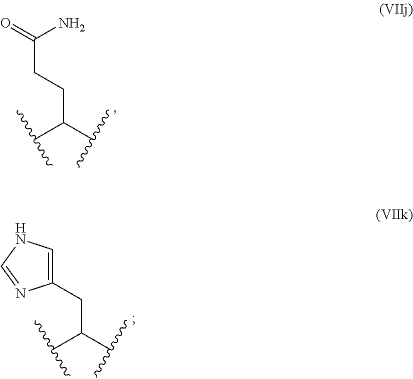
wherein K′ is equal to —COO—, —CONH—, —CH₂—O—CO—, —CH₂—O—COO— or —CH₂—O—CONH— and K″ is bound to the group A wherein A is —(Y—ONO₂) or (1a), with the proviso that when A is (1a), then K′ is —COO— or —CH₂—OCOO—;
3) —O—CO—NH—K—K*, —CH₂—O—CO—NH—K—K* or —CO—NH—K—K* wherein K is selected among K₁, K₂ or K₃ wherein:
K₁ is selected among (VIIIa-VIIId): 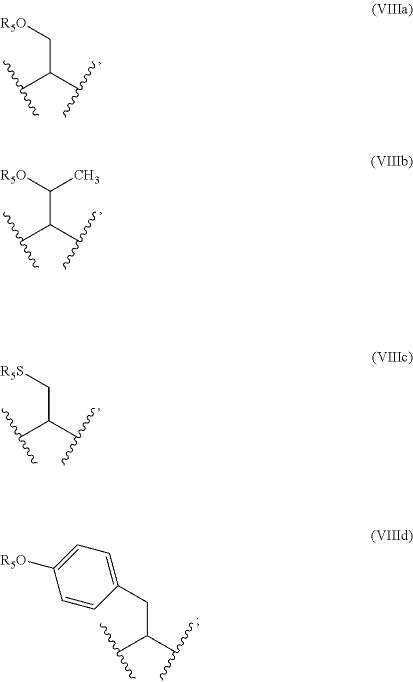
wherein R₅ is H or a group selected from —CO—, —COO— or —CONH— capable to bind a group A″ wherein A′ is —(Y—ONO₂); K₂ is selected among (VIIIe-VIIIf): 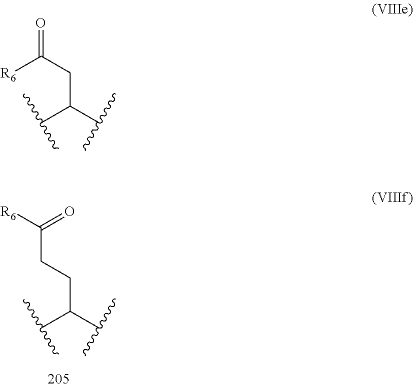
wherein R₆ is —OH or a group selected from —O— or —NH capable to bind a group A′, with the proviso that when A″ is (1a), then R₆ is —O—;
K₃ is selected among (VIIIg-VIIIh): 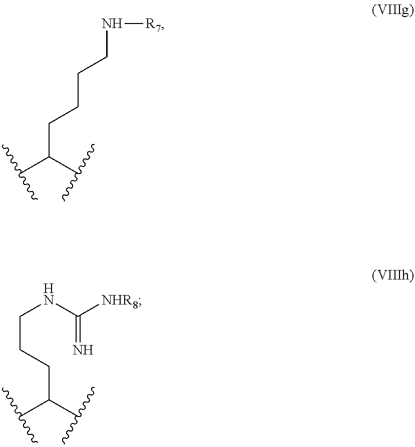
wherein R₇ and R₈ are H or a group selected from —CO— or —COO— capable to bind a group A′ wherein A′ is —(Y′—ONO₂);
K* is equal to K″ as above defined or —COOH and when K* is equal to K′ is bound to the group A, with the proviso that when A is (1a), then K′ is —COO— or —CH₂—OCOO—;
4) 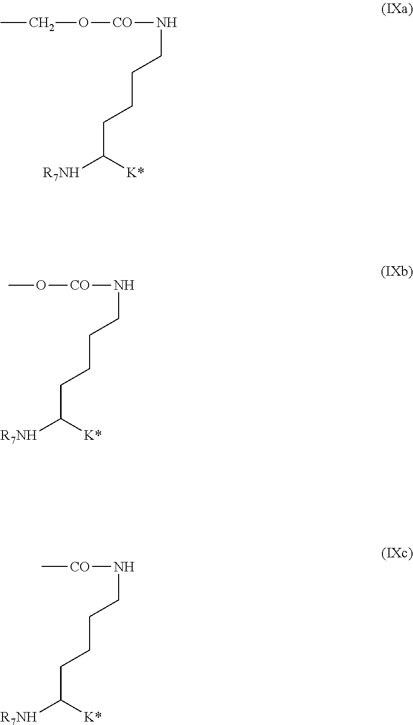
wherein R₇ and K* are as above defined;
with the proviso that:
i. when R₁ is the group (Va), then N₀ is selected from the group consisting of (VIb), (VIc) —CO—NH-J-K″,
a. —CH₂—O—CO—NH-J-K″, —CO—NH—K—K*, —CH₂—O—CO—NH—K—K*, (IXc) and (IXa);
ii. when R₁ is selected from the groups (Vb), (Vc) or (Ve), then N₀ is selected from the group consisting of (VIb), —CO—NH-J-K′, —CO—NH—K—K* and (IXc),
iii. when R₁ is the group (Vd), then N₀ is selected from the group consisting of (VIa), —OCO—NH-J-K″, —O—CO—NH—K—K* and (IXb);
iv. when R is selected from the residue (III), then N₀ is selected from the group consisting of (VIb), —CO—NH—K—K* and (IXc);
v. when R is selected from the residue (II) and R₀ is N₀, then R₁ is the group (Ve)
vi. when R is selected from the residue (II), then s is 1 and s″ is 0 or 1;
vii. when R is selected from the residue (III), then s is 2 and s′ is 0 or 2.
Y and Y′ independently are bivalent radicals having the following meaning:
a)
straight or branched C₁-C₂₀ alkylene, preferably C₁-C₁₀, being optionally substituted with one or more of the substituents selected from the group consisting of: halogen atoms, hydroxy, —ONO₂ or R¹, wherein R¹ is —OC(O)(C₁-C₁₀ alkyl)-ONO₂ or —O(C₁-C₁₀ alkyl)-ONO₂; 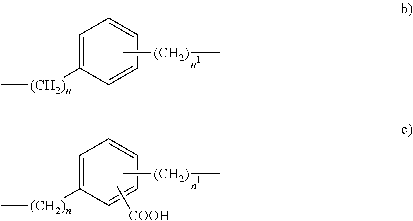
wherein n is an integer from 0 to 20, and n¹ is an integer from 1 to 20; 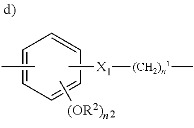
wherein;
n¹ is as defined above and n² is an integer from 0 to 2; X₁=—OCO— or —COO— and R² is H or CH₃; 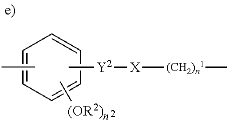
wherein:
n¹, n², R² and X₁ are as defined above;
Y² is —CH₂—CH₂— or —CH═CH—(CH₂)ₙ²—;
with the proviso that when Y or Y″ is selected from the bivalent radicals mentioned under b)-e), the —ONO₂ group is linked to a —(CH₂)ₙ¹ group; 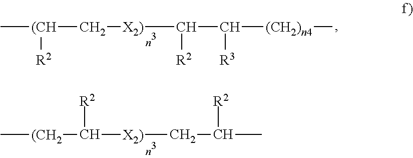
wherein X₂ is —O— or —S—, n³ is an integer from 1 to 6, preferably from 1 to 4, R² is as defined above,
R³ is H or —ONO₂ and n⁴ is 0 or 1.

2. The compounds of formula (I) according to claim 1 or a pharmaceutically acceptable salt or stereoisomer thereof wherein R is the residue of formula (II), R₀ is the group of formula (IV), R₁ is the group of formula (Va), R₂ is n-butyl, R₃ is C1 and all other variables are as defined in claim 1.

3. The compounds of formula (I) according to claim 1 or a pharmaceutically acceptable salt or stereoisomer thereof wherein R is the residue of formula (II), R₀ is the group of formula (IV), R₁ is the group of formula (Va), R₂ is n-propyl, R₃ is the group —C(CH₃)₂OH and all other variables are as defined in claim 1.

4. The compounds of formula (I) according to claim 1 or a pharmaceutically acceptable salt or stereoisomer thereof wherein R is the residue of formula (II), R₀ is the group of formula (IV), R₁ is the group of formula (Vc) as defined in claim 1, R₄ is —OEt, and all other variables are as defined in claim 1.

5. The compounds of formula (I) according to claim 1 or a pharmaceutically acceptable salt or stereoisomer thereof wherein s₁ is 0 and A is the group (VIa) or (VIb) or (VI_{c}) wherein K″ is —COO—, and all other variables are as defined in claim 1.

6. The compounds of formula (I) according to claim 1 or a pharmaceutically acceptable salt or stereoisomer thereof wherein s₁ is 0 and A is —CO—NH-J-K′ or —CH₂—O—CO—NH-J-K″, J is the group (VIIa) or (VIIb), wherein K′ is —COO—, and all other variables are as defined in claim 1.

7. The compounds of formula (I) according to claim 1 or a pharmaceutically acceptable salt or stereoisomer thereof wherein s₁ is 0 and A is —CH₂—O—CO—NH—K—K* or —CO—NH—K—K*, K is K₃ which is the group (VIIIg) or (VIIIₕ), and all other variables are as defined in claim 1.

8. A compound of general formula (I) or a pharmaceutically acceptable salt or stereoisomer thereof according to claim 1, wherein Y and Y″ independently are a bivalent radical having the following meaning:
a)
straight or branched C₁-C₁₀ alkylene, being optionally substituted with one or more —ONO₂; 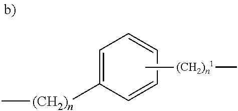
wherein n is an integer from 0 to 5, and n¹ is an integer from 1 to 5;
with the proviso that when Y or Y′ is selected from the bivalent radical b), the —ONO₂ group is linked to a —(CH₂)ₙ¹ group; 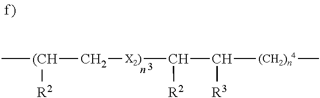
wherein X₂ is —O— or —S—, n³ is 1, R² is H, R³ is H or —ONO₂ and n⁴ is 0 or 1.

9. A compound according to claim 1, selected from the group consisting of: 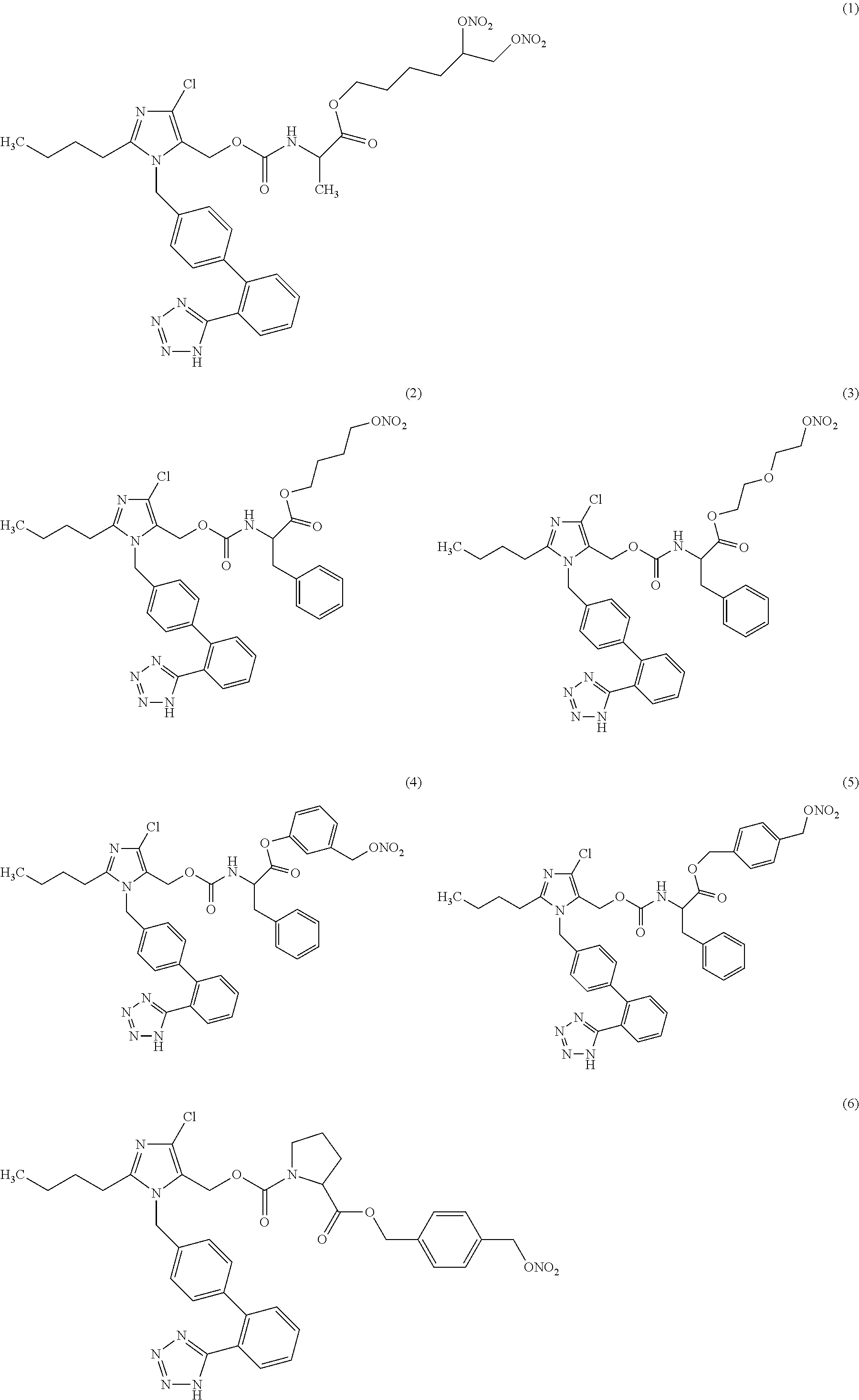 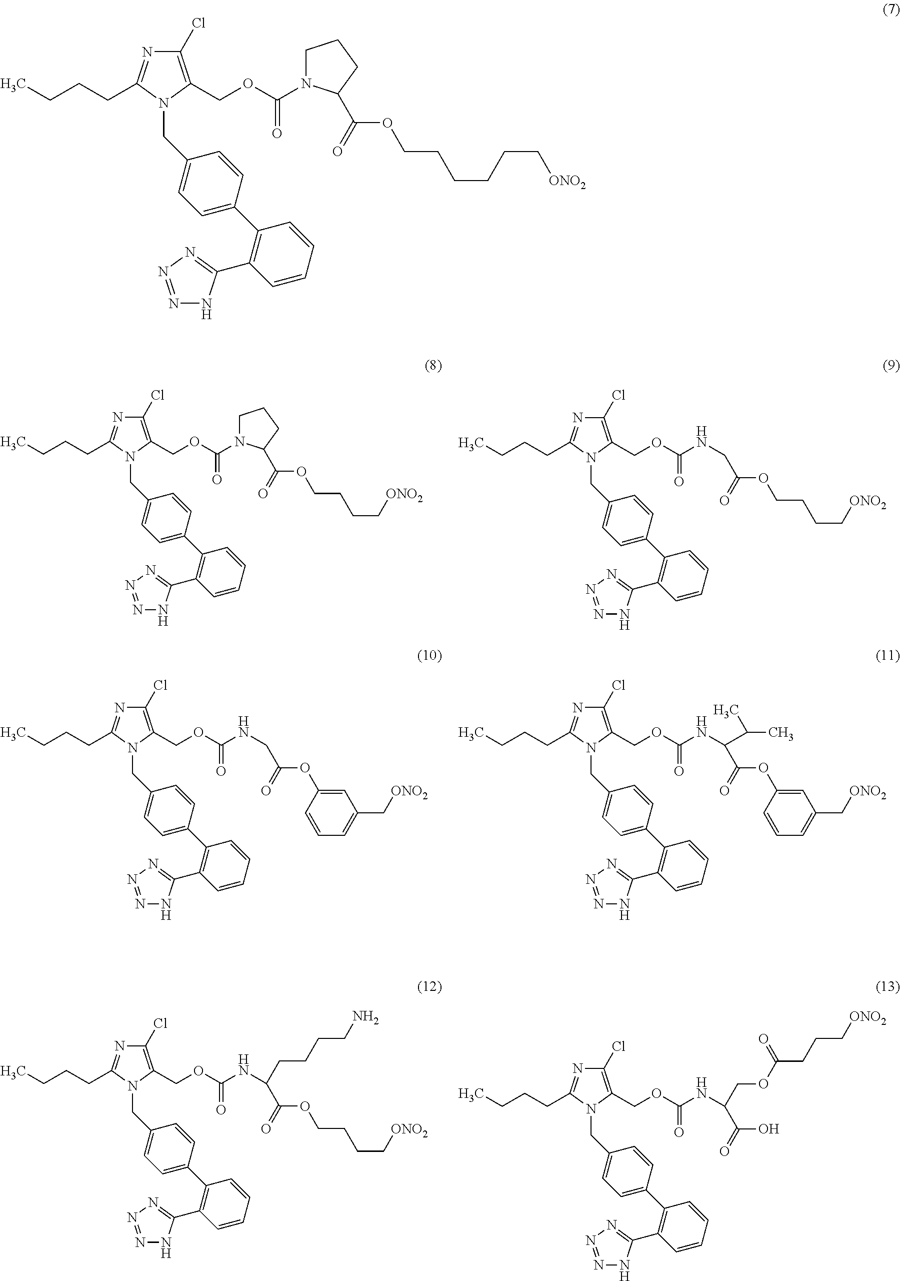 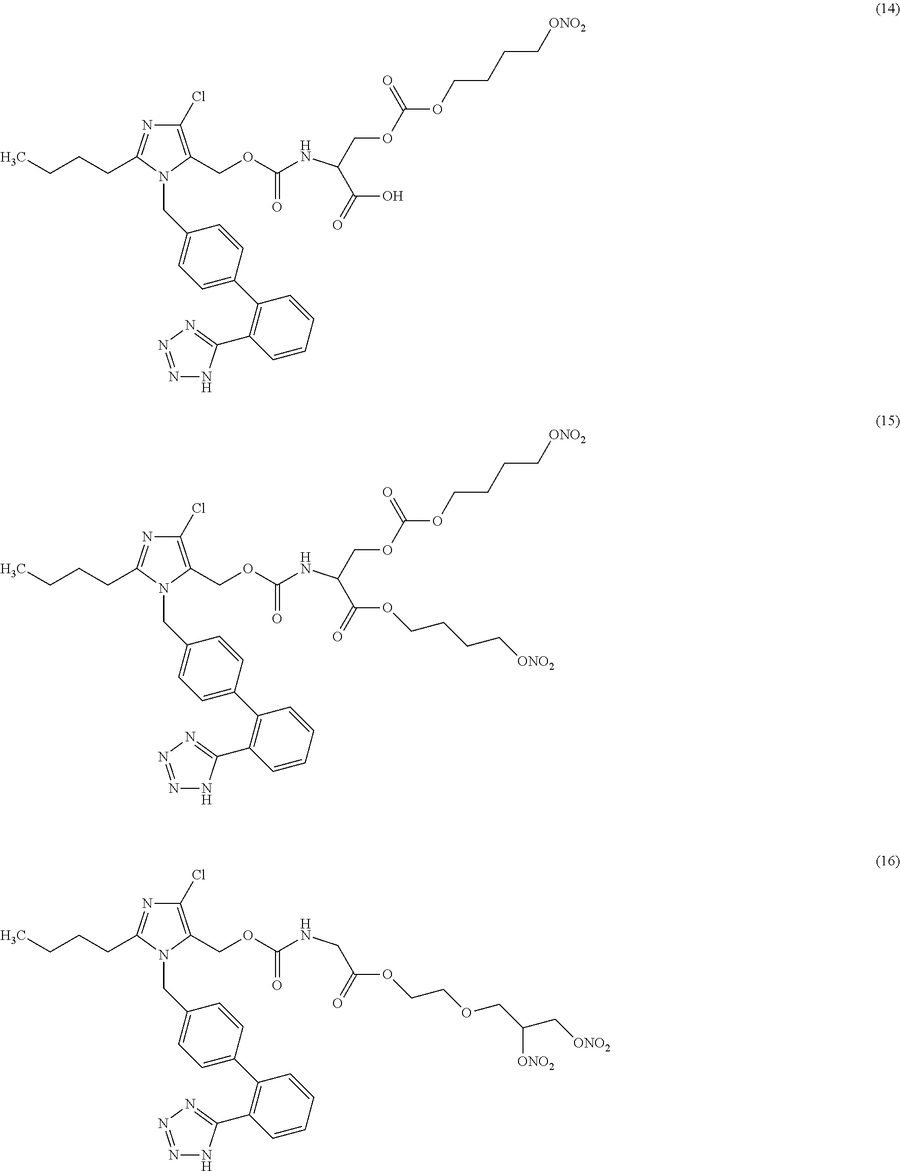 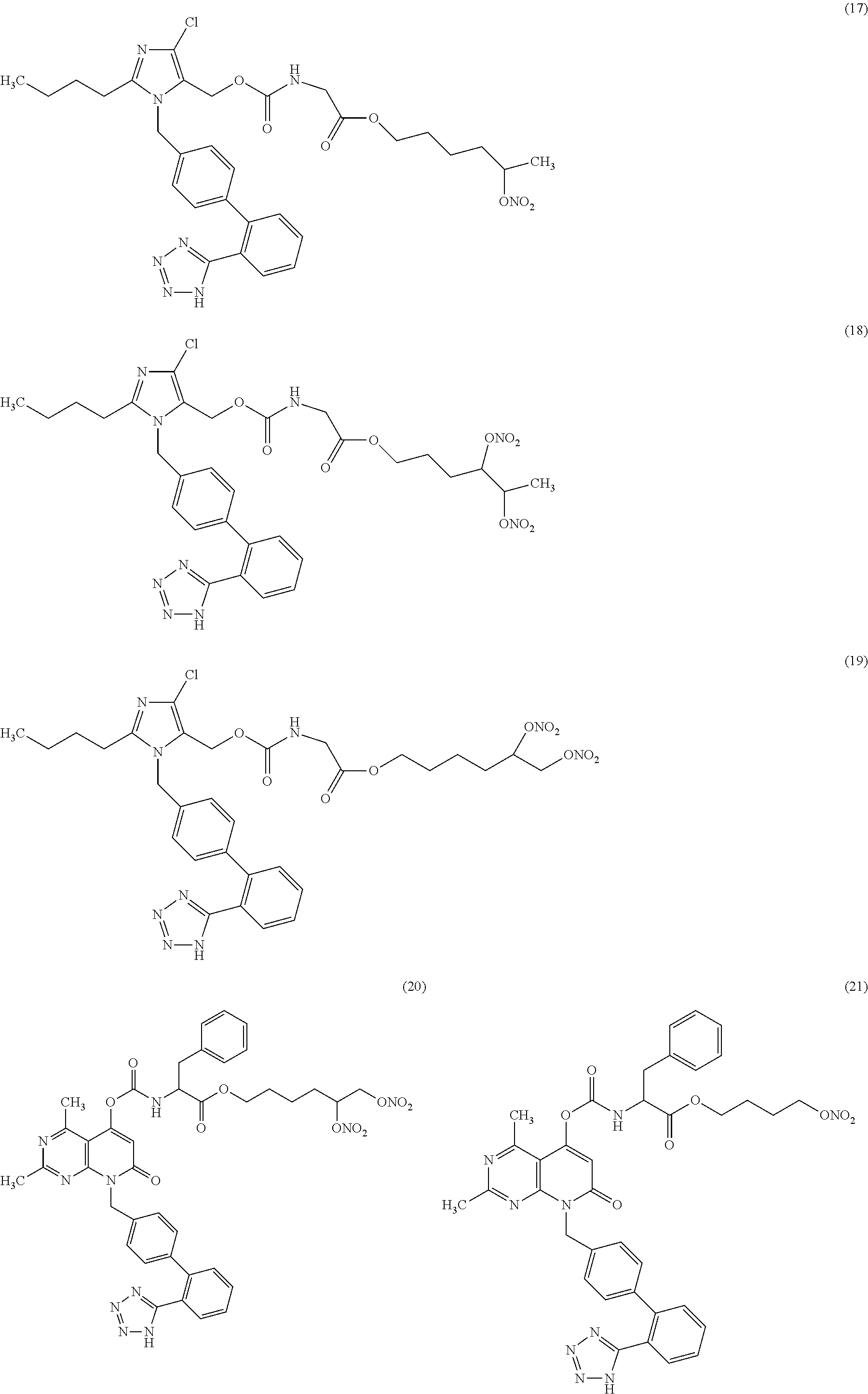 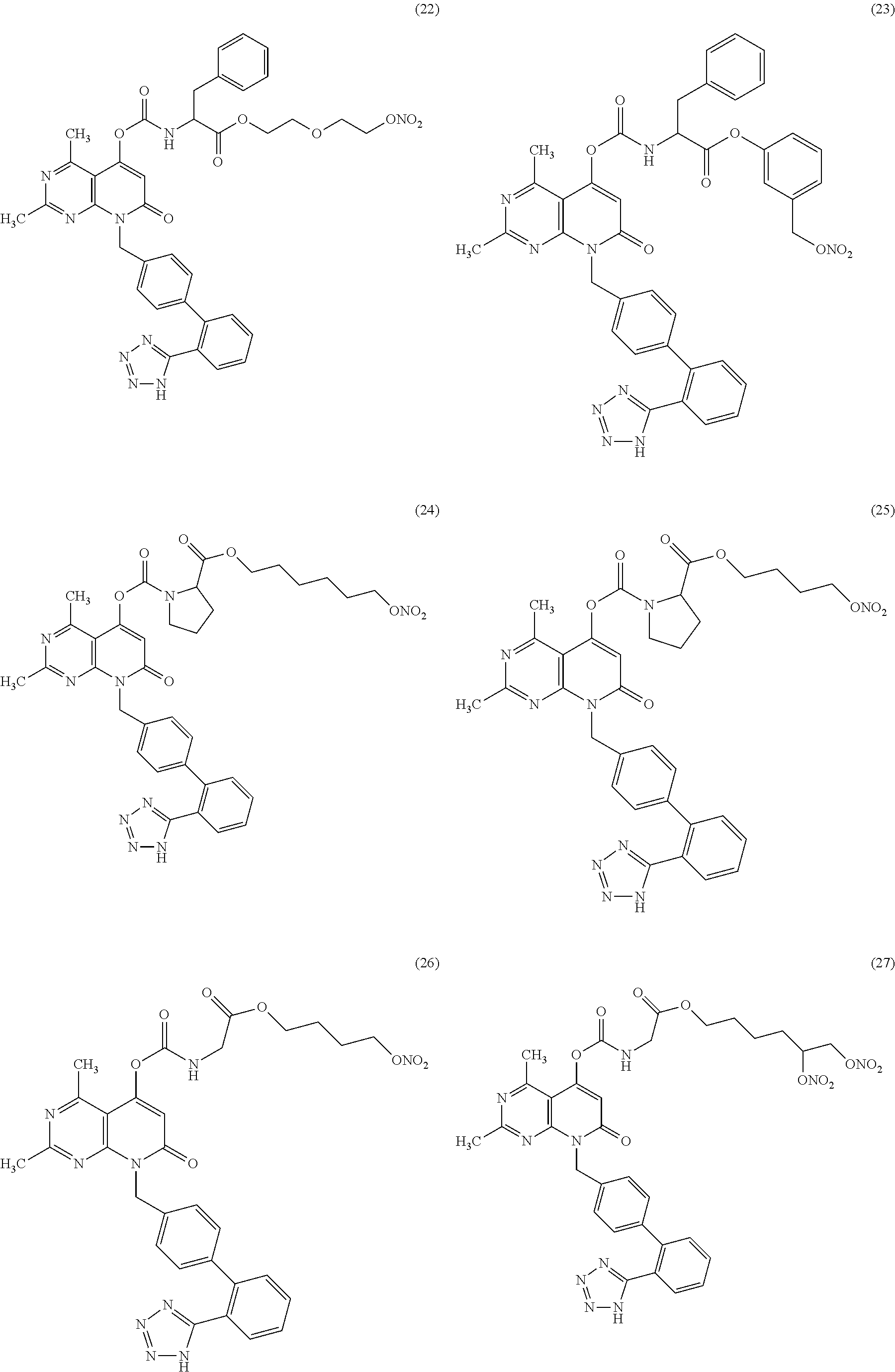 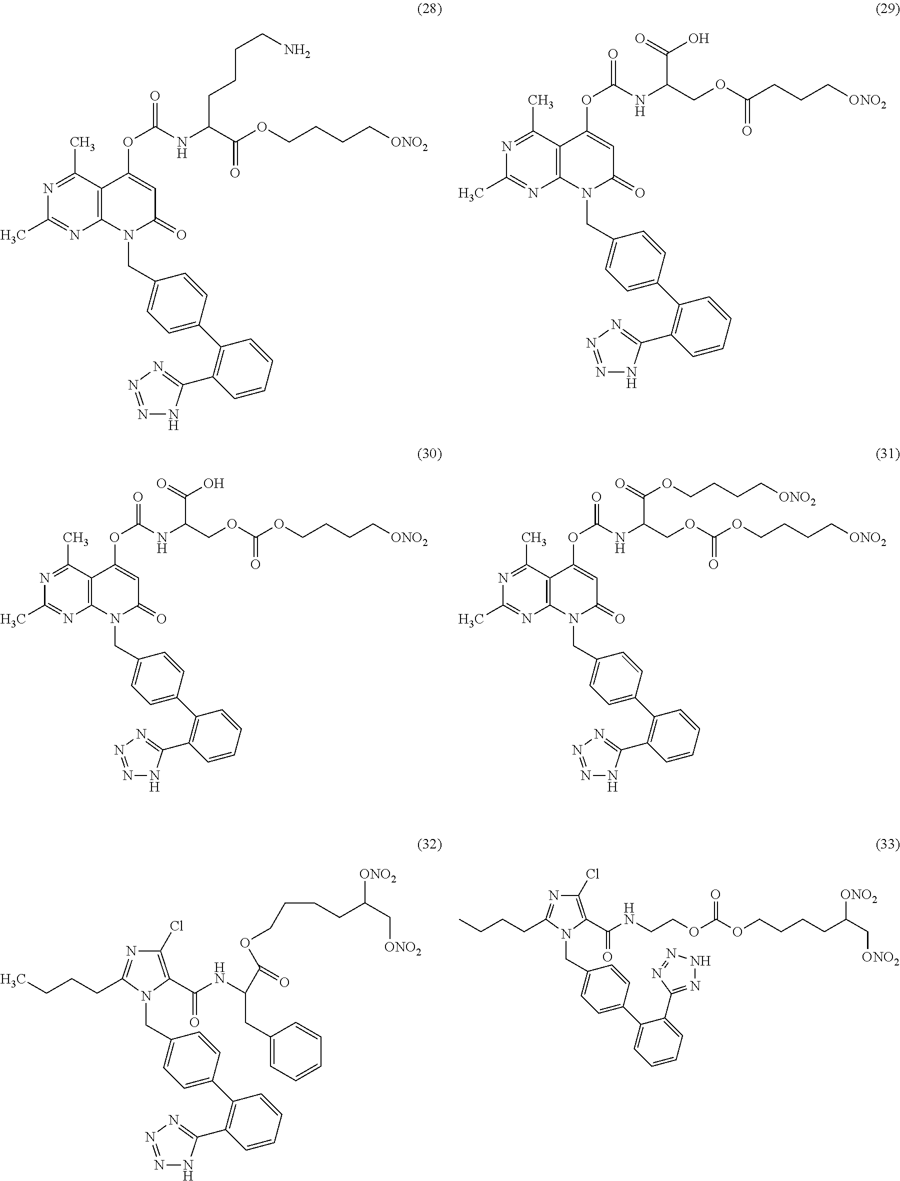 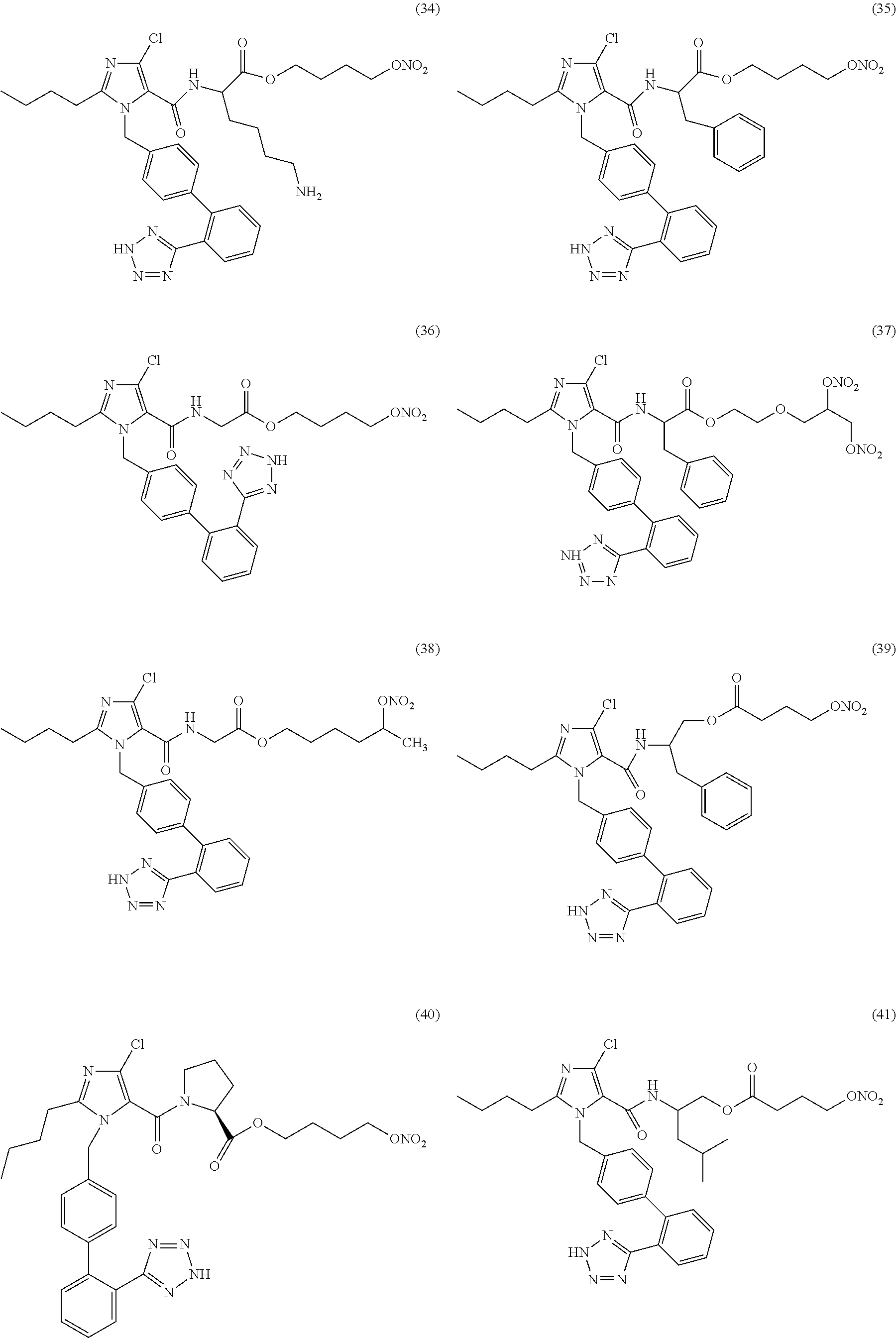 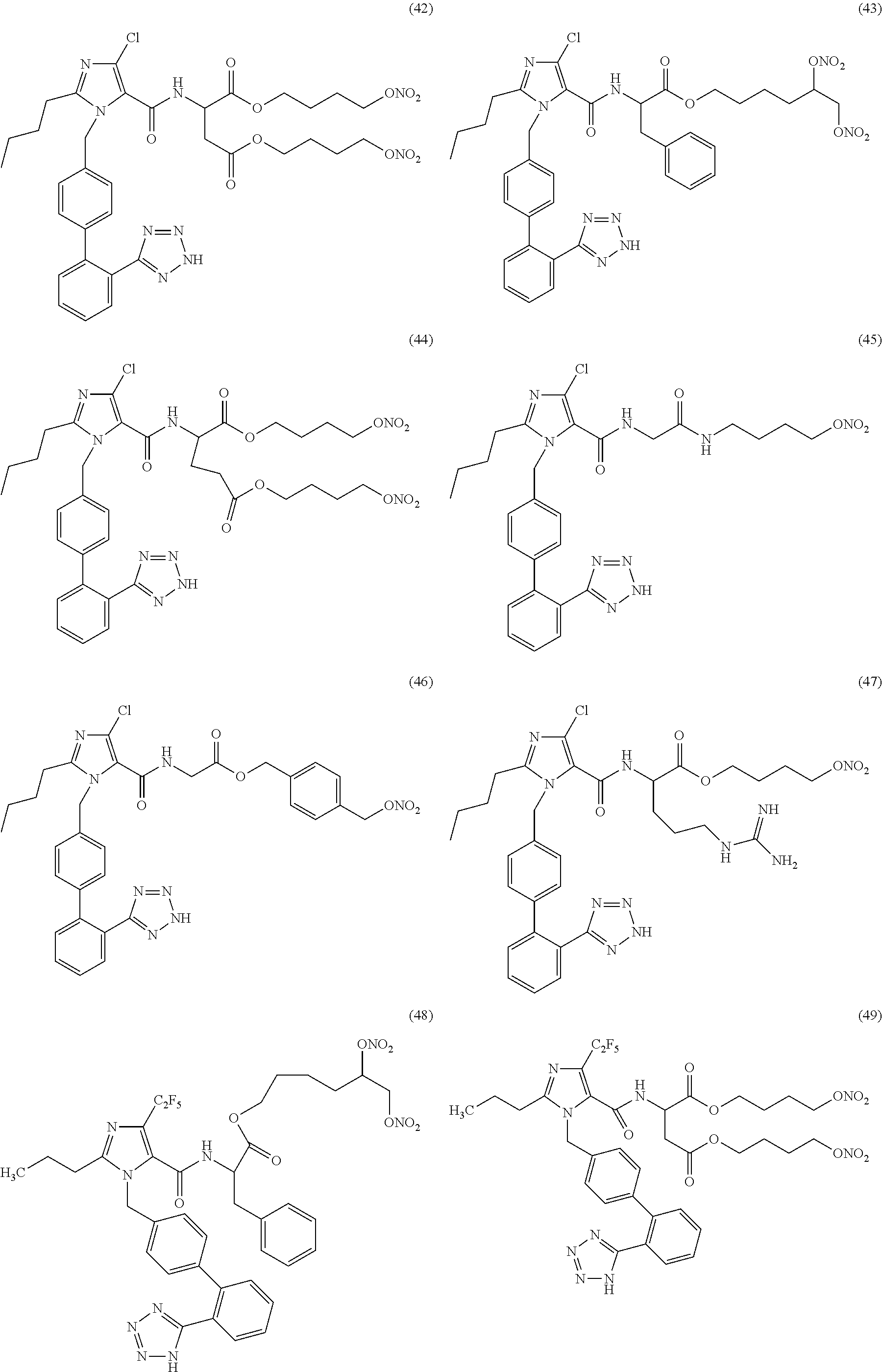 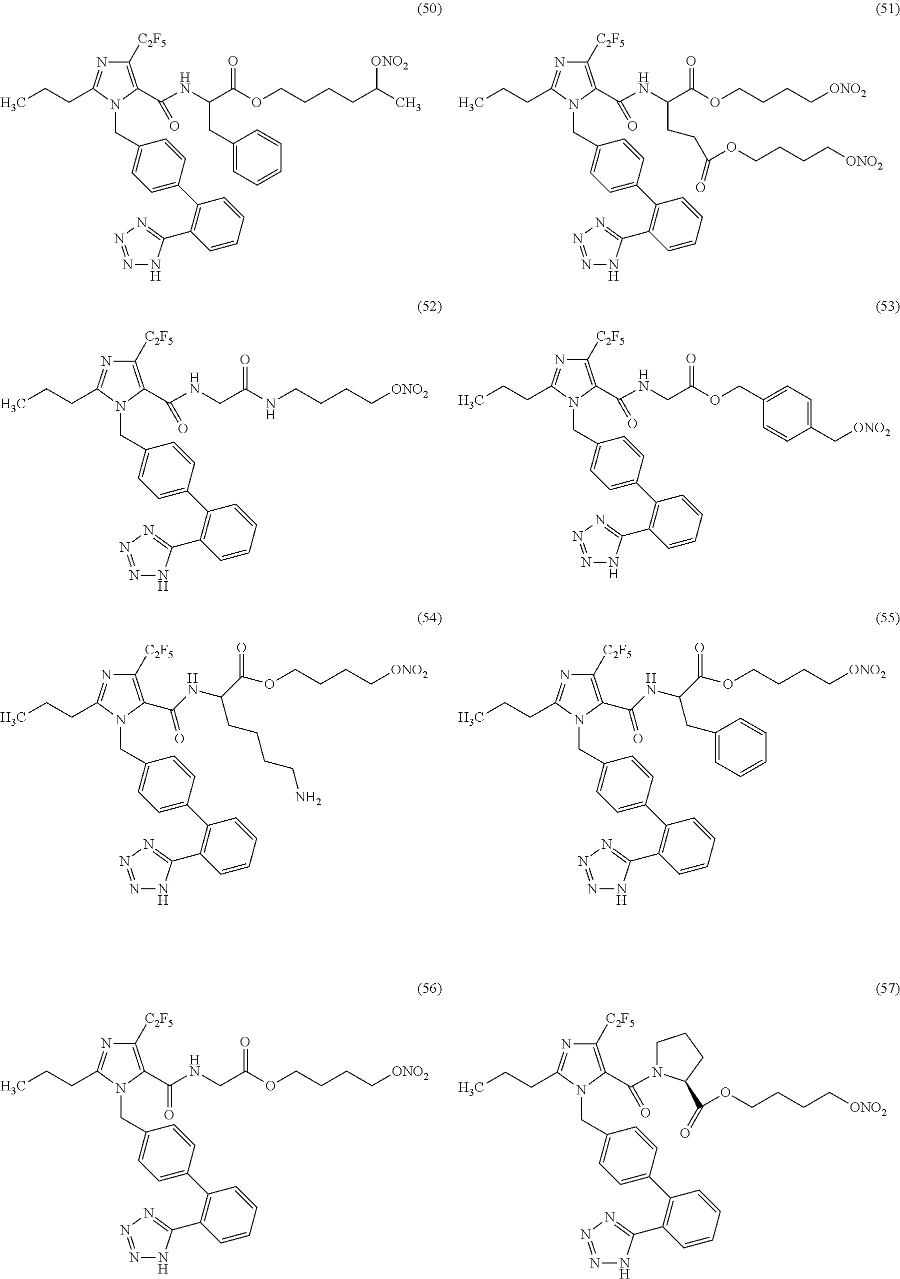 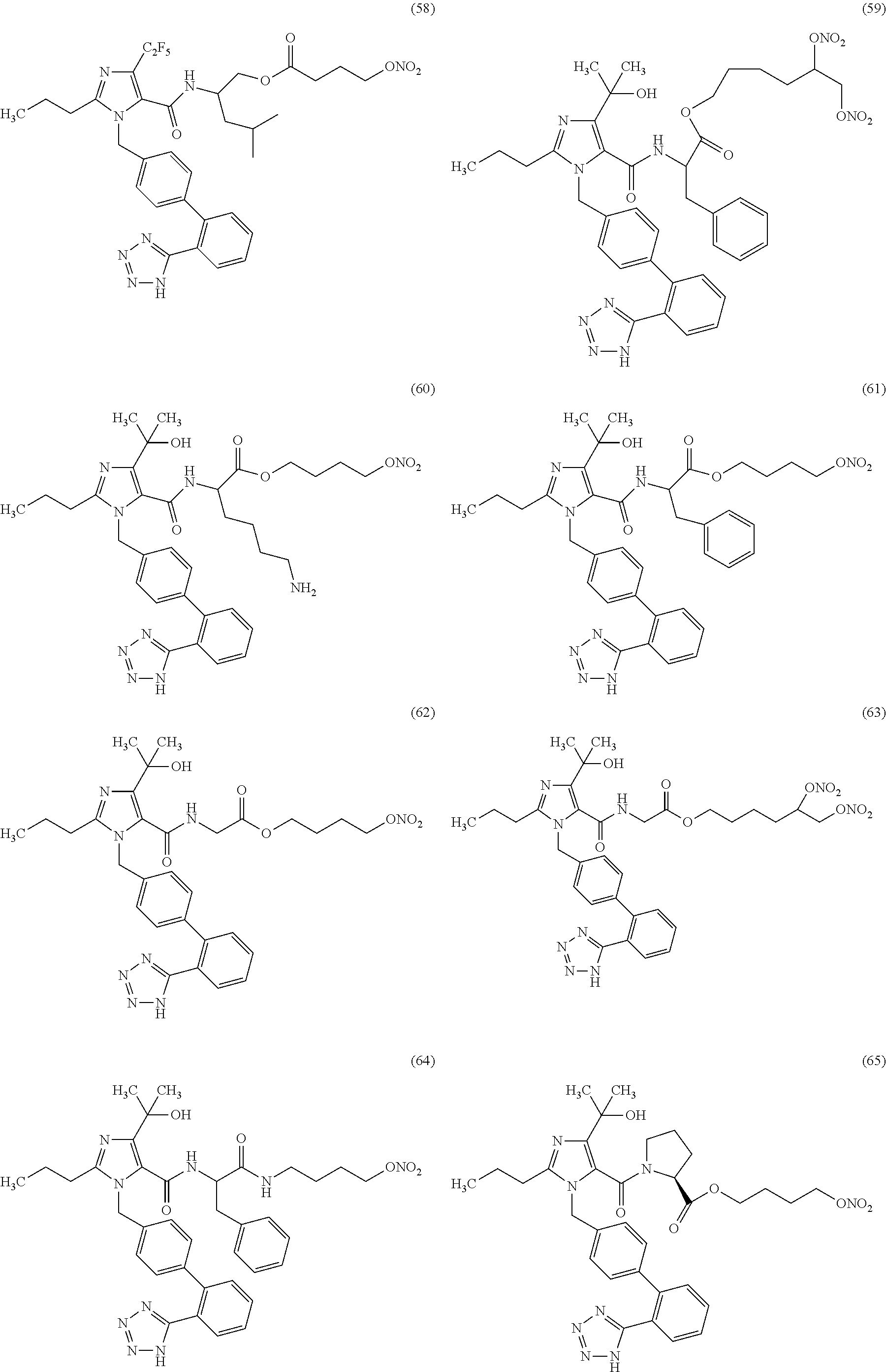 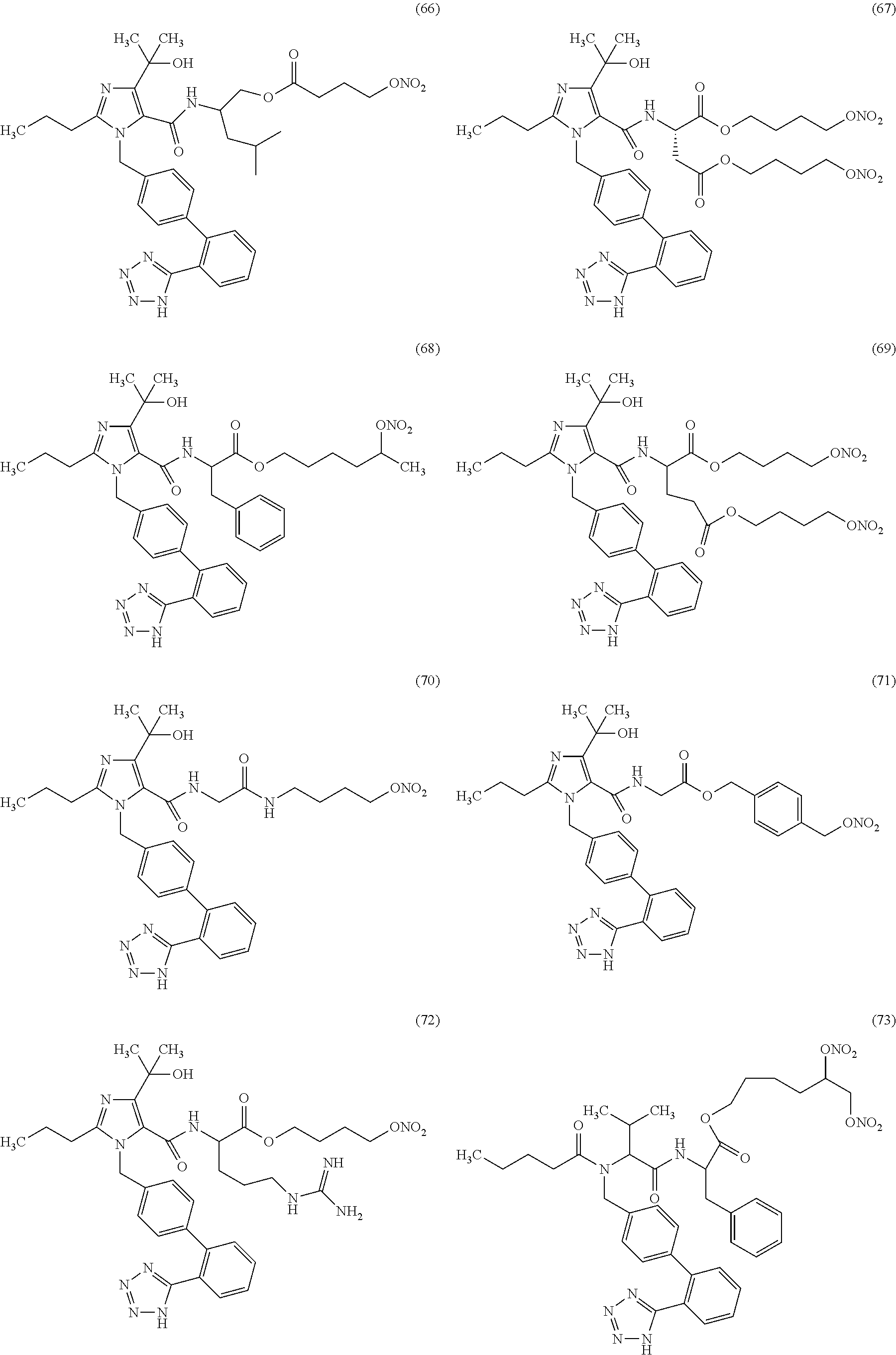 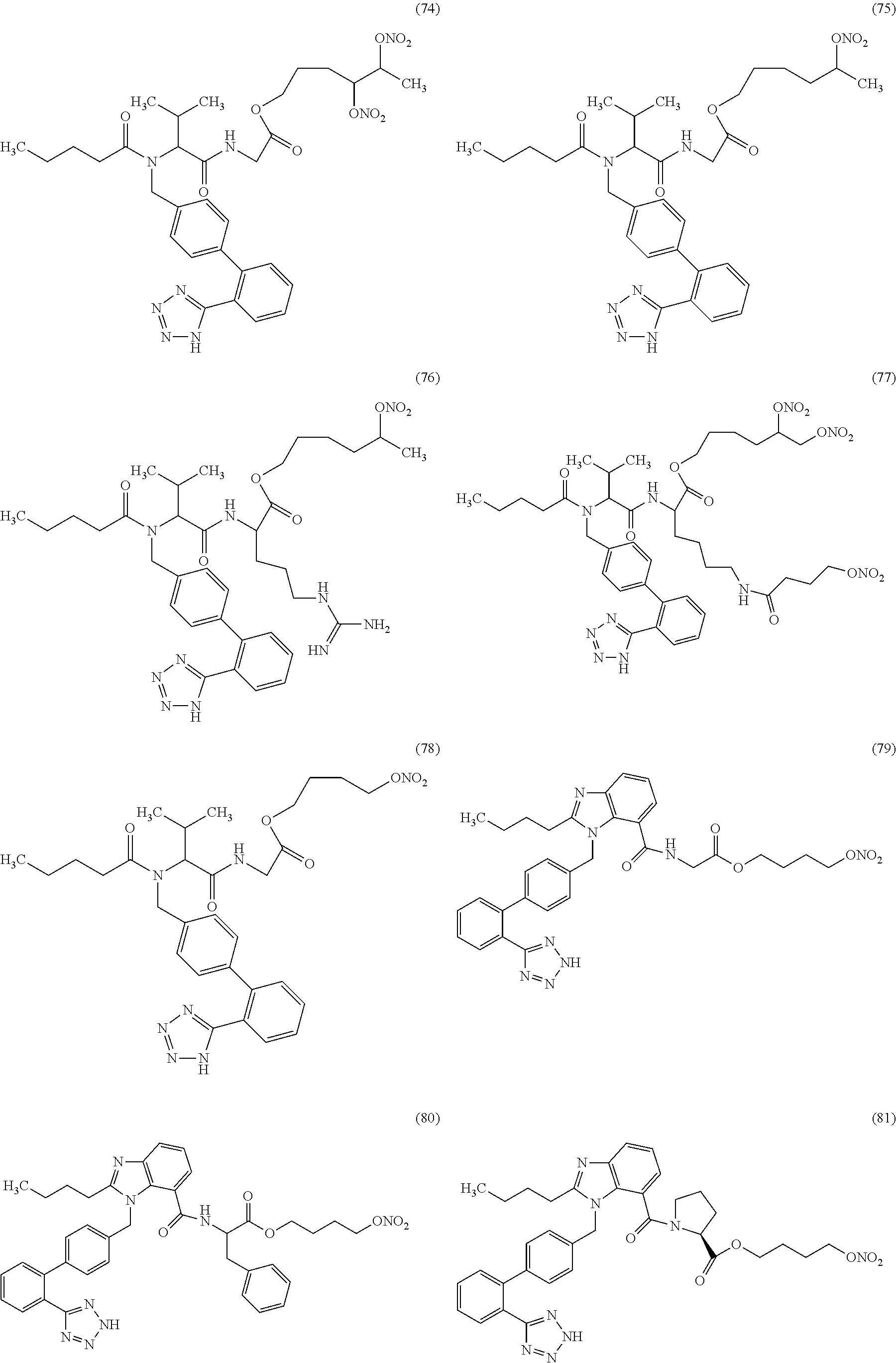 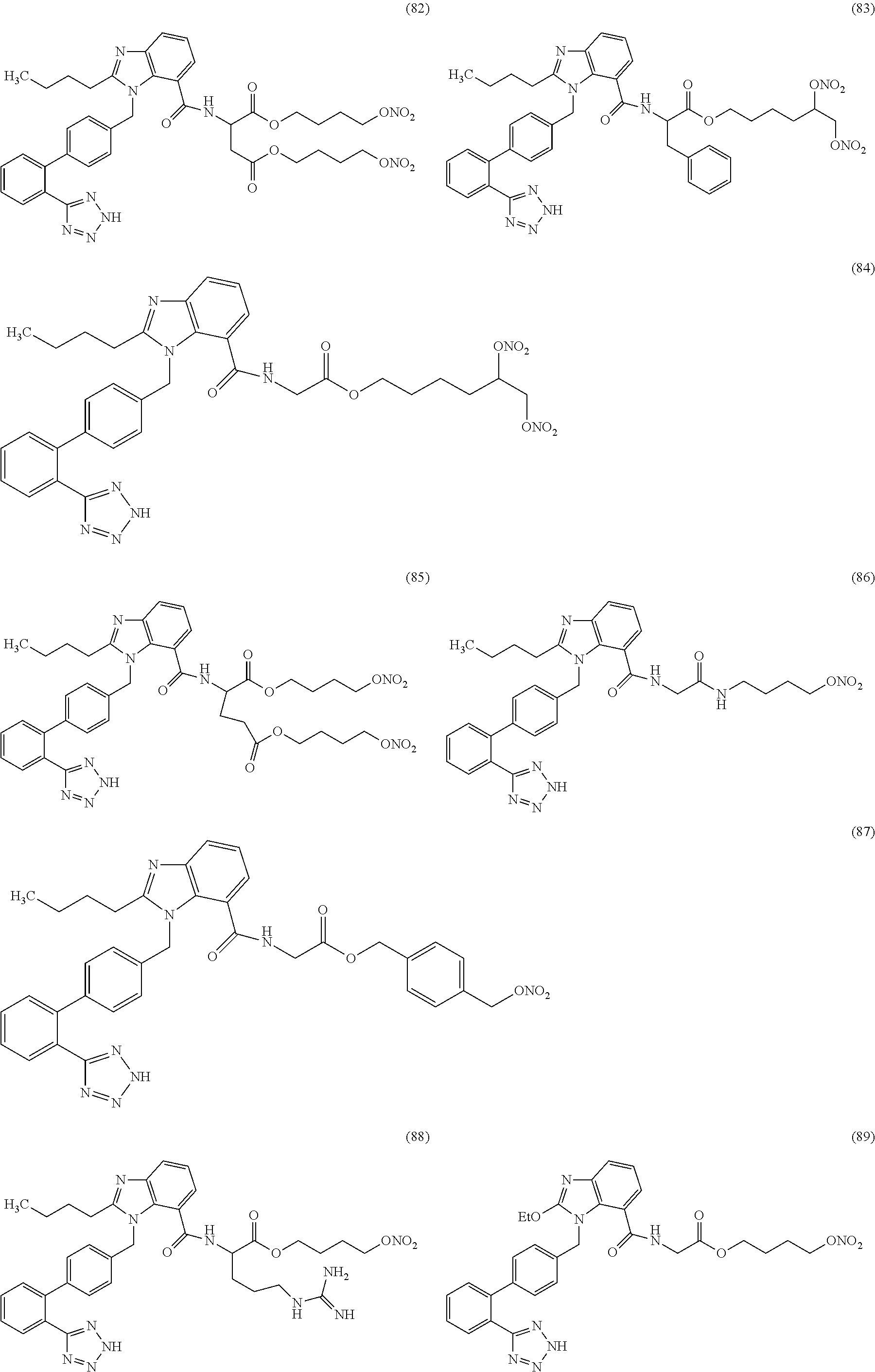 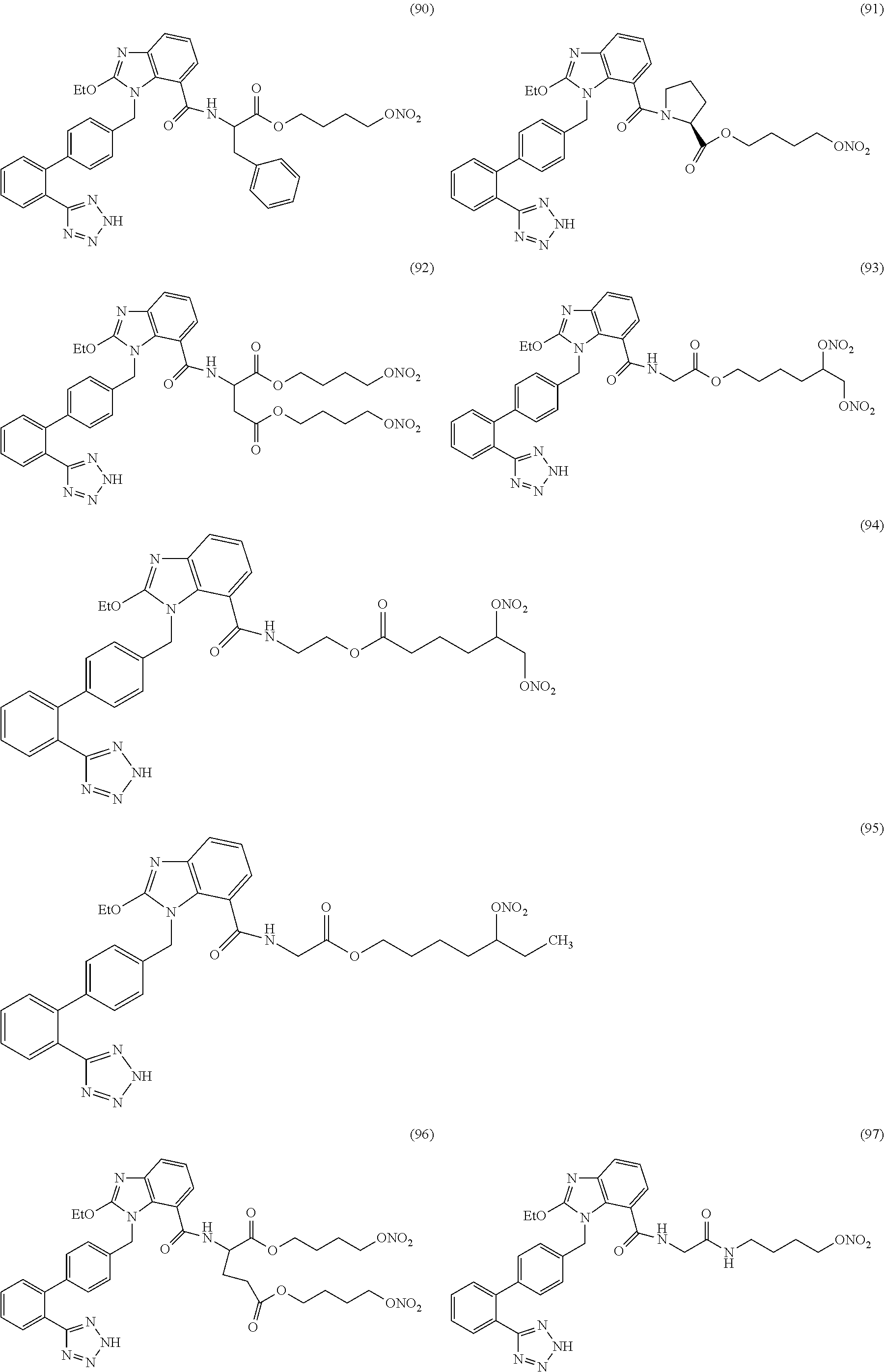 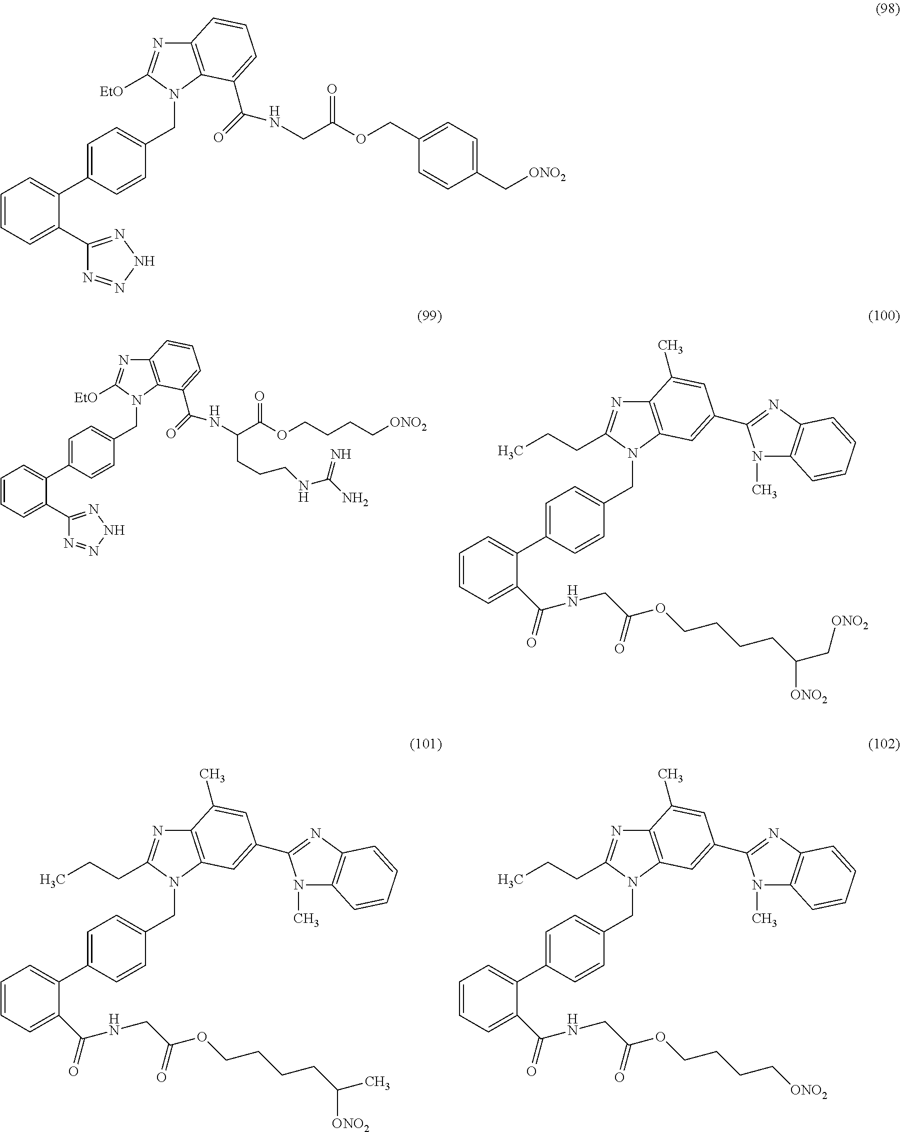 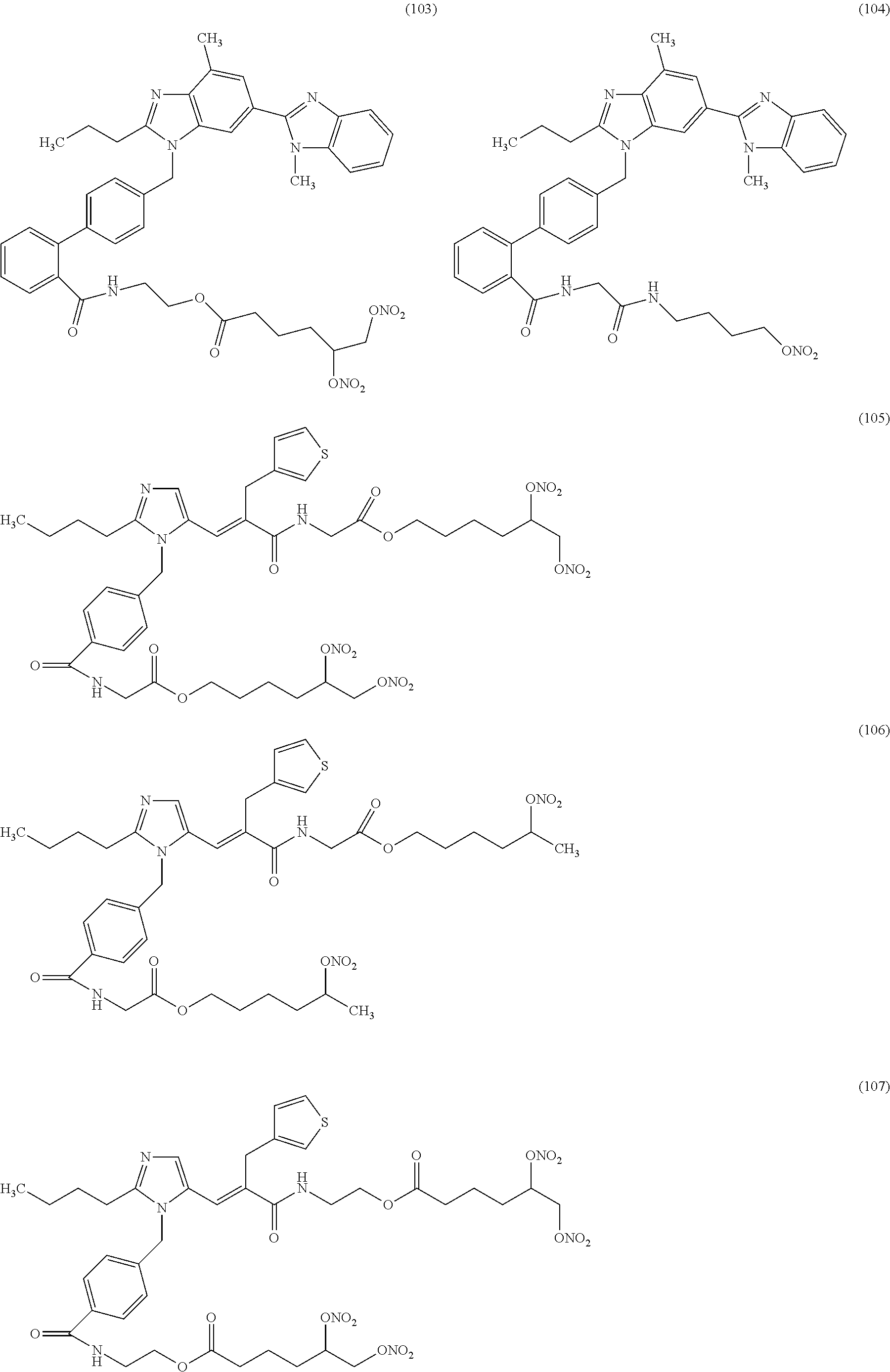 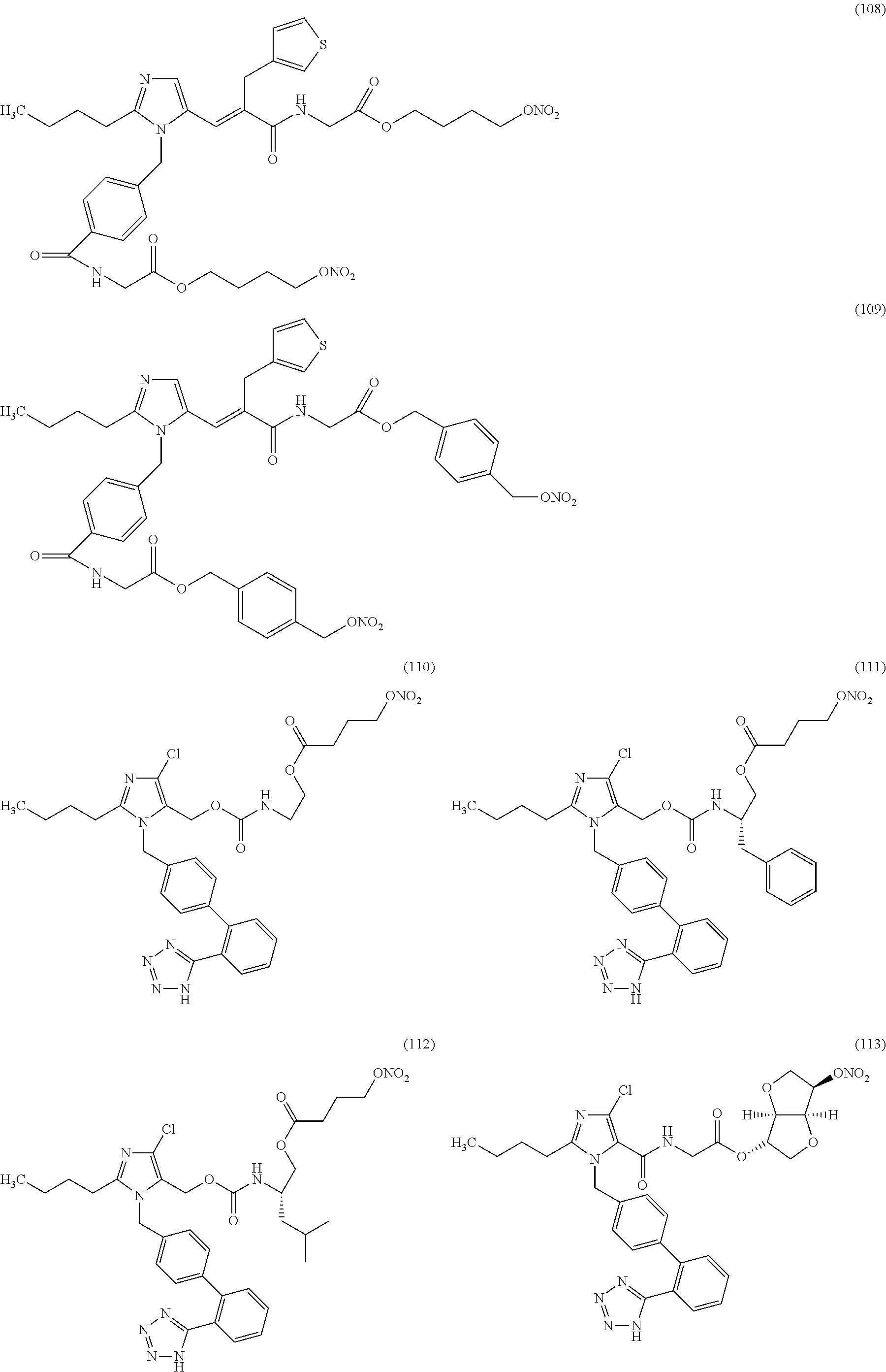 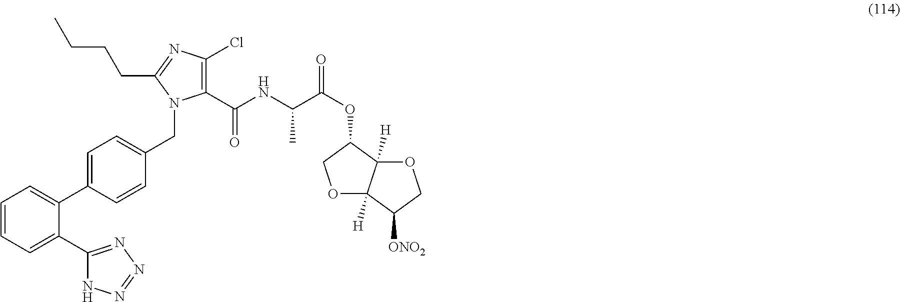
or a pharmaceutically acceptable salt or stereoisomer thereof.

10. A compound of formula R_{IIc}: 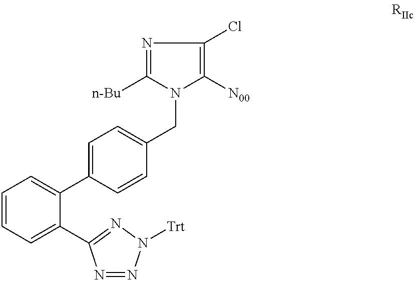
wherein Trt is the trityl protecting group, t-But is the t-Butyl protecting group and N₀₀ is —COOAct with Act=

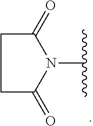

11. A process for preparing the compound R_{IIc} according to claim 10, by reacting a compound A with the commercially available compound B: 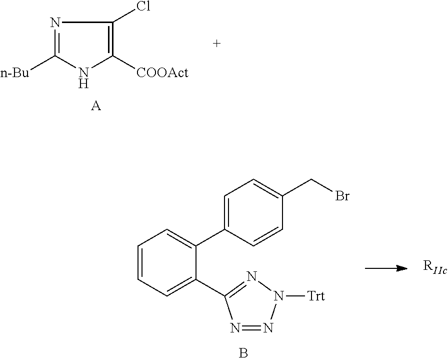
in presence of a base in an aprotic polar/non-polar solvent such as DMF, THE or CH₂Cl₂ at temperatures range between −15°-+80° C. or in a double phase system H₂O/Et₂O at temperatures range between 20°-40° C.

12. A compound of general formula (I) according to claim 1 for use as a medicament.

13. A compound according to claim 1 for use as a drug having anti-inflammatory, antithrombotic and antiplatelet activity.

14. A compound according to claim 1, for use in the treatment or prophylaxis of cardiovascular, renal and chronic liver diseases, inflammatory processes and metabolic syndrome.

15. A compound according to claim 1, for use in the treatment or prophylaxis of hypertension, congestive heart failure, pulmonary hypertension, renal insufficiency, renal ischemia, renal failure, renal fibrosis, liver fibrosis, portal hypertension, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, metabolic syndromes and other diseases known to be related to the renin-angiotensin system.

16. A pharmaceutical composition comprising a pharmaceutically acceptable carrier and a pharmaceutically effective amount of a compound of general formula (I) or a salt or stereoisomer thereof according to claim 1.

17. A pharmaceutical composition according to claim 16 in a suitable form for the oral, parenteral, rectal, topic and transdermic administration, by inhalation spray or aerosol or iontophoresis devices.

18. Liquid or solid pharmaceutical composition for oral, parenteral, rectal, topic and transdermic administration or inhalation in the form of tablets, capsules and pills eventually with enteric coating, powders, granules, gels, emulsions, solutions, suspensions, syrups, elixir, injectable forms, suppositories, in transdermal patches or liposomes, containing a compound of formula (I) or a salt or stereoisomer thereof according to claim 1 and a pharmaceutically acceptable carrier.

19. A pharmaceutical composition comprising a compound of general formula (I) according to claim 1, at least a compound used to treat cardiovascular disease and a pharmaceutically acceptable carrier.

20. Pharmaceutical composition according to claim 19 wherein the compound used to treat cardiovascular disease is selected from the group consisting of: aldosterone antagonists, renin inhibitors, ACE inhibitors, HMGCoA reductase inhibitors, beta-adrenergic blockers, alpha-adrenergic antagonists, sympatholytics, calcium channel blockers, endothelin antagonists, neutral endopeptidase inhibitors, potassium activators, diuretics, vasodilators, antithrombotics such as aspirin or nitrosated compounds thereof.

21. A pharmaceutical kit comprising a compound of general formula (I) as defined in claim 1, a compound used to treat cardiovascular disease as combined preparation for simultaneous, separated or sequential use for the treatment of cardiovascular disease.

22. A pharmaceutical kit according to claim 21 wherein the compound used to treat cardiovascular disease is selected from the group consisting of: aldosterone antagonists, renin inhibitors, ACE inhibitors, HMGCoA reductase inhibitors, beta-adrenergic blockers, alpha-adrenergic antagonists, sympatholytics, calcium channel blockers, endothelin antagonists, neutral endopeptidase inhibitors, potassium activators, diuretics, vasodilators, antithrombotics such as aspirin or nitrosated compounds thereof.
